(19) United States
(12) Patent Application Publication
(10) Pub. No.: US 2021/0275456 A1
(43) Pub. Date: Sep. 9, 2021

(54) AGGREGATED MICROPARTICLES

(71) Applicant: Graybug Vision, Inc., Redwood City, CA (US)

(72) Inventors: Ming Yang, Lutherville-Timonium, MD (US); Daniel Louis Salain, Marco Island, FL (US); Charles Pauling Semba, Palo Alto, CA (US); Yun Yu, Baltimore, MD (US); Weiling Yu, Perry Hall, MD (US); Joshua Kays, Baltimore, MD (US); Jane Chisholm, Baltimore, MD (US); Qingyun Lu, Nottingham, MD (US); Jeffrey L. Cleland, San Carlos, CA (US)

(73) Assignee: Graybug Vision, Inc., Redwood City, CA (US)

(21) Appl. No.: 17/319,971

(22) Filed: May 13, 2021

Related U.S. Application Data
(63) Continuation of application No. PCT/US2019/061859, filed on Nov. 15, 2019.
(60) Provisional application No. 62/803,273, filed on Feb. 8, 2019.
(60) Provisional application No. 62/783,936, filed on Dec. 21, 2018.
(60) Provisional application No. 62/767,911, filed on Nov. 15, 2018.

(51) Int. Cl. A61K 9/16; A61K 9/00; A61K 31/404; A61K 31/5377; A61K 47/54; A61K 47/60; A61K 47/36; A61K 47/14; A61K 9/08

(52) U.S. Cl. CPC A61K 9/1647; A61K 9/0048; A61K 31/404; A61K 31/5377; A61K 9/08; A61K 47/60; A61K 47/36; A61K 47/14; A61K 47/54

(57) ABSTRACT

Compositions and methods are provided that include aggregating microparticles which include an active agent that exhibit an increased hardness and/or durability of the resulting aggregated microparticles in vivo, which can lead to more stable, long-term ocular therapy.

### CROSS-REFERENCE TO RELATED APPLICATIONS

This application is a continuation of International Patent Application No. PCT/US2019/061859, filed in the U.S. Receiving Office on Nov. 15, 2019, which claims priority to provisional U.S. Application No. 62/767,911, filed Nov. 15, 2018; U.S. Application No. 62/783,936, filed Dec. 21, 2018; and U.S. Application 62/803,273, filed Feb. 8, 2019. The entirety of each these applications is hereby incorporated by reference herein for all purposes.

### FIELD OF THE INVENTION

This invention is in the area of improved methods and compositions that produce in vivo aggregated microparticles that can be loaded with an active drug or a prodrug of an active drug for ocular therapy.

### BACKGROUND

The structure of the eye can be divided into two segments referred to as the anterior and posterior. The anterior segment comprises the front third of the eye and includes the structures in front of the vitreous humor: the cornea, iris, ciliary body (including the pars plana), and lens. The posterior segment includes the back two-thirds of the eye and includes the sclera, choroid, retinal pigment epithelium, neural retina, optic nerve, and vitreous humor.

Important diseases affecting the anterior segment of the eye include glaucoma, allergic conjunctivitis, anterior uveitis, and cataracts. Diseases affecting the posterior segment of the eye include dry and wet age-related macular degeneration (AMD), cytomegalovirus (CMV) infection, diabetic retinopathy, choroidal neovascularization, acute macular neuroretinopathy, macular edema (such as cystoid macular edema and diabetic macular edema), Behcet's disease, retinal disorders, diabetic retinopathy (including proliferative diabetic retinopathy), retinal arterial occlusive disease, central retinal vein occlusion, uveitis retinal disease, retinal detachment, ocular trauma, damage caused by ocular laser treatment or photodynamic therapy, photocoagulation, radiation retinopathy, epiretinal membrane disorders, branch retinal vein occlusion, anterior ischemic optic neuropathy, non-retinopathy diabetic retinal dysfunction and retinitis pigmentosa. Glaucoma is sometimes also considered a posterior ocular condition because a therapeutic goal of glaucoma treatment is to prevent or reduce the loss of vision due to damage or loss of retinal cells or optic nerve cells.

Typical routes of drug administration to the eye include topical, systemic, intravitreal, intraocular, intracameral, subconjunctival, sub-tenon, retrobulbar, and posterior juxtascleral. (Gaudana, R., et al., “Ocular Drug Delivery”, The American Association of Pharmaceutical Scientist Journal, 12(3)348-360, 2010).

A number of types of delivery systems have been developed to deliver therapeutic agents to the eye. Such delivery systems include conventional (solution, suspension, emulsion, ointment, inserts, and gels), vesicular (liposomes, niosomes, discomes, and pharmacosomes), advanced materials (scleral plugs, gene delivery, siRNA, and stem cells), and controlled-release systems (implants, hydrogels, dendrimers, iontophoresis, collagen shields, polymeric solutions, therapeutic contact lenses, cyclodextrin carriers, microneedles, microemulsions, and particulates (microparticles and nanoparticles)).

Treatment of posterior segment diseases remains a daunting challenge for formulation scientists. Drug delivery to the posterior segment of the eye is typically achieved via an intravitreal injection, the periocular route, implant, or by systemic administration. Drug delivery to the posterior segment by way of the periocular route can involve the application of a drug solution to the close proximity of the sclera, resulting in high retinal and vitreal concentrations.

Intravitreal injection is often carried out with a 30 gauge or less needle. While intravitreal injections offer high concentrations of drug to the vitreous chamber and retina, they can be associated with various short-term complications such as retinal detachment, endophthalmitis, and intravitreal hemorrhages. Experience shows that injection of small particles can lead to the rapid dispersal of the particles that can obstruct vision (experienced by the patient as “floaties” or “floaters”) and the rapid removal of the particles from the injection site (which can occur via the lymphatic drainage system or by phagocytosis). In addition, immunogenicity can occur upon recognition of the microspheres by macrophages and other cells and mediators of the immune system.

Complications in periocular injections include increased intraocular pressure, cataract, lur, strabismus, and corneal decompensation. Transscleral delivery with periocular administration is seen as an alternative to intravitreal injections. However, ocular barriers such as the sclera, choroid, retinal pigment epithelium, lymphatic flow, and general blood flow can compromise efficacy. Systemic administration, which is not advantageous given the ratio of the volume of the eye to the entire body, can lead to potential systemic toxicity.

Johns Hopkins University has filed a number of patents claiming formulations for ocular injections including WO2013/138343 titled “Controlled Release Formulations for the Delivery of HIF-1 Inhibitors”, WO2013/138346 titled “Non-linear Multiblock Copolymer-drug Conjugates for the Delivery of Active Agents”, WO2011/106702 titled “Sustained Delivery of Therapeutic Agents to an Eye Compartment”, WO2016/025215 titled “Glucorticoid-loaded Nanoparticles for Prevention of Corneal Allograft Rejection and Neovascularization”, WO2016/100392 titled “Sunitinib Formulations and Methods for Use Thereof in Treatment of Ocular Disorders”, WO2016/100380 titled “Sunitinib Formulation and Methods for Use Thereof in Treatment of Glaucoma”, WO2016/118506 titled “Compositions for the Sustained Release of Anti-Glaucoma Agents to Control IntraocularPressure”, WO2013/166385 titled “Nanocrystals, Compositions, and Methods that Aid Particle Transport in Mucus”, WO2005/072710 titled “Drug and Gene Carrier Particles that Rapidly move Through Mucus Barriers,” WO2008/030557 titled “Compositions and Methods for Enhancing Transport through Mucus”, WO2012/061703 titled “Compositions and Methods Relating to Reduced Mucoadhesion,” WO2012/039979 titled “Large Nanoparticles that Penetrate Tissue,” WO2012/109363 titled “Mucus Penetrating Gene Carriers”, WO2013/090804 titled “Biodegradable Stealth Nanoparticles Prepared by a Novel Self-Assembly Emulsification Method,” WO2013/110028 titled “Nanoparticles Formulations with Enhanced Mucosal Penetration”, and WO2013/166498 titled “Lipid-based Drug Carriers for Rapid Penetration through Mucus Linings”.

GrayBug Vision, Inc. discloses prodrugs for the treatment of ocular therapy in granted U.S. Pat. Nos. 9,808,531; 9,956,302; 10,098,965; 10,117,950; 10,111,964; 10,159,747; and, 10,458,876 and in PCT Applications WO2017/053638; WO2018/175922; WO 2019/118924; and WO 2019/210215. Graybug Vision, Inc. has also invented new technology to create microparticles that aggregate in vivo to at least one pellet of at least 500 μm. Aggregating microparticles for ocular therapy are described in U.S. Pat. No. 10,441,548 and U.S. Application No. US 2018-0326078. PCT Applications WO2017/083779; WO2018/209155; and, WO 2019/209883 describe aggregating microparticles and processes for making aggregating microparticles.

To treat ocular diseases, and in particular diseases of the posterior segment, the drug must be delivered in therapeutic levels and for a sufficient duration to achieve efficacy. This seemingly straightforward goal is difficult to achieve in practice.

The object of this invention is improved compositions and methods to produce an in vivo aggregated microparticle for controlled drug delivery of an active agent to the eye, and the use of such particles for therapeutic purposes.

### SUMMARY

Compositions and methods are provided that include aggregating microparticles which include an active agent, that exhibit an increased hardness and/or durability of the resulting aggregated microparticles in vivo, which can lead to more stable, long-term ocular therapy.

It has been discovered that it requires the coordinated control of a combination of a number of factors when used together to achieve this significantly harder and/or durable aggregated microparticle in vivo. The factors include, for example, (i) improved methods for surface treatment (e.g., by careful selection of the amount of base and alcohol or other solvent, as taught herein); (ii) removing microparticles of less than approximately 5, 10 or 15 um from the aggregating microparticles, for example by centrifugation; (iii) providing the surface treated microparticles in a diluent for injection that includes an agent or additive that softens the surface of the microparticle before administration to prepare it for aggregation, also as taught herein; and/or (iv) providing an advantageous method of administration into the vitreous of the patient that maximizes the ability of the aggregating microparticles to efficiently aggregate in vivo to a particle of at least 500 um and a hardness of at least 5 g/force needed to compress the particle at 30% of strain (as confirmed by in vitro analyses as described using an assay below).

Smaller microparticles may be accomplished with one or more, including serial centrifugation steps, for example, 2, 3, 4, or 5 centrifugation steps in serial or continuous fashion. Surface treatment conditions that utilize about 1.0 or 1.5 mM and less than 10 mM NaOH in a solvent of between about 55% and 75% ethanol in water and the use of a diluent that includes additive that upon injection in vivo assists in the aggregation, for example benzyl alcohol or triethyl citrate.

The improved methods of administration are advantageous to typical intravitreal methods of administration because the microparticles are administered in such a way that the microparticles are injected toward the bottom of the vitreous, which minimizing sliding and tailing as the patient reorients his or her eye and the microparticles settle at the bottom of the vitreous. This allows for enhanced aggregation in vivo. This combination of factors achieves an improved aggregated microparticle of at least 500 microns.

In one embodiment, the improved aggregated microparticle of at least 500 microns exhibits a hardness rating in vivo in the vitreous of the eye, for example a human eye, of at least 5, and in some embodiments, at least about 10, 15, 20 or 25 gram-force needed to compress the particle at 30% of strain. In one embodiment, the hardness of the improved aggregated microparticle, upon injection in the vitreous, increases at least two-fold, at least three-fold, at least four-fold, at least five-fold, or more in four hours or less following injection compared to microparticles administered immediately after injection (for example, less than one minute or even 30 seconds after administration). In one embodiment, the hardness increases in three hours or less, in two hours or less, in one hour or less, in thirty minutes or less, in fifteen minutes or less, in ten minutes or less, in five minutes or less, in two minutes or less, or in one minute or less.

The hardness of the aggregated microparticle can be confirmed in vitro in vitreous fluid, in phosphate buffered saline, or in water or other physiologically acceptable aqueous solution, including an aqueous solution that includes one or more components of the vitreous, which are well-known. The vitreous humor fluid in vivo typically contains 98-99% water, salts, sugars, vitrosin, fibrils with glycosaminoglycan, hyaluronan (i.e., hyaluronic acid), opticin, and various proteins. The vitreous humor typically has a viscosity of approximately 2-4 times that of water. In one embodiment, the hardness is tested in a hyaluronic acid-based solution with a viscosity that in one embodiment approximately mimicks that of the vitreous. In one embodiment, the hardness is measured in a fluid selected from vitreous, water, phosphate buffered saline, or an aqueous physiologically acceptable solution with a viscosity not more than about 4 times that of water.

Since ocular disorders increase with age, it is important to provide a particle suspension that achieves aggregation to a pellet of at least 500 microns in lower viscosity vitreous fluid. This invention improves the ability to produce hard and/or durable aggregated microparticles in eyes with lower viscosity vitreous fluid, and thus is especially useful for older patients with ocular disorders.

### Optimized Microparticle Preparation

In one embodiment, an optimized microparticle preparation and composition is provided that includes, in the course of preparing the microparticles that will be aggregated in vivo, substantially removing smaller microparticles less than about 5 or 10 microns from the microparticle suspension or solution after preparation of the microparticles, for example, by centrifugation or other small particle-removing means such that the smaller microparticles are substantially eliminated. In certain embodiments, the removal of the smaller microparticles may optionally be accomplished with one or more, including serial centrifugation steps, for example, 2, 3, 4, or 5 centrifugation steps in serial or continuous fashion. In general, the centrifugation is carried out under any conditions that achieve the desired results, and are often dependent on batch size. For certain batch sizes, for example, the centrifugation may be carried out at about 1000-3000 rpm, and more typically between 1500-2500 rpm. The time of centrifugation is also a function of batch size. The larger the batch, the longer the process of centrifugation is needed. In some cases, the one or multiple centrifugation steps are conducted until the supernatant achieves the desired level of clarity, and in some cases, if the drug has color, the desired level of colorlessness. This can be measured by light transmittance, as further described below.

Another factor that contributes to the ability to create an aggregated microparticle in vivo in the vitreous of the eye is the surface treatment of the microparticle during individual microparticle formation, which assists the aggregation process. It has been found that individual microparticles treated with greater than about 1.0 or 1.5 mM and less than 10 mM NaOH in a solvent of between about 55% and 75% ethanol in water, and more typically between about 60% and 75% ethanol in water, or even about 70% ethanol, for more than 20 minutes, produce improved hardened aggregated microparticles in vivo in the vitreous. In certain embodiments, the surface treatment is carried out for a time of greater than about 60, 90 or 120 minutes. The surface treatment is carried out at less than 18° C., and often less than 15, 12, 10 or 5° C.

In another embodiment, the suspension of mildly surface-treated solid biodegradable microparticles is placed in a diluent comprising an additive that upon injection in vivo assists in the aggregation to form a larger particle or pellet. It has been discovered that the addition of an additive that softens the surface of the microparticle can be included in the microparticle suspension in the diluent prior to injection. For example, an additive that increases the plasticity, decreases the viscosity or glass transition temperature of the surface polymer or partially dissolves the surface polymer can be useful to assist in the in vivo aggregation process. Non-limiting examples include benzyl alcohol and triethyl citrate. The additive added to the diluent of a suspension of mildly surface-treated microparticles results in improved in vivo particle aggregation of the surface-treated microparticles. In one embodiment, the microparticles suspended in diluent comprising an additive aggregate faster and stronger in vivo compared to microparticles suspended in diluent without such additive. Example 14 discusses the effect of benzyl alcohol in the diluent on particle aggregation of mildly-surface treated microparticles and Example 15 discusses the effect of triethyl citrate in the diluent on particle aggregation. As discussed in these Examples and shown in FIG. 16, FIG. 17, FIG. 18, FIG. 19A, FIG. 19B, FIG. 20, FIG. 21A, and FIG. 21B, the inclusion of these additives in the suspension of surface-treated microparticles results in better and faster aggregation.

In one embodiment, the invention is thus a suspension of solid aggregating microparticles comprising surface surfactant, at least one biodegradable polymer, and a therapeutic agent in a diluent comprising an additive that improves in vivo particle aggregation wherein the solid aggregating microparticles:
(i) have a solid core with less than 10% porosity by ratio of void space to total volume;
(ii) contain from about 0.001 percent to about 1 percent surfactant and have been surface-modified to contain less surfactant than a microparticle prior to the surface modification wherein the surface has been modified at a temperature less than about 18° C.;
(iii) have a mean diameter between 10 um and 60 um;
(iv) are capable of aggregating in vivo to form at least one pellet of at least 500 m in vivo capable of sustained drug delivery in vivo for at least three months; and
(v) optionally wherein the suspension has been treated with vacuum at a pressure of less than 40 Torr, less than 30 Torr, less than 25 Torr, less than 20 Torr, less than 10 Torr, or less than 5 Torr for between 1 and 90 minutes.

In an alternative embodiment, the invention is a suspension of aggregating microparticles comprising surface surfactant, at least one biodegradable polymer, and a therapeutic agent in a diluent comprising an additive that improves in vivo particle aggregation wherein the solid aggregating microparticles:
(i) contain from about 0.001 percent to about 1 percent surfactant and have been surface-modified to contain less surfactant than a microparticle prior to the surface modification wherein the surface has been modified at a temperature less than about 18° C.;
(ii) have a mean diameter between 10 um and 60 um;
(iii) are capable of aggregating in vivo to form at least one pellet of at least 500 m in vivo capable of sustained drug delivery in vivo for at least three months; and
(iv) optionally wherein the suspension has been treated with vacuum at a pressure of less than 40 Torr, less than 30 Torr, less than 25 Torr, less than 20 Torr, less than 10 Torr, or less than 5 Torr for between 1 and 90 minutes.

In one embodiment, the improved aggregating microparticle suspension or solution comprises microparticles with a mean diameter between 20 and 40 microns that produce an improved aggregated microparticle of at least 500 microns that exhibit a hardness rating in vivo in the vitreous of the eye of at least 5 gram-force, 10 gram-force, 15 gram-force, or 20-gram force needed to compress the particle at 30% of strain. In an alternative embodiment, the hardness of the improved aggregated microparticle, upon injection in the vitreous, increases at least two-fold, at least three-fold, at least four-fold, at least five-fold, at least six-fold, at least seven-fold, at least eight-fold, at least nine-fold, at least ten-fold or more in four hours or less, in three hours or less, in two hours or less, in one hour or less, in thirty minutes or less, in fifteen minutes or less, in ten minutes or less, in five minutes or less, in two minutes or less, or in one minute or less following injection compared to microparticles administered immediately after injection (for example, less than one minute or even 30 seconds after administration).

In one embodiment, the improved aggregating microparticle comprises PLGA-PEG. In one embodiment, the improved aggregating microparticle comprises PLGA. In one embodiment, the improved aggregating microparticle comprises PLGA and PLGA-PEG. In one embodiment, the improved aggregating microparticle comprises PLGA, PLA, and PLGA-PEG.

In one embodiment, the improved aggregating microparticle is biodegradable.

In one embodiment, the microparticle comprises an active agent. In one embodiment, the active agent is a tyrosine kinase inhibitor. In one embodiment, the active agent is sunitinib or a pharmaceutically acceptable salt thereof. In one embodiment, the active agent is sunitinib malate. In one embodiment, the active agent is selected from furosemide, bumetanide, piretanide, ethacrynic acid, etozolin, and ozolinone or a pharmaceutically acceptable salt thereof. In one embodiment, the active agent selected from timolol, brimonidine, brinzolamide, dorzolamide, or pharmaceutically acceptable salt thereof. In one embodiment, the active agent is a prodrug of sunitinib, timolol, brimonidine, brinzolamide, dorzolamide, or pharmaceutically acceptable salt thereof.

### Optimized Method of Administration

The coordinated control of the combination of factors resulting in the significantly harder and/or durable aggregated microparticle upon injection in vivo includes improved methods of administration.

In one method of administration, the optimized solution or suspension of aggregating microparticles is loaded into a means for injection comprising a needle with a length of less than 7 mm and injected into the eye of a patient looking up at least 15°, and typically at least 20°, such that around 3-5 mm of the needle is in the vitreous. The needle is injected through the pars plana of the eye between about 3 and 6 mm, sometimes about 4 mm, from the limbus at an angle that deposits the solution or suspension at or near the bottom of the vitreous chamber (FIG. 1 is an illustration of the eye with the plans plana, the limbus, and vitreous chamber labeled). The method is illustrated in FIG. 10A, FIG. 10B, FIG. 10C, and FIG. 10D.

In an alternative method of administration, the optimized solution or suspension of aggregating microparticles is loaded into a means for injection comprising a needle with a length of between about 13-18 mm and injected into the eye of a patient looking up between about 200 to about 30°, typically about 25°, such that around 10-15 mm of the needle is in the vitreous. The needle is injected through the pars plana of the eye between about 3 and 6 mm, sometimes about 4 mm, from the limbus at an angle that deposits the solution or suspension at or near the bottom of the vitreous chamber. The method is illustrated in FIG. 9A, FIG. 9B, and FIG. 9C.

These improved methods of administration assist with particle aggregation and minimize sliding, spreading, and dispersion of the individual microparticles prior to aggregation (Example 11 and Example 12, FIGS. 12C-12F and FIGS. 13A-13B). In certain embodiments, the means for injection is a syringe and the needle is approximately 31, 30, 29, 28, 27, 26 or 25 gauge with either normal or thin wall.

It has been discovered that this aggregation can be improved when the microparticles are injected via the methods described herein and/or suspended in a diluent comprising an additive.

In certain embodiments, a method is provided for increasing the hardness and/or durability of an in vivo aggregated microparticle for controlled delivery of an active agent to the eye of a patient for therapeutic purposes.

In one embodiment, the hardness increases, upon injection in the vitreous, relative to the microparticle immediately after injection (for example, less than one minute or even 30 seconds after administration). In one embodiment, upon injection in the vitreous, the hardness of the microparticle increases at least two-fold in about two hours or less following injection relative to microparticles administered immediately after injection (for example, less than one minute or even 30 seconds after administration). One non-limiting method for administering a microparticle that aggregates into a microparticle of at least about 500 microns of advantageous hardness and/or durability in vivo includes:
(a) Providing a solution or suspension of optimized aggregating microparticles as described herein;
(b) Loading a means for injection that comprises a needle of less than about 7 mm with the selected amount of the solution or suspension of the aggregating microparticles;
(c) Positioning the patient in an approximate sitting position looking up with at least an approximate 15 degree angle;
(d) Injecting the solution or suspension of aggregating microparticles:
   i. through the pars plana of the eye between 3 and 6 mm posterior to the limbus of the eye;
   ii. wherein the point of needle entry is between about 5:30 o'clock and 9 o'clock with respect to the pupil of the eye looking straight, and typically between about 6 and 8 o'clock;
   iii. at an angle that deposits the solution or suspension at or near the bottom of the vitreous chamber and in a manner that not more than about 4 mm of the needle is in the vitreous; and
(e) After a short movement period to change chairs if necessary, maintaining the patient in a sitting position for a sufficient time to allow the aggregating microparticles to aggregate to at least one aggregated microparticle of at least 500 microns.

An additional non-limiting method for administering a microparticle that aggregates into a microparticle of at least about 500 microns of advantageous hardness and/or durability in vivo includes:
(a) Providing a solution or suspension of optimized aggregating microparticles as described herein;
(b) Loading a means for injection that comprises a needle of between about 10 mm and about 18 mm with the selected amount of the solution or suspension of the aggregating microparticles;
(c) Positioning the patient in an approximate sitting position looking up with at least an approximate 15 degree angle;
(d) Injecting the solution or suspension of aggregating microparticles:
   i. through the pars plana of the eye between 3 and 6 mm posterior to the limbus of the eye;
   ii. wherein the point of needle entry is between about 4:00 o'clock and 8 o'clock with respect to the pupil of the eye looking straight, and typically about 6 o'clock;
   iii. at an angle that deposits the solution or suspension at or near the bottom of the vitreous chamber; and
(e) After a short movement period to change chairs if necessary, maintaining the patient in a sitting position for a sufficient time to allow the aggregating microparticles to aggregate to at least one aggregated microparticle of at least 500 microns.

In one embodiment, the aggregated exhibit a hardness rating in vivo in the vitreous of the eye of at least 5 gram-force, 10 gram-force, 15 gram-force, or 20-gram force needed to compress the particle at 30% of strain.

In an alternative embodiment, the hardness of the aggregated microparticles, upon injection in the vitreous, increase at least two-fold, at least three-fold, at least four-fold, at least five-fold, at least six-fold, at least seven-fold, at least eight-fold, at least nine-fold, at least ten-fold or more in four hours or less, in three hours or less, in two hours or less, in one hour or less, in thirty minutes or less, in fifteen minutes or less, in ten minutes or less, in five minutes or less, in two minutes or less, or in one minute or less following injection compared to microparticles administered immediately after injection (for example, less than one minute or even 30 seconds after administration).

In one embodiment, the optimized aggregating microparticles in step (a) comprise at least one biodegradable polymer and a therapeutic agent that is encapsulated in the biodegradable polymer wherein the microparticles have a mean diameter between 10 μm and 60 μm that:
(i) have a solid core with less than 10% porosity by ratio of void space to total 10 volume;
(ii) contain from about 0.001 percent to about 1 percent surfactant and have been surface-modified to contain less surfactant than a microparticle prior to the surface modification wherein the surface has been modified at a temperature less than about 18° C.; and
(iii) are capable of aggregating in vivo to form at least one pellet of at least 500 m 15 in vivo capable of sustained drug delivery in vivo for at least one month.

In an alternative embodiment, the optimized aggregating microparticles of step (a) comprise at least one biodegradable polymer and a therapeutic agent that is encapsulated in the biodegradable polymer wherein the microparticles have a mean diameter between 10 μm and 60 μm that:
(ii) contain from about 0.001 percent to about 1 percent surfactant and have been surface-modified to contain less surfactant than a microparticle prior to the surface modification wherein the surface has been modified at a temperature less than about 18° C.; and
(iii) are capable of aggregating in vivo to form at least one pellet of at least 500 m in vivo capable of sustained drug delivery in vivo for at least one month.

In one embodiment, the active agent is sunitinib or a pharmaceutically acceptable salt thereof. In one embodiment, the active agent is sunitinib malate. In one embodiment, the active agent is a prodrug as described herein, for example a prodrug described in Tables A-K.

In one embodiment, the solution or suspension of aggregating microparticles has been treated with vacuum at a pressure of less than 40 Torr, less than 30 Torr, less than 25 Torr, less than 20 Torr, less than 10 Torr, or less than 5 Torr for between 1 and 90 minutes.

Thus, according the present invention, compositions and methods for increasing hardness and/or durability of an in vivo aggregated microparticle for controlled drug delivery to the eye of a patient in need thereof are provided.

The method is the injection of a solution or suspension of an aggregating microparticle wherein the solution or suspension is loaded into a means for injection that comprises a needle of less than about 7 mm and injected into the eye of a patient looking upward. The solution or suspension is injected through the pars plana between 3 mm and 6 mm posterior to the limbus at an angle that deposits the solution or suspension at or near the vitreous chamber. In an alternative method, the needle is between about 13 mm and 18 mm and the solution or suspension is injected through the pars plana between 10 mm and 15 mm posterior to the limbus at an angle that deposits the solution or suspension at or near the vitreous chamber.

As discussed in Example 11, previous methods of administration taught an administration method wherein the patient tilted his/her head back or lay down and this deposited the microparticles toward the back of the eye and not at the vitreous chamber. The microparticles were then required to “slide” toward the bottom the vitreous chamber, which created spreading and tailing of the aggregating microparticle as it moved from the back of the eye to the vitreous chamber.

In one embodiment of the invention, the suspension of microparticles for the method of administration described herein or the microparticles in a diluent comprising additive are vacuum treated as described in US 2018-0326078, following reconstitution in an appropriate diluent comprising additive. In one embodiment of the invention, the suspension of microparticles for the method of administration described herein or the microparticles in a diluent comprising additive are sonicated as described in US 2018-0326078, following reconstitution in an appropriate diluent comprising additive.

In one aspect of the invention, the process for preparing a microparticle suspension leading to an aggregated pellet in vivo can be used in combination with a selected method for forming aggregating microparticles described in US 2017-0135960, PCT/US16/61706, US 2018-0326078, and PCT/US18/32167 (and the resulting materials thereof).

In one aspect of the invention, the process for preparing a microparticle can be used in combination with continuous centrifugation for the separation of small particles in addition to washing and concentrating particles. In one embodiment, the microparticles are subjected to one, two, or three rounds of centrifugation. In one embodiment, the continuous centrifugation removes particles of less than approximately 2, 5, 10, or 15 μm.

As an illustration, the present invention further includes a process for the preparation of surface-modified solid aggregating microparticle suspensions that provide microparticles that aggregate in vivo to form pellets described herein that include:
A. a first step of preparing microparticles comprising one or more biodegradable polymers by dissolving or dispersing the polymer(s) and a therapeutic agent in one or more solvents to form a polymer and therapeutic agent solution or dispersion, mixing the polymer and the therapeutic agent solution or dispersion with an aqueous phase containing a surfactant to produce solvent-laden microparticles and then removing the solvent(s) to produce polymer microparticles that contain the therapeutic agent, polymer and surfactant; and
B. a second step of mildly treating the surface of microparticles of step (i) at a temperature at or below about 18, 15, 10, 8 or 5° C. optionally up to about 1, 2, 3, 4, 5, 10, 30, 40, 50, 60, 70, 80, 90 100, 11, 120 or 140 minutes (wherein each alternative is considered individually as described as if separately written out) with an agent that removes or partially removes surface surfactant, surface polymer, or surface oligomer in a manner that does not significantly produce internal pores;
C. washing the microparticles with a solution comprising an excipient, optionally mannitol;
D. isolating and lyophilizing the surface-treated microparticles;
E. resuspending the surface-treated microparticles in an appropriate diluent that comprises additive that improves in vivo particle aggregation; F. optionally further improving the aggregation potential of the particles by subjecting the particles to at least one process selected from 1) vacuum treatment and 2) sonication.

In one embodiment, step B) partially removes surfaces surfactant, surface polymer, or surface oligomer resulting in a microparticle that contains less surfactant than a microparticle prior to surface-treatment. The process of these steps can be achieved in a continuous manufacturing line or via one step or in step-wise fashion as appropriate. The process of step F) above can be carried out following isolation of the microparticles and/or upon reconstitution prior to injection. In one embodiment, the surface treated solid biodegradable microparticles do not significantly aggregate during the manufacturing process. In another embodiment, the surface treated solid biodegradable microparticles do not significantly aggregate when resuspended and loaded into a syringe.

In one embodiment, step B) is followed by continuous centrifugation to remove particles of approximately less than 10 μm.

### BRIEF DESCRIPTION OF THE FIGURES

FIG. 1 is a labeled image of the anatomy of the eye. The pars plana, the limbus, and the vitreous chamber have been labeled in addition to the other parts of the eye. The pars plana is about 4 mm long and is located near the junction of the iris and the sclera. It is scalloped and adjacent to the vitreous chamber. In the method of administration of the present invention, the needle is injected through the pars plana. The limbus is the border where the cornea and the sclera meet. In the methods of administration of the present invention, the needle is injected 3 to 6 mm posterior to the limbus at the 6 o'clock position as shown in FIG. 9G. The vitreous chamber is the space occupied by the vitreous humor. It includes the space between the behind the lens and before the optic nerve. The methods of administration of the present invention deposit the microparticles at the bottom of the vitreous chamber as shown in FIG. 9C and FIG. 10D. The pupil is the hole located in the center of the iris. In the present invention, the pupil is the point of reference for the needle entry as shown in FIG. 9G.

FIG. 2A is an illustration of the reconstitution of the microparticles prior to injection. Step 1 is the injection of the diluent via a syringe into a vial of microparticles. The second step is the vacuum treatment of the microparticles suspended or dissolved in diluent. This is accomplished by attaching a vacuum syringe to the vial via an adapter and applying vacuum pressure. The vacuum pressure is followed by vortexing for approximately 3 seconds. The third step is loading the microparticle suspension or solution into a syringe for injection.

FIG. 2B is an illustration comparing the method of administration of the present invention (Method C) with the prior method of administration (Method A). In the method of administration of the present invention, the dispersion is minimized due in part to the shorter needle of 3-6 mm. In the previous method of administration (Method A) the angle was 13 mm and as shown, the particle dispersion was significant.

FIG. 2C is a graph comparing the hardness of microparticles from Lot AA and Lot H as described in Example 5. Lot H was 700% more hard than Lot AA at the 2-hour incubation period. The x-axis is labeled with the lot. The y-axis is the force at 30% of strain measured in gram-force (g). The left bar for each lot is the hardness after 15 minutes of incubation and the right bar for each lot is the hardness after 2 hours of incubation

FIG. 2D is a graph comparing the drug release of microparticles from Lot AA and Lot H as described in Example 5. The drug release was similar for the two lots. The drug release kinetics are similar for the two lots. The x-axis is time measured in days and the y-axis is drug release measured in percent.

FIG. 3A is a graph illustrating the hardness of the aggregated microparticles prepared by the method as described in Example 5 following incubation for 15 minutes or 2 hours (the detail for each lot is given in Table 1). The x-axis is labeled with the concentration of NaOH (mM), the percentage of EtOH (%), and the lot. The y-axis is the force at 30% of strain measured in gram-force (gf). The left bar for each lot is the hardness after 15 minutes of incubation and the right bar for each lot is the hardness after 2 hours of incubation. Each of the lots were subjected to surface-treatment at a temperature of approximately 12° C.

FIG. 3B is a graph comparing the hardness of microparticles from Lot AA and Lot H as described in Example 5 as the microparticles were incubated short-term (a maximum of 24 hours). The x-axis is labeled with incubation time measured in hours. The y-axis is the force at 30% of strain measured in gram-force (gf).

FIG. 3C is a graph comparing the hardness of microparticles from Lot AA and Lot H as described in Example 5 as the microparticles were incubated long-term (a maximum of 4 weeks). The x-axis is labeled with incubation time measured in weeks. The y-axis is the force at 30% of strain measured in gram-force (gf).

FIG. 4A is a diagram illustrating the impact of continuous centrifugation as described in Example 7. After each centrifugation, the volume of microparticles with diameters less than 10 m decreases. Before any centrifugation, particles less than 10 μm comprised 8.6% of the total size distribution, but after four rounds of centrifugation, a 68% reduction in the percent of particles smaller than 10 μm was observed. The x-axis is particle diameter measured in m and the y-axis is the differential volume of microparticles of different sizes measured in percent.

FIG. 4B is a diagram illustrating the impact of continuous centrifugation on the supernatant of the microparticle suspension as described in Example 7. After each round of centrifugation, the percentage of particles smaller than 10 μm was observed. The x-axis is particle diameter measured in m and the y-axis is the differential volume of microparticles of different sizes measured in percent.

FIG. 4C is a diagram illustrating the impact of continuous centrifugation as described in Example 7. After continuous centrifugation, the volume of microparticles with diameters less than m decreases. The amount of small particles less than 10 μm in the final product was 69% lower than that prior to centrifugation. The x-axis is particle diameter measured in m and the y-axis is the differential volume of microparticles of different sizes measured in percent.

FIG. 5A is a cuvette of a suspension of microparticles from Sample 1 (dose of 2 mg) in sodium hyaluronate solution as described in Example 8. The microparticles exhibited a light transmittance of 85.9%.

FIG. 5B is a cuvette of a suspension of microparticles from Sample 2 (dose of 2 mg) in sodium hyaluronate solution as described in Example 8. The microparticles exhibited a light transmittance of 99.6%.

FIG. 5C is the drug release of microparticles from Sample 1, Sample 2, and Sample 3 as described in Example 8. All three samples exhibited comparable drug release. The x-axis is time measured in days and the y-axis is drug release measured in percent.

FIG. 6A is an image of a glass eye containing liquefied vitreous that was injected with Lot AA microparticles as described in Example 9. The image was taken post-injection and spreading of the aggregated microparticle was observed.

FIG. 6B is an image of a glass eye containing liquefied vitreous that was injected with Lot AA microparticles as described in Example 9. The image was taken post-movement and dispersion of the aggregated microparticle was observed.

FIG. 6C is an image of a glass eye containing liquefied vitreous that was injected with Lot E microparticles as described in Example 9. The image was taken post-injection and spreading of the microparticles was not observed.

FIG. 6D is an image of a glass eye containing liquefied vitreous that was injected with Lot E microparticles as described in Example 9. The image was taken post-movement and dispersion of the aggregated microparticle was not observed.

FIG. 7A is an image taken of the in-situ porcine vitreous liquefaction model as described in Example 10. The eye was injected with Lot AA microparticles. The microparticles were deposited within the inferior vitreous cavity (left), but readily broke to pieces and could not be readily picked up with forceps (right).

FIG. 7B is an image taken of the in-situ porcine vitreous liquefaction model as described in Example 10. The eye was injected with Lot E microparticles. The microparticles were one solid piece (left) that could be picked up with forceps (right).

FIG. 8A is an image depicting the injection site, tilt of the eye, and needle size of Method A as described in Example 11.

FIG. 8B is scheme depicting the steps for injection of Method A as described in Example 11. The first step of the method of administration is wherein the patient has tilted his head back and looked up. In the second step, the patient sits up and reorients his eyes to the vertical position. At this point, the microparticles move from the back of the eye (point A) to the bottom vitreous chamber (point B). The final depot location is shown in the last image. In this image the microparticle has spread as it moved from point A to point B.

FIG. 8C is an image of the eye during Method A as described in Example 11. A is the site of injection, B is the bottom vitreous chamber, and C is the back of the eye. The needle is injected approximately 20 degrees from the limbus (about 3-5 mm posterior to the limbus). The arrows represent the angles at which the needles could be injected. As shown in the image, the microparticles are injected and are deposited at the back of the eye.

FIG. 8D is an image of the eye post-injection for Method A as described in Example 11. A is the site of injection, B is the bottom vitreous chamber, and C is the back of the eye. Post-injection the microparticles remain at the back of the eye and must move forward to the bottom vitreous chamber.

FIG. 9A is scheme depicting the steps for injection of Method B as described in Example 11. The first step of the method of administration is wherein the patient has looked up. In the second step, the patient sits up and reorients his eyes to the vertical position. At this point, the microparticles move from point A to the bottom vitreous chamber (point B). The final depot location is shown in the last image.

FIG. 9B is an image of the eye during Method B as described in Example 11. A is the site of injection, B is the bottom vitreous chamber, and C is the back of the eye. The needle is injected approximately 20 degrees from the limbus (about 3-5 mm posterior to the limbus). The arrows represent the angles at which the needles could be injected. As shown in the image, the microparticles are injected and are deposited toward the bottom of the vitreous.

FIG. 9C is an image of the eye post-injection for Method B as described in Example 11. A is the site of injection, B is the bottom vitreous chamber, and C is the back of the eye. Post-injection the microparticles are already close to the bottom vitreous chamber and only slide minimally when the patient reorients her eye post injection.

FIG. 9D is an image of the eye during Method B as described in Example 11. The needle is injected at an approximate 10° angle at the 6 o'clock position with the patient looking upward approximately 20-30°. As show in the image, the microparticles are deposited close to the bottom of the vitreous and require minimal sliding to reach the bottom of the vitreous when the patient returns his pupil to a vertical position.

FIG. 9E is an image of the eye during Method B as described in Example 11. The needle is injected at an approximate 10° angle at the 6 o'clock position with the patient looking upward approximately 20-30°. As show in the image, the microparticles are deposited close to the bottom of the vitreous and require minimal sliding to reach the bottom of the vitreous when the patient returns his pupil to a vertical position.

FIG. 9F is a picture of a patient being injected via Method B as described in Example 11. The patient is looking up approximately 20-30° and the injection site is at the 6 o'clock position.

FIG. 9G is an image showing the 6 o'clock position as the site of injection for Method B and Method C as described in Example 11. This position is with respect to the eye looking straight.

FIG. 10A is an image depicting the injection site, tilt of the eye, and needle size of Method C, the injection method of the present invention, as described in Example 11.

FIG. 10B is scheme depicting the steps of Method C as described in Example 11. The first step of the method of administration is wherein the patient has looked up. In the second step, the patient sits up and reorients his eyes to the vertical position. At this point, the microparticles move slightly, or not at all, from point A to the bottom vitreous chamber (point B). The final depot location is shown in the last image. In this image the microparticle has minimal spreading compared to FIG. 8B.

FIG. 10C is an image of the eye during Method C as described in Example 11. A is the site of injection, B is the bottom vitreous chamber, and C is the back of the eye. The needle is injected approximately 20 degrees from the limbus (about 3-5 mm posterior to the limbus). The arrows represent the angles at which the needles could be injected. As shown in the image, the microparticles are injected and are deposited at or near the bottom vitreous chamber.

FIG. 10D is an image of the eye post-injection for Method C as described in Example 11. A is the site of injection and B is the bottom vitreous chamber. Post-injection the microparticles are at or very near to the bottom of the vitreous chamber and only require minimal or no movement to reach the bottom vitreous chamber.

FIG. 11A is an image of a left eye and an alternative method for administering a suspension of aggregating microparticles. In this alternative method, the patient is sitting upright with no head tilt. The patient turns his eye horizontally toward the nose (adduction movement) prior to injection with a 13 mm needle. The eye can be entered between approximately 2 o'clock and 3 o'clock with respect to the pupil of the left eye looking straight. The needle is injected at approximately 30-45° downward. In one embodiment, the needle is injected into the left eye at approximately 2 o'clock and the needle is pointed 45° downward. In one embodiment, the needle is injected into the left eye at approximately 3 o'clock and the needle is pointed 30° downward. Alternatively, the needle is injected into the right eye at approximately 10 o'clock and the needle is pointed 45° downward or the needle is injected into the right at approximately 9 o'clock and injected 30° downward.

FIG. 11B is an image of a left eye and an alternative method for administering a suspension of aggregating microparticles wherein the eye is turned horizontally toward the nose (adduction movement) and a 13 mm needle is injected away from the pupil. The eye can be entered between approximately 2 o'clock and 3 o'clock with respect to the pupil of the left eye looking straight. The needle is injected at approximately 30-50° downward.

FIG. 12A is the bottom view of a glass eye containing liquefied vitreous that was injected using Method A as described in Example 11. The arrow is pointing to spreading that has resulted due to Method A.

FIG. 12B is the side view of a glass eye containing liquefied vitreous that was injected using Method A as described in Example 11. The arrow is pointing to tailing that has resulted due to Method A.

FIG. 12C is the bottom view of a glass eye containing liquefied vitreous that was injected using Method B as described in Example 11.

FIG. 12D is the side view of a glass eye containing liquefied vitreous that was injected using Method B as described in Example 11.

FIG. 12E is the bottom view of a glass eye containing liquefied vitreous that was injected using Method C as described in Example 11.

FIG. 12F is the side view of a glass eye containing liquefied vitreous that was injected using Method C as described in Example 11.

FIG. 13A is an image of the microparticle deposition and aggregation of microparticles injected via Method A as described in Example 12.

FIG. 13B is an image of the microparticle deposition and aggregation of microparticles injected via Method B as described in Example 12.

FIG. 13C is an image of the microparticle deposition and aggregation of microparticles injected via Method C as described in Example 12.

FIG. 14A is a schematic representation of the locking mechanism of the VacLock syringe highlighting the locking fins and stopping pin as described in Example 13.

FIG. 14B is a schematic representation of the VacLock syringe when the apparatus is being used for normal sliding use. The stopping pin is positioned in such a way that the pin does not make contact with a locking fin as described in Example 13.

FIG. 14C is a schematic representation of the VacLock syringe when the apparatus is capable of being locked to hold vacuum. The stopping pin is positioned to make contact with the locking fins as described in Example 13.

FIG. 15 is an image of a 60 mL VacLok syringe attached to a suspension vial through a vial adapter as described in Example 13. The syringe plunger is locked at 50 mL to create a pressure of approximately 40 Torr inside the vial. The parts of the apparatus are as follows: 1) syringe plunger, which can be locked in different positions to create different pressures inside the glass vial; 2) 60 mL lockable syringe; 3) vial adapter; and, 4) 2 mL glass vial containing the particle suspension.

FIG. 16 illustrates the aggregate strength over time of a representative lot of surface treated microparticles (STMP) suspended at 200 mg/ml with various concentrations of benzyl alcohol (BA) added to the diluent as described in Example 14. The x-axis is incubation time measured in minutes and hours and the y-axis is the force at 30% of strain measured in gram-force (g).

FIG. 17 illustrates the aggregate strength over time of a representative lot of surface treated microparticles (STMP) suspended at 400 mg/ml with various concentrations of benzyl alcohol (BA) added to the diluent as described in Example 14. The x-axis is incubation time measured in minutes and hours and the y-axis is the force at 30% of strain measured in gram-force (g).

FIG. 18 illustrates the in vitro drug release profile of a representative lot of surface treated microparticles (STMP) containing 0% or 0.5% benzyl alcohol (BA) in the diluent as described in Example 15. The x-axis is time measured in days and the y-axis is cumulative release measured in percent.

FIG. 19A illustrates the effect of benzyl alcohol (BA) on the aggregation of non-surface treated microparticles (NSTMP) suspended at 200 mg/ml after injection into PBS and incubation at 37° C. for 15 minutes as described in Example 14. Samples from left to right are 0%, 0.5%, 1%, and 2% BA in the diluent (S-A, S-B, S-C, and S-D, respectively).

FIG. 19B illustrates the effect of benzyl alcohol (BA) on the aggregation of non-surface treated microparticles (NSTMP) suspended at 400 mg/ml after injection into PBS and incubation at 37° C. for 15 minutes as described in Example 14. Samples from left to right are 0%, 0.5%, and 1% BA in the diluent (S-E, S-F, and S-G, respectively).

FIG. 20 illustrates the aggregate strength over time of a representative lot of surface treated microparticles (STMP) suspended at 200 mg/ml with various concentrations of triethyl citrate (TEC) added to the diluent as described in Example 15. The x-axis is incubation time measured in minutes and the y-axis is the force at 30% of strain measured in gram-force (g).

FIG. 21A illustrates the effect of triethyl citrate (TEC) on the aggregation of non-surface treated microparticles (NSTMP) suspended at 200 mg/ml after injection into PBS and incubation at 37° C. for 15 minutes as described in Example 15. Samples from left to right are 0% and 0.5% TEC in the diluent (S-H and S-I, respectively).

FIG. 21B illustrates the effect of triethyl citrate (TEC) on the aggregation of non-surface treated microparticles (NSTMP) after injection into PBS and incubation at 37° C. for 15 minutes as described in Example 15. Samples from left to right are 0%, 0.5%, 1%, and 2% TEC in the diluent (S-J, S-K, S-L, and S-M, respectively).

FIG. 22 is scheme with the steps for producing the suspensions of surface-treated aggregating microparticles of the present invention. Briefly, the microparticles are produced by mixing the dispersed phase (DP) and continuous phase (CP) as described herein (Step 1) and then subjected to surface-treatment as described herein (Step 2). The microparticles are then lyophilized and transferred to vials (Step 3). The lyophilized microparticles are reconstituted in the appropriate diluent to afford reconstituted product in suspension (Step 4). The suspension is then subjected to vacuum treatment (Step 5) prior to syringe loading and administration (Step 6).

### DETAILED DESCRIPTION

The present invention is improved compositions and methods of an in vivo aggregated microparticle for controlled drug delivery of an active agent to the eye.

It has been discovered that it requires the coordinated control of a combination of a number of factors when used together achieves this significantly harder and/or durable aggregated microparticle in vivo. In one embodiment, the factors include using an optimized microparticle preparation and composition in combination with an improved method of administration.

The microparticle preparation factors include the removal of the smaller microparticles that may be accomplished with one or more, including serial centrifugation steps, for example, 2, 3, 4, or 5 centrifugation steps in serial or continuous fashion. Additional factors include surface treatment conditions that utilize about 1.0 or 1.5 mM and less than 10 mM NaOH in a solvent of between about 55% and 75% ethanol in water and the use of a diluent that includes additive that upon injection in vivo assists in the aggregation, for example benzyl alcohol or triethyl citrate.

The improved methods of administration are advantageous to typical intravitreal methods of administration because the microparticles are administered in such a way that the microparticles are injected toward the bottom of the vitreous, which minimizing sliding and tailing as the patient reorients his or her eye and the microparticles settle at the bottom of the vitreous. This allows for enhanced aggregation in vivo. This combination of factors achieves an improved aggregated microparticle of at least 500 microns.

In one aspect, an improved aggregated microparticle of at least 500 microns is provided that exhibits a hardness rating in vivo in the vitreous of the eye, for example a human eye, of at least 5, and in some embodiments, at least about 10, 15, 20 or 25 gram-force needed to compress the particle at 30% of strain. In one embodiment, the hardness of the microparticle increases, upon injection in the vitreous, at least two-fold, at least three-fold, at least four-fold, at least five-fold, or more in four hours or less, in three hours or less, in two hours or less, in one hour or less, in thirty minutes or less, in fifteen minutes or less, in ten minutes or less, in five minutes or less, in two minutes or less, or in one minute or less following injection compared to microparticles administered immediately after injection (for example, less than one minute or even 30 seconds after administration).

The hardness of the aggregated microparticle can be confirmed in vitro in vitreous fluid, in phosphate buffered saline, or in water or other physiologically acceptable aqueous solution, including an aqueous solution that includes one or more components of the vitreous, which are well-known. The vitreous humor fluid in vivo typically contains 98-99% water, salts, sugars, vitrosin, fibrils with glycosaminoglycan, hyaluronan (i.e., hyaluronic acid), opticin, and various proteins. The vitreous humor typically has a viscosity of approximately 2-4 times that of water. In one embodiment, the hardness is tested in a hyaluronic acid-based solution with a viscosity approximately mimicking that of the vitreous. In one embodiment, the hardness is measured in a fluid selected from vitreous, water, phosphate buffered saline, or an aqueous physiologically acceptable solution with a viscosity not more than about 4 times that of water.

### I. Optimized Method of Administration

In certain embodiments, a method is provided for increasing the hardness and/or durability of an in vivo aggregated microparticle for controlled delivery of an active agent to the eye of a patient for therapeutic purposes.

In one embodiment, the hardness increases, upon injection in the vitreous, relative to the microparticle immediately after injection (for example, less than one minute or even 30 seconds after administration). In one embodiment, upon injection in the vitreous, the hardness of the microparticle increases at least two-fold in about two hours or less following injection relative to microparticles administered immediately after injection (for example, less than one minute or even 30 seconds after administration). One non-limiting method for administering a microparticle that aggregates into a microparticle of at least about 500 microns of advantageous hardness and/or durability in vivo includes:
(a) Providing a solution or suspension of optimized aggregating microparticles as described herein;
(b) Loading a means for injection that comprises a needle of less than about 7 mm with the selected amount of the solution or suspension of the aggregating microparticles;
(c) Positioning the patient in an approximate sitting position looking up with at least an approximate 15 degree angle;
(d) Injecting the solution or suspension of aggregating microparticles:
   i. through the pars plana of the eye between 3 and 6 mm posterior to the limbus of the eye;
   ii. wherein the point of needle entry is between about 5:30 o'clock and 9 o'clock with respect to the pupil of the eye looking straight, and typically between about 6 and 8 o'clock;
   iii. at an angle that deposits the solution or suspension at or near the bottom of the vitreous chamber and in a manner that not more than about 4 mm of the needle is in the vitreous; and
(e) After a short movement period to change chairs if necessary, maintaining the patient in a sitting position for a sufficient time to allow the aggregating microparticles to aggregate to at least one aggregated microparticle of at least 500 microns.

An additional non-limiting method for administering a microparticle that aggregates into a microparticle of at least about 500 microns of advantageous hardness and/or durability in vivo includes:
(a) Providing a solution or suspension of optimized aggregating microparticles as described herein;
(b) Loading a means for injection that comprises a needle of between about 10 mm and about 18 mm with the selected amount of the solution or suspension of the aggregating microparticles;
(c) Positioning the patient in an approximate sitting position looking up with at least an approximate 15 degree angle;
(d) Injecting the solution or suspension of aggregating microparticles:
   i. through the pars plana of the eye between 3 and 6 mm posterior to the limbus of the eye;
   ii. wherein the point of needle entry is between about 4:00 o'clock and 8 o'clock with respect to the pupil of the eye looking straight, and typically about 6 o'clock;
   iii. at an angle that deposits the solution or suspension at or near the bottom of the vitreous chamber; and
(e) After a short movement period to change chairs if necessary, maintaining the patient in a sitting position for a sufficient time to allow the aggregating microparticles to aggregate to at least one aggregated microparticle of at least 500 microns.

In one embodiment, the improved aggregated microparticle of at least 500 microns exhibits a hardness rating in vivo in the vitreous of the eye of at least 5 gram-force, 10 gram-force, 15 gram-force, or 20-gram force needed to compress the particle at 30% of strain. In one embodiment, the hardness of the microparticle increases, upon injection in the vitreous, at least three-fold, at least four-fold, at least five-fold, at least six-fold, at least seven-fold, at least eight-fold, at least nine-fold, at least ten-fold or more in three hours or less, in two hours or less, in one hour or less, in thirty minutes or less, in fifteen minutes or less, in ten minutes or less, in five minutes or less, in two minutes or less, or in one minute or less following injection compared to immediately after injection (for example, less than one minute or even 30 seconds after administration).

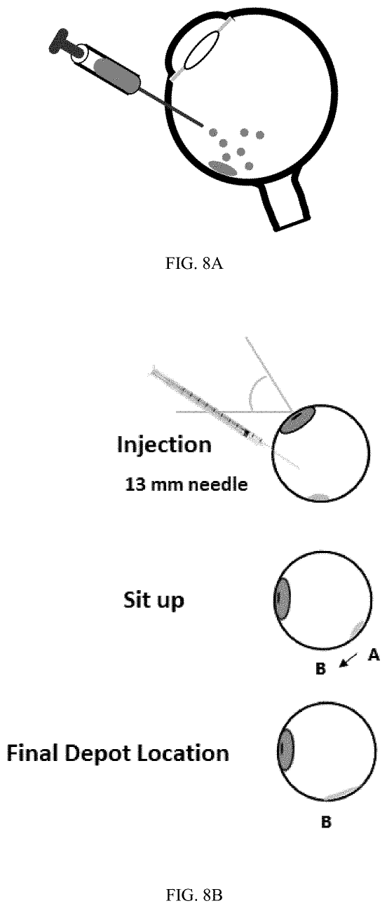

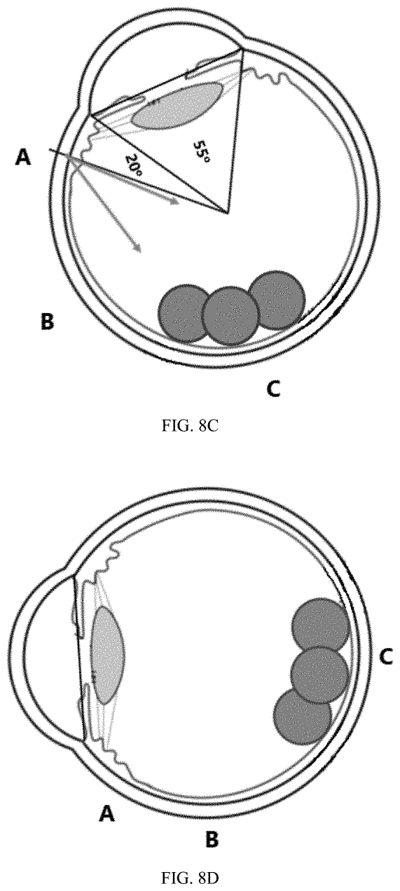

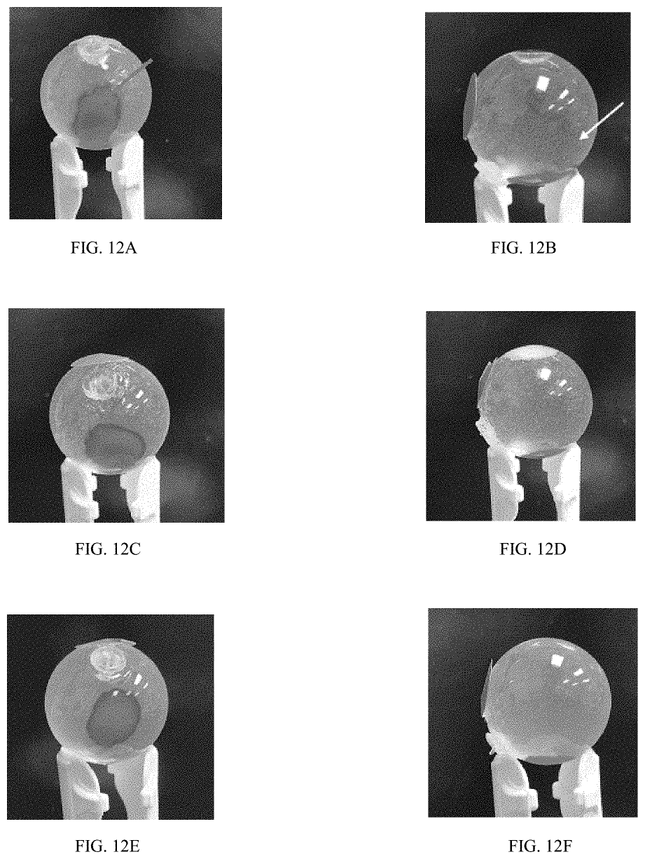

As described in Example 11, the methods of administration of the current invention are superior to the previous method of administration for injecting microparticles. In the previous method of administration, the patient was directed to tilt her head approximately 450 upward and to look up an additional 20-30°. The solution or suspension of aggregating microparticles was then injected with a 13 mm needle 10 mm inside the eye through the para plana (FIG. 8A and FIG. 8B). Due to the length of the needle and the angle of the patient's eye (caused by the head tilt and upward position of the eye), the microparticles were deposited toward the back of the eye. To reach the bottom of the vitreous chamber, the microparticles have to move from the initial deposit in the back of the eye when the patient sat up and reoriented her eye back to a vertical position (FIG. 8B). This would result in spreading and tailing of the aggregated microparticle. Example 11 and FIGS. 12A and 12B discuss and depict the spreading and tailing that was a result of the previous injection method (described as Method A). FIG. 12A is the bottom view of a glass eye and the arrow is pointing to the spreading. FIG. 12B is a side view of the glass eye and the arrow is pointing to the tailing of the microparticle.

In contrast to the previous method of administration, the methods of administration of the present invention were surprisingly discovered to not result in tailing or spreading of the microparticle post-injection.

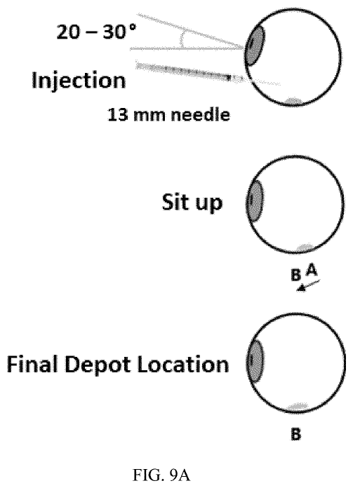

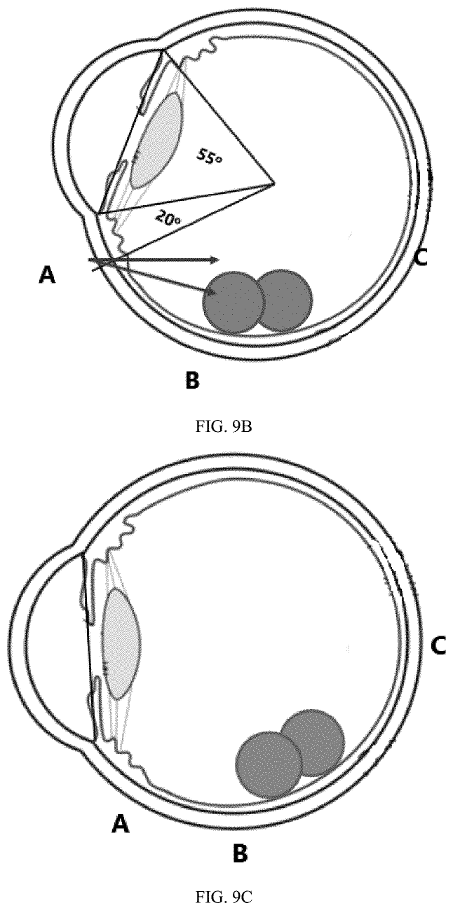

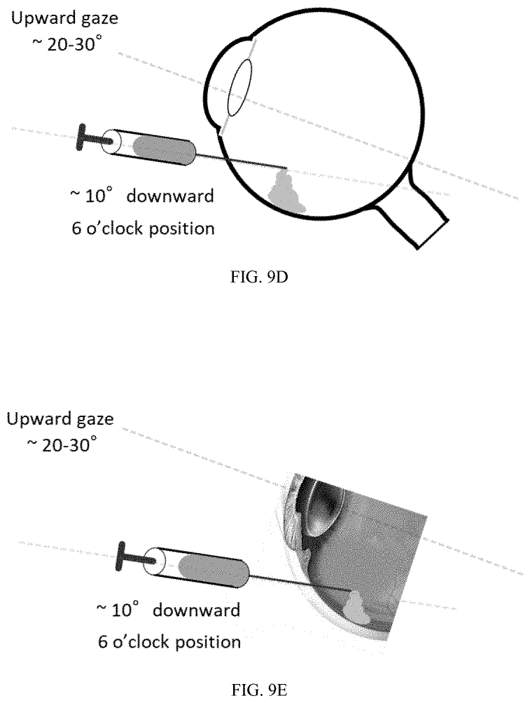

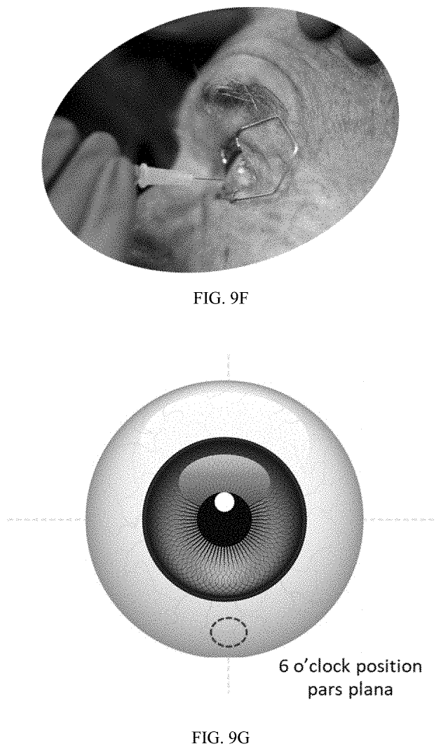

As discussed in Example 11 and shown in FIGS. 9A-9F, one of the methods of administering the therapeutic aggregating microparticles of the current invention (referred to as Method B) in Example 11 requires that the patient sit up and only look upward 20-30⁰, but not tilt her/his head upward (FIG. 9A). The suspension or solution of microparticles is then injected with a 10-18 mm needle 3-6 mm inside the eye through the pars plana. Since the patient is sitting up and not tilting her head back, the microparticles are not deposited at the back of the eye, but are instead deposited at or near the bottom of the vitreous chamber (FIGS. 9C-9F).

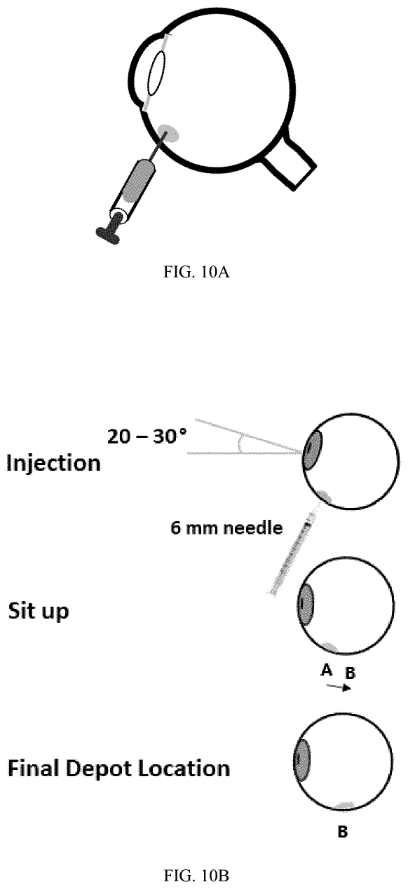

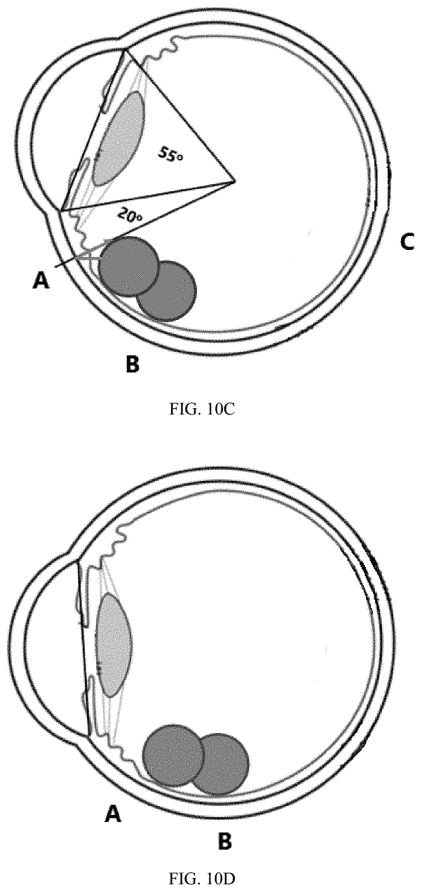

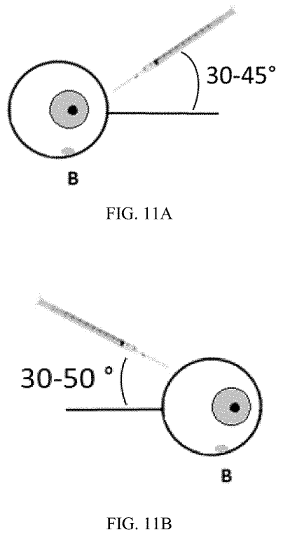

The second method of administering the therapeutic aggregating microparticles of the current invention (referred to as Method C) in Example 11 requires that the patient only look upward 20-30°, but not tilt her/his head upward (FIGS. 10A-10B). The suspension or solution of microparticles is then injected with a needle less than 7 mm 3-6 mm inside the eye through the pars plana. Since the needle is short and the patient is not tilting her head back, the microparticles are not deposited at the back of the eye, but are instead deposited at or near the bottom of the vitreous chamber (FIG. 10D). The deposition of the microparticles at the bottom of the eye is not heavily dependent on the angle of the needle because the needle is short.

Once the patient reorients her eye back to the vertical position when the microparticles are administered using Method B or Method C, the microparticles only minimally move, or not at all, to reach the bottom vitreous chamber. This minimal movement results in minimal sliding and tailing of the microparticles or no sliding and tailing of the microparticles.

The superiority of the present invention is evident when comparing FIGS. 12A-12B and FIGS. 12C-12F. As discussed above, FIGS. 12A-12B are a result of the previous method of administration (Method A in Example 11). Sliding and tailing are shown. In contrast, FIGS. 12C-12D and FIGS. 12E-12F are images of glass eyes that has been injected with a solution of microparticles via the methods of the current invention (Method B and Method C in Example 11, respectively). FIG. 12C and FIG. 12E are the bottom views of the glass eyes after Method B and Method C, respectively. Spreading of the microparticle is not observed. FIG. 12D and FIG. 12F are the side views of the glass eyes after Method B and Method C administration, respectively. No tailing is observed.

In one embodiment, the means for injection is a syringe and the needle is approximately 31, 30, 29, 28, 27, 26 or 25 gauge with either normal or thin wall. In one embodiment, the needle is about 3 mm, about 4 mm, about 5 mm, about 6 mm, or about 7 mm in length. In one embodiment, the needle is 6 mm in length. In an alternative embodiment, the needle is about 8 mm, about 9 mm, about 10 mm, about 11 mm, about 12 mm, about 13 mm, about 14 mm, about 15 mm, about 16 mm, about 17 mm, or about 18 mm. In one embodiment the needle is 6 mm in length and has a 27 gauge. In one embodiment, the needle is 13 mm in length and has a 27 gauge.

In some embodiments, the short needle of less than 7 mm in length is surprisingly advantageous over needles of longer length. When the needle of less than 7 mm in length is injected into the eye 3 mm to 6 mm posterior to the limbus, the short length allows for delivery of the aggregating microparticles to the bottom of the vitreous chamber regardless of the angle that the needle is positioned. During the injection, the patient is looking upward and the eye is tilted upward at an angle of approximately 15°, but following the injection, the patient returns his or her eye to a position with no angle. Because the microparticles are already deposited at the bottom of the vitreous chamber, when the patient returns his eye to a normal position looking straight, there is minimal sliding or dispersion of the aggregated microparticles, resulting in improved in vivo aggregation of the microparticles compared to injection methods that utilize longer needles. In one embodiment, the needle is injected in a manner that allows for not more than about 4 mm of the needle is in the vitreous. In one embodiment, the needle is injected in a manner that allows for not more than about 3 mm of the needle is in the vitreous.

In one embodiment, the needle is injected about 3 mm, about 3.5 mm, about 4 mm, about 4.5 mm, about 5 mm, about 5.5 mm, or about 6 mm posterior to the limbus.

In one embodiment, the solution or suspension of microparticles has been treated with vacuum before loading into the injection means at a pressure of less than 40 Torr, less than 30 Torr, less than 25 Torr, less than 20 Torr, less than 10 Torr, or less than 5 Torr for between 1 and 90 minutes.

In one embodiment, the solution or suspension of microparticles has been vortexed or shaken before loading into the injection means. In one embodiment, the vortexing or shaking occurs for less than 10 minutes, less than 8 minutes, less than 5 minutes, less than 3 minutes, or less than 1 minute.

In one embodiment, the injection of the solution or suspension of microparticles takes place over about 3 to 10 seconds. In one embodiment, the injection of the solution or suspension of microparticles takes place over about 3 to 8 seconds. In one embodiment, the injection of the solution or suspension of microparticles takes place over about 3 to 5 seconds.

In one embodiment, the point of needle entry for administration is between 5:30 o'clock and 9 o'clock with respect to the pupil of the eye looking straight. In one embodiment, the point of needle entry for administration is between 4:00 o'clock and 8:00 o'clock with respect to the pupil of the eye looking straight. In one embodiment, the point of needle entry for administration is between 3:30 o'clock and 7:00 o'clock with respect to the pupil of the eye looking straight. In one embodiment, the point of needle entry for administration is between 3:00 o'clock and 6:00 o'clock with respect to the pupil of the eye looking straight. In one embodiment, the point of needle entry for administration is about 6:00 o'clock with respect to the pupil of the eye looking straight.

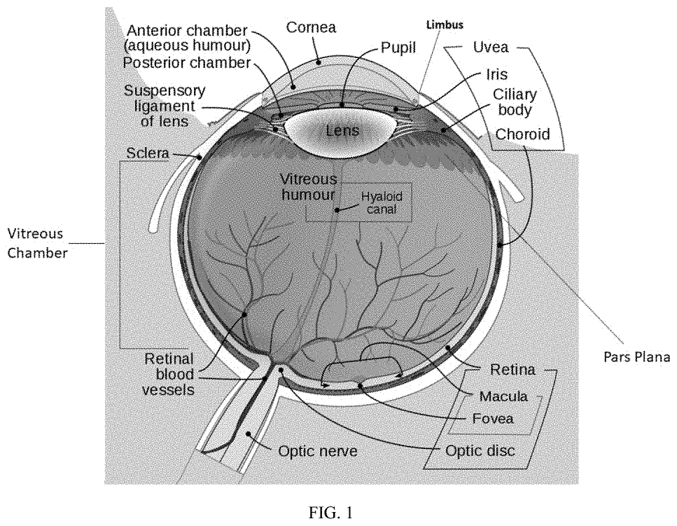

FIG. 1 is a labeled image of the anatomy of the eye. The method of administration of the present invention deposits the microparticles at the bottom of the vitreous chamber as shown in FIG. 10D. The pupil, which is the point of reference for the site of the needle injection, is the hole located in the center of the iris.

In one embodiment, the optimized aggregating microparticles of step (a) comprise at least one biodegradable polymer and a therapeutic agent that is encapsulated in the biodegradable polymer wherein the microparticles have a mean diameter between 10 μm and 60 μm that:
(i) have a solid core with less than 10% porosity by ratio of void space to total volume;
(ii) contain from about 0.001 percent to about 1 percent surfactant and have been surface-modified to contain less surfactant than a microparticle prior to the surface modification wherein the surface has been modified at a temperature less than about 18° C.; and
(iii) are capable of aggregating in vivo to form at least one pellet of at least 500 m in vivo capable of sustained drug delivery in vivo for at least one month.

In an alternative embodiment, the optimized aggregating microparticles of step (a) comprise at least one biodegradable polymer and a therapeutic agent that is encapsulated in the biodegradable polymer wherein the microparticles have a mean diameter between 10 μm and 60 μm that:
(i) contain from about 0.001 percent to about 1 percent surfactant and have been surface-modified to contain less surfactant than a microparticle prior to the surface modification wherein the surface has been modified at a temperature less than about 18° C.; and
(ii) are capable of aggregating in vivo to form at least one pellet of at least 500 m in vivo capable of sustained drug delivery in vivo for at least one month.

In one embodiment, the microparticles comprise PLGA. In one embodiment, the microparticles comprise PLA. In one embodiment, the microparticles comprise PLGA-PEG. In one embodiment, the microparticles comprise PLGA and PLGA-PEG. In one embodiment, the microparticles comprise PLGA, PLA, and PLGA-PEG.

In one embodiment, the microparticles of the present invention have a light transmittance percentage greater than about 90%, 92%, 94%, 96%, 98% or 99%.

In one embodiment, the microparticles of the present invention have drug loading of greater than about 5%, about 10%, or 15%.

In one embodiment, the microparticles are between about 10 μm and about 60 μm. In one embodiment, the microparticles are between about 20 μm and about 40 μm. In one embodiment, the microparticles are between about 20 μm and about 35 μm. In one embodiment, the microparticles are between about 20 μm and about 30 μm.

Another aspect of the invention includes a drug delivery system for ocular injection into the vitreous chamber of the eye comprising
(a) lyophilized mildly surface-treated microparticles comprising at least one biodegradable polymer, surfactant, and a therapeutic agent in a vial;
(b) a diluent comprising additive;
(c) a vacuum pressure syringe comprising a plunger that locks in position to create vacuum;
(d) an adapter that connects vacuum syringe (c) and vial (a).

In an alternative embodiment, a method for administering a microparticle that aggregates into a microparticle of at least 500 microns of advantageous hardness and/or durability in vivo includes the patient is sitting upright with no head tilt. The patient turns his eye horizontally toward the nose (adduction movement) prior to injection with a 13 mm needle. The eye can be entered between approximately 2 o'clock and 3 o'clock with respect to the pupil of the left eye looking straight. The needle is injected at approximately 30-45° downward. In one embodiment, the needle is injected into the left eye at approximately 2 o'clock and the needle is pointed 45° downward. In one embodiment, the needle is injected into the left eye at approximately 3 o'clock and the needle is pointed 30° downward. Alternatively, the needle is injected into the right eye at approximately 10 o'clock and the needle is pointed 45° downward or the needle is injected into the right at approximately 9 o'clock and injected 30° downward. This method is shown in FIGS. 12A and 12B. This method of administration is advantageous because it deposits microparticles close to the bottom of the vitreous, which minimizes tailing and sliding when the patient reorients her head back to the vertical position. It also helps minimize patient head or eye movements after injection.

### II. Optimized Microparticle Preparation

The present invention also includes the following aspects related to optimized microparticle preparation:
I. A suspension of mildly surface-treated microparticles in a diluent wherein the microparticles aggregate in vivo to form a larger pellet and wherein the diluent comprises additive that improves the particle aggregation in vivo.
II. A suspension of mildly surface-treated aggregating microparticles in a diluent wherein the diluent comprises additive that improves in vivo particle aggregation, and a method for manufacture thereof wherein the microparticles are loaded with a pharmaceutically active agent, including those listed below, which can be active in the form delivered or as a prodrug, with non-limiting examples provided herein, for in vivo treatment of a patient in need thereof.
III. An aggregated polymeric microparticle of at least 500 microns that exhibits a hardness rating in vivo in the vitreous of the eye of at least 5 gram-force needed to compress the particle at 30% of strain, which is optionally biodegradable and optionally comprises a therapeutic agent.

In one embodiment, the invention is thus a suspension of solid aggregating microparticles comprising surface surfactant, at least one biodegradable polymer, and a therapeutic agent in a diluent comprising an additive that improves in vivo particle aggregation wherein the solid aggregating microparticles:
(i) have a solid core with less than 10% porosity by ratio of void space to total volume;
(ii) have been surface-modified to contain less surfactant on the surface than a microparticle prior to surface modification and wherein the surface has been modified at a temperature less than about 18° C.;
(iii) have a mean diameter between 10 um and 60 um; and
(iv) are capable of aggregating in vivo to form at least one pellet of at least 500 μm in vivo capable of sustained drug delivery in vivo for at least three months.

In an alternative embodiment, the invention includes aggregating microparticles comprising surface surfactant, at least one biodegradable polymer, and a therapeutic agent selected from pilocarpine and alpha lipoic acid wherein the solid aggregating microparticles:
(i) have been surface-modified to contain less surfactant on the surface than a microparticle prior to surface modification and wherein the surface has been modified at a temperature less than about 18° C.;
(ii) have a mean diameter between 10 um and 60 um; and
(iii) are capable of aggregating in vivo to form at least one pellet of at least 500 m in vivo capable of sustained drug delivery in vivo for at least three months.

In one embodiment, the invention is solid aggregating microparticles comprising surface surfactant, at least one biodegradable polymer, and a therapeutic wherein the solid aggregating microparticles:
(i) have been surface-modified to contain less surfactant on the surface than a microparticle prior to surface modification and wherein the surface has been modified at a temperature less than about 18° C.;
(ii) have a mean diameter between 10 um and 60 um; and
(iii) are capable of aggregating in vivo to form at least one pellet of at least 500 μm in vivo capable of sustained drug delivery in vivo for at least three months; and
(iv) are capable of aggregating to a pellet of at least 500 microns that exhibits a hardness rating in vivo in the vitreous of the eye of at least 5 gram-force needed to compress the particle at 30% of strain.

In one embodiment, the suspension has been treated with vacuum at a pressure of less than 40 Torr, less than 30 Torr, less than 25 Torr, less than 20 Torr, less than 10 Torr, or less than 5 Torr for between 1 and 90 minutes.

In one embodiment, the therapeutic agent is selected from pilocarpine and alpha lipoic acid.

In one embodiment, the solid aggregating microparticles contain from about 0.001 percent to about 1 percent surfactant. In one embodiment, the microparticle contains from about 0.01 percent to about 0.5 percent surfactant, about 0.05 percent to about 0.5 percent surfactant, about 0.1 percent to about 0.5 percent surfactant, or about 0.25 percent to about 0.5 percent surfactant. In one embodiment, the microparticle contains from about 0.001 percent to about 1 percent surfactant, about 0.005 percent to about 1 percent surfactant, about 0.075 percent to about 1 percent surfactant, or about 0.085 percent to about 1 percent surfactant. In one embodiment, the microparticle contains from about 0.01 percent to about 5.0 percent surfactant, about 0.05 percent to about 5.0 percent surfactant, about 0.1 percent to about 5.0 percent surfactant, about 0.50 percent to about 5.0 percent surfactant. In one embodiment, the microparticle contains from about 0.10 percent to about 1.0 percent surfactant or about 0.50 percent to about 1.0 percent. In one embodiment, the microparticle contains up to about 0.10, 0.15, 0.20, 0.25, 0.30, 0.40 or 0.5% surfactant.

Thus, according to the present invention, a suspension of solid aggregating microparticles in a diluent comprising additive are provided that have improved aggregation to a pellet for medical therapy. Examples of non-limiting additives include triethyl citrate, benzyl alcohol, polyethylene glycol, N-methyl-2-pyrrolidone (NMP), 2-pyrrolidone, DMSO, triacetin, benzyl acetate, benzyl benzoate, acetyltributyl citrate, dibutyl sebacate, dimethylphthalate, tributyl O-acetylcitrate, ethanol, methanol, polysorbate 80, ethyl acetate, propylene carbonate, isopropyl acetate, methyl acetate, methyl ethyl ketone, butyl lactate, and isovaleric acid.

The microparticles of the invention can be used for the controlled administration of active compounds to the eye, over a period of at least two, three, four, five or six months or more in a manner that maintains at least a concentration in the eye that is effective for the disorder to be treated. In one embodiment, the microparticle provides a controlled release that is substantially linear. In another embodiment, the release is not substantially linear; however, even the lowest concentration of release over the designated time period is at or above a therapeutically effective dose. In one embodiment, this is achieved by microparticles comprising moieties of at least lactic acid, glycolic acid, propylene oxide or ethylene oxide. In a particular embodiment, the microparticle includes PLGA, PLA or PGA with or without covalently attached or admixed polyethylene glycol. For example, the microparticle is a mixture of PLGA and PLGA-PEG, PEG, PLA, or PLA-PEG. The microparticle may be a mixture of PLA and PLGA-PEG, PEG, PLGA, or PLA-PEG.

In certain embodiments, the prodrug of the present invention is delivered in a microparticle or nanoparticle that is a blend of two polymers, for example (i) a PLGA polymer or PLA polymer as described herein and (ii) a PLGA-PEG or PLA-PEG copolymer. In another embodiment, the microparticle or nanoparticle is a blend of three polymers, such as, for example, (i) a PLGA polymer; (ii) a PLA polymer; and, (iii) a copolymer of PLGA-PEG or PLA-PEG. In an additional embodiment, the microparticle or nanoparticle is a blend of (i) a PLA polymer; (ii) a PLGA polymer; (iii) a PLGA polymer that has a different ratio of lactide and glycolide monomers than the PLGA in (ii); and, (iv) a PLGA-PEG or PLA-PEG copolymer. Any ratio of lactide and glycolide in the PLGA can be used that achieves the desired therapeutic effect. In certain illustrative non-limiting embodiments, the ratio of PLA to PLGA by weight in a polymer blend as described is 77/22, 69/30, 49/50, 54/45, 59/40, 64/35, 69/30, 74/25, 79/20, 84/15, 89/10, 94/5, or 99/1.

In certain embodiments, a blend of three polymers that has (i) PLA (ii) PLGA (iii) PLGA with a different ratio of lactide and glycolide monomers than PLGA in (ii) wherein the ratio is 74/20/5 by weight, 69/20/10 by weight, 69/25/5 by weight, or 64/20/15 by weight. In certain embodiments, the PLGA in (ii) has a ratio of lactide to glycolide of 85/15, 75/25, or 50/50. In certain embodiments the PLGA in (iii) has a ratio of lactide to glycolide of 85/15, 75/25, or 50/50.

In certain aspects, the drug may be delivered in a blend of PLGA or PLA and PEG-PLGA, including but not limited to (i) PLGA+ approximately by weight 1% PEG-PLGA or (ii) PLA+ approximately by weight 1% PEG-PLGA. In certain aspects, the drug may be delivered in a blend of (iii) PLGA/PLA+ approximately by weight 1% PEG-PLGA. In certain embodiments, the blend of PLA, PLGA, or PLA/PGA with PLGA-PEG contains approximately from about 0.5% to about 10% by weight of a PEG-PLGA, from about 0.5% to about 5% by weight of PEG-PLGA, from about 0.5% to about 4% by weight of PEG-PLGA, from about 0.5% to about 3% by weight of PEG-PLGA, from about 1.0% to about 3.0% by weight of PEG-PLGA, from about 0.1% to about 10% of PEG-PLGA, from about 0.1% to about 5% of PEG-PLGA, from about 0.1% to about 1% PEG-PLGA, or from about 0.1% to about 2% PEG-PLGA.

In certain non-limiting embodiments, the ratio by weight percent of PLGA to PEG-PLGA in a two polymer blend as described is in the range of about or between the ranges of 40/1, 45/1, 50/1, 55/1, 60/1, 65/1, 70/1, 75/1, 80/1, 85/1, 90/1, 95/1, 96/1, 97/1, 98/1, 99/1. The PLGA can be acid or ester capped. In non-limiting aspects, the drug can be delivered in a two polymer blend of PLGA75:25 4 A+ approximately 1% PEG-PLGA50:50; PLGA85:15 5 A+ approximately 1% PEG-PLGA5050; PLGA75:25 6E+ approximately 1% PEG-PLGA50:50; or, PLGA50:50 2 A+ approximately 1% PEG-PLGA50:50.

In certain non-limiting embodiments, the ratio by weight percent of PLA/PLGA-PEG in a polymer blend as described is in the range of about or between the ranges of 40/1, 45/1, 50/1, 55/1, 60/1, 65/1, 70/1, 75/1, 80/1, 85/1, 90/1, 95/1, 96/1, 97/1, 98/1, 99/1. The PLA can be acid capped or ester capped. In certain aspects, the PLA is PLA 4.5 A. In non-limiting aspects, the drug is delivered in a blend of PLA 4.5 A+1% PEG-PLGA.

The PEG segment of the PEG-PLGA may have, for example, in non-limiting embodiments, a molecular weight of at least about or between 1 kDa, 2 kDa, 3 kDa, 4 kDa, 5 kDa, 6 kDa, 7 kDa, 8 kDa, 9 kDa, or 10 kDa, and typically not greater than 10 kDa, 15 kDa, 20 kDa, or 50 kDa, or in some embodiments, 6 kDa, 7 kDa, 8 kDa, or 9 kDa. In certain embodiment, the PEG segment of the PEG-PLGA has a molecular weight between about 3 kDa and about 7 kDa or between about 2 kDa and about 7 kDa. Non-limiting examples of the PLGA segment of the PEG-PLGA is PLGA50:50, PLGA75:25, or PLGA85:15. In one embodiment, the PEG-PLGA segment is PEG (5 kDa)-PLGA50:50.

When the drug is delivered in a blend of PLGA+PEG-PLGA, any ratio of lactide and glycolide in the PLGA or the PLGA-PEG can be used that achieves the desired therapeutic effect. Non-limiting illustrative embodiments of the ratio of lactide/glycolide in the PLGA or PLGA-PEG are in the range of about or between the ranges of 5/95, 10/90, 15/85, 20/80, 25/75, 30/70, 35/65, 40/60, 45/55, 50/50, 55/45, 60/40, 65/35, 70/30, 75/25, 80/20, 85/15, 90/10, or 95/5. In one embodiment, the PLGA is a block co-polymer, for example, diblock, triblock, multiblock, or star-shaped block. In one embodiment, the PLGA is a random co-polymer. In certain aspects, the PLGA is PLGA75:25 4 A; PLGA85:15 5 A; PLGA75:25 6E; or, PLGA50:50 2 A.

In another embodiment, the microparticle includes polyethylene oxide (PEO) or polypropylene oxide (PPO). In certain aspects, the polymers of the microparticles can be a random, block, diblock, triblock or multiblock copolymer (for example, a polylactide, a polylactide-co-glycolide, polyglycolide or Pluronic). For injection into the eye, the polymer is pharmaceutically acceptable and typically biodegradable so that it does not have to be removed.

In one aspect of the invention, a pharmaceutically acceptable kit for ocular injection into the vitreous chamber of the eye is provided comprising
(a) lyophilized mildly surface-treated microparticles comprising at least one biodegradable polymer, surfactant, and a therapeutic agent in a vial;
(b) a diluent comprising additive that enhances aggregation;
(c) a vacuum pressure syringe comprising a plunger that locks in position to create vacuum; and
(d) an adapter that connects vacuum syringe (c) and vial (a).

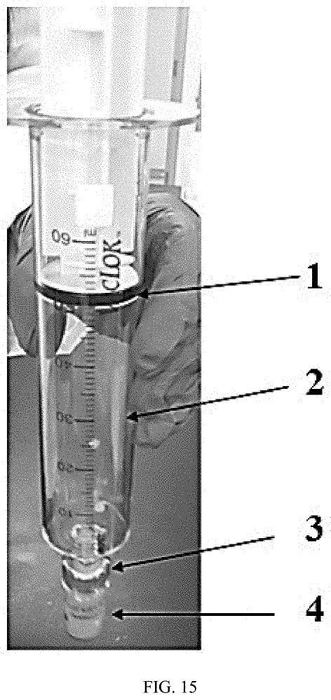

In one embodiment, the diluent comprises additive that improves in vivo particle aggregation. In one embodiment, vacuum syringe (c) is a 60 mL VacLok syringe containing a plunger (as illustrated in FIG. 15). In one embodiment, the diluent is in a vial. In an alternative embodiment, the diluent is in a syringe.

In one aspect of the invention, the improved process for preparing a microparticle suspension leading to an aggregated pellet in vivo can be used in combination with a selected method for forming aggregating microparticles described in U.S. Ser. No. 15/349,985, PCT/US16/61706, U.S. Ser. No. 15/976,847, PCT/US18/32167, and PCT/US2019/028803 (and the resulting materials thereof). For example, methods include providing suspensions of solid aggregating microparticles in a diluent comprising additive wherein the microparticles include at least one biodegradable polymer, have a solid core, include a therapeutic agent, have a modified surface which has been treated under mild conditions at a temperature that may optionally be at or less than about 18° C. to remove surface surfactant or to partially remove surface surfactant, are sufficiently small to be injected in vivo, and are capable of aggregating in vivo to form at least one pellet of at least 500 μm in vivo to provide sustained drug delivery in vivo for at least three months, four months, five months, six months seven months, eight months, nine months or more. In certain embodiments, sustained drug deliver in vivo is provided for up to one year. The solid aggregating microparticles are suitable, for example, for an intravitreal injection, implant, including an ocular implant, periocular delivery, or delivery in vivo outside of the eye. In certain embodiments, the therapeutic agent is a prodrug as described herein.

As an illustration, the present invention includes a process for the preparation of surface-modified solid aggregating microparticle suspensions that provide microparticles that aggregate in vivo to form pellets as described herein that includes:
A. a first step of preparing microparticles comprising one or more biodegradable polymers by dissolving or dispersing the polymer(s) and a therapeutic agent in one or more solvents to form a polymer and therapeutic agent solution or dispersion, mixing the polymer and the therapeutic agent solution or dispersion with an aqueous phase containing a surfactant to produce solvent-laden microparticles and then removing the solvent(s) to produce polymer microparticles that contain the therapeutic agent, polymer and surfactant; and
B. a second step of mildly treating the surface of microparticles of step (i) at a temperature at or below about 18, 15, 10, 8 or 5° C. optionally up to about 1, 2, 3, 4, 5, 10, 30, 40, 50, 60, 70, 80, 90 100, 11, 120 or 140 minutes (wherein each alternative is considered individually as described as if separately written out) with an agent that removes or partially removes surface surfactant, surface polymer, or surface oligomer in a manner that does not significantly produce internal pores;
C. washing the microparticles with a solution comprising an excipient, optionally mannitol;
D. isolating and lyophilizing the surface-treated microparticles;
E. resuspending the surface-treated microparticles in an appropriate diluent that comprises additive that improves in vivo particle aggregation;
F. optionally further improving the aggregation potential of the particles by subjecting the particles to at least one process selected from 1) vacuum treatment; 2) sonication; and 3) vortex.

In one embodiment, step (F) includes vacuum treatment and vortexing.

In one embodiment, the process also includes continuous centrifugation prior to step (C).

In another non-limiting embodiment, a process for preparing a suspension comprising a microparticle and a pharmaceutically active compound encapsulated in the microparticle and the resulting materials thereof; which process comprises:
(a) preparing a solution or suspension (organic phase) comprising: (i) PLGA or PLA or PLA and PLGA, (ii) PLGA-PEG or PLA-PEG (iii) a pharmaceutically active compound, for example, as described herein and (iv) one or more organic solvents;
(b) preparing an emulsion in an aqueous polyvinyl alcohol (PVA) solution (aqueous phase) by adding the organic phase into the aqueous phase and mixing them until particle formation (for example at about 3,000 to about 10,000 rpm for about 1 to about 30 minutes);
(c) removing additional solvent as necessary using known techniques;
(d) centrifuging or causing the sedimentation of the microparticle that is loaded with a pharmaceutically active compound or prodrug thereof,
(e) optionally removing additional solvent and/or washing the microparticle loaded with the pharmaceutically active compound or prodrug thereof with water;
(f) filtering the microparticle loaded with pharmaceutically active compound or prodrug thereof to remove aggregates or particles larger than the desired size;
(g) optionally lyophilizing the microparticle comprising the pharmaceutically active compound and storing the microparticle as a dry powder in a manner that maintains stability for up to about 6, 8, 10, 12, 20, 22, or 24 months or more;
(h) resuspending the surface-treated microparticles in an appropriate diluent that comprises additive that improves in vivo particle aggregation;
(i) optionally further improving the aggregation potential of the particles by subjecting the particles to at least one process selected from 1) vacuum treatment; 2) sonication; and 3) vortex.

In one embodiment, step (e) includes washing the microparticle loaded with a pharmaceutically active compound or prodrug with a solution comprising sugar, optionally mannitol.

In one embodiment, the diluent for suspending particles is ProVisc. In one embodiment, the diluent for suspending particles is sodium hyaluronate. In one embodiment, the diluent for suspending particles is hyaluronic acid. In some embodiments, the microparticles are diluted from about 10-fold to about 40-fold, from about 15-fold to about 35-fold, or from about 20-fold to about 25-fold. In some embodiments, the diluent for suspending particles is a 10×-diluted ProVisc (0.1% HA in PBS) solution, a 20×-diluted ProVisc (0.05% HA in PBS) solution, or a 40×-diluted ProVisc (0.025% HA in PBS) solution. In some embodiment, the particles are suspended in the diluent at a concentration of at least about 100 mg/mL, 200 mg/mL, 300 mg/mL, 400 mg/mL, or 500 mg/mL.

In one embodiment, the additive is benzyl alcohol. In one embodiment, the additive is triethyl citrate. In one embodiment, the additive is selected from polyethylene glycol, N-methyl-2-pyrrolidone (NMP), 2-pyrrolidone, and DMSO. In one embodiment, the additive is selected from triacetin, benzyl acetate, benzyl benzoate, and acetyltributyl citrate. In one embodiment, the additive is selected from dibutyl sebacate, dimethylphthalate, tributyl O-acetylcitrate, ethanol, and methanol. In one embodiment, the additive is selected from polysorbate 80, ethyl acetate, propylene carbonate, and isopropyl acetate. In one embodiment, the additive is selected from methyl acetate, methyl ethyl ketone, butyl lactate, and isovaleric acid.

In certain embodiments, the microparticles in step (h) are resuspended in ProVisc comprising benzyl alcohol. In certain embodiments, the microparticles in step (h) are resuspended in ProVisc comprising triethyl citrate. In certain embodiments, the microparticles in step (h) are resuspended in sodium hyaluronate comprising benzyl alcohol. In certain embodiments, the microparticles in step (h) are resuspended in sodium hyaluronate comprising triethyl citrate. In certain embodiments, the microparticles in step (h) are resuspended in hyaluronic acid comprising benzyl alcohol. In certain embodiments, the microparticles in step (h) are resuspended in hyaluronic acid comprising triethyl citrate.

In certain embodiments, the diluent contains approximately from about 0.01% to about 10% by weight of additive, from about 0.01% to about 0.1% by weight of additive, from about 0.05% to about 0.5% by weight of additive, from about 0.1% to about 1.0% by weight of additive, from about 0.1% to about 10% by weight of additive, from about 0.5% to about 5% by weight of additive, from about 0.5% to about 4% by weight of additive, from about 0.5% to about 3% by weight of additive, from about 0.5% to about 2.0% by weight of additive, from about 0.1% to about 0.5% by weight of additive, from about 0.1% to about 0.25% by weight of additive, from about 0.2% to about 2% by weight of additive, or from about 0.01% to about 0.05% by weight of additive.

In one embodiment, a process for preparing an improved suspension of microparticles includes suspending the lyophilized microparticles in a diluent comprising additive and subjecting the particles to vacuum treatment at a pressure of about less than about 500, 400, 300, 200, 150, 100, 75, 50, 40, 35, 34, 33, 32, 31, 30, 29, 28 or 25 Torr for a suitable amount of time to substantially remove air attached to the particles, which in some embodiments can be up to 3, 5, 8, 10, 20, 30, 40, 50, 60, 70, 80, or 90 minutes or up to 2, 3, 4, 5, or 6, 10, 15 or 24 or more hours. In one embodiment, the vacuum treatment is conducted with a VacLock syringe in a size of up to at least 10, 20, 30, or 60 mL.

In certain non-limiting embodiments, the microparticles are vacuumed at a strength of less than 40 Torr for about 3, 5, 8, 10, 20, 30, 45, 60, 75, or 90 minutes. In certain non-limiting embodiments, the microparticles are vacuumed at a strength less than 40 Torr from about 1 to 90 minutes, from about 1 to 60 minutes, from about 1 to 45 minutes, from about 1 to 30 minutes, from about 1 to 15 minutes, or from about 1 to 5 minutes.

### III. Terminology

Although specific terms are employed herein, they are used in a generic and descriptive sense only and not for purposes of limitation. Unless otherwise defined, all technical and scientific terms used herein have the same meaning as commonly understood by one of ordinary skill in the art to which this presently described subject matter belongs.

Compounds are described using standard nomenclature. Unless defined otherwise, all technical and scientific terms used herein have the same meaning as is commonly understood by one of skill in the art to which this invention belongs.

The terms “a” and “an” do not denote a limitation of quantity, but rather denote the presence of at least one of the referenced items.

Recitation of ranges of values are merely intended to serve as a shorthand method of referring individually to each separate value falling within the range, unless otherwise indicated herein, and each separate value is incorporated into the specification as if it were individually recited herein. The endpoints of all ranges are included within the range and are independently combinable. All methods described herein can be performed in a suitable order unless otherwise indicated herein or otherwise clearly contradicted by context. The use of examples, or exemplary language (e.g., “such as”), is intended merely to better illustrate the invention and does not pose a limitation on the scope of the invention unless otherwise claimed.

The term “carrier” refers to a diluent, excipient, or vehicle.

A “dosage form” means a unit of administration of a composition that includes a surface treated microparticle and a therapeutically active compound (i.e, wherein the therapeutically advantageous compound is in the microparticle). Examples of dosage forms include injections, suspensions, liquids, emulsions, implants, particles, spheres, creams, ointments, inhalable forms, transdermal forms, buccal, sublingual, topical, gel, mucosal, and the like. A “dosage form” can also include, for example, a surface treated microparticle comprising a pharmaceutically active compound in a carrier.

The term “microparticle” means a particle whose size is measured in micrometers (m). Typically, the microparticle has an average diameter of from about 1 μm to 100 μm. In some embodiments, the microparticle has an average diameter of from about 1 μm to 60 μm, for instance from about 1 μm to 40 μm; from about 10 μm to 40 μm; from about 20 μm to 40 μm; from about m to 40 μm; from about 25 μm to about 30 μm; from about 20 μm to 35 μm. For example, the microparticle may have an average diameter of from 20 μm to 40 μm, and in certain embodiments, 20, 21, 22, 23, 24, 25, 26, 27, 28, 29, 30, 31, 32 or 33. As used herein, the term “microsphere” means a substantially spherical microparticle.

A “patient” or “host” or “subject” is typically a human, however, may be more generally a mammal. In an alternative embodiment it can refer to, for example, a cow, sheep, goat, horse, dog, cat, rabbit, rat, mouse, bird and the like. Unless otherwise stated, the subject is a human.

The term “mild” or “mildly” when used to describe the surface modification of the microparticles means that the modification (typically the removal, or partial removal, of surfactant from the surface, as opposed to the inner core, of the particle) is less severe, pronounced or extensive than when carried out at room temperature with the otherwise same conditions. In general, the surface modification of the solid microparticles of the present invention is carried out in a manner that does not create significant channels or large pores that would significantly accelerate the degradation of the microparticle in vivo, yet serves to soften and decrease the hydrophilicity of the surface to facilitate in vivo aggregation.

The term “solid” as used to characterize the mildly surface treated microparticle means that the particle is substantially continuous in material structure as opposed to heterogeneous with significant channels and large pores that would undesirably shorten the time of biodegradation.

The term “sonicate” means to subject the microparticle suspension to ultrasonic vibration or high frequency sound waves.

The term “vortex” means to mix by means of a rapid whirling or circular motion.

The term “additive” is used to describe any reagent or solvent that increases the plasticity of a polymer, decreases the viscosity or the glass transition temperature of a polymer, or partially dissolves a polymer. In some embodiments, the additive is a plasticizer. Non-limiting examples of additives of the present invention include triethyl citrate, benzyl alcohol, polyethylene glycol, N-methyl-2-pyrrolidone (NMP), 2-pyrrolidone, DMSO, triacetin, benzyl acetate, benzyl benzoate, acetyltributyl citrate, dibutyl sebacate, dimethylphthalate, tributyl O-acetylcitrate, ethanol, methanol, polysorbate 80, ethyl acetate, propylene carbonate, isopropyl acetate, methyl acetate, methyl ethyl ketone, butyl lactate, and isovaleric acid.

“Hardness,” is a measure of resistance to deformation in units of the gram-force (gf) required to compress the microparticle aggregate at 30% strain. In one embodiment, the aggregated microparticle of the present invention exhibits a hardness of at least about 5 gram-force, at least 10 gram-force, or 15 gram-force, at least about 20 gram-force, or at least about 25 gram-force.

“Durability” is a measurable increase in the ability to significantly withstand environmental damage.

“Gram-force” is a metric unit of force (gf), and is used in this application as a measure of microparticle hardness.

“Aggregated microparticle”, as used herein, is a solid aggregation of individual microparticles wherein the individual microparticles prior to aggregation have a mean diameter between about 10 μm and about 60 microns, and more typically between about 20 and about 40 microns (or between about 15 and about 40 or between about 25 and about 40 microns). The aggregated microparticles of the present invention are distinct from ocular implants or other polymeric inserts that are injected in vivo an already formed shape, and also are distinct from microparticles that are held together by a depot-forming material such as a gel, or other material intended to hold the microparticles together other than the microparticles themselves.

“Light transmittance” is the percentage of light that is transmitted through the solution of microparticles suspended in a diluent, for example hyaluronate solution as described in Example 7. In one embodiment, a solution of microparticles suspended in a diluent has a light transmittance of greater than about 90%, greater than about 92%, greater than about 94%, greater than about 96%, greater than 98%, or greater than 99%.

### IV. Diluents Comprising Additives that Improve Particle Aggregation

In one embodiment, the microparticles are suspended in a diluent of 10× ProVisc-diluted (0.1% HA in PBS) solution comprising additive that improves particle aggregation. In one embodiment, the microparticles are suspended in a diluent of 20×-diluted ProVisc (0.05% HA in PBS) comprising additive that improves particle aggregation. In one embodiment, the microparticles are suspended in a diluent of 40×-diluted ProVisc (0.025% HA in PBS) comprising additive that improves particle aggregation.

Non-limiting examples of additives include triethyl citrate, benzyl alcohol, polyethylene glycol, N-methyl-2-pyrrolidone (NMP), 2-pyrrolidone, DMSO, triacetin, benzyl acetate, benzyl benzoate, acetyltributyl citrate, dibutyl sebacate, dimethylphthalate, tributyl O-acetylcitrate, ethanol, methanol, polysorbate 80, ethyl acetate, propylene carbonate, isopropyl acetate, methyl acetate, methyl ethyl ketone, butyl lactate, and isovaleric acid.

In one embodiment, the microparticles are suspended in a diluent of 10× ProVisc-diluted (0.1% HA in PBS) solution comprising benzyl alcohol. In one embodiment, the microparticles are suspended in a diluent of 20×-diluted ProVisc (0.05% HA in PBS) comprising benzyl alcohol. In one embodiment, the microparticles are suspended in a diluent of 40×-diluted ProVisc (0.025% HA in PBS) comprising benzyl alcohol.

In one embodiment, the microparticles are suspended in a diluent of 10× ProVisc-diluted (0.1% HA in PBS) solution comprising triethyl citrate. In one embodiment, the microparticles are suspended in a diluent of 20×-diluted ProVisc (0.05% HA in PBS) comprising triethyl citrate. In one embodiment, the microparticles are suspended in a diluent of 40×-diluted ProVisc (0.025% HA in PBS) comprising triethyl citrate.

In one embodiment, the particles are suspended in the diluent comprising additive that improves particle aggregation at a concentration of 100 mg/mL, 150 mg/mL, 200 mg/mL, 250 mg/mL, 300 mg/mL, 350 mg/mL, 400 mg/mL, 450 mg/mL or 500 mg/mL. In one embodiment, the particles are suspended in 10×-diluted ProVisc (0.1% HA in PBS) solution comprising additive that improves particle aggregation and the suspension has a final concentration of 200 mg/mL. In one embodiment, the particles are suspended in 10×-diluted ProVisc (0.1% HA in PBS) solution comprising additive that improves particle aggregation and the suspension has a final concentration of 400 mg/mL. In one embodiment, the particles are suspended in a 20×-diluted ProVisc (0.05% HA in PBS) solution comprising additive that improves particle aggregation and the suspension has a final concentration of 200 mg/mL. In one embodiment, the particles are suspended in a 20×-diluted ProVisc (0.05% HA in PBS) solution comprising additive that improves particle aggregation and the suspension has a final concentration of 400 mg/mL. In one embodiment, the particles are suspended in a 40×-diluted ProVisc (0.025% HA in PBS) solution comprising additive that improves particle aggregation and the suspension has a concentration of 200 mg/mL. In one embodiment, the particles are suspended in a 40×-diluted ProVisc (0.025% HA in PBS) solution comprising additive that improves particle aggregation and the suspension has a concentration of 400 mg/mL.

In certain embodiments, the diluent for suspending particles is ProVisc comprising additive that improves particle aggregation. In one embodiment, the diluent for suspending particles is sodium hyaluronate comprising additive that improves particle aggregation. In some embodiments, the microparticles are diluted from about 10-fold to about 40-fold, from about 15-fold to about 35-fold, or from about 20-fold to about 25-fold. In some embodiments, the diluent for suspending particles is a 10×-diluted ProVisc (0.1% HA in PBS) solution, a 20×-diluted ProVisc (0.05% HA in PBS) solution, or a 40×-diluted ProVisc (0.025% HA in PBS) solution comprising additive. In some embodiment, the particles are suspended in the diluent comprising additive at a concentration of at least about 100 mg/mL, 200 mg/mL, 300 mg/mL, 400 mg/mL, or 500 mg/mL. In further embodiments, the additive is benzyl alcohol. In further embodiments, the additive is triethyl citrate. In some embodiments, the diluent comprises more than one additive, for example benzyl alcohol and triethyl citrate.

In one embodiment, the additive is benzyl alcohol. In one embodiment, the additive is triethyl citrate. In one embodiment, the additive is selected from polyethylene glycol, N-methyl-2-pyrrolidone (NMP), 2-pyrrolidone, and DMSO. In one embodiment, the additive is selected from triacetin, benzyl acetate, benzyl benzoate, and acetyltributyl citrate. In one embodiment, the additive is selected from dibutyl sebacate, dimethylphthalate, tributyl O-acetylcitrate, ethanol, and methanol. In one embodiment, the additive is selected from polysorbate 80, ethyl acetate, propylene carbonate, and isopropyl acetate. In one embodiment, the additive is selected from methyl acetate, methyl ethyl ketone, butyl lactate, and isovaleric acid.

In certain embodiments, the diluent contains approximately from about 0.01% to about 10% by weight of additive, from about 0.01% to about 0.1% by weight of additive, from about 0.05% to about 0.5% by weight of additive, from about 0.1% to about 1.0% by weight of additive, from about 0.1% to about 10% by weight of additive, from about 0.5% to about 5% by weight of additive, from about 0.5% to about 4% by weight of additive, from about 0.5% to about 3% by weight of additive, from about 0.5% to about 2.0% by weight of additive, from about 0.1% to about 0.5% by weight of additive, from about 0.1% to about 0.25% by weight of additive, from about 0.2% to about 2% by weight of additive, or from about 0.01% to about 0.05% by weight of additive.

The diluent is present in an amount in a range of from about 0.5 wt percent to about 95 wt percent of the drug delivery particles. The diluent can also be an aqueous diluent. Examples of aqueous diluent include, but are not limited to, an aqueous solution or suspension, such as saline, plasma, bone marrow aspirate, buffers, such as Hank's Buffered Salt Solution (HBSS), HEPES (4-(2-hydroxyethyl)-1-piperazineethanesulfonic acid), Ringers buffer, ProVisc®, diluted ProVisc®, ProVisc® diluted with PBS, Krebs buffer, Dulbecco's PBS, normal PBS; sodium hyaluronate solution (HA, 5 mg/mL in PBS), simulated body fluids, plasma platelet concentrate and tissue culture medium or an aqueous solution or suspension comprising an organic solvent. ProVisc® is a sterile, non-pyrogenic, high molecular weight, non-inflammatory highly purified fraction of sodium hyaluronate, dissolved in physiological sodium chloride phosphate buffer.

In one embodiment, the diluent is PBS.

In one embodiment, the diluent is HA, 5 mg/mL in PBS.

In one embodiment, the diluent is ProVisc® diluted with water.

In one embodiment, the diluent is ProVisc® dilution in PBS.

In one embodiment, the diluent is ProVisc® 5-fold diluted with water.

In one embodiment, the diluent is ProVisc® 5-fold dilution in PBS.

In one embodiment, the diluent is ProVisc® 10-fold diluted with water.

In one embodiment, the diluent is ProVisc® 10-fold dilution in PBS.

In one embodiment, the diluent is ProVisc® 20-fold dilution with water.

In one embodiment, the diluent is ProVisc® 20-fold dilution in PBS.

In one embodiment, the diluent is HA, 1.25 mg/mL in an isotonic buffer solution with neutral pH.

In one embodiment, the diluent is HA, 0.625 mg/mL in an isotonic buffer solution with neutral pH.

In one embodiment, the diluent is HA, 0.1-5.0 mg/mL in PBS.

In one embodiment, the diluent is HA, 0.5-4.5 mg/mL in PBS.

In one embodiment, the diluent is HA, 1.0-4.0 mg/mL in PBS.

In one embodiment, the diluent is HA, 1.5-3.5 mg/mL in PBS.

In one embodiment, the diluent is HA, 2.0-3.0 mg/mL in PBS.

In one embodiment, the diluent is HA, 2.5-3.0 mg/mL in PBS.

### V. Processes for Producing Improved Suspensions of Surface-Treated Aggregating Microparticles for Therapeutic Purposes

In one embodiment, the present invention provides processes for producing suspensions of surface-treated aggregating microparticles for therapeutic purposes that aggregate in vivo to form pellet(s). The processes includes suspending mildly surface-treated microparticles in a diluent comprising additive that improves in vivo particle aggregation. Optionally, the suspension is also subjected to at least one process selected from 1) vacuum treatment and 2) sonication.

In one embodiment, the invention is thus suspensions of solid aggregating microparticles in diluent comprising additive that improves in vivo particle aggregation wherein the surface-modified solid aggregating microparticles comprise at least one biodegradable polymer, have a solid core, include a therapeutic agent, have a modified surface which has been treated under mild conditions at a temperature at or less than about 18° C. to remove all or some of the surface surfactant or cause surface polymer to partially degrade, are sufficiently small to be injected in vivo, and aggregate in vivo to form at least one pellet of at least 500 μm in vivo in a manner that provides sustained drug delivery in vivo for at least one, two, three, four, five, six or seven months or more. In one embodiment, the suspension of microparticles have been treated by at least one or more processes selected from vacuum treatment and sonication to further improve wettability upon injection. The surface modified solid aggregating microparticles are suitable, for example, for an intravitreal injection, implant, including an ocular implant, periocular delivery or delivery in vivo outside of the eye.

The present invention further includes a process for the preparation of surface-modified solid aggregating microparticles that have also been treated for enhanced wettability that includes
A. a first step of preparing microparticles comprising one or more biodegradable polymers by dissolving or dispersing the polymer(s) and a therapeutic agent in one or more solvents to form a polymer and therapeutic agent solution or dispersion, mixing the polymer and the therapeutic agent solution or dispersion with an aqueous phase containing a surfactant to produce solvent-laden microparticles and then removing the solvent(s) to produce polymer microparticles that contain the therapeutic agent, polymer and surfactant; and
B. a second step of mildly treating the surface of microparticles of step (i) at a temperature at or below about 18, 15, 10, 8 or 5° C. optionally up to about 1, 2, 3, 4, 5, 10, 30, 40, 50, 60, 70, 80, 90 100, 11, 120 or 140 minutes with an agent that removes or partially removes surface surfactant, surface polymer, or surface oligomer in a manner that does not significantly produce internal pores;
C. washing the microparticles with a solution comprising an excipient, optionally mannitol;
D. isolating and lyophilizing the surface-treated microparticles;
E. resuspending the surface-treated microparticles in an appropriate diluent that comprises additive that improves in vivo particle aggregation;
F. further improving the aggregation potential of the particles by subjecting the particles to at least one process selected from 1) vacuum treatment and 2) sonication.

In certain embodiments step (ii) above is carried out at a temperature below 17° C., 15° C., 10° C., or 5° C. Further, step (iii) is optionally carried out at a temperature below 25° C., below 17° C., 15° C., 10° C., 8° C. or 5° C. Step (ii), for example, can be carried out for less than 8, less than 6, less than 4, less than 3, less than 2, or less than 1 minutes. In one embodiment, step (ii) is carried out for less than 60, 50, 40, 30, 20, or 10 minutes.

In one embodiment, step (ii) above is carried out for a time of about less than 140, 120, 110, 100, 90, 60, 40, 30, 20, 10, 3, 2, or 1 minutes.

### VI. Mildly Surface Treated Aggregating Microparticles and Methods

The improved microparticle suspensions are made from mildly surface-treated solid biodegradable microparticles that upon injection in vivo, aggregate to a larger particle (pellet) in a manner that reduces unwanted side effects of the smaller particles and are suitable for long term (for example, up to or at least three month, up to four month, up to five month, up to six months, up to seven months, up to eight months, up to nine months or longer) sustained delivery of a therapeutic agent. In one embodiment, the lightly surface treated solid biodegradable microparticles are suitable for ocular injection, at which point the particles aggregate to form a pellet and thus remains outside the visual axis as not to significantly impair vision. The particles can aggregate into one or several pellets. The size of the aggregate depends on the mass (weight) of the particles injected.

The mildly surface treated biodegradable microparticles provided herein are distinguished from “scaffold” microparticles, which are used for tissue regrowth via pores that cells or tissue material can occupy. In contrast, the present microparticles are designed to be solid materials of sufficiently low porosity so that they can aggregate to form a larger combined particle that erodes primarily by surface erosion for long-term controlled drug delivery.

The surface modified solid aggregating microparticles of the present invention are suitable, for example, for intravitreal injection, implant, periocular delivery, or delivery in vivo outside the eye.

The surface modified solid aggregating microparticles of the present invention are also suitable for systemic, parenteral, transmembrane, transdermal, buccal, subcutaneous, endosinusial, intra-abdominal, intra-articular, intracartilaginous, intracerebral, intracoronal, dental, intradiscal, intramuscular, intratumor, topical, or vaginal delivery in any manner useful for in vivo delivery.

In one embodiment, the invention is thus a suspension of surface-modified solid aggregating microparticles that include at least one biodegradable polymer in a diluent comprising additive that improves in vivo particle aggregation, wherein the surface-modified solid aggregating microparticles have a solid core, include a therapeutic agent, have a modified surface which has been treated under mild conditions at a temperature at or less than about 18° C. to remove, or partially remove, surface surfactant or cause surface polymer to partially degrade, are sufficiently small to be injected in vivo, and aggregate in vivo to form at least one pellet of at least 500 μm in vivo in a manner that provides sustained drug delivery in vivo for at least one, two, three, four, five, six seven, eight, nine, ten, eleven, twelve months or more. The surface modified solid aggregating microparticles are suitable, for example, for an intravitreal injection, implant, including an ocular implant, periocular delivery or delivery in vivo outside of the eye. In certain embodiments, the therapeutic agent is a prodrug as described herein. In certain embodiments, microparticles have also been treated for enhanced wettability by subjecting the microparticle suspensions to vacuum or sonication.

In some embodiments, the surface treatment is carried out at a temperature of not more than 5, 6, 7, 8, 9, 10, 11, 12, 13, 14, 15, 16, 17 or 18° C., at a reduced temperature of about 5 to about 18° C., about 5 to about 16° C., about 5 to about 15° C., about 0 to about 10° C., about 0 to about 8° C., or about 1 to about 5° C., about 5 to about 20° C., about 1 to about 10° C., about 0 to about 15° C., about 0 to about 10° C., about 1 to about 8° C., or about 1 to about 5° C. Each combination of each of these conditions is considered independently disclosed as if each combination were separately listed. Alternatively, the surface treatment is conducted at a temperature at or less than about 10° C., 8° C. or 5° C.

The pH of the surface treatment will of course vary based on whether the treatment is carried out in basic, neutral or acidic conditions. When carrying out the treatment in base, the pH may range from about 7.5 to about 14, including not more than about 8, 9, 10, 11, 12, 13 or 14. When carrying out the treatment in acid, the pH may range from about 6.5 to about 1, including not less than 1, 2, 3, 4, 5, 6, 7, or 8. When carrying out under neutral conditions, the pH may typically range from about 6.4 or 6.5 to about 7.4 or 7.5. In one embodiment, the surface treatment is carried out a pH from 6.5 to 8.5, from 7.5 to 9.5, or from 8.5-10.5.

The surface treatment can be carried out at any pH that achieves the desired purpose. Non-limiting examples of the pH are between about 6 and about 8, 6.5 and about 7.5, about 1 and about 4; about 4 and about 6; and 6 and about 8. In one embodiment, the surface treatment can be conducted at a pH between about 8 and about 10. In one embodiment, the surface treatment can be conducted at a pH between about 10.0 and about 13.0. In one embodiment, the surface treatment can be conducted at a pH between about 10.0 and about 12.0. In one embodiment, the surface treatment can be conducted at a pH between about 12 and about 14. In one embodiment, the surface treatment can be conducted with an organic solvent. In one embodiment, the surface treatment can be conducted with ethanol. In other various embodiments, the surface treatment is carried out in a solvent selected from methanol, ethyl acetate and ethanol. Non-limiting examples are ethanol with an aqueous organic base; ethanol and aqueous inorganic base; ethanol and sodium hydroxide; ethanol and potassium hydroxide; an aqueous acidic solution in ethanol; aqueous hydrochloric acid in ethanol; and aqueous potassium chloride in ethanol.

In one embodiment, the surface treatment includes treating microparticles with aqueous base, for example, sodium hydroxide and a solvent (such as an alcohol, for example ethanol or methanol, or an organic solvent such as DMF, DMSO or ethyl acetate) as otherwise described above. More generally, a hydroxide base is used, for example, potassium hydroxide. An organic base can also be used. In other embodiments, the surface treatment as described above is carried out in aqueous acid, for example hydrochloric acid. In one embodiment, the surface treatment includes treating microparticles with phosphate buffered saline and ethanol.

A key aspect is that the treatment, whether done in basic, neutral or acidic conditions, includes a selection of the combination of the time, temperature, pH agent and solvent that causes a mild treatment that does not significantly damage the particle in a manner that forms pores, holes or channels. Each combination of each of these conditions described herein is considered independently disclosed as if each combination were separately listed.

The treatment conditions should simply mildly treat the surface in a manner that allows the particles to remain as solid particles, be injectable without undue aggregation or clumping, and form at least one aggregate particle of at least 500 μm. In one embodiment, the treatment partially removes the surface surfactant.

Examples of solid cores included in the present invention include solid cores comprising a biodegradable polymer with less than 10 percent porosity, 8 percent porosity, 7 percent porosity, 6 percent porosity, 5 percent porosity, 4 percent porosity, 3 percent porosity, or 2 percent porosity. Porosity as used herein is defined by ratio of void space to total volume of the surface-modified solid aggregating microparticle.

The surface-modified solid aggregating microparticles of the present invention provides sustained delivery for at least one month, or at least two months, or at least three months, or at least four months, or at least five months, or at least six months, or at least seven months, or at least eight months, or at least nine months, or at least ten months, or at least eleven months, or at least twelve months.

In one embodiment the surface-modified solid aggregating microparticle has a mean diameter between 10 and 60 μm, 20 and 50 μm, 20 and 40 μm, 20 and 30 μm, 25 and 40 μm, or 25 and 35 μm.

Further, the surface-modified solid aggregating microparticles of the disclosed invention can aggregate to produce at least one pellet when administered in vivo that has a diameter of at least about 300 μm, 400 μm, 500 μm, 600 am, 700 am, 1 mm, 1.5 mm, 2 mm, 3 mm, 4 mm, or 5 mm.

In one embodiment, the surface-modified solid aggregating microparticles of the present invention produces a pellet in vivo that releases the therapeutic agent without a burst of more than about 10 percent or 15 percent of the total payload of the therapeutic agent over a one week, or a five, four, three, two day or one day period.

In some embodiments, the long-term controlled drug delivery is accomplished by a combination of surface erosion of an aggregated microparticle over several months (for example, one, two, three, or four months or more) followed by erosion of remaining parts of the aggregated microparticle, followed by slow release of active material from in vivo proteins to which it has bound over the period of long term release from the aggregated particle. In another embodiment, the microparticle degrades substantially by surface erosion over a period of at least about one, two, three, four, five or six months or more.

In another embodiment, the surface-modified solid aggregating microparticles of the present invention have a drug loading of 1-40 percent, 5-25 percent, or 5-15 percent weight/weight.

In one embodiment, the aggregated microparticles of the present invention of at least 500 microns exhibit a hardness rating in vivo in the vitreous of the eye of at least 5 gram-force needed to compress the particle at 30% of strain. In one embodiment, the aggregated microparticles of the present invention of at least 500 microns exhibit a hardness rating in vivo in the vitreous of the eye of at least 10 gram-force needed to compress the particle at 30% of strain. In one embodiment, the aggregated microparticles of the present invention of at least 500 microns exhibit a hardness rating in vivo in the vitreous of the eye of at least 15 gram-force needed to compress the particle at 30% of strain. In one embodiment, the aggregated microparticles of the present invention of at least 500 microns exhibit a hardness rating in vivo in the vitreous of the eye of at least 20 gram-force needed to compress the particle at 30% of strain. In one embodiment, the aggregated microparticles of the present invention of at least 500 microns exhibit a hardness rating in vivo in the vitreous of the eye of at least 25 gram-force needed to compress the particle at 30% of strain. In one embodiment, the aggregated microparticles of the present invention of at least 500 microns exhibit a hardness rating in vivo in the vitreous of the eye of at least 30 gram-force needed to compress the particle at 30% of strain. In one embodiment, the aggregated microparticles of the present invention of at least 500 microns exhibit a hardness rating in vivo in the vitreous of the eye of at least 10 gram-force needed to compress the particle at 35% of strain.

In one embodiment, the hardness of an aggregated microparticle, upon injection in the vitreous, increases at least two-fold in four hours or less, in three hours or less, in two hours or less, in one hour or less, in thirty minutes or less, in fifteen minutes or less, in ten minutes or less, or in five minutes, or in two minutes or less following injection compared to microparticles administered immediately after injection (for example, less than one minute or even 30 seconds after administration).

In one embodiment, the hardness of an aggregated microparticle, upon injection in the vitreous, increases at least three-fold in four hours or less, in three hours or less, in two hours or less, in one hour or less, in thirty minutes or less, in fifteen minutes or less, in ten minutes or less, or in five minutes, or in two minutes or less following injection compared to microparticles administered immediately after injection (for example, less than one minute or even 30 seconds after administration).

In one embodiment, the hardness of an aggregated microparticle, upon injection in the vitreous, increases at least four-fold in four hours or less, in three hours or less, in two hours or less, in one hour or less, in thirty minutes or less, in fifteen minutes or less, in ten minutes or less, or in five minutes, or in two minutes or less following injection compared to microparticles administered immediately after injection (for example, less than one minute or even 30 seconds after administration).

In one embodiment, the hardness of an aggregated microparticle, upon injection in the vitreous, increases at least five-fold in four hours or less, in three hours or less, in two hours or less, in one hour or less, in thirty minutes or less, in fifteen minutes or less, in ten minutes or less, or in five minutes, or in two minutes or less following injection compared to microparticles administered immediately after injection (for example, less than one minute or even 30 seconds after administration).

In one embodiment, the hardness of an aggregated microparticle, upon injection in the vitreous, increases at least six-fold in four hours or less, in three hours or less, in two hours or less, in one hour or less, in thirty minutes or less, in fifteen minutes or less, in ten minutes or less, or in five minutes, or in two minutes or less following injection compared to microparticles administered immediately after injection (for example, less than one minute or even 30 seconds after administration).

In one embodiment, the hardness of an aggregated microparticle, upon injection in the vitreous, increases at least seven-fold in four hours or less, in three hours or less, in two hours or less, in one hour or less, in thirty minutes or less, in fifteen minutes or less, in ten minutes or less, or in five minutes, or in two minutes or less following injection compared to microparticles administered immediately after injection (for example, less than one minute or even 30 seconds after administration).

In one embodiment, the hardness of an aggregated microparticle, upon injection in the vitreous, increases at least eight-fold in four hours or less, in three hours or less, in two hours or less, in one hour or less, in thirty minutes or less, in fifteen minutes or less, in ten minutes or less, or in five minutes, or in two minutes or less following injection compared to microparticles administered immediately after injection (for example, less than one minute or even 30 seconds after administration).

In one embodiment, the hardness of an aggregated microparticle, upon injection in the vitreous, increases at least nine-fold in four hours or less, in three hours or less, in two hours or less, in one hour or less, in thirty minutes or less, in fifteen minutes or less, in ten minutes or less, or in five minutes, or in two minutes or less following injection compared to microparticles administered immediately after injection (for example, less than one minute or even 30 seconds after administration).

In one embodiment, the hardness of an aggregated microparticle, upon injection in the vitreous, increases at least ten-fold in four hours or less, in three hours or less, in two hours or less, in one hour or less, in thirty minutes or less, in fifteen minutes or less, in ten minutes or less, or in five minutes, or in two minutes or less following injection compared to microparticles administered immediately after injection (for example, less than one minute or even 30 seconds after administration).

Examples of polymeric compositions included in surface-modified solid aggregating microparticles of the present invention include, but are not limited to: poly(lactide co-glycolide); poly(lactide-co-glycolide) covalently linked to polyethylene glycol; more than one biodegradable polymer or copolymer mixed together, for example, a mixture of poly(lactide-co-glycolide) and poly(lactide-co-glycolide) covalently linked to polyethylene glycol, a mixture of poly(lactic acid) and poly(lactide-co-glycolide) covalently linked to polyethylene glycol, or a mixture of poly(lactic acid), poly(lactide-co-glycolide) and poly(lactide-co-glycolide) covalently linked to polyethylene glycol; poly(lactic acid); a surfactant, such as polyvinyl alcohol (which can be hydrolyzed polyvinyl acetate).

In one embodiment, the diluent has a range of concentration of the surface-modified solid aggregating microparticles of about 50-700 mg/ml, 500 or less mg/ml, 400 or less mg/ml, 300 or less mg/ml, 200 or less mg/ml, or 150 or less mg/ml.

In one embodiment, the surface treatment includes treating microparticles with an aqueous solution of pH=6.6 to 7.4 or 7.5 and ethanol at a reduced temperature of about 1 to about 10° C., about 1 to about 15° C., about 5 to about 15° C., or about 0 to about 5° C. In one embodiment, the surface treatment includes treating microparticles with an aqueous solution of pH=6.6 to 7.4 or 7.5 and an organic solvent at a reduced temperature of about 0 to about 10° C., about 5 to about 8° C., or about 0 to about 5° C. In one embodiment, the surface treatment includes treating microparticles with an aqueous solution of pH=1 to 6.6 and ethanol at a reduced temperature of about 0 to about 10° C., about 0 to about 8° C., or about 0 to about 5° C. In one embodiment, the surface treatment includes treating microparticles with an organic solvent at a reduced temperature of about 0 to about 18° C., about 0 to about 16° C., about 0 to about 15° C., about 0 to about 10° C., about 0 to about 8° C., or about 0 to about 5° C. The decreased temperature of processing (less than room temperature, and typically less than 18° C.) assists to ensure that the particles are only “mildly” surface treated.

In one embodiment, the process of manufacturing surface-modified solid aggregating microparticles includes using an agent that removes or partially removes surface surfactant. Non-limiting examples include for example, those selected from: aqueous acid, phosphate buffered saline, water, aqueous NaOH, aqueous hydrochloric acid, aqueous potassium chloride, alcohol or ethanol.

In one embodiment, the process of manufacturing surface-modified solid aggregating microparticles includes using an agent that removes surface surfactant which comprises, for example, a solvent selected from an alcohol, for example, ethanol; ether, acetone, acetonitrile, DMSO, DMF, THF, dimethylacetamide, carbon disulfide, chloroform, 1,1-dichloroethane, dichloromethane, ethyl acetate, heptane, hexane, methanol, methyl acetate, methyl t-butyl ether (MTBE), pentane, propanol, 2-propanol, toluene, N-methyl pyrrolidinone (NMP), acetamide, piperazine, triethylenediamine, diols, and CO₂.

The agent that removes the surface surfactant can comprise a basic buffer solution. Further, the agent that removes surface surfactant can comprise a base selected from sodium hydroxide, lithium hydroxide, potassium hydroxide, calcium hydroxide, magnesium hydroxide, lithium amide, sodium amide, barium carbonate, barium hydroxide, barium hydroxide hydrate, calcium carbonate, cesium carbonate, cesium hydroxide, lithium carbonate, magnesium carbonate, potassium carbonate, sodium carbonate, strontium carbonate, ammonia, methylamine, ethylamine, propylamine, isopropylamine, dimethylamine, diethylamine, dipropylamine, diisopropylamine, trimethylamine, triethylamine, tripropylamine, triisopropylamine, aniline, methylaniline, dimethylaniline, pyridine, azajulolidine, benzylamine, methylbenzylamine, dimethylbenzylamine, DABCO, 1,5-diazabicyclo[4.3.0]non-5-ene, 1,8-diazabicyclo[5.4.0]non-7-ene, 2,6-lutidine, morpholine, piperidine, piperazine, Proton-sponge, 1,5,7-Triazabicyclo[4.4.0]dec-5-ene, tripelennamine, ammonium hydroxide, triethanolamine, ethanolamine, and Trizma.

In one embodiment, the process of manufacturing surface-modified solid aggregating microparticles includes using an agent that removes surface surfactant, for example, those selected from the following: aqueous acid, phosphate buffered saline, water, or NaOH in the presence of a solvent such as an alcohol, for example, ethanol, ether, acetone, acetonitrile, DMSO, DMF, THF, dimethylacetamide, carbon disulfide, chloroform, 1,1-dichloroethane, dichloromethane, ethyl acetate, heptane, hexane, methanol, methyl acetate, methyl t-butyl ether (MTBE), pentane, ethanol, propanol, 2-propanol, toluene, N-methyl pyrrolidinone (NMP), acetamide, piperazine, triethylenediamine, diols, and CO₂.

In one embodiment, the agent that removes the surface surfactant can comprise an aqueous acid. The agent that removes the surface surfactant can comprise an acid derived from inorganic acids including, but not limited to, hydrochloric, hydrobromic, sulfuric, sulfamic, phosphoric, nitric and the like; or organic acids including, but not limited to, acetic, propionic, succinic, glycolic, stearic, lactic, malic, tartaric, citric, ascorbic, pamoic, maleic, hydroxymaleic, phenylacetic, glutamic, benzoic, salicylic, mesylic, esylic, besylic, sulfanilic, 2-acetoxybenzoic, fumaric, toluenesulfonic, methanesulfonic, ethane disulfonic, oxalic, isethionic, HOOC—(CH₂)ₙ—COOH where n is 0-4, and the like.

In one embodiment, the agent that removes surface surfactant is not a degrading agent of the biodegradable polymer under the conditions of the reaction. The hydrophilicity of the microparticles can be decreased by removing surfactant.

In one embodiment, the process of manufacturing surface-modified solid aggregating microparticles comprises using an agent that removes surface surfactant that comprises a solvent selected from an alcohol, for example, ethanol, ether, acetone, acetonitrile, DMSO, DMF, THF, dimethylacetamide, carbon disulfide, chloroform, 1,1-dichloroethane, dichloromethane, ethyl acetate, heptane, hexane, methanol, methyl acetate, methyl t-butyl ether (MTBE), pentane, ethanol, propanol, 2-propanol, toluene, N-methyl pyrrolidinone (NMP), acetamide, piperazine, triethylenediamine, diols, and CO₂. In a typical embodiment the process of surface treating, comprises an agent that removes surface surfactant that comprises ethanol.

The encapsulation efficiency of the process of manufacturing surface-modified solid aggregating microparticles depends on the microparticle forming conditions and the properties of the therapeutic agent. In certain embodiments, the encapsulation efficiency can be greater than about 50 percent, greater than about 75 percent, greater than about 80 percent, or greater than about 90 percent.

In one embodiment, the process of manufacturing surface-modified solid aggregating microparticles includes 5/95, 10/90, 15/85, 20/80, 25/75, 30/70, 35/65, 40/60, 45/55, 50/50, 55/45, 60/40, 65/35, 70/30, 75/25, 80/20, 85/15, 90/10, 95/5 PLGA as a biodegradable polymer. In one embodiment, the process of manufacturing surface-modified solid aggregating microparticles includes 50/50 PLGA as a biodegradable polymer.

In one embodiment, the process of manufacturing surface-modified solid aggregating microparticles includes PLA as a biodegradable polymer. In one embodiment, the process of manufacturing surface-modified solid aggregating microparticles includes PLA and PLGA as a biodegradable polymer. In one embodiment, the process of manufacturing surface-modified solid aggregating microparticles includes PLA and 75/25 PLGA as a biodegradable polymer. In one embodiment, the process of manufacturing surface-modified solid aggregating microparticles includes PLA and 50/50 PLGA as a biodegradable polymer. In one embodiment, the process of manufacturing surface-modified solid aggregating microparticles includes PLGA as a biodegradable polymer.

In one embodiment, the process of manufacturing surface-modified solid aggregating microparticles is carried out below about a pH of 14 and above a pH of 12, below a pH of 12 and above a pH of 10, below a pH of about 10 and above a pH of 8, below about a pH of 8 and above a pH of about 6, neutral pH, below about a pH of 7 and above a pH of 4, below about a pH of 4 and above a pH of about 1.0.

In yet another embodiment, a method for the treatment of an ocular disorder is provided that includes administering to a host in need thereof a suspension that comprises additive that improves in vivo particle aggregation and solid aggregating microparticles described herein that include an effective amount of a therapeutic agent, wherein the solid aggregating microparticles are injected into the eye and aggregate in vivo to form at least one pellet of at least 500 μm that provides sustained drug delivery for at least approximately one, two, three, four, five, six, seven, eight, nine, ten, eleven, twelve or more months in such a manner that the pellet stays substantially outside the visual axis so as not to significantly impair vision. In one embodiment, the solid biodegradable microparticles release about 1 to about 20 percent, about 1 to about 15 percent, about 1 to about 10 percent, or about 5 to 20 percent, for example, up to about 1, 5, 10, 15 or 20 percent, of the therapeutic agent over the first twenty-four-hour period.

In an alternative embodiment, the weight percent of surface-modified solid aggregating microparticles that are not aggregated into a larger pellet in vivo is about 10 percent or less, 7 percent or less, 5 percent or less, or 2 percent or less by total weight administered.

In one embodiment, the surface-modified solid aggregating microparticles do not cause substantial inflammation in the eye.

In another embodiment, the surface-modified solid aggregating microparticles do not cause an immune response in the eye.

In one embodiment, the surface-modified solid aggregating microparticles are capable of releasing a therapeutic agent over a longer period of time compared to a non-surface treated microparticle.

In one embodiment, the surface-modified solid aggregating microparticles contain less surfactant than the microparticles prior to the surface modification.

In one embodiment, the surface-modified solid aggregating microparticles are more hydrophobic than the microparticles prior to the surface modification.

In one embodiment, the surface-modified microparticles of the present invention, as described herein, are used to treat a medical disorder which is glaucoma, a disorder mediated by carbonic anhydrase, a disorder or abnormality related to an increase in intraocular pressure (IOP), a disorder mediated by nitric oxide synthase (NOS), or a disorder requiring neuroprotection such as to regenerate/repair optic nerves. In another embodiment more generally, the disorder treated is allergic conjunctivitis, anterior uveitis, cataracts, wet or dry age-related macular degeneration, neovascular age-related macular degeneration, or diabetic retinopathy.

Another embodiment is provided that includes the administration of suspension of surface treated microparticle comprising an effective amount of a pharmaceutically active compound or a pharmaceutically acceptable salt thereof, in a diluent comprising additive that improves in vivo particle aggregation to a host to treat an ocular or other disorder that can benefit from topical or local delivery. The therapy can be delivered to the anterior or posterior chamber of the eye. In specific aspects, a surface treated microparticle comprising an effective amount of a pharmaceutically active compound is administered to treat a disorder of the cornea, conjunctiva, aqueous humor, iris, ciliary body, lens sclera, choroid, retinal pigment epithelium, neural retina, optic nerve, or vitreous humor.

Any of the compositions described can be administered to the eye as described further herein in any desired form of administration, including via intravitreal, intrastromal, intracameral, subtenon, sub-retinal, retrobulbar, peribulbar, suprachoroidal, subchoroidal, conjunctival, subconjunctival, episcleral, posterior juxtascleral, circumcorneal, tear duct injections, or through a mucus, mucin, or a mucosal barrier, in an immediate or controlled release fashion.

The therapeutic agent delivered by the surface-modified solid aggregating microparticle is in one embodiment a pharmaceutical drug or a biologic. In nonlimiting examples, the pharmaceutical drugs include sunitinib, another tyrosine kinase inhibitor, an anti-inflammatory drug, an antibiotic, an immunosuppressing agent, an anti-VEGF agent, an anti-PDGF agent, or other therapeutic agents as described below. In one embodiment, the tyrosine kinase inhibitor is selected from Tivosinib, Imatinib, Gefitinib, Erlotinib, Lapatinib, Canertinib, Semaxinib, Vatalaninib, Sorafenib, Axitinib, Pazopanib, Dasatinib, Nilotinib, Crizotinib, Ruxolitinib, Vandetanib, Vemurafenib, Bosutinib, Cabozantinib, Regorafenib, Vismodegib, and Ponatinib. In one embodiment, the pharmaceutical drug is selected from Atropine, Pilocarpine, and Alpha lipoic acid or a pharmaceutically acceptable salt thereof.

In one embodiment, the disclosure provides a beta-adrenergic antagonist for ocular therapy, which can be released from a surface treated microparticle while maintaining efficacy over an extended time such as up to at least 1, 2, 3, 4, 5, 6, 7, 8, 9, 10, 11, or 12 months.

In one embodiment, the disclosure provides a prostaglandin or a prodrug thereof for ocular therapy, which can be released from a surface treated microparticle while maintaining efficacy over an extended time such as up to at least 1, 2, 3, 4, 5, 6, 7, 8, 9, 10, 11, or 12 months.

In one embodiment, the disclosure provides an adrenergic agonist for ocular therapy, which can be released from a surface treated microparticle while maintaining efficacy over an extended time such as up to at least 1, 2, 3, 4, 5, 6, 7, 8, 9, 10, 11, or 12 months.

In one embodiment, the disclosure provides a carbonic anhydrase inhibitor for ocular therapy, which can be released from a surface treated microparticle while maintaining efficacy over an extended time such as up to at least 1, 2, 3, 4, 5, 6, 7, 8, 9, 10, 11, or 12 months.

In one embodiment, the disclosure provides a parasympathomimetic agent for ocular therapy, which can be released from a surface treated microparticle while maintaining efficacy over an extended time such as up to at least 1, 2, 3, 4, 5, 6, 7, 8, 9, 10, 11, or 12 months.

In one embodiment, the disclosure provides a dual anti-VEGF/anti-PDGF agent for ocular therapy, which can be released from a surface treated microparticle while maintaining efficacy over an extended time such as up to at least 1, 2, 3, 4, 5, 6, 7, 8, 9, 10, 11, or 12 months.

In one embodiment, the disclosure provides a loop diuretic for ocular therapy, which can be released from a surface treated microparticle while maintaining efficacy over an extended time such as up to at least 1, 2, 3, 4, 5, 6, 7, 8, 9, 10, 11, or 12 months.

In one embodiment, the disclosure provides a Rho kinase (ROCK) inhibitor for ocular therapy, which can be released from a surface treated microparticle while maintaining efficacy over an extended time such as up to at least 1, 2, 3, 4, 5, 6, 7, 8, 9, 10, 11, or 12 months.

In one embodiment, the disclosure provides a prodrug as described herein for ocular therapy, which can be released from a surface treated microparticle while maintaining efficacy over an extended time such as up to at least 1, 2, 3, 4, 5, 6, 7, 8, 9, 10, 11, or 12 months.

Methods of treating or preventing ocular disorders, including glaucoma, myopia, presbyopia, a disorder mediated by carbonic anhydrase, a disorder or abnormality related to an increase in intraocular pressure (IOP), a disorder mediated by nitric oxide synthase (NOS), a disorder requiring neuroprotection such as to regenerate/repair optic nerves, allergic conjunctivitis, anterior uveitis, cataracts, dry or wet age-related macular degeneration (AMD) or diabetic retinopathy are disclosed comprising administering a therapeutically effective amount of a surface treated microparticle comprising a pharmaceutically active compound to a host, including a human, in need of such treatment. In one embodiment, the host is a human.

In another embodiment, an effective amount of a surface treated microparticle comprising a pharmaceutically active compound is provided to decrease intraocular pressure (IOP) caused by glaucoma. In an alternative embodiment, the surface treated microparticle comprising a pharmaceutically active compound can be used to decrease intraocular pressure (IOP), regardless of whether it is associated with glaucoma.

In one embodiment, the disorder is associated with an increase in intraocular pressure (IOP) caused by potential or previously poor patient compliance to glaucoma treatment. In yet another embodiment, the disorder is associated with potential or poor neuroprotection through neuronal nitric oxide synthase (NOS). The surface treated microparticle comprising a pharmaceutically active compound provided herein may thus dampen or inhibit glaucoma in a host, by administration of an effective amount in a suitable manner to a host, typically a human, in need thereof.

Methods for the treatment of a disorder associated with glaucoma, increased intraocular pressure (IOP), optic nerve damage caused by either high intraocular pressure (IOP) or neuronal nitric oxide synthase (NOS) are provided that includes the administration of an effective amount of a surface treated microparticle comprising a pharmaceutically active compound are also disclosed.

In one aspect of the present invention, an effective amount of a pharmaceutically active compound as described herein is incorporated into a surface treated microparticle, e.g., for convenience of delivery and/or sustained release delivery. The use of materials in micrometer scale provides one the ability to modify fundamental physical properties such as solubility, diffusivity, and drug release characteristics. These micrometer scale agents may provide more effective and/or more convenient routes of administration, lower therapeutic toxicity, extend the product life cycle, and ultimately reduce healthcare costs. As therapeutic delivery systems, surface treated microparticles can allow targeted delivery and sustained release.

In another aspect of the present invention, the surface treated microparticle is coated with a surface agent. The present invention further comprises a method of producing surface treated microparticles comprising a pharmaceutically active compound. The present invention further comprises methods of using the surface treated microparticles comprising a pharmaceutically active compound to treat a patient.

In one embodiment, surface treated microparticles including a pharmaceutically active compound are obtained by forming an emulsion and using a bead column as described in, for example, U.S. Pat. No. 8,916,196.

In one embodiment, surface treated microparticles including a pharmaceutically active compound are obtained by using a vibrating mesh or microsieve.

In one embodiment, surface treated microparticles including a pharmaceutically active compound are obtained by using slurry sieving.

The processes of producing microspheres described herein are amenable to methods of manufacture that narrow the size distribution of the resultant particles. In one embodiment, the particles are manufactured by a method of spraying the material through a nozzle with acoustic excitation (vibrations) to produce uniform droplets. A carrier stream can also be utilized through the nozzle to allow further control of droplet size. Such methods are described in detail in: Berkland, C., K. Kim, et al. (2001). “Fabrication of PLG microspheres with precisely controlled and monodisperse size distributions.” J Control Release 73(1): 59-74; Berkland, C., M. King, et al. (2002). “Precise control of PLG microsphere size provides enhanced control of drug release rate.” J Control Release 82(1): 137-147; Berkland, C., E. Pollauf, et al. (2004). “Uniform double-walled polymer microspheres of controllable shell thickness.” J Control Release 96(1): 101-111.

In another embodiment, microparticles of uniform size can be manufactured by methods that utilize microsieves of the desired size. The microsieves can either be used directly during production to influence the size of microparticles formed, or alternatively post production to purify the microparticles to a uniform size. The microsieves can either be mechanical (inorganic material) or biological in nature (organic material such as a membrane). One such method is described in detail in U.S. Pat. No. 8,100,348.

In one embodiment, the surface treated microparticles comprise a therapeutically active compound and have a particle size of 25<Dv50<40 μm, Dv90<45 μm.

In one embodiment, the surface treated microparticles comprise a therapeutically active compound and have a particle size of Dv10>10 μm.

In one embodiment, the surface treated microparticles comprise a therapeutically active compound and have only residual solvents that are pharmaceutically acceptable.

In one embodiment, the surface treated microparticles comprise a therapeutically active compound and afford a total release of greater than eighty percent by day 14.

In one embodiment, the surface treated microparticles comprise a therapeutically active agent and have syringeability with a regular-walled 26-, 27-, 28-, 29- or 30-gauge needle of 200 mg/ml with no clogging of the syringe.

In one embodiment, the surface treated microparticles comprise a therapeutically active agent and have syringeability with a thin-walled 26-, 27-, 28-, 29- or 30-gauge needle of 200 mg/ml with no clogging of the syringe.

In one embodiment, the surface treated microparticles comprises sunitinib have a particle size of 25<Dv50<40 μm, Dv90<45 μm.

In one embodiment, the surface treated microparticles comprising sunitinib have a particle size of Dv10>10 μm.

In one embodiment, the surface treated microparticles comprising sunitinib have only residual solvents that are pharmaceutically acceptable.

In one embodiment, the surface treated microparticles comprising sunitinib afford a total release of greater than eighty percent by day 14.

In one embodiment, the surface treated microparticles comprising sunitinib have syringeability with a regular-walled 26-, 27-, 28-, 29- or 30-gauge needle of 200 mg/ml with no clogging of the syringe.

In one embodiment, the surface treated microparticles comprising sunitinib have syringeability with a thin-walled 26-, 27-, 28-, 29- or 30-gauge needle of 200 mg/ml with no clogging of the syringe.

In one embodiment, the surface treated microparticles comprising sunitinib have an endotoxin level of less than 0.02 EU/mg.

In one embodiment, the surface treated microparticles comprising sunitinib have a bioburden level of less than 10 CFU/g.

### VI. The Addition of an Excipient During Washing

In one embodiment, the process for preparing an improved microparticle suspension prior to injection is the addition of at least one excipient via washing, typically prior to lyophilization, that reduces the amount of air adhering to the particles. In one embodiment, the particles are suspended in an aqueous sugar solution that is about 1%, 2%, 3%, 4%, 5%, 6%, 7%, 8%, 9%, 10%, 11%, 12%, 13%, 14%, or 15% sugar. In one embodiment, the sugar is sucrose. In one embodiment, the sugar is mannitol. In one embodiment, the sugar is trehalose. In one embodiment, the sugar is glucose. In one embodiment, the sugar is selected from arabinose, fucose, mannose, rhamnose, xylose, D-xylose, glucose, fructose, ribose, D-ribose, galactose, dextrose, dextran, lactose, maltodextrin, maltose, glycerol, erythritol, threitol, arabitol, xylitol, ribitol, sorbitol, galactitol, fucitol, iditol, inositol, volemitol, isomalt, maltitol, lactitol, maltotriitol, maltotetraitol, and polyglycitol. In an alternative embodiment, the sugar is selected from aspartame, saccharin, stevia, sucralose, acesulfame potassium, advantame, alitame, neotame, and sucralose. In one embodiment, the particles are suspended in an aqueous sugar solution that is about 1%, 2%, 3%, 4%, 5%, 6%, 7%, 8%, 9%, 10%, 11%, 12%, 13%, 14%, or 15% sucrose. In one embodiment, the particles are suspended in a 1% sucrose solution. In one embodiment, the particles are suspended in a 10% sucrose solution. In one embodiment, the particles are suspended in an aqueous sugar solution that is about 1%, 2%, 3%, 4%, 5%, 6%, 7%, 8%, 9%, 10%, 11%, 12%, 13%, 14%, or 15% mannitol. In one embodiment, the particles are suspended in a 1% mannitol solution. In one embodiment, the particles are suspended in a 10% mannitol solution. In one embodiment, the particles are suspended in a 1% trehalose solution. In one embodiment, the particles are suspended in a 10% trehalose solution. In one embodiment, the particles are suspended in an aqueous sugar solution that is about 1%, 2%, 3%, 4%, 5%, 6%, 7%, 8%, 9%, 10%, 11%, 12%, 13%, 14%, or 15% trehalose. In an alternative embodiment, the particles are suspended in a small surfactant molecule, including, but not limited to tween 20 or tween 80. In one embodiment, particles are suspended in an aqueous solution and sonicated before being flash frozen in −80° C. ethanol and lyophilized overnight. In an alternative embodiment, the particles are flash frozen in −80° C. methanol or isopropanol.

### VIII. Vacuum Treatment

In one embodiment, the process for providing an improved microparticle suspension prior to injection includes vacuum treatment wherein the particles are suspended in a diluent that comprises additive that improves in vivo particle aggregation and subjected to negative pressure to remove unwanted air at the surface of the microparticles. Nonlimiting examples of the negative pressure can be about or less than 300, 200, 100, 150, 145, 143, 90, 89, 88, 87, 86, 85, 75, 50, 35, 34, 33, 32, 31, or 30 Torr for any appropriate time to achieve the desired results, including but not limited to 120, 110, 100, 90, 80, 70, 60, 50, 40, 30, 20, 10, 8, 5, or 3 minutes.

In one embodiment, the particles are suspended in a diluent that comprises additive and are subjected to vacuum treatment at a strength of about 143 Torr for about at least 10 minutes, 20 minutes, 30 minutes, 40 minutes, 50 minutes, 60 minutes, 70 minutes, 80 minutes, 90 minutes, 100 minutes, or 120 minutes. In one embodiment, the particles are suspended in a diluent that comprises additive and are subjected to vacuum treatment at a strength of at least about 90, 89, 88, 87, 86, or 85 Torr for at least about at 10 minutes, 20 minutes, 30 minutes, or 40 minutes. In one embodiment, the particles are suspended in a diluent that comprises additive and are subjected to vacuum treatment at a strength of at least about 87 Torr for at least about 10 minutes, 20 minutes, 30 minutes, 40 minutes, 60 minutes, 90 minutes, or 120 minutes. In one embodiment, the particles are suspended in a diluent that comprises additive and are subjected to vacuum treatment at a strength of at least about 35, 34, 33, 32, 31, or 30 Torr for at least 5 minutes. In one embodiment, the particles are suspended in a diluent that comprises additive and are subjected to vacuum treatment at a strength of at least about 35, 34, 33, 32, 31, or 30 Torr for at least 8 minutes. In one embodiment, the particles are suspended in a diluent that comprises additive and are subjected to vacuum treatment at a strength of at least about 35, 34, 33, 32, 31, or 30 Torr for at least 10 minutes. In one embodiment, the particles are suspended in a diluent that comprises additive and are subjected to vacuum treatment at a strength of at least about 35, 34, 33, 32, 31, or 30 Torr for at least 20 minutes. In one embodiment, the particles are suspended in a diluent that comprises additive and are subjected to vacuum treatment at a strength of at least about 35, 34, 33, 32, 31, or 30 Torr for at least 30 minutes. In one embodiment, the particles are suspended in a diluent that comprises additive and are subjected to vacuum treatment at a strength of at least about 35, 34, 33, 32, 31, or 30 Torr for at least 40 minutes. In one embodiment, the particles are suspended in a diluent that comprises additive and are subjected to 30 Torr for at least 5, 10, 15, 20, 25, 30, 35, 40, 45, 50, 55, 60, 65, 70, 75, 80, 85, or 90 minutes. In one embodiment, the particles are suspended in a diluent that comprises additive and are subjected to vacuum treatment at a strength of about 35 Torr for at least 90 minutes. In one embodiment, the particles are suspended in a diluent that comprises additive and are subjected to vacuum treatment at a strength of about 35 Torr for at least 60 minutes. In one embodiment, the particles are suspended in a diluent that comprises additive and are subjected to vacuum treatment at a strength of about 35 Torr for at least 30 minutes. In one embodiment, the particles are suspended in a diluent that comprises additive and are subjected to vacuum treatment at a strength of about 35 Torr for at least 15 minutes. In one embodiment, the particles are suspended in a diluent that comprises additive and are subjected to vacuum treatment at a strength of about 35 Torr for at least 5 minutes. In one embodiment, the particles are suspended in a diluent that comprises additive and are subjected to vacuum treatment at a strength of about 32 Torr for at least 30 minutes. In one embodiment, the particles are suspended in a diluent that comprises additive and are subjected to vacuum treatment at a strength of about 32 Torr for at least 15 minutes. In one embodiment, the particles are suspended in a diluent that comprises additive and subjected to vacuum treatment at a strength of about 32 Torr for at least 5 minutes. In one embodiment, the particles are suspended in a diluent that comprises additive and are subjected to vacuum treatment at a strength of about 30 Torr for at least 30 minutes. In one embodiment, the particles are suspended in a diluent that comprises additive and are subjected to vacuum treatment at a strength of about 30 Torr for at least 15 minutes. In one embodiment, the particles are suspended in a diluent that comprises additive and are subjected to vacuum treatment at a strength of about 30 Torr for at least 5 minutes. In one embodiment, the particles are suspended in a diluent that comprises additive and are subjected to vacuum treatment at a strength of less than 30 Torr for at least 30 minutes. In one embodiment, the particles are suspended in a diluent that comprises additive and are subjected to vacuum treatment at a strength of less than 30 Torr for at least 15 minutes. In one embodiment, the particles are suspended in a diluent that comprises additive and subjected to vacuum treatment at a strength of less than 30 Torr for at least 5 minutes. In one embodiment, the particles are suspended in a diluent that comprises additive and are subjected to vacuum treatment at a strength of about 20 Torr for at least 30 minutes. In one embodiment, the particles are suspended in a diluent that comprises additive and are subjected to vacuum treatment at a strength of about 20 Torr for at least 15 minutes. In one embodiment, the particles are suspended in a diluent that comprises additive and are subjected to vacuum treatment at a strength of about 20 Torr for at least 5 minutes.

In one embodiment, the particles are suspended in a diluent comprising additive in a vial attached to a vial adapter that is further attached to a 60 mL VacLok syringe containing a plunger (as shown in FIG. 15) wherein the plunger is pulled to the 50 mL mark and locked to create a negative pressure of approximately 30 Torr and the pressure is held for at least about 3, 5, 8, 10, 15, 20, 25, 30, or 35 minutes. In an alternative embodiment, the particles are suspended in a diluent comprising additive in a vial attached to a vial adapter that is further attached to a 60 mL VacLok syringe containing a plunger wherein the plunger is pulled to the 45 mL mark, locked, and held for at least about 3, 5, 8, 10, 15, 20, 25, 30, 35, 40, 45, 50, 55, 60, 65, 70, 75, 80, 85, or 90 minutes. In an alternative embodiment, the particles are suspended in a diluent comprising additive in a vial attached to a vial adapter that is further attached to a 60 mL VacLok syringe containing a plunger wherein the plunger is pulled to the 40 mL mark, locked, and the pressure is held for at least about 3, 5, 8, 10, 15, 20, 25, 30, or 35 minutes. In an alternative embodiment, the particles are suspended in a diluent comprising additive in a vial attached to a vial adapter that is further attached to a 60 mL VacLok syringe containing a plunger wherein the plunger is pulled to the 35 mL mark, locked, and held for about at least 3, 5, 8, 10, 15, 20, 25, 30, or 35 minutes. In an alternative embodiment, the particles are suspended in a diluent comprising additive in a vial attached to a vial adapter that is further attached to a 60 mL VacLok syringe containing a plunger wherein the plunger is pulled to the 30 mL mark, locked, and held for at least about 3, 5, 8, 10, 15, 20, 25, 30, or 35 minutes. In an alternative embodiment, the particles are suspended in a diluent comprising additive in a vial attached to a vial adapter that is further attached to a 60 mL VacLok syringe containing a plunger wherein the plunger is pulled to the 25 mL mark, locked, and held for at least about 3, 5, 8, 10, 15, 20, 25, 30, or 35 minutes.

In certain embodiments, the particles are suspended in a diluent comprising additive and the suspension is exposed to a pressure of less than 40 Torr for between about 90 minutes and 1 minute, between about 60 minutes and 1 minute, between about 45 minutes and 1 minute, between about 30 minutes and 1 minute, between about 15 minutes and 1 minute, or between about 5 minutes and 1 minute.

In certain embodiments, the particles are suspended in a diluent comprising additive and the suspension is exposed to a pressure of less than 30 Torr for between about 90 minutes and 1 minute, between about 60 minutes and 1 minute, between about 45 minutes and 1 minute, between about 30 minutes and 1 minute, between about 15 minutes and 1 minute, or between about 5 minutes and 1 minute.

In certain embodiments, the lyophilized microparticles are suspended in a diluent comprising additive and subjected to vacuum treatment at least 1 hour prior to in vivo injection, at least 45 minutes prior to in vivo injection, at least 30 minutes prior to in vivo injection, at least 25 minutes prior to in vivo injection, at least 20 minutes prior to injection, at least 15 minutes prior to in vivo injection, at least 10 minutes prior to in vivo injection, or at least 5 minutes prior to in vivo injection. In one embodiment, the vacuum is conducted immediately before in vivo injection.

In one embodiment, the particles are suspended in a diluent comprising additive and are vacuumed at a strength of less than 35 Torr for less than 30 minutes and are immediately injected in vivo. In an alternative embodiment, the particles are vacuumed at a strength of less than 35 Torr for less than 20 minutes and are immediately injected in vivo. In an alternative embodiment, the particles are vacuumed at a strength of less than 35 Torr for less than 15 minutes and are immediately injected in vivo. In an alternative embodiment, the particles are vacuumed at a strength of less than 35 Torr for less than 10 minutes and are immediately injected in vivo.

### IX. Sonication

In an alternative embodiment, a process for providing an improved microparticle suspension prior to injection is sonication wherein particles are suspended in a diluent comprising additive that improves particle aggregation and the suspension of microparticles is sonicated for at least 30 minutes, at least 25 minutes, at least 20 minutes, at least 15 minutes, at least 10 minutes, at least 8 minutes, at least 5 minutes, or at least 3 minutes. In one embodiment, the particle suspensions are sonicated at a frequency of 40 kHz. In one embodiment, the particles are suspended in the diluent at a concentration of 100 mg/mL, 150 mg/mL, 200 mg/mL, 250 mg/mL, 300 mg/mL, 350 mg/mL, 400 mg/mL, 450 mg/mL or 500 mg/mL. In one embodiment, the diluent is hyaluronic acid. In an alternative embodiment, the diluent is selected from hyaluronic acid, hydroxypropyl methylcellulose, chondroitin sulfate, or a blend of at least two diluents selected from hyaluronic acid, hydroxypropyl methylcellulose, and chondroitin sulfate. In an alternative embodiment, the diluent is selected from acacia, tragacanth, alginic acid, carrageenan, locust bean gum, gellan gum, guar gum, gelatin, starch, methylcellulose, sodium carboxymethylcellulose, hydroxyethylcellulose, hydroxypropyl cellulose, Carbopol® homopolymers (acrylic acid crosslinked with allyl sucrose or allyl pentaerythritol), and Carbopol® copolymers (acrylic acid and C₁₀-C₃₀ alkyl acrylate crosslinked with allyl pentaerythritol).

In certain embodiments, the lyophilized microparticles are suspended in a diluent comprising additive and subjected to sonication at least 1 hour prior to in vivo injection, at least 45 minutes prior to in vivo injection, at least 30 minutes prior to in vivo injection, at least 25 minutes prior to in vivo injection, at least 20 minutes prior to injection, at least 15 minutes prior to in vivo injection, at least 10 minutes prior to in vivo injection, or at least 5 minutes prior to in vivo injection. In one embodiment, the vacuum is conducted immediately before in vivo injection.

In certain embodiments, a combination of vacuum treatment and sonication can be used following isolation and reconstitution of the microparticles.

### X. Biodegradable Polymers

The surface treated microparticles can include one or more biodegradable polymers or copolymers. The polymers should be biocompatible in that they can be administered to a patient without an unacceptable adverse effect. Biodegradable polymers are well known to those in the art and are the subject of extensive literature and patents. The biodegradable polymer or combination of polymers can be selected to provide the target characteristics of the microparticles, including the appropriate mix of hydrophobic and hydrophilic qualities, half-life and degradation kinetics in vivo, compatibility with the therapeutic agent to be delivered, appropriate behavior at the site of injection, etc.

For example, it should be understood by one skilled in the art that by manufacturing a microparticle from multiple polymers with varied ratios of hydrophobic, hydrophilic, and biodegradable characteristics that the properties of the microparticle can be designed for the target use. As an illustration, a microparticle manufactured with 90 percent PLGA and 10 percent PEG is more hydrophilic than a microparticle manufactured with 95 percent PLGA and 5 percent PEG. Further, a microparticle manufactured with a higher content of a less biodegradable polymer will in general degrade more slowly. This flexibility allows microparticles of the present invention to be tailored to the desired level of solubility, rate of release of pharmaceutical agent, and rate of degradation.

In certain embodiments, the microparticle includes a poly(α-hydroxyacid). Examples of poly(α-hydroxyacids) include poly lactic acid (PLA), polyglycolic acid (PGA), poly(D,L-lactide-co-glycolide)(PLGA), and poly D,L-lactic acid (PDLLA). polyesters, poly (ε-caprolactone), poly (3-hydroxy-butyrate), poly (s-caproic acid), poly (p-dioxanone), poly (propylene fumarate), poly (ortho esters), polyol/diketene acetals, polyanhydrides, poly (sebacic anhydride) (PSA), poly (carboxybis-carboxyphenoxyphosphazene) (PCPP), poly [bis (p-carboxyphenoxy) methane](PCPM), copolymers of SA, CPP and CPM (as described in Tamat and Langer in Journal of Biomaterials Science Polymer Edition, 3, 315-353, 1992 and by Domb in Chapter 8 of The Handbook of Biodegradable Polymers, Editors Domb A J and Wiseman R M, Harwood Academic Publishers), and poly (amino acids).

In one embodiment, the microparticle includes about at least 90 percent hydrophobic polymer and about not more than 10 percent hydrophilic polymer. Examples of hydrophobic polymers include polyesters such as poly lactic acid (PLA), polyglycolic acid (PGA), poly(D,L-lactide-co-glycolide)(PLGA), and poly D,L-lactic acid (PDLLA); polycaprolactone; polyanhydrides, such as polysebacic anhydride, poly(maleic anhydride); and copolymers thereof. Examples of hydrophilic polymers include poly(alkylene glycols) such as polyethylene glycol (PEG), polyethylene oxide (PEO), and poly(ethylene glycol) amine; polysaccharides; poly(vinyl alcohol) (PVA); polypyrrolidone; polyacrylamide (PAM); polyethylenimine (PEI); poly(acrylic acid); poly(vinylpyrolidone) (PVP); or a copolymer thereof.

In one embodiment, the microparticle includes about at least 85 percent hydrophobic polymer and at most 15 percent hydrophilic polymer.

In one embodiment, the microparticle includes about at least 80 percent hydrophobic polymer and at most 20 percent hydrophilic polymer.

In one embodiment, the microparticle includes PLA. In one embodiment, the PLA is acid-capped. In one embodiment, the PLA is ester-capped.

In one embodiment, the microparticle includes PLGA and PLGA-PEG.

In one embodiment, the microparticle includes PLA and PLGA-PEG.

In one embodiment, the microparticle includes PLGA, PLGA-PEG and PVA.

In one embodiment, the microparticle includes PLA, PLGA-PEG and PVA.

In one embodiment, the microparticle includes PLGA, PLA, and PLGA-PEG.

In one embodiment, the microparticle includes PLGA, PLA, PLGA-PEG and PVA.

In one embodiment, the microparticle includes PLGA.

In one embodiment, the microparticle includes a copolymer of PLGA and PEG.

In one embodiment, the microparticle includes a copolymer of PLA and PEG.

In one embodiment, the microparticle comprises PLGA and PLGA-PEG, and combinations thereof.

In one embodiment, the microparticle comprises PLA and PLA-PEG.

In one embodiment, the microparticle includes PVA.

In one embodiment, the microparticles include PLGA, PLGA-PEG, PVA, or combinations thereof.

In one embodiment, the microparticles include the biocompatible polymers PLA, PLA-PEG, PVA, or combinations thereof.

In one embodiment, the microparticles have a mean size of about 20 μm to about 50 μm, 25 μm to about 45 μm, 25 μm to about 30 μm and a median size of about 29 μm to about 31 μm before surface treatment.

In one embodiment, the microparticles after surface treatment have about the same mean size and median size. In another embodiment, the microparticles after surface treatment have a mean size which is larger than the median size. In another embodiment, the microparticles after surface treatment have a mean size which is smaller than the median size.

In one embodiment, the microparticles have a mean size of about 20 μm to about 50 μm, 25 μm to about 45 μm, 25 μm to about 30 μm, or 30 to 33 μm and a median size of about 31 μm to about 33 μm after surface treatment with approximately 0.0075 M NaOH/ethanol to 0.75 M NaOH/ethanol (30:70, v:v).

In one embodiment, the microparticles have a mean size of about 20 μm to about 50 μm, 25 μm to about 45 μm, 25 μm to about 30 μm or 30 to 33 μm and a median size of about 31 μm to about 33 μm after surface treatment with approximately 0.75 M NaOH/ethanol to 2.5 M NaOH/ethanol (30:70, v:v).

In one embodiment, the microparticles have a mean size of about 20 μm to about 50 μm, about 25 μm to about 45 μm, about 25 μm to about 30 μm or 30 to 33 μm and a median size of about 31 μm to about 33 μm after surface treatment with approximately 0.0075 M HCl/ethanol to 0.75 M NaOH/ethanol (30:70, v:v).

In one embodiment, the microparticles have a mean size of about 20 μm to about 50 μm, about 25 μm to about 45 μm, about 25 μm to about 30 μm or 30 to 33 μm and a median size of about 31 μm to about 33 μm after surface treatment with approximately 0.75 M NaOH/ethanol to 2.5 M HCl/ethanol (30:70, v:v).

In one embodiment, a surface-modified solid aggregating microparticle is manufactured using a wet microparticle.

In one embodiment, the surface-modified solid aggregating microparticle can release a therapeutic agent over a longer period of time when compared to a non-surface treated microparticle.

In one embodiment, a surface-modified solid aggregating microparticle contains less surfactant than a microparticle prior to the surface modification.

In one embodiment, a surface-modified solid aggregating microparticle is more hydrophobic than a microparticle prior to the surface modification.

In one embodiment, a surface-modified solid aggregating microparticle is less inflammatory than a non-surface treated microparticle.

In one embodiment, the agent that removes the surface surfactant of a surface-modified solid aggregating microparticle comprises a solvent that partially dissolves or swells the surface-modified solid aggregating microparticle.

In one aspect of the present invention, an effective amount of a pharmaceutically active compound as described herein is incorporated into a surface treated microparticle, e.g., for convenience of delivery and/or sustained release delivery. The use of materials provides the ability to modify fundamental physical properties such as solubility, diffusivity, and drug release characteristics. These micrometer scale agents may provide more effective and/or more convenient routes of administration, lower therapeutic toxicity, extend the product life cycle, and ultimately reduce healthcare costs. As therapeutic delivery systems, surface treated microparticles can allow targeted delivery and sustained release.

### XI. Surfactants

In one embodiment, the manufacture of the microparticle includes a surfactant. Examples of surfactants include, for example, polyoxyethylene glycol, polyoxypropylene glycol, decyl glucoside, lauryl glucoside, octyl glucoside, polyoxyethylene glycol octylphenol, Triton X-100, glycerol alkyl ester, glyceryl laurate, cocamide MEA, cocamide DEA, dodecyldimethylamine oxide, and poloxamers. Examples of poloxamers include, poloxamers 188, 237, 338 and 407. These poloxamers are available under the trade name Pluronic® (available from BASF, Mount Olive, N.J.) and correspond to Pluronic® F-68, F-87, F-108 and F-127, respectively. Poloxamer 188 (corresponding to Pluronic® F-68) is a block copolymer with an average molecular mass of about 7,000 to about 10,000 Da, or about 8,000 to about 9,000 Da, or about 8,400 Da. Poloxamer 237 (corresponding to Pluronic® F-87) is a block copolymer with an average molecular mass of about 6,000 to about 9,000 Da, or about 6,500 to about 8,000 Da, or about 7,700 Da. Poloxamer 338 (corresponding to Pluronic® F-108) is a block copolymer with an average molecular mass of about 12,000 to about 18,000 Da, or about 13,000 to about 15,000 Da, or about 14,600 Da. Poloxamer 407 (corresponding to Pluronic® F-127) is a polyoxyethylene-polyoxypropylene triblock copolymer in a ratio of between about E101 P56 E101 to about E106 P70 E106, or about E101 P56E101, or about E106 P70 E106, with an average molecular mass of about 10,000 to about 15,000 Da, or about 12,000 to about 14,000 Da, or about 12,000 to about 13,000 Da, or about 12,600 Da.

Additional examples of surfactants that can be used in the invention include, but are not limited to, polyvinyl alcohol (which can be hydrolyzed polyvinyl acetate), polyvinyl acetate, Vitamin E-TPGS, poloxamers, cholic acid sodium salt, dioctyl sulfosuccinate sodium, hexadecyltrimethyl ammonium bromide, saponin, TWEEN® 20, TWEEN® 80, sugar esters, Triton X series, L-a-phosphatidylcholine (PC), 1,2-dipalmitoylphosphatidycholine (DPPC), oleic acid, sorbitan trioleate, sorbitan mono-oleate, sorbitan monolaurate, polyoxyethylene (20) sorbitan monolaurate, polyoxyethylene (20) sorbitan monooleate, natural lecithin, oleyl polyoxyethylene (2) ether, stearyl polyoxyethylene (2) ether, lauryl polyoxyethylene (4) ether, block copolymers of oxyethylene and oxypropylene, synthetic lecithin, diethylene glycol dioleate, tetrahydrofurfuryl oleate, ethyl oleate, isopropyl myristate, glyceryl monooleate, glyceryl monostearate, glyceryl monoricinoleate, cetyl alcohol, stearyl alcohol, cetylpyridinium chloride, benzalkonium chloride, olive oil, glyceryl monolaurate, corn oil, cotton seed oil, sunflower seed oil, lecithin, oleic acid, and sorbitan trioleate.

In on embodiment, the surfactant is polyvinyl alcohol (PVA). Any molecular weight PVA can be used that achieves the desired results. In one embodiment, the PVA has a molecular weight of up to about 10, 15, 20, 25, 30, 35 or 40 kd. In some embodiments, the PVA is partially hydrolyzed polyvinyl acetate, including but not limited to, up to about 70, 75, 80, 85, 88, 90 or even 95% hydrolyzed polyvinyl acetate. In one embodiment, the PVA is about 88% hydrolyzed polyvinyl acetate. In one embodiment, the PVA polymer has a molecule weight of 20,000 to 40,000 g/mol. In one embodiment, the PVA polymer has a molecular weight of 24,000 to 35,000 g/mol.

It should be recognized by one skilled in the art that some surfactants can be used as polymers in the manufacture of the microparticle. It should also be recognized by one skilled in the art that in some manufacture the microparticle may retain a small amount of surfactant which allows further modification of properties as desired.

### XII. Biodegradable Polymeric-Containing Microparticles

In certain aspects, solid aggregating microparticles are provided that include a poly(α-hydroxyacid) biodegradable polymer, for example poly-lactic acid (PLA) biodegradable polymer, and a hydrophobic polymer covalently bound to a hydrophilic polymer, for example PLGA-PEG biodegradable polymer, wherein the solid aggregating microparticles have a solid core, include a therapeutic agent, are sufficiently small to be injected in vivo, and are capable of aggregating in vivo. In one embodiment, the microparticles aggregate in vivo to form at least one pellet of at least 500 μm in vivo to provide sustained drug delivery in vivo for at least one month, two months, three months, four months, five months, six months, seven months, eight months, nine months, or more. In one embodiment, the microparticles are about 10 μm to about 50 μm, from about 20 μm to about m, from about 25 μm to about 35 μm.

It has been discovered that the inclusion of PLA in certain microparticle formulations allows for the achievement of long-term slow substantially surface erosion for example, 9 months, 10 months, 11 months, 12 months or greater. In some embodiments, nearly zero-order or linear release drug delivery in vivo, can be achieved.

As contemplated herein, PLA for use in the present invention can include any known variant, for example, but not limited to, PLLA (Poly-L-lactic Acid), racemic PLLA (Poly-L-lactic Acid), PDLA (Poly-D-lactic Acid), and PDLLA (Poly-DL-lactic Acid), or a mixture thereof. In one embodiment, the PLA is Poly-L-lactic Acid. The PLA can be ester end-capped or acid end-capped.

In one embodiment, the PLA comprises at least about 40%, 45%, 50%, 55%, 60%, 65%, 70%, 75%, 80%, 85%, 90%, 95%, 96%, 97%, 98%, 99%, 99.5%, or 99.9% of the microparticle. In one embodiment, the PLA has a molecular weight between about 30 and 60 kD, about 35 and 55 kD, or about 40 and 50 kD. The microparticle further includes a hydrophobic polymer covalently bound to a hydrophilic biodegradable polymer. Hydrophobic degradable polymers are known in the art, and include, but are not limited to, polyglycolic acid (PGA), poly(D,L-lactide-co-glycolide)(PLGA), and poly D,L-lactic acid (PDLLA); polycaprolactone; polyanhydrides, such as polysebacic anhydride, poly(maleic anhydride); and copolymers thereof. Hydrophilic polymers are known in the art and include, for example poly(alkylene glycols) such as polyethylene glycol (PEG), polyethylene oxide (PEO), and poly(ethylene glycol) amine; polysaccharides; poly(vinyl alcohol) (PVA); polypyrrolidone; polyacrylamide (PAM); polyethylenimine (PEI); poly(acrylic acid); poly(vinylpyrolidone) (PVP); or a copolymer thereof. Hydrophobic polymers covalently bound to hydrophilic polymers include, for example, PLGA-PEG, PLA-PEG, PCL-PEG in an amount from about 0.5 percent to about 10 percent, about 0.5 percent to about 5 percent, about 0.5 percent to about 4 percent, about 0.5 percent to about 3 percent, or about 0.1 percent to about 1, 2, 5, or 10 percent. In one embodiment, the hydrophobic polymer covalently bound to the hydrophilic polymer is PLGA-PEG.

In one embodiment, the ratio of PLA/hydrophobic polymer covalently bound to a hydrophilic polymer in the microparticle is between about 40/1 to about 120/1 by weight. In one embodiment, the ratio by weight of PLA/hydrophobic polymer covalently bound to hydrophilic polymer in the microparticle is about 45/1, 50/1, 55/1, 60/1, 65/1, 70/1, 75/1, 80/1, 85/1, 90/1, 95/1, 96/1, 97/1, 98/1, 99/1, 99.5/1, 99.9/1, 100/1, 101/1, 102/1, 103/1, 104/1, 105/1, or greater than 105/1. In one embodiment, the hydrophobic polymer covalently bound to a hydrophilic polymer is PLGA-PEG.

In one embodiment, the PLA/hydrophobic polymer covalently bound to hydrophilic polymer microparticle further comprises an additional hydrophobic biodegradable polymer, for example polyglycolic acid (PGA), poly(D,L-lactide-co-glycolide)(PLGA), and poly D,L-lactic acid (PDLLA); polycaprolactone; polyanhydrides, such as polysebacic anhydride, poly(maleic anhydride); and copolymers thereof. In one embodiment, the additional hydrophobic biodegradable polymer comprises about 1%, 5%, 10%, 15%, 20%, 25%, 30%, 35%, 40%, 45%, 50%, or 55% of the microparticle. In one embodiment, the additional hydrophobic polymer is PLGA. In one embodiment, the ratio by weight of lactide/glycolide in the PLGA is about 5/95, 10/90, 15/85, 20/80, 25/75, 30/70, 35/65, 40/60, 45/55, 50/50, 55/45, 60/40, 65/35, 70/30, 75/25, 80/20, 85/15, 90/10, or 95/5. The PLGA can be acid end-capped or ester end-capped. The PLA can be acid end-capped or ester end-capped.

In one embodiment, the microparticle contains PLA, PLGA, and PLGA-PEG. In one embodiment, the ratio by weight of PLA/PLGA/PLGA-PEG in the microparticle is about 5/95/1, 10/90/1, 15/85/1, 20/80/1, 25/75/1, 30/70/1, 35/65/1, 40/60/1, 45/55/1, 40/60/1, 45/55/1, 50/50/1, 55/45/1, 60/40/1, 65/35/1, 70/30/1, 75/25/1, 80/20/1, 85/15/1, 90/10/1, 95/5/1, or 100/1/1. In one embodiment, PLA-PEG or PLC-PEG is substituted for PLGA-PEG.

In one embodiment, the microparticles comprise PLA/PLGA45k-PEG5k. The PLA can be ester or acid end-capped. In one embodiment, the PLA is acid end-capped. In one embodiment, the microparticles comprise PLA/PLGA45k-PEG5k in a ratio by weight of between about 100/1 to 80/20, about 100/1, 95/5, 90/10, 85/15, or 80/20. In one embodiment, the microparticles comprise PLA/PLGA7525/PLGA45k-PEG5k in a ratio of between about 99/1/1 to 1/99/1, about 99/1/1, 95/5/1, 90/10/1, 85/15/1, 80/20/1, 75/25/1, 70/30/1, 65/35/1, 60/40/1, 55/45/1, 50/50/1, 45/55/1, 40/60/1, 35/65/1, 30/70/1, 25/75/1, 20/80/1, 15/85/1, 10/90/1, 5/95/1, or 1/99/1. The PLGA7525 and PLA can be acid or ester end capped. In one embodiment, both the PLGA7525 and PLA are acid end-capped. In one embodiment, the microparticles comprise PLA/PLGA5050/PLGA45k-PEG5k. In one embodiment, the microparticles comprise PLA/PLGA5050/PLGA45k-PEG5k in a ratio by weight of about 99/1/1, 95/5/1, 90/10/1, 85/15/1, 80/20/1, 75/25/1, 70/30/1, 65/35/1, 60/40/1, 55/45/1, 50/50/1, 45/55/1, 40/60/1, 35/65/1, 30/70/1, 25/75/1, 20/80/1, 15/85/1, 10/90/1, 5/95/1, or 1/99/1. The PLA and PLGA5050 can be acid or ester end-capped. In one embodiment, both the PLA and PLGA are acid end-capped.

In one embodiment, the PLA microparticles described herein is surface modified. In one embodiment, the microparticles have a modified surface which has been treated under mild conditions at a temperature at or less than about 18° C. to remove surface surfactant or cause surface polymer to partially degrade. The solid aggregating microparticles are suitable, for example, for an intravitreal injection, implant, including an ocular implant, periocular delivery, or delivery in vivo outside of the eye.

In one embodiment, the aggregate formed in vivo is a blend or mix of microparticles, wherein at least one of the microparticles includes a poly-lactic acid (PLA) biodegradable polymer and a hydrophobic biodegradable polymer covalently linked to a hydrophilic polymer, for example PLGA-PEG biodegradable polymer. In one embodiment, the mix or blend includes one or more microparticles comprised of a non-PLA polymer. In one embodiment, the mix or blend includes PLA/PLGA-PEG microparticles and PLGA/PLGA-PEG microparticles. In one embodiment, the mix or blend comprises a ratio by weight of PLA/PLGA-PEG to PLGA/PLGA-PEG of about 1/99, 5/95, 10/90, 15/85, 20/80, 25/75, 30/70, 35/65, 40/60, 45/55, 50/50, 55/45, 60/40, 65/35, 70/30, 75/25, 80/20, 85/15, 90/10, 95/5, or 99/1. In one embodiment, the ratio of lactide/glycolide in the PLGA or PLGA-PEG is by weight about 5/95, 10/90, 15/85, 20/80, 25/75, 30/70, 35/65, 40/60, 45/55, 50/50, 55/45, 60/40, 65/35, 70/30, 75/25, 80/20, 85/15, 90/10, or 95/5. The PLGA can be acid end capped or ester end capped. In one embodiment, the PLGA is a block co-polymer, for example, diblock, triblock, multiblock, or star-shaped block. In one embodiment, the PLGA is a random co-polymer.

In one embodiment, the mix or blend includes PLA/PLGA-PEG microparticles and PLA/PLGA/PLGA-PEG microparticles. In one embodiment, the mix or blend comprises a ratio by weight of PLA/PLGA-PEG to PLA/PLGA/PLGA-PEG of about 1/99, 5/95, 10/90, 15/85, 20/80, 25/75, 30/70, 35/65, 40/60, 45/55, 50/50, 55/45, 60/40, 65/35, 70/30, 75/25, 80/20, 85/15, 90/10, 95/5, or 99/1. In one embodiment, the ratio by weight of lactide/glycolide in the PLGA or PLGA-PEG is about 5/95, 10/90, 15/85, 20/80, 25/75, 30/70, 35/65, 40/60, 45/55, 50/50, 55/45, 60/40, 65/35, 70/30, 75/25, 80/20, 85/15, 90/10, or 95/5. In one embodiment, the PLGA is in a lactide/glycolide ratio by weight of 95/5, 90/10, 85/15, 80/20, 75/25, 70/30, 65/35, 60/40, 55/45, 50/50, 45/55, 40/60, 35/65, 30/70, 25/75, 20/80, 15/85, 10/90, 5/95. The PLGA can be acid end capped or ester end capped. In one embodiment, the PLGA is a block co-polymer, for example, diblock, triblock, multiblock, or star-shaped block. In one embodiment, the PLGA is a random co-polymer.

In one embodiment, the blend or mix of microparticles is comprised of a PLA/PLGA-PEG microparticle, wherein the PLA comprises from about 80% to 99.9% of the microparticle, and a PLGA/PLGA-PEG microparticle, wherein the PLGA comprises from about 80% to 99.9% of the microparticle. In one embodiment, the blend or mix of microparticles is comprised of a PLA/PLGA45k-PEG5k microparticle and a PLGA7525/PLGA45k-PEG5k microparticle in a ratio by weight of from about 1/99 to about 99/1, about 1.99, 5/95, 10/90, 15/85, 20/80, 25/75, 30/70, 35/65, 40/60, 45/55, 50/50, 55/45, 60/40, 65/35, 70/30, 75/25, 80/20, 85/15, 90/10, 95/5, or 99/1. In one embodiment, the ratio by weight of PLA/PLGA45k-PEG5k microparticle to PLGA7525/PLGA45k-PEG5k microparticles is between about 20/80 to 40/60, about 20/80, 25/75, 30/70, 35/65, or 40/60. The PLA and PLGA can be ester or acid end capped. In one embodiment, the blend or mix of microparticles is comprised of a PLA 4 A/PLGA45k-PEG5k microparticle and a PLGA7525 4 A/PLGA45k-PEG5k microparticle in a ratio by weight of from about 1/99 to about 99/1, about 1.99, 5/95, 10/90, 15/85, 20/80, 25/75, 30/70, 35/65, 40/60, 45/55, 50/50, 55/45, 60/40, 65/35, 70/30, 75/25, 80/20, 85/15, 90/10, 95/5, or 99/1. In one embodiment, the ratio by weight of PLA 4 A/PLGA45k-PEG5k microparticle to PLGA7525 4 A/PLGA45k-PEG5k microparticles is between about 20/80 to 40/60, about 20/80, 25/75, 30/70, 35/65, or 40/60.

In one embodiment, the blend or mix of microparticles is comprised of a PLA/PLGA45k-PEG5k microparticle and a PLGA5050/PLGA45k-PEG5k microparticle in a ratio by weight of from about 1/99 to about 99/1, about 1.99, 5/95, 10/90, 15/85, 20/80, 25/75, 30/70, 35/65, 40/60, 45/55, 50/50, 55/45, 60/40, 65/35, 70/30, 75/25, 80/20, 85/15, 90/10, 95/5, or 99/1. In one embodiment, the ratio by weight of PLA/PLGA45k-PEG5k microparticle to PLGA5050/PLGA45k-PEG5k microparticles is between about 20/80 to 40/60, about 20/80, 25/75, 30/70, 35/65, or 40/60. The PLA and PLGA can be ester or acid end capped. In one embodiment, the blend or mix of microparticles is comprised of a PLA 4 A/PLGA45k-PEG5k microparticle and a PLGA5050 4 A/PLGA45k-PEG5k microparticle in a ratio by weight of from about 1/99 to about 99/1, about 1.99, 5/95, 10/90, 15/85, 20/80, 25/75, 30/70, 35/65, 40/60, 45/55, 50/50, 55/45, 60/40, 65/35, 70/30, 75/25, 80/20, 85/15, 90/10, 95/5, or 99/1. In one embodiment, the ratio by weight of PLA 4 A/PLGA45k-PEG5k microparticle to PLGA5050 4 A/PLGA45k-PEG5k microparticles is between about 20/80 to 40/60, about 20/80, 25/75, 30/70, 35/65, or 40/60.

In one embodiment, the blend or mix of microparticles is comprised of a PLA/PLGA/PLGA-PEG microparticle and a PLGA/PLGA-PEG microparticle, In one embodiment, the blend or mix of microparticles is comprised of a PLA/PLGA/PLGA45k-PEG5k microparticle and a PLGA/PLGA45k-PEG5k microparticle in a ratio by weight of from about 1/99 to about 99/1, about 1.99, 5/95, 10/90, 15/85, 20/80, 25/75, 30/70, 35/65, 40/60, 45/55, 50/50, 55/45, 60/40, 65/35, 70/30, 75/25, 80/20, 85/15, 90/10, 95/5, or 99/1. In one embodiment, the blend or mix of microparticles is comprised of a PLA/PLGA7525/PLGA45k-PEG5k microparticle and a PLGA7525/PLGA45k-PEG5k microparticle in a ratio by weight of from about 1/99 to about 99/1, about 1.99, 5/95, 10/90, 15/85, 20/80, 25/75, 30/70, 35/65, 40/60, 45/55, 50/50, 55/45, 60/40, 65/35, 70/30, 75/25, 80/20, 85/15, 90/10, 95/5, or 99/1. In one embodiment, the blend or mix of microparticles is comprised of a PLA/PLGA7525/PLGA45k-PEG5k microparticle and a PLGA5050/PLGA45k-PEG5k microparticle in a ratio by weight of from about 1/99 to about 99/1, about 1.99, 5/95, 10/90, 15/85, 20/80, 25/75, 30/70, 35/65, 40/60, 45/55, 50/50, 55/45, 60/40, 65/35, 70/30, 75/25, 80/20, 85/15, 90/10, 95/5, or 99/1. In one embodiment, the blend or mix of microparticles is comprised of a PLA/PLGA5050/PLGA45k-PEG5k microparticle and a PLGA7525/PLGA45k-PEG5k microparticle in a ratio by weight of from about 1/99 to about 99/1, about 1.99, 5/95, 10/90, 15/85, 20/80, 25/75, 30/70, 35/65, 40/60, 45/55, 50/50, 55/45, 60/40, 65/35, 70/30, 75/25, 80/20, 85/15, 90/10, 95/5, or 99/1. The PLA and PLGA can be acid end capped or ester end capped.

In one embodiment, the microparticle comprises a blend or mix of ratio by weight of PLA4 A/PLGA45k-PEG5k and PLGA7525 4 A/PLGA45k-PEG5k. In one embodiment, the blend or mix of microparticles comprise a ratio by weight of PLA 4 A/PLGA45k-PEG5k microparticle to PLGA7525 4 A/PLGA45k-PEG5k microparticles is between about 20/80 to 40/60, about 20/80, 25/75, 30/70, 35/65, or 40/60. In one embodiment, the blend or mix of microparticles is comprised of a PLA 4 A/PLGA45k-PEG5k microparticle and a PLGA5050 4 A/PLGA45k-PEG5k microparticle in a ratio by weight of from about 1/99 to about 99/1, about 1.99, 5/95, 10/90, 15/85, 20/80, 25/75, 30/70, 35/65, 40/60, 45/55, 50/50, 55/45, 60/40, 65/35, 70/30, 75/25, 80/20, 85/15, 90/10, 95/5, or 99/1. In one embodiment, the ratio by weight of PLA 4 A/PLGA45k-PEG5k microparticle to PLGA5050 4 A/PLGA45k-PEG5k microparticles is between about 20/80 to 50/50, about 20/80, 25/75, 30/70, 35/65, 40/60, or 50/50.

As contemplated herein, PLA, as utilized herein, can be replaced with a different poly(α-hydroxyacid) biodegradable polymer, for example, polyglycolic acid (PGA), poly(D,L-lactide-co-glycolide)(PLGA), and poly D,L-lactic acid (PDLLA). polyesters, poly (F-caprolactone), poly (3-hydroxy-butyrate), poly (s-caproic acid), poly (p-dioxanone), poly (propylene fumarate), poly (ortho esters), polyol/diketene acetals, polyanhydrides, poly (sebacic anhydride) (PSA), poly (carboxybis-carboxyphenoxyphosphazene) (PCPP), poly [bis (p-carboxyphenoxy) methane](PCPM), copolymers of SA, CPP, CPM, and poly(amino acids).

### XIII. Examples of Disorders to be Treated

In one embodiment, the microparticles described herein and a pharmaceutically active compound encapsulated in the microparticle optionally in combination with a pharmaceutically acceptable carrier, excipient, or diluent are used for the treatment of a disorder, including a human disorder. In one embodiment, the composition is a pharmaceutical composition for treating an eye disorder or eye disease.

Non-limiting exemplary eye disorders or diseases treatable with the composition include age related macular degeneration, alkaline erosive keratoconjunctivitis, allergic conjunctivitis, allergic keratitis, anterior uveitis, Behcet's disease, blepharitis, blood-aqueous barrier disruption, chorioiditis, chronic uveitis, conjunctivitis, contact lens-induced keratoconjunctivitis, corneal abrasion, corneal trauma, corneal ulcer, crystalline retinopathy, cystoid macular edema, dacryocystitis, diabetic keratopathy, diabetic macular edema, diabetic retinopathy, dry eye disease, dry age-related macular degeneration, eosinophilic granuloma, episcleritis, exudative macular edema, Fuchs' Dystrophy, giant cell arteritis, giant papillary conjunctivitis, glaucoma, glaucoma surgery failure, graft rejection, herpes zoster, inflammation after cataract surgery, iridocorneal endothelial syndrome, iritis, keratoconjunctivitis sicca, keratoconjunctivitis inflammatory disease, keratoconus, lattice dystrophy, map-dot-fingerprint dystrophy, necrotic keratitis, neovascular diseases involving the retina, uveal tract or cornea, for example, neovascular glaucoma, corneal neovascularization, neovascularization resulting following a combined vitrectomy and lensectomy, neovascularization of the optic nerve, and neovascularization due to penetration of the eye or contusive ocular injury, neuroparalytic keratitis, non-infectious uveitis ocular herpes, ocular lymphoma, ocular rosacea, ophthalmic infections, ophthalmic pemphigoid, optic neuritis, panuveitis, papillitis, pars planitis, persistent macular edema, phacoanaphylaxis, posterior uveitis, post-operative inflammation, proliferative diabetic retinopathy, proliferative sickle cell retinopathy, proliferative vitreoretinopathy, retinal artery occlusion, retinal detachment, retinal vein occlusion, retinitis pigmentosa, retinopathy of prematurity, rubeosis iritis, scleritis, Stevens-Johnson syndrome, sympathetic ophthalmia, temporal arteritis, thyroid associated ophthalmopathy, uveitis, vernal conjunctivitis, vitamin A insufficiency-induced keratomalacia, vitritis, wet age-related macular degeneration, neovascular age-related macular degeneration, myopia, and presbyonia.

### XIV. Therapeutically Active Agents to be Delivered

A wide variety of therapeutic agents can be delivered in a long term sustained manner in vivo using the present invention.

A “therapeutically effective amount” of a pharmaceutical composition/combination of this invention means an amount effective, when administered to a patient, to provide a therapeutic benefit such as an amelioration of symptoms of the selected disorder, typically an ocular disorder. In certain aspects, the disorder is glaucoma, a disorder mediated by carbonic anhydrase, a disorder or abnormality related to an increase in intraocular pressure (IOP), a disorder mediated by nitric oxide synthase (NOS), a disorder requiring neuroprotection such as to regenerate/repair optic nerves, allergic conjunctivitis, anterior uveitis, cataracts, dry or wet age-related macular degeneration (AMD), neovascular AMD, neovasular or diabetic retinopathy.

A “pharmaceutically acceptable salt” is formed when a therapeutically active compound is modified by making an inorganic or organic, non-toxic, acid or base addition salt thereof. Salts can be synthesized from a parent compound that contains a basic or acidic moiety by conventional chemical methods. Generally, such a salt can be prepared by reacting a free acid form of the compound with a stoichiometric amount of the appropriate base (such as Na, Ca, Mg, or K hydroxide, carbonate, bicarbonate, or the like), or by reacting a free base form of the compound with a stoichiometric amount of the appropriate acid. Such reactions are typically carried out in water or in an organic solvent, or in a mixture of the two. Generally, non-aqueous media like ether, ethyl acetate, ethanol, isopropanol, or acetonitrile are typical, where practicable. Examples of pharmaceutically acceptable salts include, but are not limited to, mineral or organic acid salts of basic residues such as amines; alkali or organic salts of acidic residues such as carboxylic acids; and the like. The pharmaceutically acceptable salts include the conventional non-toxic salts and the quaternary ammonium salts of the parent compound formed, for example, from non-toxic inorganic or organic acids. For example, conventional non-toxic acid salts include those derived from inorganic acids such as hydrochloric, hydrobromic, sulfuric, sulfamic, phosphoric, nitric and the like; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, mesylic, esylic, besylic, sulfanilic, 2-acetoxybenzoic, fumaric, toluenesulfonic, methanesulfonic, ethane disulfonic, oxalic, isethionic, HOOC—(CH₂)ₙ—COOH where n is 0-4, and the like. Lists of additional suitable salts may be found, e.g., in Remington's Pharmaceutical Sciences, 17th ed., Mack Publishing Company, Easton, Pa., p. 1418 (1985).

In one embodiment, the microparticles of the present invention can comprise a compound for the treatment of glaucoma, for instance a beta-adrenergic antagonists, a prostaglandin analog, an adrenergic agonist, a carbonic anhydrase inhibitor, a parasympathomimetic agent, a dual anti-VEGF/Anti-PDGF therapeutic or a dual leucine zipper kinase (DLK) inhibitor. In another embodiment, the microparticles of the present invention can comprise a compound for the treatment of diabetic retinopathy. Such compounds may be administered in lower doses according to the invention as they may be administered at the site of the ocular disease.

Examples of loop diuretics include furosemide, bumetanide, piretanide, ethacrynic acid, etozolin, and ozolinone.

Examples of beta-adrenergic antagonists include, but are not limited to, timolol (Timoptic®), levobunolol (Betagan®), carteolol (Ocupress®), Betaxolol (Betoptic), and metipranolol (OptiPranolol®).

Examples of prostaglandin analogs include, but are not limited to, latanoprost (Xalatan®), travoprost (Travatan®), bimatoprost (Lumigan®) and tafluprost (Zioptan™) Examples of adrenergic agonists include, but are not limited to, brimonidine (Alphagan®), epinephrine, dipivefrin (Propine®) and apraclonidine (Lopidine®).

Examples of carbonic anhydrase inhibitors include, but are not limited to, dorzolamide (Trusopt®), brinzolamide (Azopt®), acetazolamide (Diamox®) and methazolamide (Neptazane®), see structures below.

Example of antioxidants include Alpha Lipoic Acid (ALA).

Examples of tyrosine kinase inhibitors include Tivosinib, Imatinib, Gefitinib, Erlotinib, Lapatinib, Canertinib, Semaxinib, Vatalaninib, Sorafenib, Axitinib, Pazopanib, Dasatinib, Nilotinib, Crizotinib, Ruxolitinib, Vandetanib, Vemurafenib, Bosutinib, Cabozantinib, Regorafenib, Vismodegib, and Ponatinib. In one embodiment, the tyrosine kinase inhibitor is selected from Tivosinib, Imatinib, Gefitinib, and Erlotinib. In one embodiment, the tyrosine kinase inhibitor is selected from Lapatinib, Canertinib, Semaxinib, and Vatalaninib. In one embodiment, the tyrosine kinase inhibitor is selected from Sorafenib, Axitinib, Pazopanib, and Dasatinib. In one embodiment, the tyrosine kinase inhibitor is selected from Nilotinib, Crizotinib, Ruxolitinib, Vandetanib, and Vemurafenib. In one embodiment, the tyrosine kinase inhibitor is selected from Bosutinib, Cabozantinib, Regorafenib, Vismodegib, and Ponatinib.

An example of parasympathomimetics include, but is not limited to, pilocarpine and atropine.

DLK inhibitors include, but are not limited to, Crizotinib, KW-2449 and Tozasertib, see structure below.

Drugs used to treat diabetic retinopathy include, but are not limited to, ranibizumab (Lucentis®).

In one embodiment, the dual anti-VEGF/Anti-PDGF therapeutic is sunitinib malate (Sutent®).

In one embodiment, the compound is a treatment for glaucoma and can be used as an effective amount to treat a host in need of glaucoma treatment.

In another embodiment, the compound acts through a mechanism other than those associated with glaucoma to treat a disorder described herein in a host, typically a human.

In one embodiment, the therapeutic agent is selected from a phosphoinositide 3-kinase (PI3K) inhibitor, a Bruton's tyrosine kinase (BTK) inhibitor, or a spleen tyrosine kinase (Syk) inhibitor, or a combination thereof.

PI3K inhibitors that may be used in the present invention are well known. Examples of PI3 kinase inhibitors include but are not limited to Wortmannin, demethoxyviridin, perifosine, idelalisib, Pictilisib, Palomid 529, ZSTK474, PWT33597, CUDC-907, and AEZS-136, duvelisib, GS-9820, BKM120, GDC-0032 (Taselisib) (2-[4-[2-(2-Isopropyl-5-methyl-1,2,4-triazol-3-yl)-5,6-dihydroimidazo[1,2-d][1,4]benzoxazepin-9-yl]pyrazol-1-yl]-2-methylpropanamide), MLN-1117 ((2R)-1-Phenoxy-2-butanyl hydrogen (S)-methylphosphonate; or Methyl(oxo) {[(2R)-1-phenoxy-2-butanyl]oxy}phosphonium)), BYL-719 ((2S)—N1-[4-Methyl-5-[2-(2,2,2-trifluoro-1,1-dimethylethyl)-4-pyridinyl]-2-thiazolyl]-1,2-pyrrolidinedicarboxamide), GSK2126458 (2,4-Difluoro-N-{2-(methyloxy)-5-[4-(4-pyridazinyl)-6-quinolinyl]-3-pyridinyl}benzenesulfonamide) (omipalisib), TGX-221 ((±)-7-Methyl-2-(morpholin-4-yl)-9-(1-phenylaminoethyl)-pyrido[1,2-a]-pyrimidin-4-one), GSK2636771 (2-Methyl-1-(2-methyl-3-(trifluoromethyl)benzyl)-6-morpholino-1H-benzo[d]imidazole-4-carboxylic acid dihydrochloride), KIN-193 ((R)-2-((1-(7-methyl-2-morpholino-4-oxo-4H-pyrido[1,2-a]pyrimidin-9-yl)ethyl)amino)benzoic acid), TGR-1202/RP5264, GS-9820 ((S)-1-(4-((2-(2-aminopyrimidin-5-yl)-7-methyl-4-mohydroxypropan-1-one), GS-1101 (5-fluoro-3-phenyl-2-([S)]-1-[9H-purin-6-ylamino]-propyl)-3H-quinazolin-4-one), AMG-319, GSK-2269557, SAR245409 (N-(4-(N-(3-((3,5-dimethoxyphenyl)amino)quinoxalin-2-yl)sulfamoyl)phenyl)-3-methoxy-4 methylbenzamide), BAY80-6946 (2-amino-N-(7-methoxy-8-(3-morpholinopropoxy)-2,3-dihydroimidazo[1,2-c]quinaz), AS 252424 (5-[1-[5-(4-Fluoro-2-hydroxy-phenyl)-furan-2-yl]-meth-(Z)-ylidene]-thiazolidine-2,4-dione), CZ 24832 (5-(2-amino-8-fluoro-[1,2,4]triazolo[1,5-a]pyridin-6-yl)-N-tert-butylpyridine-3-sulfonamide), Buparlisib (5-[2,6-Di(4-morpholinyl)-4-pyrimidinyl]-4-(trifluoromethyl)-2-pyridinamine), GDC-0941 (2-(1H-Indazol-4-yl)-6-[[4-(methylsulfonyl)-1-piperazinyl]methyl]-4-(4-morpholinyl)thieno[3,2-d]pyrimidine), GDC-0980 ((S)-1-(4-((2-(2-aminopyrimidin-5-yl)-7-methyl-4-morpholinothieno[3,2-d]pyrimidin-6 yl)methyl)piperazin-1-yl)-2-hydroxypropan-1-one (also known as RG7422)), SF1126 ((8S,14S,17S)-14-(carboxymethyl)-8-(3-guanidinopropyl)-17-(hydroxymethyl)-3,6,9,12,15-pentaoxo-1-(4-(4-oxo-8-phenyl-4H-chromen-2-yl)morpholino-4-ium)-2-oxa-7,10,13,16-tetraazaoctadecan-18-oate), PF-05212384 (N-[4-[[4-(Dimethylamino)-1-piperidinyl]carbonyl]phenyl]-N-[4-(4,6-di-4-morpholinyl-1,3,5-triazin-2-yl)phenyl]urea) (gedatolisib), LY3023414, BEZ235 (2-Methyl-2-{4-[3-methyl-2-oxo-8-(quinolin-3-yl)-2,3-dihydro-1H-imidazo[4,5-c]quinolin-1-yl]phenyl}propanenitrile) (dactolisib), XL-765 (N-(3-(N-(3-(3,5-dimethoxyphenylamino)quinoxalin-2-yl)sulfamoyl)phenyl)-3-methoxy-4-methylbenzamide), and GSK1059615 (5-[[4-(4-Pyridinyl)-6-quinolinyl]methylene]-2,4-thiazolidenedione), PX886 ([(3aR,6E,9S,9aR,10R,11aS)-6-[[bis(prop-2-enyl)amino]methylidene]-5-hydroxy-9-(methoxymethyl)-9a,11a-dimethyl-1,4,7-trioxo-2,3,3a,9,10,11-hexahydroindeno[4,5h]isochromen-10-yl] acetate (also known as sonolisib)), LY294002, AZD8186, PF-4989216, pilaralisib, GNE-317, PI-3065, PI-103, NU7441 (KU-57788), HS 173, VS-5584 (SB2343), CZC24832, TG100-115, A66, YM201636, CAY10505, PIK-75, PIK-93, AS-605240, BGT226 (NVP-BGT226), AZD6482, voxtalisib, alpelisib, IC-87114, TGI100713, CH5132799, PKI-402, copanlisib (BAY 80-6946), XL 147, PIK-90, PIK-293, PIK-294, 3-MA (3-methyladenine), AS-252424, AS-604850, apitolisib (GDC-0980; RG7422), and the structure described in WO2014/071109 having the formula:

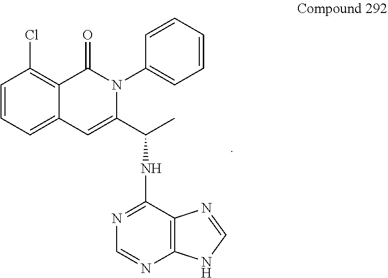 [SMILES: C[C@H](Nc1ncnc2[nH]cnc12)c1cc2cccc(Cl)c2c(=O)n1-c1ccccc1]

BTK inhibitors for use in the present invention are well known. Examples of BTK inhibitors include ibrutinib (also known as PCI-32765)(Imbruvica™)(1-[(3R)-3-[4-amino-3-(4-phenoxy-phenyl)pyrazolo[3,4-d]pyrimidin-1-yl]piperidin-1-yl]prop-2-en-1-one), dianilinopyrimidine-based inhibitors such as AVL-101 and AVL-291/292 (N-(3-((5-fluoro-2-((4-(2-methoxyethoxy)phenyl)amino)pyrimidin-4-yl)amino)phenyl)acrylamide) (Avila Therapeutics) (US Patent publication No 2011/0117073, incorporated herein in its entirety), Dasatinib ([N-(2-chloro-6-methylphenyl)-2-(6-(4-(2-hydroxyethyl)piperazin-1-yl)-2-methylpyrimidin-4-ylamino)thiazole-5-carboxamide], LFM-A13 (alpha-cyano-beta-hydroxy-beta-methyl-N-(2,5-ibromophenyl) propenamide), GDC-0834 ([R—N-(3-(6-(4-(1,4-dimethyl-3-oxopiperazin-2-yl)phenylamino)-4-methyl-5-oxo-4,5-dihydropyrazin-2-yl)-2-methylphenyl)-4,5,6,7-tetrahydrobenzo[b]thiophene-2-carboxamide], CGI-560 4-(tert-butyl)-N-(3-(8-(phenylamino)imidazo[1,2-a]pyrazin-6-yl)phenyl)benzamide, CGI-1746 (4-(tert-butyl)-N-(2-methyl-3-(4-methyl-6-((4-(morpholine-4-carbonyl)phenyl)amino)-5-oxo-4,5-dihydropyrazin-2-yl)phenyl)benzamide), CNX-774 (4-(4-((4-((3-acrylamidophenyl)amino)-5-fluoropyrimidin-2-yl)amino)phenoxy)-N-methylpicolinamide), CTA056 (7-benzyl-1-(3-(piperidin-1-yl)propyl)-2-(4-(pyridin-4-yl)phenyl)-1H-imidazo[4,5-g]quinoxalin-6(5H)-one), GDC-0834 ((R)—N-(3-(6-((4-(1,4-dimethyl-3-oxopiperazin-2-yl)phenyl)amino)-4-methyl-5-oxo-4,5-dihydropyrazin-2-yl)-2-methylphenyl)-4,5,6,7-tetrahydrobenzo[b]thiophene-2-carboxamide), GDC-0837 ((R)—N-(3-(6-((4-(1,4-dimethyl-3-oxopiperazin-2-yl)phenyl)amino)-4-methyl-5-oxo-4,5-dihydropyrazin-2-yl)-2-methylphenyl)-4,5,6,7-tetrahydrobenzo[b]thiophene-2-carboxamide), HM-71224, ACP-196, ONO-4059 (Ono Pharmaceuticals), PRT062607 (4-((3-(2H-1,2,3-triazol-2-yl)phenyl)amino)-2-(((1R,2S)-2-aminocyclohexyl)amino)pyrimidine-5-carboxamide hydrochloride), QL-47 (1-(1-acryloylindolin-6-yl)-9-(1-methyl-1H-pyrazol-4-yl)benzo[h][1,6]naphthyridin-2(1H)-one), and RN486 (6-cyclopropyl-8-fluoro-2-(2-hydroxymethyl-3-{1-methyl-5-[5-(4-methyl-piperazin-1-yl)-pyridin-2-ylamino]-6-oxo-1,6-dihydro-pyridin-3-yl}-phenyl)-2H-isoquinolin-1-one), and other molecules capable of inhibiting BTK activity, for example those BTK inhibitors disclosed in Akinleye et ah, Journal of Hematology & Oncology, 2013, 6:59, the entirety of which is incorporated herein by reference.

Syk inhibitors for use in the present invention are well known, and include, for example, Cerdulatinib (4-(cyclopropylamino)-2-((4-(4-(ethylsulfonyl)piperazin-1-yl)phenyl)amino)pyrimidine-5-carboxamide), entospletinib (6-(1H-indazol-6-yl)-N-(4-morpholinophenyl)imidazo[1,2-a]pyrazin-8-amine), fostamatinib ([6-({5-Fluoro-2-[(3,4,5-trimethoxyphenyl)amino]-4-pyrimidinyl}amino)-2,2-dimethyl-3-oxo-2,3-dihydro-4H-pyrido[3,2-b][1,4]oxazin-4-yl]methyl dihydrogen phosphate), fostamatinib disodium salt (sodium (6-((5-fluoro-2-((3,4,5-trimethoxyphenyl)amino)pyrimidin-4-yl)amino)-2,2-dimethyl-3-oxo-2H-pyrido[3,2-b][1,4]oxazin-4(3H)-yl)methyl phosphate), BAY 61-3606 (2-(7-(3,4-Dimethoxyphenyl)-imidazo[1,2-c]pyrimidin-5-ylamino)-nicotinamide HCl), R09021 (6-[(1R,2S)-2-Amino-cyclohexylamino]-4-(5,6-dimethyl-pyridin-2-ylamino)-pyridazine-3-carboxylic acid amide), imatinib (Gleevac; 4-[(4-methylpiperazin-1-yl)methyl]-N-(4-methyl-3-{[4-(pyridin-3-yl)pyrimidin-2-yl]amino}phenyl)benzamide), staurosporine, GSK143 (2-(((3R,4R)-3-aminotetrahydro-2H-pyran-4-yl)amino)-4-(p-tolylamino)pyrimidine-5-carboxamide), PP2 (1-(tert-butyl)-3-(4-chlorophenyl)-1H-pyrazolo[3,4-d]pyrimidin-4-amine), PRT-060318 (2-(((1R,2S)-2-aminocyclohexyl)amino)-4-(m-tolylamino)pyrimidine-5-carboxamide), PRT-062607 (4-((3-(2H-1,2,3-triazol-2-yl)phenyl)amino)-2-(((1R,2S)-2-aminocyclohexyl)amino)pyrimidine-5-carboxamide hydrochloride), R112 (3,3′-((5-fluoropyrimidine-2,4-diyl)bis(azanediyl))diphenol), R348 (3-Ethyl-4-methylpyridine), R406 (6-((5-fluoro-2-((3,4,5-trimethoxyphenyl)amino)pyrimidin-4-yl)amino)-2,2-dimethyl-2H-pyrido[3,2-b][1,4]oxazin-3(4H)-one), piceatannol (3-Hydroxyresveratol), YM193306 (Singh et al. Discovery and Development of Spleen Tyrosine Kinase (SYK) Inhibitors, J. Med. Chem. 2012, 55, 3614-3643), 7-azaindole, piceatannol, ER-27319 (Singh et al. Discovery and Development of Spleen Tyrosine Kinase (SYK) Inhibitors, J. Med. Chem. 2012, 55, 3614-3643 incorporated in its entirety herein), Compound D (Singh et al. Discovery and Development of Spleen Tyrosine Kinase (SYK) Inhibitors, J. Med Chem. 2012, 55, 3614-3643 incorporated in its entirety herein), PRT060318 (Singh et al. Discovery and Development of Spleen Tyrosine Kinase (SYK) Inhibitors, J. Med Chem. 2012, 55, 3614-3643 incorporated in its entirety herein), luteolin (Singh et al. Discovery and Development of Spleen Tyrosine Kinase (SYK) Inhibitors, J. Med Chem. 2012, 55, 3614-3643 incorporated in its entirety herein), apigenin (Singh et al. Discovery and Development of Spleen Tyrosine Kinase (SYK) Inhibitors, J. Med Chem. 2012, 55, 3614-3643 incorporated in its entirety herein), quercetin (Singh et al. Discovery and Development of Spleen Tyrosine Kinase (SYK) Inhibitors, J. Med Chem. 2012, 55, 3614-3643 incorporated in its entirety herein), fisetin (Singh et al. Discovery and Development of Spleen Tyrosine Kinase (SYK) Inhibitors, J. Med Chem. 2012, 55, 3614-3643 incorporated in its entirety herein), myricetin (Singh et al. Discovery and Development of Spleen Tyrosine Kinase (SYK) Inhibitors, J. Med Chem. 2012, 55, 3614-3643 incorporated in its entirety herein), morin (Singh et al. Discovery and Development of Spleen Tyrosine Kinase (SYK) Inhibitors, J. Med Chem. 2012, 55, 3614-3643 incorporated in its entirety herein).

In one embodiment, the therapeutic agent is a MEK inhibitor. MEK inhibitors for use in the present invention are well known, and include, for example, trametinib/GSK1120212 (N-(3-{3-Cyclopropyl-5-[(2-fluoro-4-iodophenyl)amino]-6,8-dimethyl-2,4,7-trioxo-3,4,6,7-tetrahydropyrido[4,3-d]pyrimidin-1(2H-yl}phenyl)acetamide), selumetinib (6-(4-bromo-2-chloroanilino)-7-fluoro-N-(2-hydroxyethoxy)-3-methylbenzimidazole-5-carboxamide), pimasertib/AS703026/MSC 1935369 ((S)—N-(2,3-dihydroxypropyl)-3-((2-fluoro-4-iodophenyl)amino)isonicotinamide), XL-518/GDC-0973 (1-({3,4-difluoro-2-[(2-fluoro-4-iodophenyl)amino]phenyl}carbonyl)-3-[(2S)-piperidin-2-yl]azetidin-3-ol), refametinib/BAY869766/RDEAl 19 (N-(3,4-difluoro-2-(2-fluoro-4-iodophenylamino)-6-methoxyphenyl)-1-(2,3-dihydroxypropyl)cyclopropane-1-sulfonamide), PD-0325901 (N-[(2R)-2,3-Dihydroxypropoxy]-3,4-difluoro-2-[(2-fluoro-4-iodophenyl)amino]-benzamide), TAK733 ((R)-3-(2,3-Dihydroxypropyl)-6-fluoro-5-(2-fluoro-4-iodophenylamino)-8-methylpyrido[2,3-d]pyrimidine-4,7(3H,8H)-dione), MEK162/ARRY438162 (5-[(4-Bromo-2-fluorophenyl)amino]-4-fluoro-N-(2-hydroxyethoxy)-1-methyl-1H-benzimidazole-6-carboxamide), R05126766 (3-[[3-Fluoro-2-(methylsulfamoylamino)-4-pyridyl]methyl]-4-methyl-7-pyrimidin-2-yloxychromen-2-one), WX-554, R04987655/CH4987655 (3,4-difluoro-2-((2-fluoro-4-iodophenyl)amino)-N-(2-hydroxyethoxy)-5-((3-oxo-1,2-oxazinan-2yl)methyl)benzamide), or AZD8330 (2-((2-fluoro-4-iodophenyl)amino)-N-(2 hydroxyethoxy)-1, 5-dimethyl-6-oxo-1,6-dihydropyridine-3-carboxamide), U0126-EtOH, PD184352 (CI-1040), GDC-0623, BI-847325, cobimetinib, PD98059, BIX 02189, BIX 02188, binimetinib, SL-327, TAK-733, PD318088, and additional MEK inhibitors as described below.

In one embodiment, the therapeutic agent is a Raf inhibitor. Raf inhibitors for use in the present invention are well known, and include, for example, Vemurafinib (N-[3-[[5-(4-Chlorophenyl)-1H-pyrrolo[2,3-b]pyridin-3-yl]carbonyl]-2,4-difluorophenyl]-1-propanesulfonamide), sorafenib tosylate (4-[4-[[4-chloro-3-(trifluoromethyl)phenyl]carbamoylamino]phenoxy]-N-methylpyridine-2-carboxamide; 4-methylbenzenesulfonate), AZ628 (3-(2-cyanopropan-2-yl)-N-(4-methyl-3-(3-methyl-4-oxo-3,4-dihydroquinazolin-6-ylamino)phenyl)benzamide), NVP-BHG712 (4-methyl-3-(1-methyl-6-(pyridin-3-yl)-1H-pyrazolo[3,4-d]pyrimidin-4-ylamino)-N-(3-(trifluoromethyl)phenyl)benzamide), RAF-265 (1-methyl-5-[2-[5-(trifluoromethyl)-1H-imidazol-2-yl]pyridin-4-yl]oxy-N-[4-(trifluoromethyl)phenyl]benzimidazol-2-amine), 2-Bromoaldisine (2-Bromo-6,7-dihydro-1H,5H-pyrrolo[2,3-c]azepine-4,8-dione), Raf Kinase Inhibitor IV (2-chloro-5-(2-phenyl-5-(pyridin-4-yl)-1H-imidazol-4-yl)phenol), Sorafenib N-Oxide (4-[4-[[[[4-Chloro-3(trifluoroMethyl)phenyl]aMino]carbonyl]aMino]phenoxy]-N-Methyl-2pyridinecarboxaMide 1-Oxide), PLX-4720, dabrafenib (GSK2118436), GDC-0879, RAF265, AZ 628, Sf590885, ZM336372, GW5074, TAK-632, CEP-32496, LY3009120, and GX818 (Encorafenib).

In one embodiment, the therapeutic agent is a programmed death protein 1 (PD-1) inhibitor, a programmed death protein ligand 1 (PDL1) inhibitor, or a programmed death protein ligand 2 (PDL2) inhibitor. PD-1, PDL1, and PDL2 inhibitors are known in the art, and include, for example, nivolumab (BMS), pembrolizumab (Merck), pidilizumab (CureTech/Teva), AMP-244 (Amplimmune/GSK), BMS-936559 (BMS), and MEDI4736 (Roche/Genentech), and MPDL3280 A (Genentech).

In one embodiment, a therapeutic agent can be administered in a sustained fashion.

In one embodiment, the therapeutic agent is a monoclonal antibody (MAb). Some MAbs stimulate an immune response that destroys cancer cells. Similar to the antibodies produced naturally by B cells, these MAbs “coat” the cancer cell surface, triggering its destruction by the immune system. For example, bevacizumab targets vascular endothelial growth factor (VEGF), a protein secreted by tumor cells and other cells in the tumor's microenvironment that promotes the development of tumor blood vessels. When bound to bevacizumab, VEGF cannot interact with its cellular receptor, preventing the signaling that leads to the growth of new blood vessels. Similarly, cetuximab and panitumumab target the epidermal growth factor receptor (EGFR), and trastuzumab targets the human epidermal growth factor receptor 2 (HER-2). MAbs that bind to cell surface growth factor receptors prevent the targeted receptors from sending their normal growth-promoting signals. They may also trigger apoptosis and activate the immune system to destroy tumor cells.

Other agents may include, but are not limited to, at least one of tamoxifen, midazolam, letrozole, bortezomib, anastrozole, goserelin, an mTOR inhibitor, a PI3 kinase inhibitor as described above, a dual mTOR-PI3K inhibitor, a MEK inhibitor, a RAS inhibitor, ALK inhibitor, an HSP inhibitor (for example, HSP70 and HSP 90 inhibitor, or a combination thereof), a BCL-2 inhibitor as described above, apopototic inducing compounds, an AKT inhibitor, including but not limited to, MK-2206, GSK690693, Perifosine, (KRX-0401), GDC-0068, Triciribine, AZD5363, Honokiol, PF-04691502, and Miltefosine, a PD-1 inhibitor as described above including but not limited to, Nivolumab, CT-011, MK-3475, BMS936558, and AMP-514 or a FLT-3 inhibitor, including but not limited to, P406, Dovitinib, Quizartinib (AC220), Amuvatinib (MP-470), Tandutinib (MLN518), ENMD-2076, and KW-2449, or a combination thereof. Examples of mTOR inhibitors include but are not limited to rapamycin and its analogs, everolimus (Afinitor), temsirolimus, ridaforolimus, sirolimus, and deforolimus. Examples of MEK inhibitors include but are not limited to tametinib/GSK1120212 (N-(3-{3-Cyclopropyl-5-[(2-fluoro-4-iodophenyl)amino]-6,8-dimethyl-2,4,7-trioxo-3,4,6,7-tetrahydropyrido[4,3-d]pyrimidin-1(2H-yl}phenyl)acetamide), selumetinob (6-(4-bromo-2-chloroanilino)-7-fluoro-N-(2-hydroxyethoxy)-3-methylbenzimidazole-5-carboxamide), pimasertib/AS703026/MSC1935369 ((S)—N-(2,3-dihydroxypropyl)-3-((2-fluoro-4-iodophenyl)amino)isonicotinamide), XL-518/GDC-0973 (1-({3,4-difluoro-2-[(2-fluoro-4-iodophenyl)amino]phenyl}carbonyl)-3-[(2S)-piperidin-2-yl]azetidin-3-ol) (cobimetinib), refametinib/BAY869766/RDEA119 (N-(3,4-difluoro-2-(2-fluoro-4-iodophenylamino)-6-methoxyphenyl)-1-(2,3-dihydroxypropyl)cyclopropane-1-sulfonamide), PD-0325901 (N-[(2R)-2,3-Dihydroxypropoxy]-3,4-difluoro-2-[(2-fluoro-4-iodophenyl)amino]-benzamide), TAK733 ((R)-3-(2,3-Dihydroxypropyl)-6-fluoro-5-(2-fluoro-4-iodophenylamino)-8-methylpyrido[2,3d]pyrimidine-4,7(3H,8H)-dione), MEK162/ARRY438162 (5-[(4-Bromo-2-fluorophenyl)amino]-4-fluoro-N-(2-hydroxyethoxy)-1-methyl-1H-benzimidazole-6 carboxamide), R05126766 (3-[[3-Fluoro-2-(methylsulfamoylamino)-4-pyridyl]methyl]-4-methyl-7-pyrimidin-2-yloxychromen-2-one), WX-554, R04987655/CH4987655 (3,4-difluoro-2-((2-fluoro-4-iodophenyl)amino)-N-(2-hydroxyethoxy)-5-((3-oxo-1,2-oxazinan-2 yl)methyl)benzamide), or AZD8330 (2-((2-fluoro-4-iodophenyl)amino)-N-(2-hydroxyethoxy)-1,5-dimethyl-6-oxo-1,6-dihydropyridine-3-carboxamide). Examples of RAS inhibitors include but are not limited to Reolysin and siG12D LODER. Examples of ALK inhibitors include but are not limited to Crizotinib, Ceritinib (Zykadia), AP26113, and LDK378. HSP inhibitors include but are not limited to Geldanamycin or 17-N-Allylamino-17-demethoxygeldanamycin (17AAG), and Radicicol.

In certain aspects, the therapeutic agent is an anti-inflammatory agent, a chemotherapeutic agent, a radiotherapeutic, an additional therapeutic agent, or an immunosuppressive agent.

In one embodiment, a chemotherapeutic is selected from, but not limited to, imatinib mesylate (Gleevac®), dasatinib (Sprycel®), nilotinib (Tasigna®), bosutinib (Bosulif®), trastuzumab (Herceptin®), trastuzumab-DM1, pertuzumab (Perjeta™), lapatinib (Tykerb®), gefitinib (Iressa®), erlotinib (Tarceva®), cetuximab (Erbitux®), panitumumab (Vectibix®), vandetanib (Caprelsa®), vemurafenib (Zelboraf®), vorinostat (Zolinza®), romidepsin (Istodax®), bexarotene (Tagretin®), alitretinoin (Panretin®), tretinoin (Vesanoid®), carfilizomib (Kyprolis™), pralatrexate (Folotyn®), bevacizumab (Avastin®), ziv-aflibercept (Zaltrap®), sorafenib (Nexavar®), sunitinib (Sutent®), pazopanib (Votrient®), regorafenib (Stivarga®), and cabozantinib (Cometriq™).

Additional chemotherapeutic agents include, but are not limited to, a radioactive molecule, a toxin, also referred to as cytotoxin or cytotoxic agent, which includes any agent that is detrimental to the viability of cells, and liposomes or other vesicles containing chemotherapeutic compounds. General anticancer pharmaceutical agents include: vincristine (Oncovin®) or liposomal vincristine (Marqibo®), daunorubicin (daunomycin or Cerubidine®) or doxorubicin (Adriamycin®), cytarabine (cytosine arabinoside, ara-C, or Cytosar®), L-asparaginase (Elspar®) or PEG-L-asparaginase (pegaspargase or Oncaspar®), etoposide (VP-16), teniposide (Vumon®), 6-mercaptopurine (6-MP or Purinethol®), Methotrexate, cyclophosphamide (Cytoxan®), Prednisone, dexamethasone (Decadron), imatinib (Gleevec®), dasatinib (Sprycel®), nilotinib (Tasigna®), bosutinib (Bosulif®), and ponatinib (Iclusig™). Examples of additional suitable chemotherapeutic agents include but are not limited to 1-dehydrotestosterone, 5-fluorouracil decarbazine, 6-mercaptopurine, 6-thioguanine, actinomycin D, adriamycin, aldesleukin, an alkylating agent, allopurinol sodium, altretamine, amifostine, anastrozole, anthramycin (AMC)), an anti-mitotic agent, cis-dichlorodiamine platinum (II) (DDP) cisplatin), diamino dichloro platinum, anthracycline, an antibiotic, an antimetabolite, asparaginase, BCG live (intravesical), betamethasone sodium phosphate and betamethasone acetate, bicalutamide, bleomycin sulfate, busulfan, calcium leucouorin, calicheamicin, capecitabine, carboplatin, lomustine (CCNU), carmustine (BSNU), chlorambucil, cisplatin, cladribine, colchicin, conjugated estrogens, cyclophosphamide, cyclothosphamide, cytarabine, cytarabine, cytochalasin B, cytoxan, dacarbazine, dactinomycin, dactinomycin (formerly actinomycin), daunirubicin HCL, daunorucbicin citrate, denileukin diftitox, Dexrazoxane, Dibromomannitol, dihydroxy anthracin dione, docetaxel, dolasetron mesylate, doxorubicin HCL, dronabinol, E. coli L-asparaginase, emetine, epoetin-α, Erwinia L-asparaginase, esterified estrogens, estradiol, estramustine phosphate sodium, ethidium bromide, ethinyl estradiol, etidronate, etoposide citrororum factor, etoposide phosphate, filgrastim, floxuridine, fluconazole, fludarabine phosphate, fluorouracil, flutamide, folinic acid, gemcitabine HCL, glucocorticoids, goserelin acetate, gramicidin D, granisetron HCL, hydroxyurea, idarubicin HCL, ifosfamide, interferon α-2b, irinotecan HCL, letrozole, leucovorin calcium, leuprolide acetate, levamisole HCL, lidocaine, lomustine, maytansinoid, mechlorethamine HCL, medroxyprogesterone acetate, megestrol acetate, melphalan HCL, mercaptipurine, mesna, methotrexate, methyltestosterone, mithramycin, mitomycin C, mitotane, mitoxantrone, nilutamide, octreotide acetate, ondansetron HCL, paclitaxel, pamidronate disodium, pentostatin, pilocarpine HCL, plimycin, polifeprosan 20 with carmustine implant, porfimer sodium, procaine, procarbazine HCL, propranolol, rituximab, sargramostim, streptozotocin, tamoxifen, taxol, teniposide, tenoposide, testolactone, tetracaine, thioepa chlorambucil, thioguanine, thiotepa, topotecan HCL, toremifene citrate, trastuzumab, tretinoin, valrubicin, vinblastine sulfate, vincristine sulfate, and vinorelbine tartrate.

Additional therapeutic agents can include bevacizumab, sutinib, sorafenib, 2-methoxyestradiol or 2ME2, finasunate, vatalanib, vandetanib, aflibercept, volociximab, etaracizumab (MEDI-522), cilengitide, erlotinib, cetuximab, panitumumab, gefitinib, trastuzumab, dovitinib, figitumumab, atacicept, rituximab, alemtuzumab, aldesleukine, atlizumab, tocilizumab, temsirolimus, everolimus, lucatumumab, dacetuzumab, HLL1, huN901-DM1, atiprimod, natalizumab, bortezomib, carfilzomib, marizomib, tanespimycin, saquinavir mesylate, ritonavir, nelfinavir mesylate, indinavir sulfate, belinostat, panobinostat, mapatumumab, lexatumumab, dulanermin, ABT-737, oblimersen, plitidepsin, talmapimod, P276-00, enzastaurin, tipifarnib, perifosine, imatinib, dasatinib, lenalidomide, thalidomide, simvastatin, celecoxib, bazedoxifene, AZD4547, rilotumumab, oxaliplatin (Eloxatin), PD0332991 (palbociclib), ribociclib (LEE011), amebaciclib (LY2835219), HDM201, fulvestrant (Faslodex), exemestane (Aromasin), PIM447, ruxolitinib (INC424), BGJ398, necitumumab, pemetrexed (Alimta), and ramucirumab (IMC-1121B).

In one aspect of the present invention, an immunosuppressive agent is used, preferably selected from the group consisting of a calcineurin inhibitor, e.g. a cyclosporin or an ascomycin, e.g. Cyclosporin A (NEORAL®), FK506 (tacrolimus), pimecrolimus, a mTOR inhibitor, e.g. rapamycin or a derivative thereof, e.g. Sirolimus (RAPAMUNE®), Everolimus (Certican®), temsirolimus, zotarolimus, biolimus-7, biolimus-9, a rapalog, e.g.ridaforolimus, azathioprine, campath 1H, a SiP receptor modulator, e.g. fingolimod or an analogue thereof, an anti-IL-8 antibody, mycophenolic acid or a salt thereof, e.g. sodium salt, or a prodrug thereof, e.g. Mycophenolate Mofetil (CELLCEPT®), OKT3 (ORTHOCLONE OKT3®), Prednisone, ATGAM®, THYMOGLOBULIN®, Brequinar Sodium, OKT4, T10B9.A-3 A, 33B3.1, 15-deoxyspergualin, tresperimus, Leflunomide ARAVA®, CTLAI-Ig, anti-CD25, anti-IL2R, Basiliximab (SIULECT®), Daclizumab (ZENAPAX®), mizorbine, methotrexate, dexamethasone, ISAtx-247, SDZ ASM 981 (pimecrolimus, Elidel®), CTLA4lg (Abatacept), belatacept, LFA3lg, etanercept (sold as Enbrel® by Immunex), adalimumab (Humira®), infliximab (Remicade®), an anti-LFA-1 antibody, natalizumab (Antegren®), Enlimomab, gavilimomab, antithymocyte immunoglobulin, siplizumab, Alefacept efalizumab, pentasa, mesalazine, asacol, codeine phosphate, benorylate, fenbufen, naprosyn, diclofenac, etodolac and indomethacin, aspirin and ibuprofen.

Examples of types of therapeutic agents that can be eluted from the microparticles include anti-inflammatory drugs, antimicrobial agents, anti-angiogenesis agents, immunosuppressants, antibodies, steroids, ocular antihypertensive drugs and combinations thereof. Examples of therapeutic agents include amikacin, anecortane acetate, anthracenedione, anthracycline, an azole, amphotericin B, bevacizumab, camptothecin, cefuroxime, chloramphenicol, chlorhexidine, chlorhexidine digluconate, clortrimazole, a clotrimazole cephalosporin, corticosteroids, dexamethasone, desamethazone, econazole, eftazidime, epipodophyllotoxin, fluconazole, flucytosine, fluoropyrimidines, fluoroquinolines, gatifloxacin, glycopeptides, imidazoles, itraconazole, ivermectin, ketoconazole, levofloxacin, macrolides, miconazole, miconazole nitrate, moxifloxacin, natamycin, neomycin, nystatin, ofloxacin, polyhexamethylene biguanide, prednisolone, prednisolone acetate, pegaptanib, platinum analogues, polymicin B, propamidine isethionate, pyrimidine nucleoside, ranibizumab, squalamine lactate, sulfonamides, triamcinolone, triamcinolone acetonide, triazoles, vancomycin, anti-vascular endothelial growth factor (VEGF) agents, VEGF antibodies, VEGF antibody fragments, vinca alkaloid, timolol, betaxolol, travoprost, latanoprost, bimatoprost, brimonidine, dorzolamide, acetazolamide, pilocarpine, ciprofloxacin, azithromycin, gentamycin, tobramycin, cefazolin, voriconazole, gancyclovir, cidofovir, foscarnet, diclofenac, nepafenac, ketorolac, ibuprofen, indomethacin, fluoromethalone, rimexolone, anecortave, cyclosporine, methotrexate, tacrolimus and combinations thereof.

Examples of immunosuppressive agents are calcineurin inhibitor, e.g., a cyclosporin or an ascomycin, e.g., Cyclosporin A (NEORAL®), FK506 (tacrolimus), pimecrolimus, a mTOR inhibitor, e.g., rapamycin or a derivative thereof, e.g., Sirolimus (RAPAMUNE®), Everolimus (Certican®), temsirolimus, zotarolimus, biolimus-7, biolimus-9, a rapalog, e.g., ridaforolimus, azathioprine, campath 1H, a SiP receptor modulator, e.g., fingolimod or an analogue thereof, an anti IL-8 antibody, mycophenolic acid or a salt thereof, e.g., sodium salt, or a prodrug thereof, e.g., Mycophenolate Mofetil (CELLCEPT®), OKT3 (ORTHOCLONE OKT3®), Prednisone, ATGAM®, THYMOGLOBULIN®, Brequinar Sodium, OKT4, T10B9.A-3 A, 33B3.1, 15-deoxyspergualin, tresperimus, Leflunomide ARAVA®, CTLAI-Ig, anti-CD25, anti-IL2R, Basiliximab (SVIMULECT®), Daclizumab (ZENAPAX®), mizorbine, methotrexate, dexamethasone, ISAtx-247, SDZ ASM 981 (pimecrolimus, Elidel®), CTLA4lg (Abatacept), belatacept, LFA3lg, etanercept (sold as Enbrel® by Immunex), adalimumab (Humira®), infliximab (Remicade®), an anti-LFA-1 antibody, natalizumab (Antegren®), Enlimomab, gavilimomab, antithymocyte immunoglobulin, siplizumab, Alefacept efalizumab, pentasa, mesalazine, asacol, codeine phosphate, benorylate, fenbufen, naprosyn, diclofenac, etodolac and indomethacin, aspirin and ibuprofen.

In certain embodiments, the surface-treated microparticles of the present invention can comprise a prodrug as disclosed below. In all of the polymer moieties described in this specification, where the structures are depicted as block copolymers (for example, blocks of “x” followed by blocks of “y”) it is intended that the polymer can alternately be a random or alternating copolymer (for example, “x” and “y”, are either randomly distributed or alternate). Unless stereochemistry is specifically indicated, each individual moiety of each oligomer that has a chiral center can be presented at the chiral carbon in (R) or (S) configuration or a mixture thereof, including a racemic mixture.

In addition, prodrug moieties that have repetitive units of the same or varying monomers, for example including but not limited to an oligomer of polylactic acid, polylactide-coglycolide, or polypropylene oxide, that has a chiral carbon can be used with the chiral carbons all having the same stereochemistry, random stereochemistry (by either monomer or oligomer), racemic (by either monomer or oligomer) or ordered but different stereochemistry such as a block of S enantiomer units followed by a block of R enantiomer units in each oligomeric unit. In some embodiments lactic acid is used in its naturally occurring S enantiomeric form.

Table A-Table I show illustrative prodrugs encapsulated in the microparticles of the present invention. In one aspect of the invention, a suspension of mildly surface-treated microparticles in a diluent comprising additive that improves particle aggregation where the microparticles comprise one of more biodegradable polymers and a prodrug selected from Table A-Table I encapsulated in the biodegradable polymer is provided.

An aspect of the invention is a method for the treatment of a disorder, comprising administering to a host in need thereof a suspension of solid aggregating microparticles in a diluent comprising additive wherein the microparticles comprise an effective amount of a therapeutic agent selected from a prodrug disclosed herein, wherein the therapeutic agent containing solid aggregating microparticles are injected into the body and aggregate in vivo to form at least one pellet of at least 500 μm that provides sustained drug delivery for at least one month.

TABLE A
Non-limiting Examples of Prodrugs

| Comp. # | Structure |
|---|---|
| 1 | 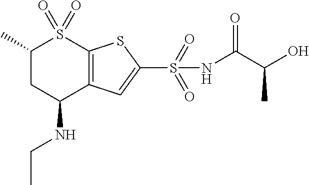 [SMILES: CCN[C@H]1C[C@H](C)S(=O)(=O)c2sc(S(=O)(=O)NC(=O)[C@H](C)O)cc21] |
| 2 | 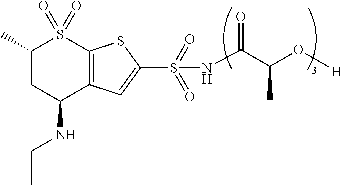 |
| 3 | 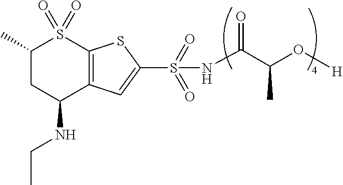 |
| 4 | 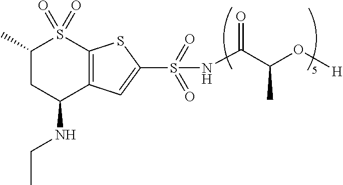 |
| 5 | 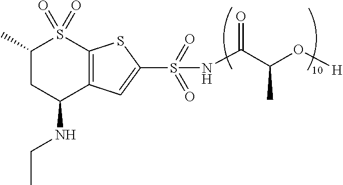 |
| 6 | 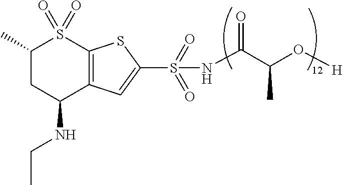 |
| 7 | 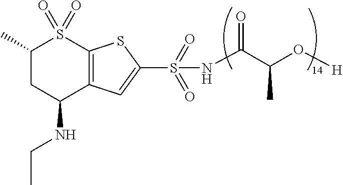 |
| 8 | 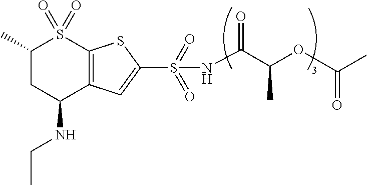 |
| 9 | 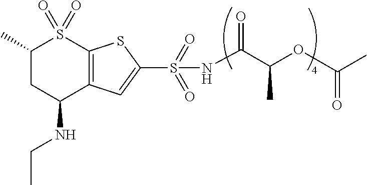 |
| 10 | 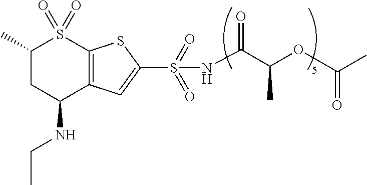 |
| 11 | 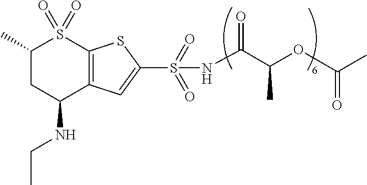 |
| 12 | 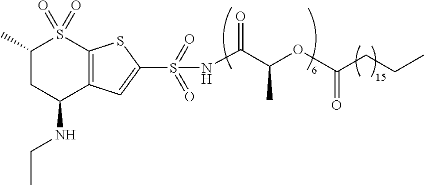 |
| 13 | 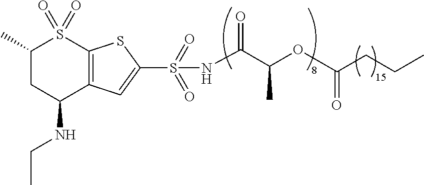 |
| 14 | 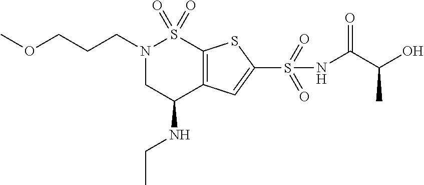 [SMILES: CCN[C@H]1CN(CCCOC)S(=O)(=O)c2sc(S(=O)(=O)NC(=O)[C@H](C)O)cc21] |
| 15 | 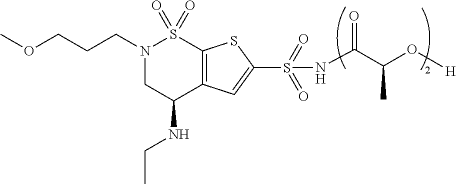 |
| 16 | 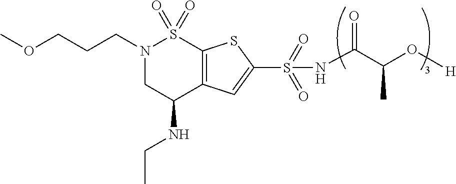 |
| 17 | 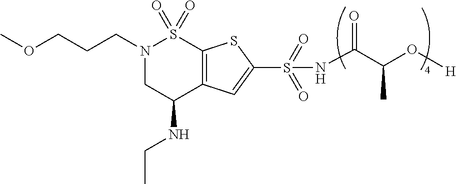 |
| 18 | 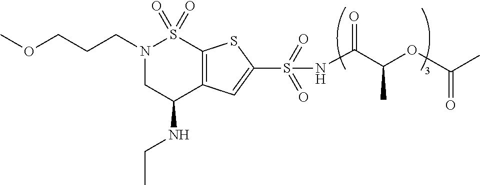 |
| 19 | 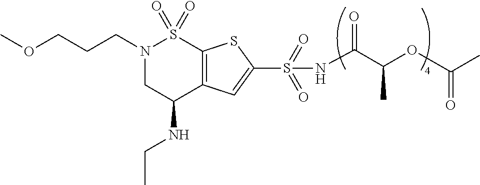 |
| 20 | 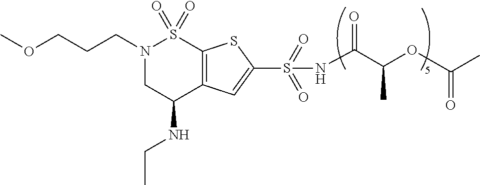 |
| 21 | 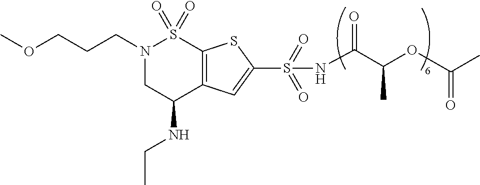 |
| 22 | 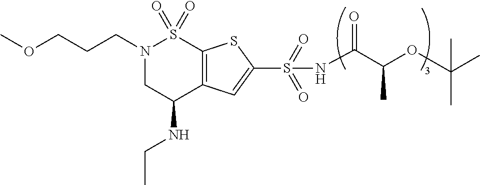 |
| 23 | 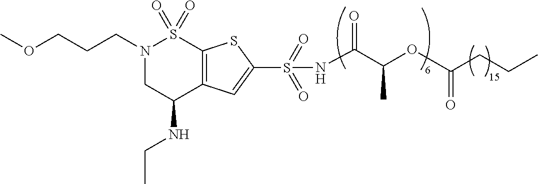 |
| 24 | 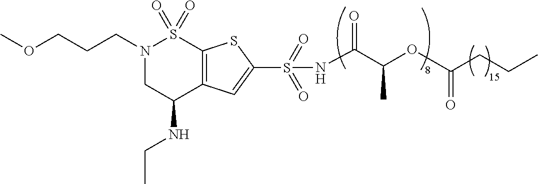 |
| 25 | 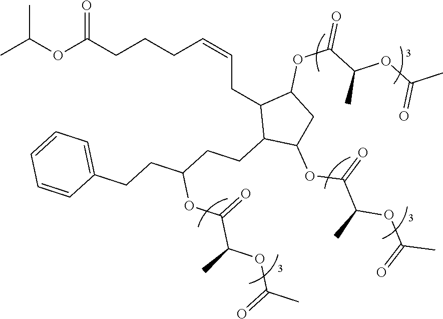 |
| 26 | 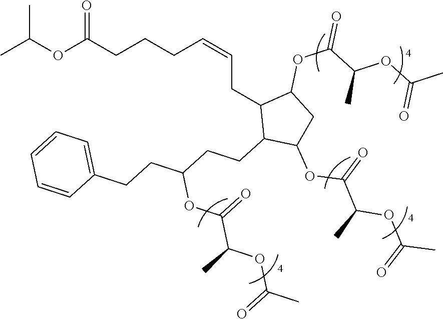 |
| 27 | 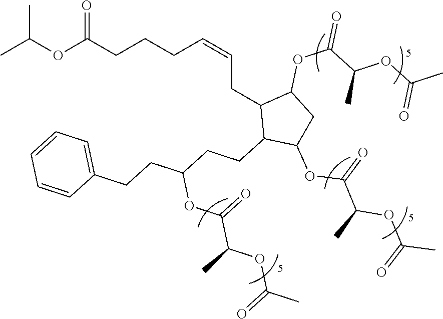 |
| 28 | 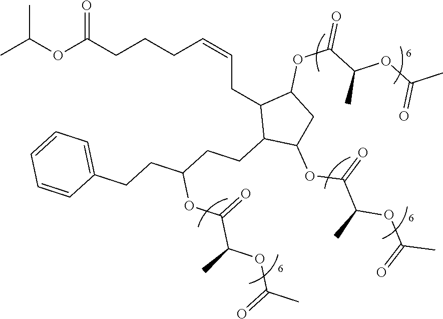 |
| 29 | 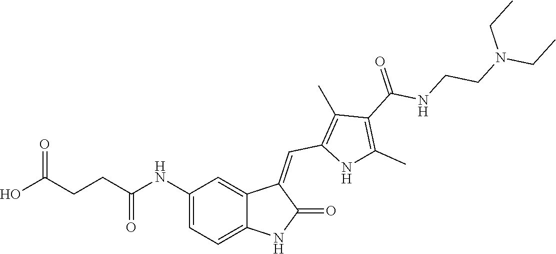 [SMILES: CCN(CC)CCNC(=O)c1c(C)[nH]c(/C=C2\C(=O)Nc3ccc(NC(=O)CCC(=O)O)cc32)c1C] |
| 30 | 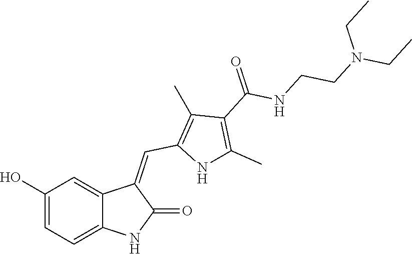 [SMILES: CCN(CC)CCNC(=O)c1c(C)[nH]c(/C=C2\C(=O)Nc3ccc(O)cc32)c1C] |
| 31 | 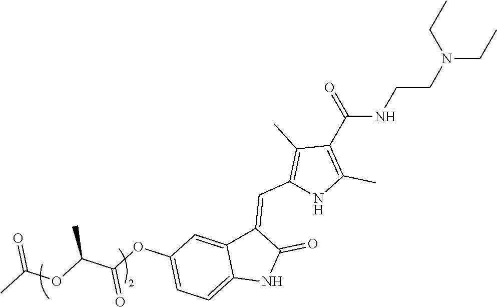 |
| 32 | 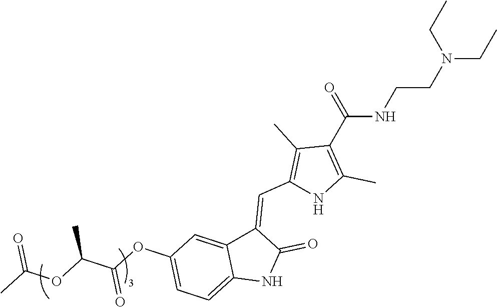 |
| 33 | 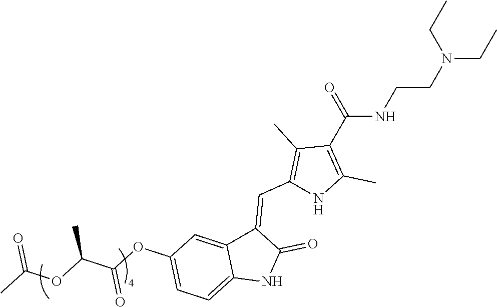 |
| 34 | 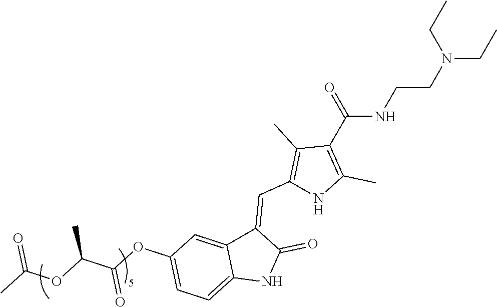 |
| 35 | 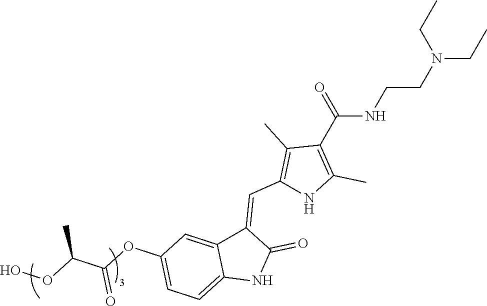 |
| 36 | 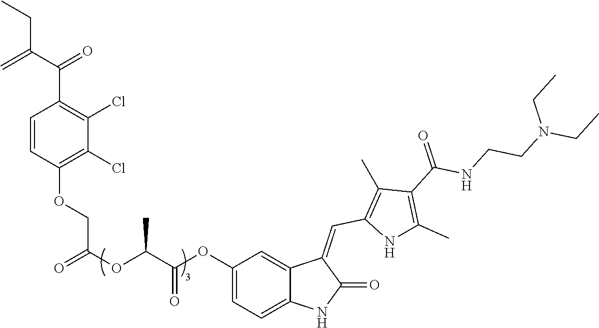 |
| 37 | 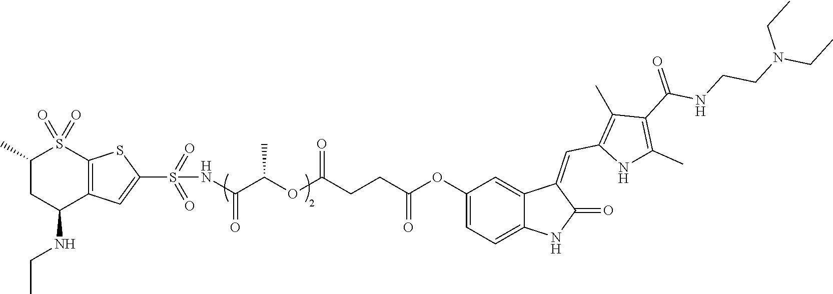 |
| 38 | 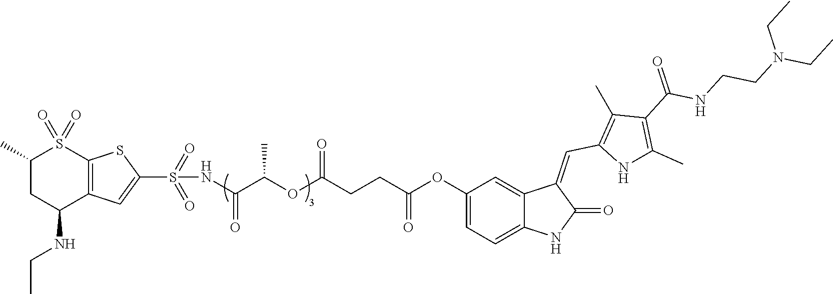 |
| 39 | 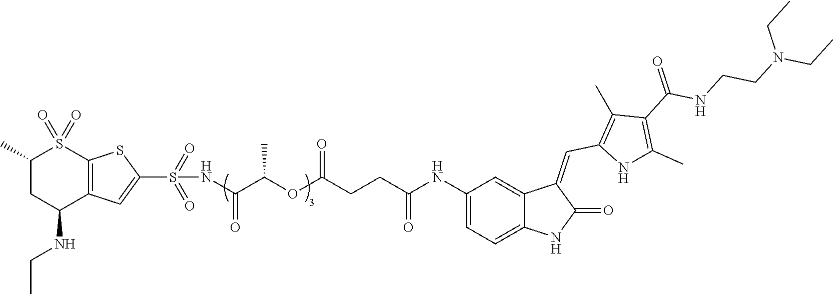 |
| 40 | 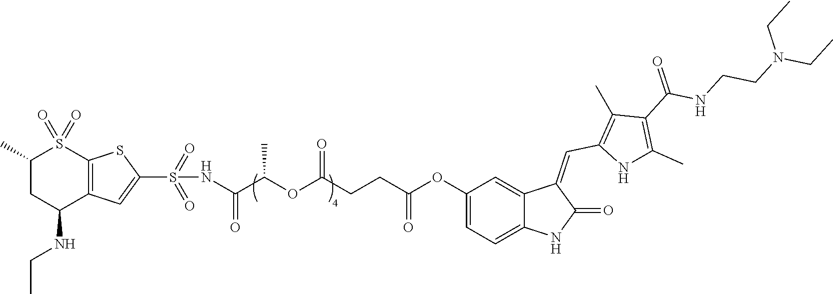 |
| 41 | 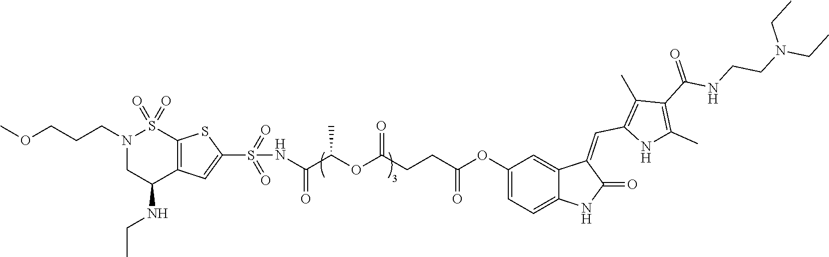 |
| 42 | 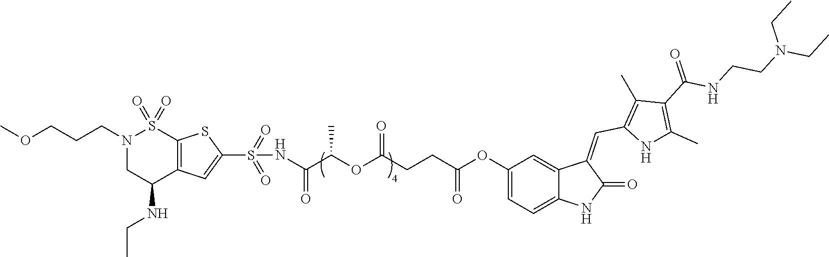 |
| 43 | 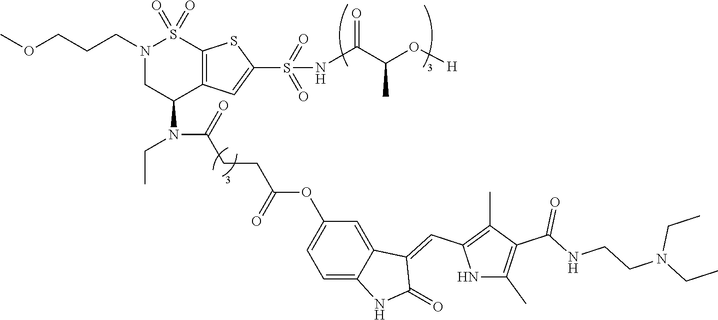 |
| 44 | 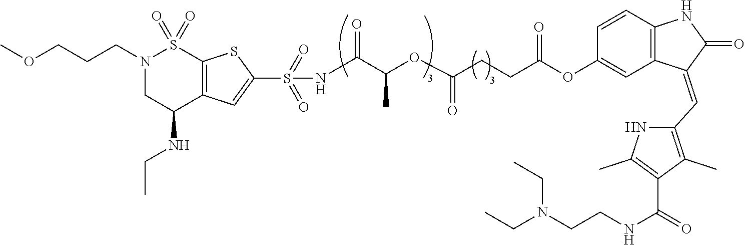 |

TABLE B
Non-limiting Examples of Prodrugs

| | |
|---|---|
| 45 | 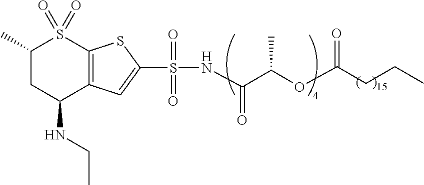 |
| 46 | 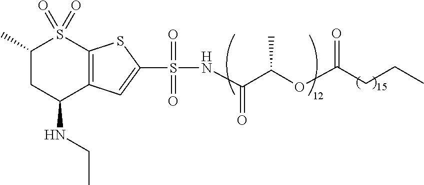 |
| 47 | 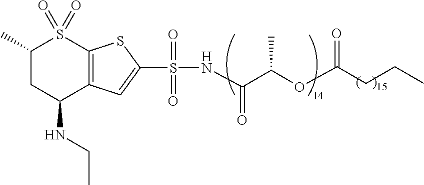 |
| 48 | 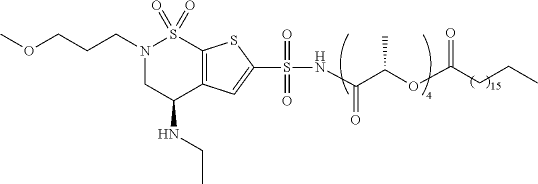 |
| 49 | 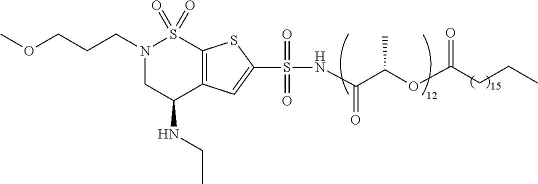 |
| 50 | 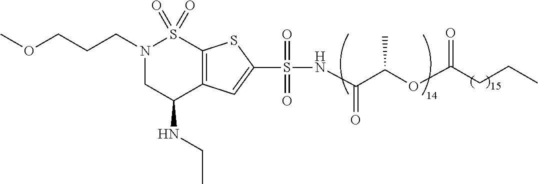 |
| 51 | 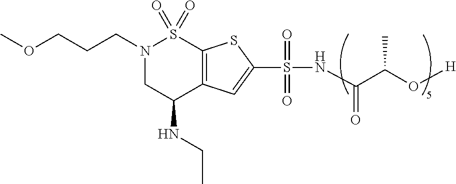 |
| 52 | 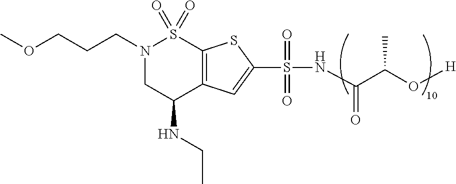 |
| 53 | 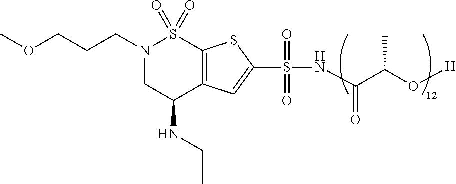 |
| 54 | 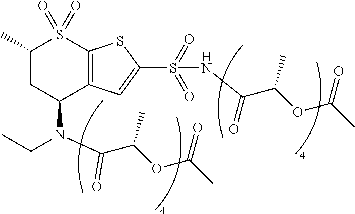 |
| 55 | 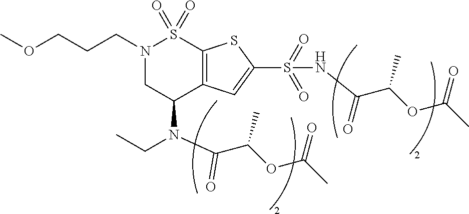 |
| 56 | 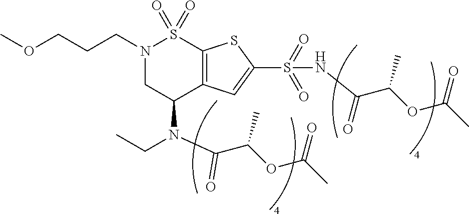 |
| 57 | 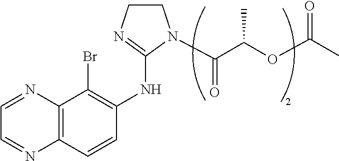 |
| 58 | 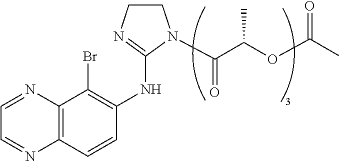 |
| 59 | 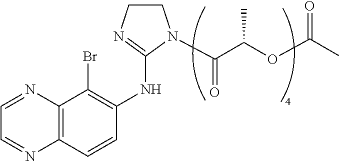 |
| 60 | 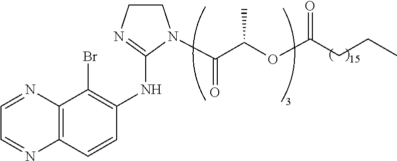 |
| 61 | 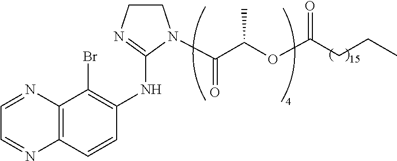 |
| 62 | 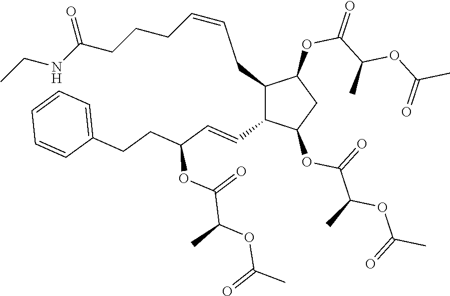 [SMILES: CCNC(=O)CCC/C=C\C[C@@H]1[C@@H](/C=C/[C@H](CCc2ccccc2)OC(=O)[C@H](C)OC(C)=O)[C@H](OC(=O)[C@H](C)OC(C)=O)C[C@@H]1OC(=O)[C@H](C)OC(C)=O] |
| 63 | 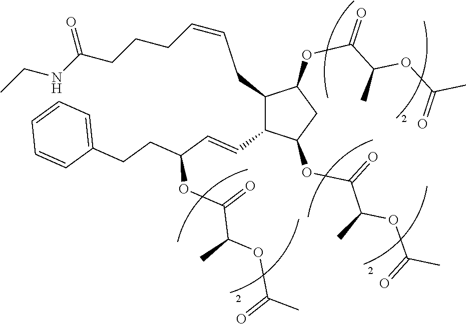 |
| 64 | 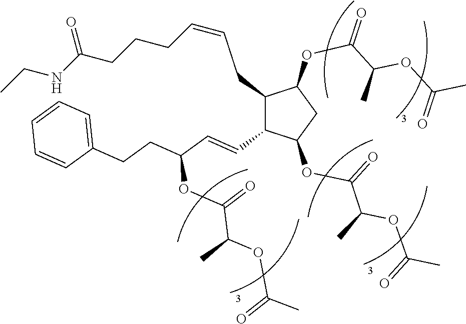 |
| 65 | 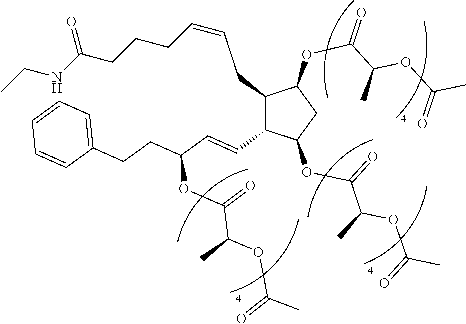 |
| 66 | 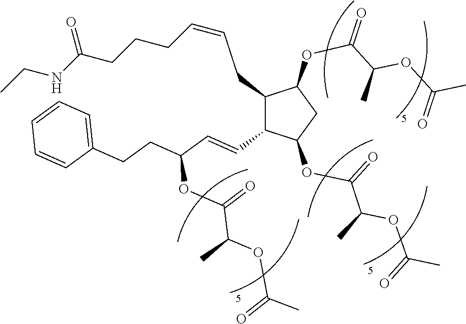 |
| 67 | 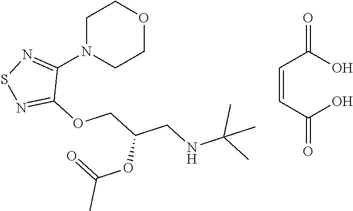 |
| 68 | 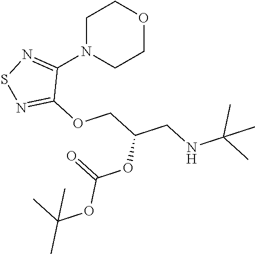 [SMILES: CC(C)(C)NC[C@@H](COc1nsnc1N1CCOCC1)OC(=O)OC(C)(C)C] |
| 69 | 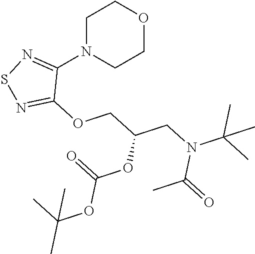 [SMILES: CC(=O)N(C[C@@H](COc1nsnc1N1CCOCC1)OC(=O)OC(C)(C)C)C(C)(C)C] |
| 70 | 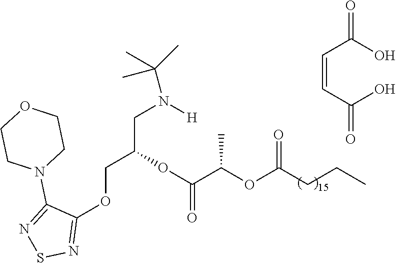 |
| 71 | 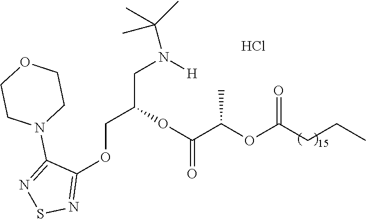 |
| 72 | 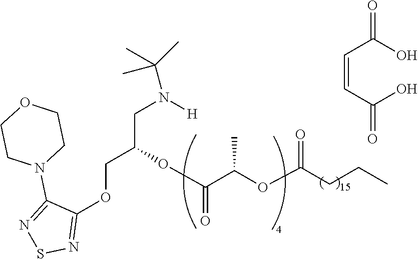 |
| 73 | 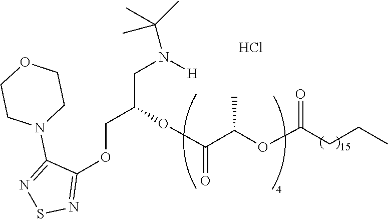 |
| 74 | 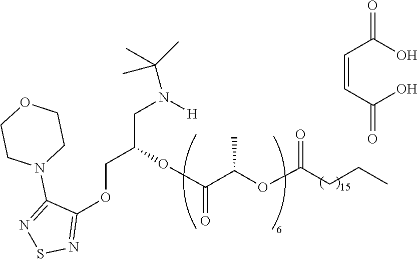 |
| 75 | 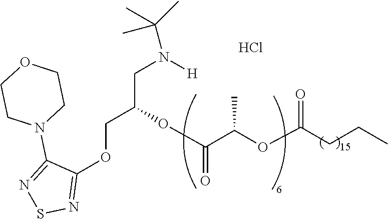 |
| 76 | 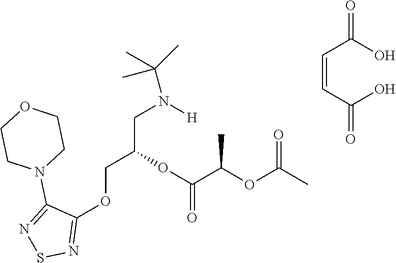 [SMILES: CC(=O)O[C@H](C)C(=O)O[C@@H](CNC(C)(C)C)COc1nsnc1N1CCOCC1 ; O=C(O)/C=C\C(=O)O] |
| 77 | 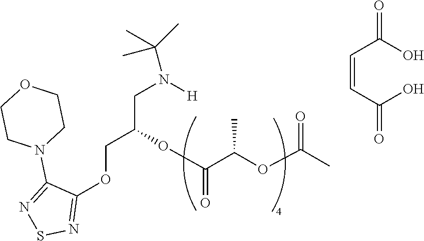 |
| 78 | 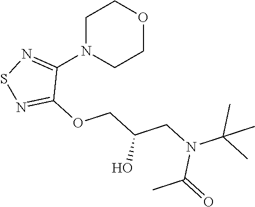 [SMILES: CC(=O)N(C[C@H](O)COc1nsnc1N1CCOCC1)C(C)(C)C] |
| 79 | 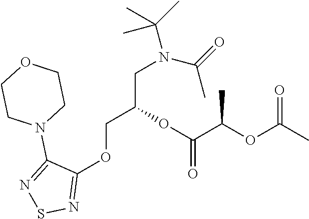 [SMILES: CC(=O)O[C@H](C)C(=O)O[C@H](COc1nsnc1N1CCOCC1)CN(C(C)=O)C(C)(C)C] |
| 80 | 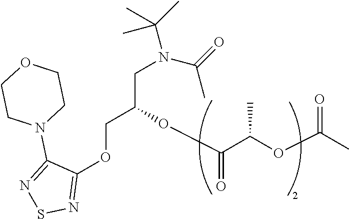 |
| 81 | 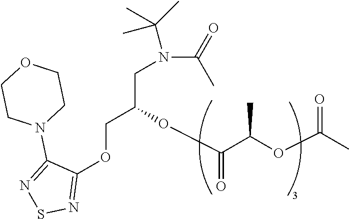 |
| 82 | 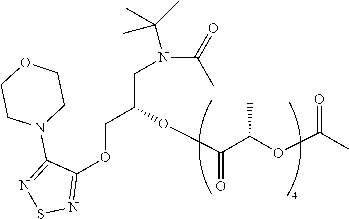 |
| 83 | 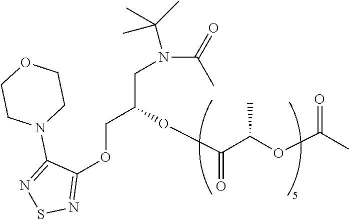 |
| 84 | 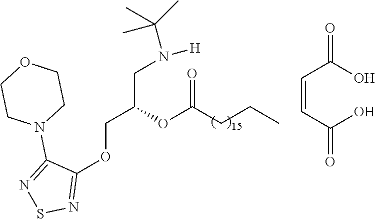 |
| 85 | 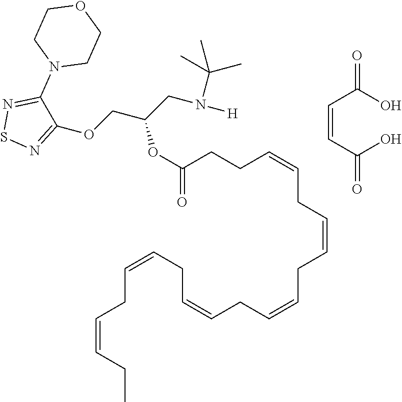 |
| 86 | 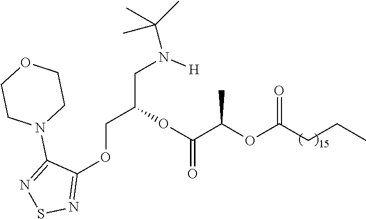 |
| 87 | 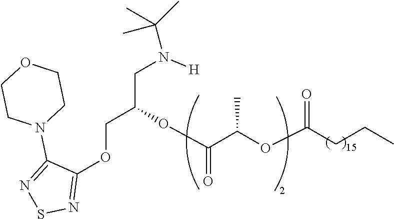 |
| 88 | 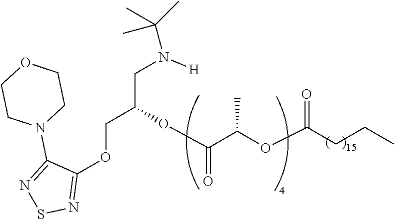 |
| 89 | 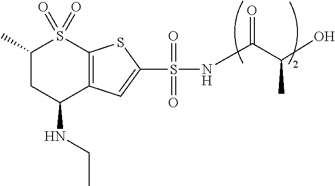 |
| 90 | 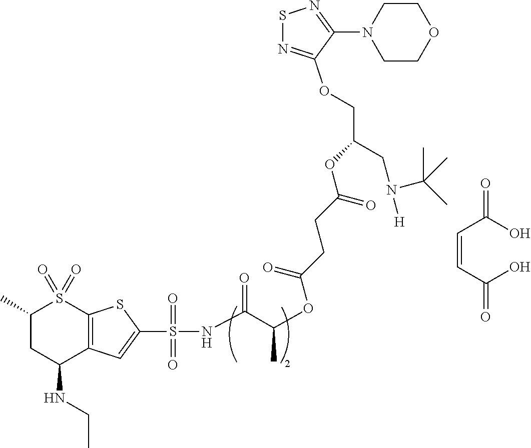 |
| 91 | 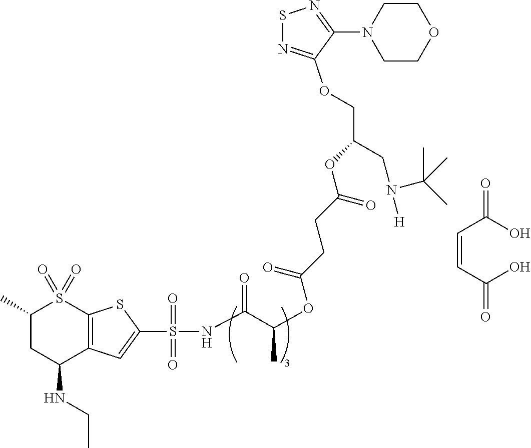 |
| 92 | 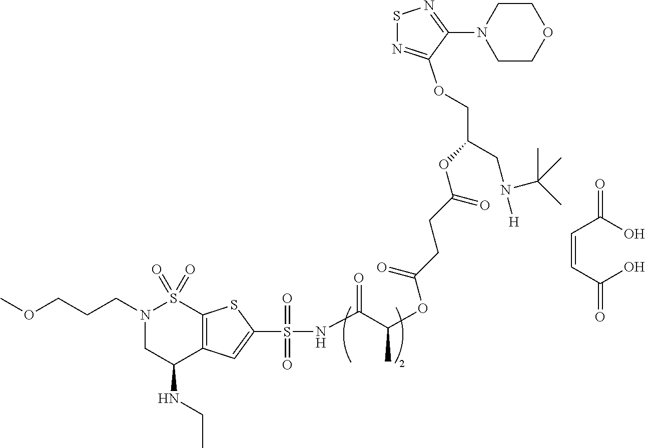 |
| 93 | 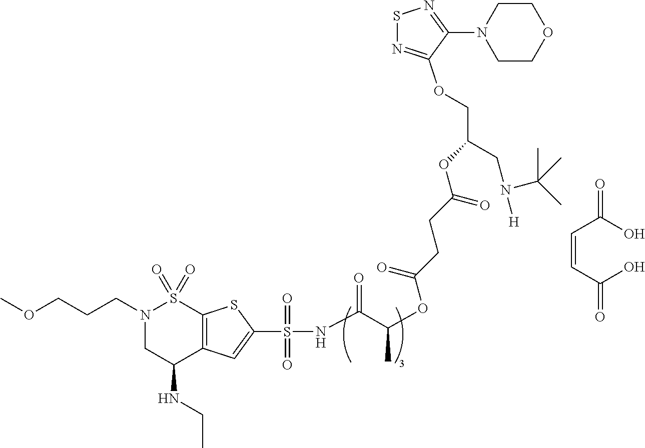 |
| 94 | 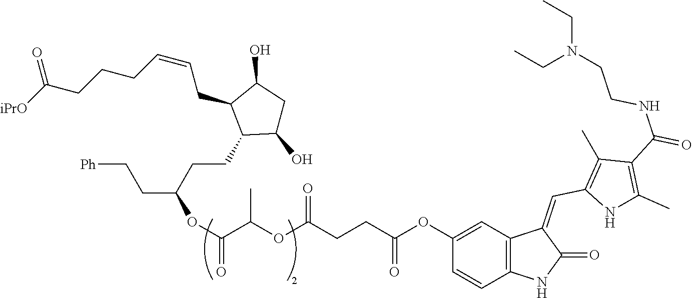 |
| 95 | 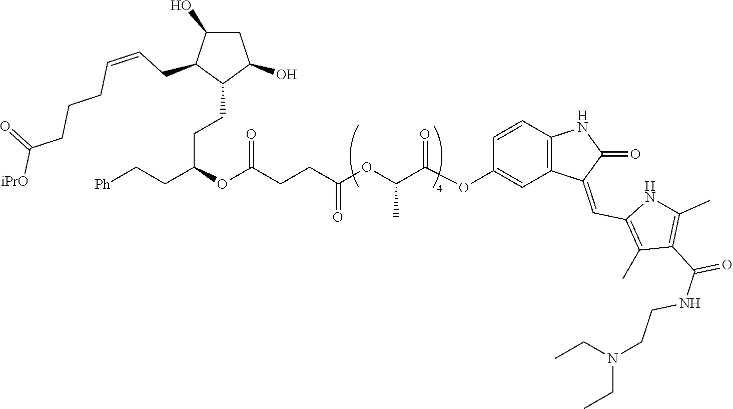 |
| 96 | 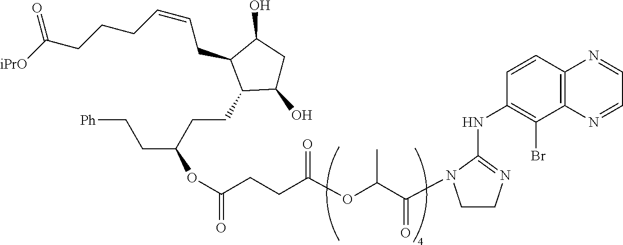 |

TABLE C
Non-limiting Examples of Prodrugs

| | |
|---|---|
| 97 | 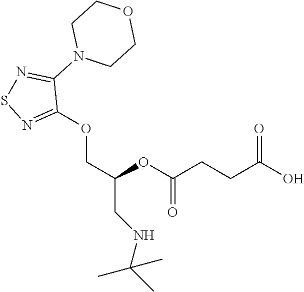 [SMILES: CC(C)(C)NC[C@@H](COc1nsnc1N1CCOCC1)OC(=O)CCC(=O)O] |
| 98 | 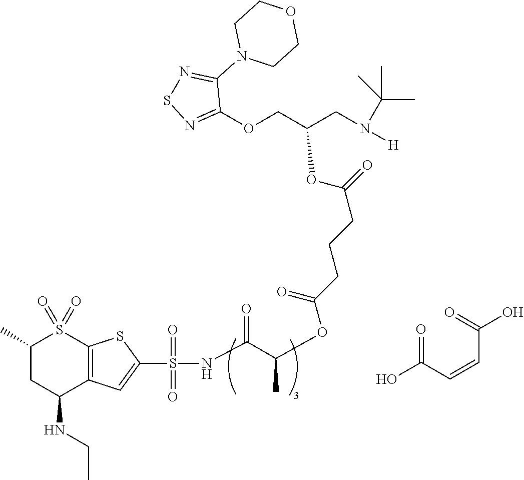 |
| 99 | 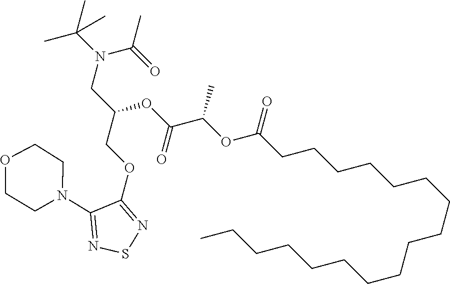 [SMILES: CCCCCCCCCCCCCCCCCC(=O)O[C@@H](C)C(=O)O[C@H](COc1nsnc1N1CCOCC1)CN(C(C)=O)C(C)(C)C] |
| 100 | 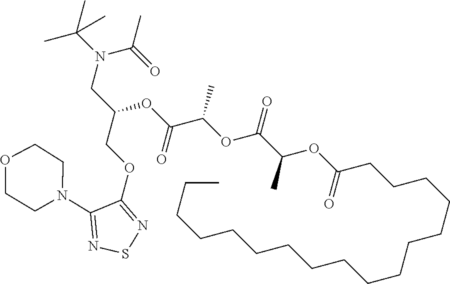 [SMILES: CCCCCCCCCCCCCCCCCC(=O)O[C@@H](C)C(=O)O[C@@H](C)C(=O)O[C@H](COc1nsnc1N1CCOCC1)CN(C(C)=O)C(C)(C)C] |
| 101 | 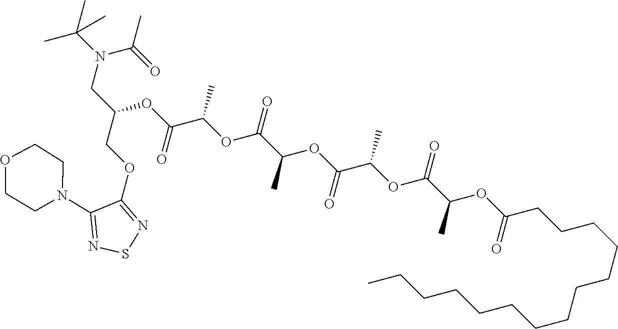 [SMILES: CCCCCCCCCCCCCCC(=O)O[C@@H](C)C(=O)O[C@@H](C)C(=O)O[C@@H](C)C(=O)O[C@@H](C)C(=O)O[C@H](COc1nsnc1N1CCOCC1)CN(C(C)=O)C(C)(C)C] |
| 102 | 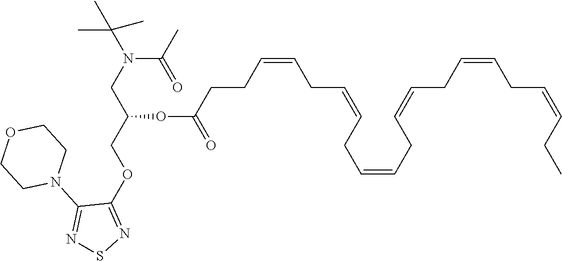 [SMILES: CC/C=C\C/C=C\C/C=C\C/C=C\C/C=C\C/C=C\CCC(=O)O[C@H](COc1nsnc1N1CCOCC1)CN(C(C)=O)C(C)(C)C] |
| 103 | 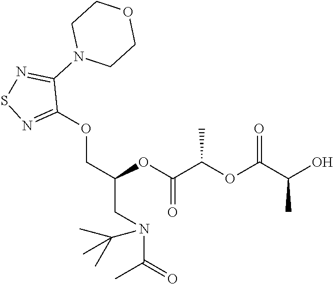 [SMILES: CC(=O)N(C[C@@H](COc1nsnc1N1CCOCC1)OC(=O)[C@H](C)OC(=O)[C@H](C)O)C(C)(C)C] |
| 104 | 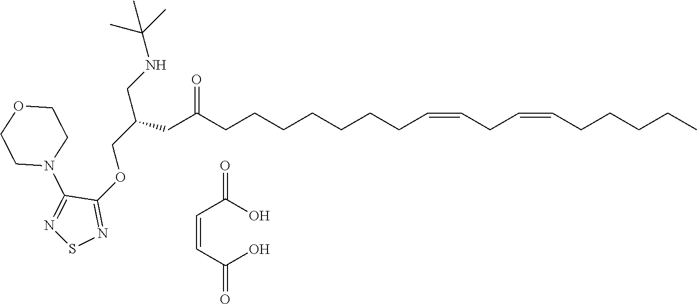 |
| 105 | 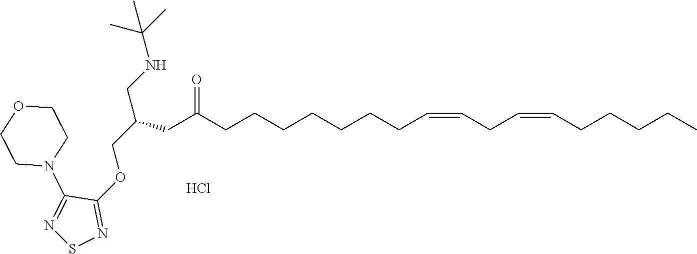 [SMILES: CCCCC/C=C\C/C=C\CCCCCCCC(=O)C[C@@H](CNC(C)(C)C)COc1nsnc1N1CCOCC1 ; Cl] |
| 106 | 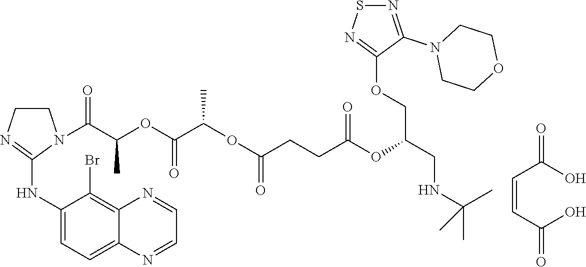 |
| 107 | 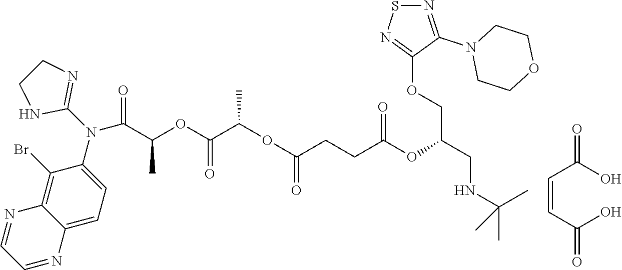 |
| 108 | 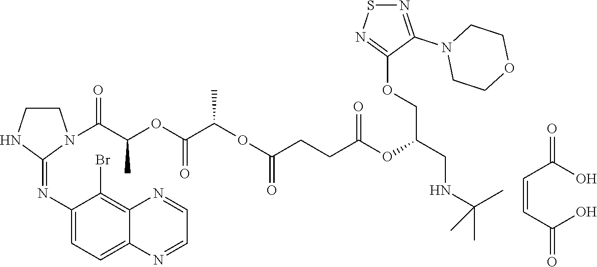 |
| 109 | 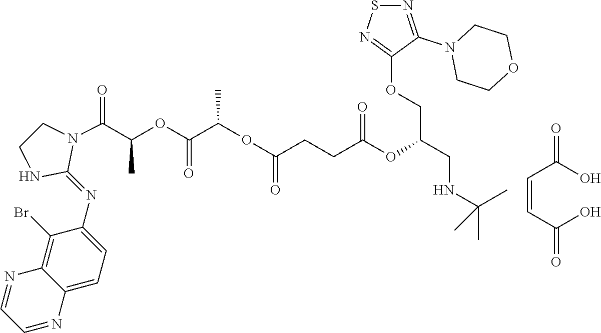 |
| 110 | 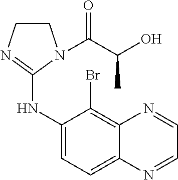 [SMILES: C[C@H](O)C(=O)N1CCN=C1Nc1ccc2nccnc2c1Br] |
| 111 | 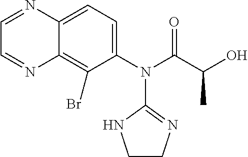 [SMILES: C[C@H](O)C(=O)N(C1=NCCN1)c1ccc2nccnc2c1Br] |
| 112 | 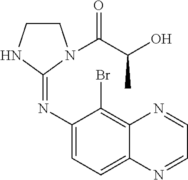 [SMILES: C[C@H](O)C(=O)N1CCN/C1=N/c1ccc2nccnc2c1Br] |
| 113 | 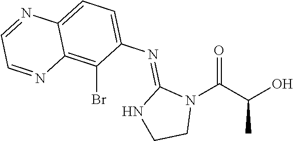 [SMILES: C[C@H](O)C(=O)N1CCN/C1=N\c1ccc2nccnc2c1Br] |
| 114 | 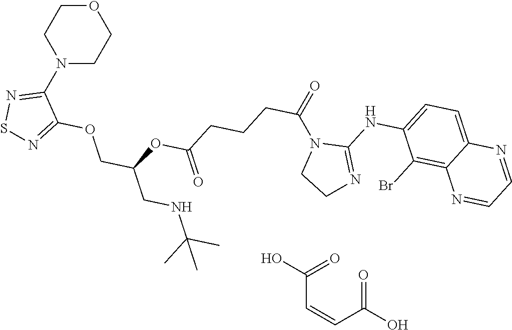 [SMILES: CC(C)(C)NC[C@@H](COc1nsnc1N1CCOCC1)OC(=O)CCCC(=O)N1CCN=C1Nc1ccc2nccnc2c1Br ; O=C(O)/C=C\C(=O)O] |
| 115 | 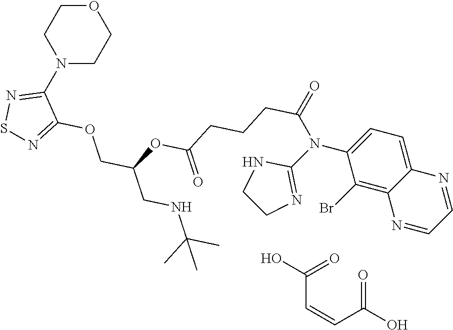 |
| 116 | 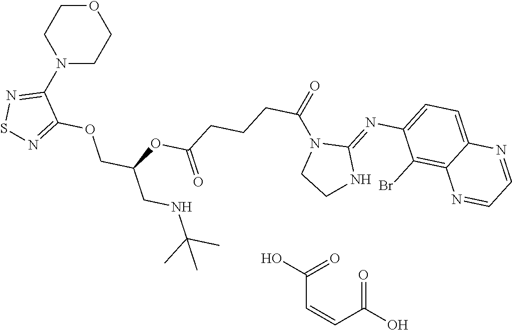 [SMILES: CC(C)(C)NC[C@@H](COc1nsnc1N1CCOCC1)OC(=O)CCCC(=O)N1CCN/C1=N\c1ccc2nccnc2c1Br ; O=C(O)/C=C\C(=O)O] |
| 117 | 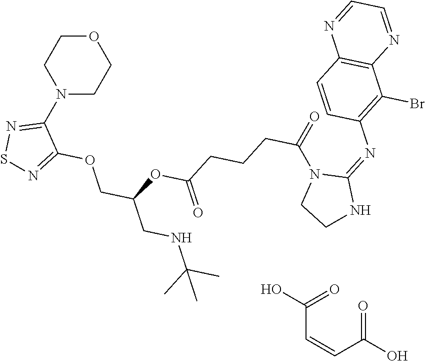 [SMILES: CC(C)(C)NC[C@@H](COc1nsnc1N1CCOCC1)OC(=O)CCCC(=O)N1CCN/C1=N/c1ccc2nccnc2c1Br ; O=C(O)/C=C\C(=O)O] |
| 118 | 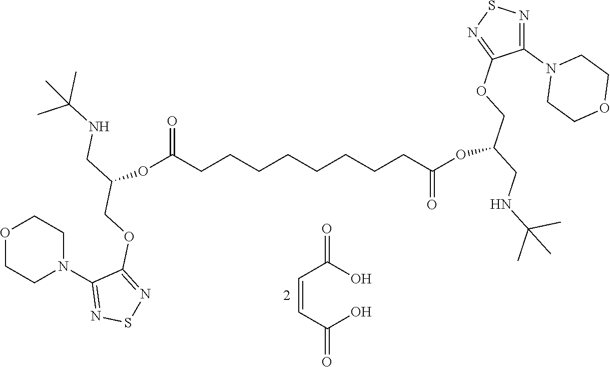 [SMILES: CC(C)(C)NC[C@@H](COc1nsnc1N1CCOCC1)OC(=O)CCCCCCCCC(=O)O[C@@H](CNC(C)(C)C)COc1nsnc1N1CCOCC1 ; O=C(O)/C=C\C(=O)O] |
| 119 | 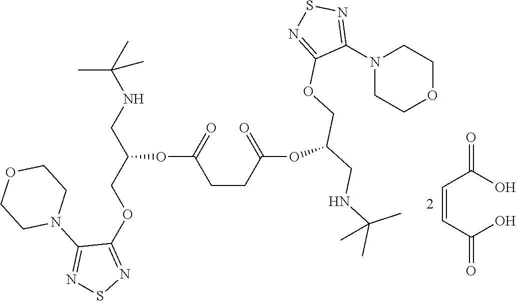 |
| 120 | 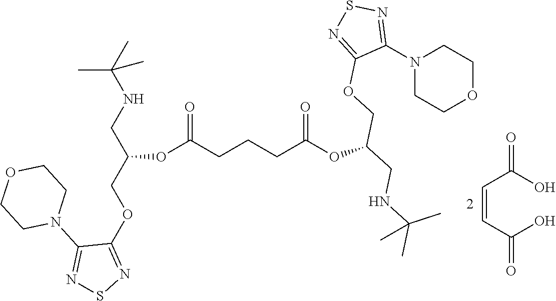 |
| 121 | 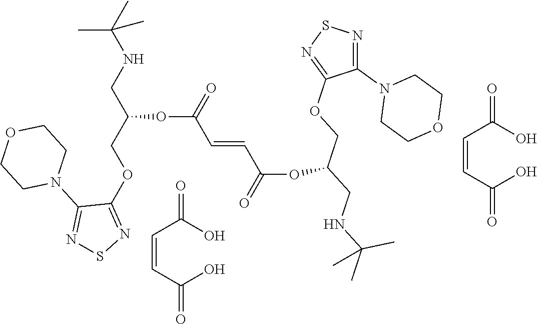 |
| 122 | 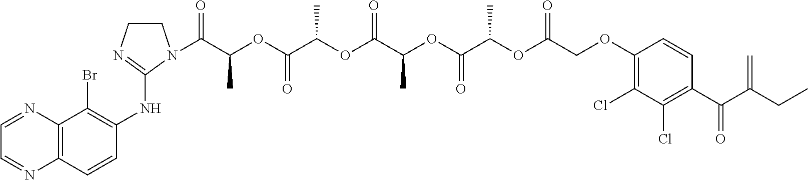 [SMILES: C=C(CC)C(=O)c1ccc(OCC(=O)O[C@@H](C)C(=O)O[C@@H](C)C(=O)O[C@@H](C)C(=O)O[C@@H](C)C(=O)N2CCN=C2Nc2ccc3nccnc3c2Br)c(Cl)c1Cl] |
| 123 | 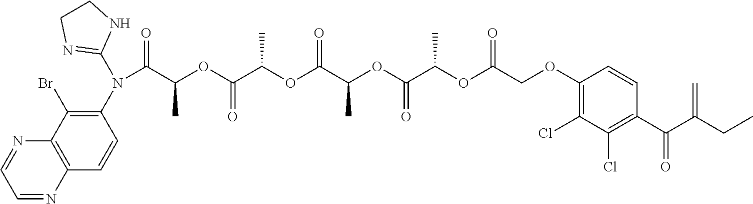 [SMILES: C=C(CC)C(=O)c1ccc(OCC(=O)O[C@@H](C)C(=O)O[C@@H](C)C(=O)O[C@@H](C)C(=O)O[C@@H](C)C(=O)N(C2=NCCN2)c2ccc3nccnc3c2Br)c(Cl)c1Cl] |
| 124 | 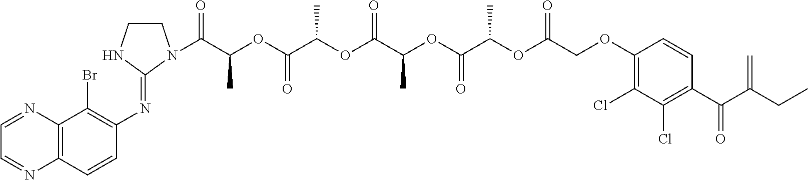 [SMILES: C=C(CC)C(=O)c1ccc(OCC(=O)O[C@@H](C)C(=O)O[C@@H](C)C(=O)O[C@@H](C)C(=O)O[C@@H](C)C(=O)N2CCN/C2=N\c2ccc3nccnc3c2Br)c(Cl)c1Cl] |
| 125 | 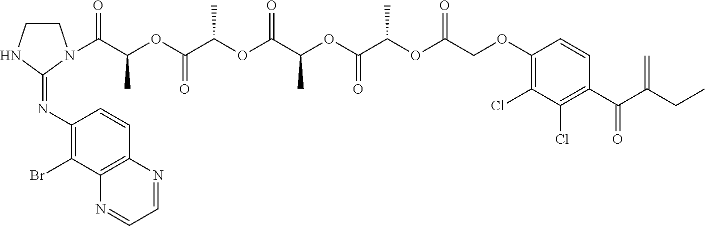 [SMILES: C=C(CC)C(=O)c1ccc(OCC(=O)O[C@@H](C)C(=O)O[C@@H](C)C(=O)O[C@@H](C)C(=O)O[C@@H](C)C(=O)N2CCN/C2=N/c2ccc3nccnc3c2Br)c(Cl)c1Cl] |
| 126 | 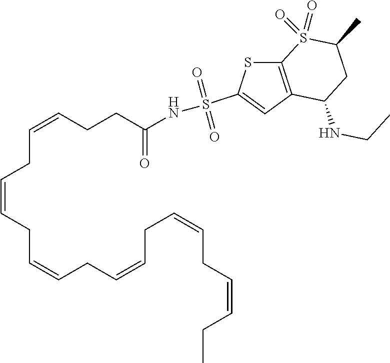 [SMILES: CC/C=C\C/C=C\C/C=C\C/C=C\C/C=C\C/C=C\CCC(=O)NS(=O)(=O)c1cc2c(s1)S(=O)(=O)[C@@H](C)C[C@@H]2NCC] |
| 127 | 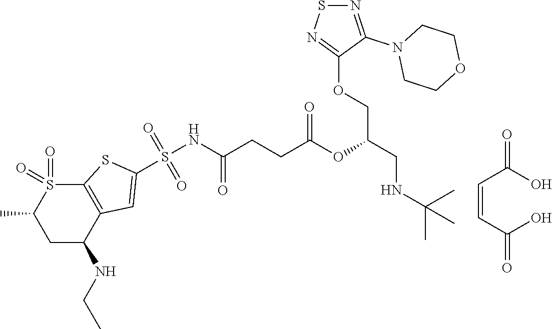 |
| 128 | 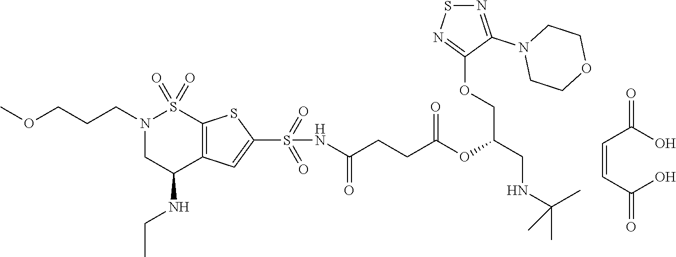 |
| 129 | 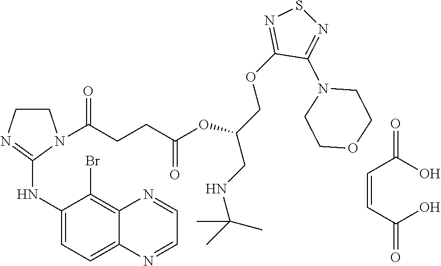 |
| 130 | 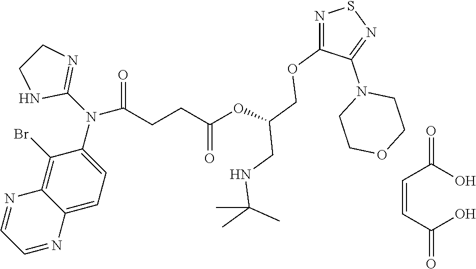 |
| 131 | 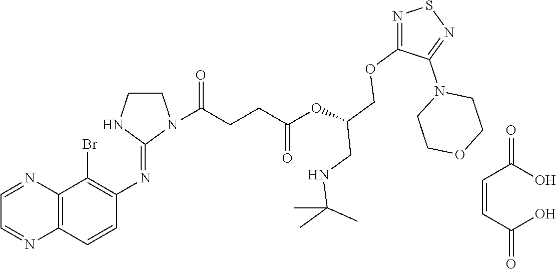 |
| 132 | 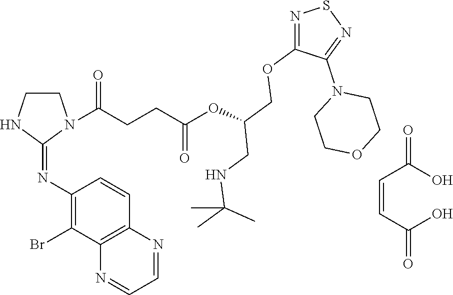 |
| 133 | 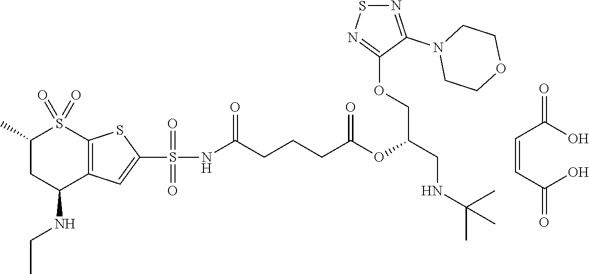 |
| 134 | 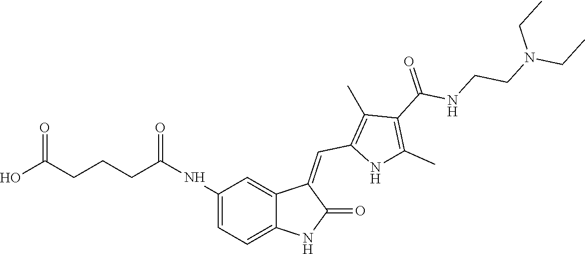 [SMILES: CCN(CC)CCNC(=O)c1c(C)[nH]c(/C=C2\C(=O)Nc3ccc(NC(=O)CCCC(=O)O)cc32)c1C] |
| 135 | 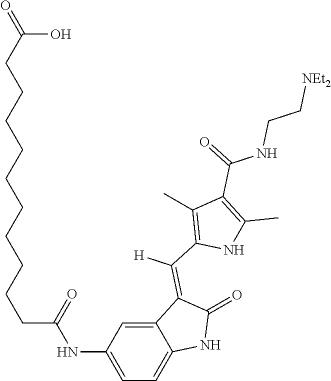 |
| 136 | 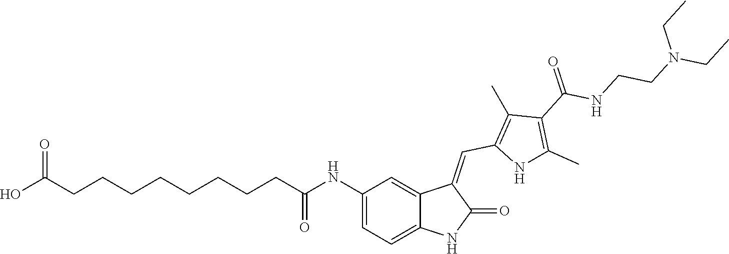 [SMILES: CCN(CC)CCNC(=O)c1c(C)[nH]c(/C=C2\C(=O)Nc3ccc(NC(=O)CCCCCCCCC(=O)O)cc32)c1C] |
| 137 | 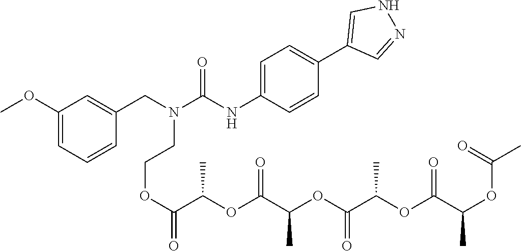 [SMILES: COc1cccc(CN(CCOC(=O)[C@H](C)OC(=O)[C@H](C)OC(=O)[C@H](C)OC(=O)[C@H](C)OC(C)=O)C(=O)Nc2ccc(-c3cn[nH]c3)cc2)c1] |
| 138 | 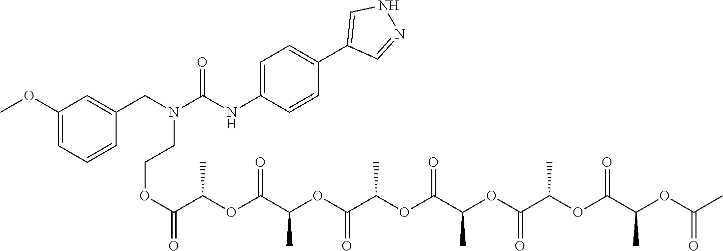 [SMILES: COc1cccc(CN(CCOC(=O)[C@H](C)OC(=O)[C@H](C)OC(=O)[C@H](C)OC(=O)[C@H](C)OC(=O)[C@H](C)OC(=O)[C@H](C)OC(C)=O)C(=O)Nc2ccc(-c3cn[nH]c3)cc2)c1] |
| 139 | 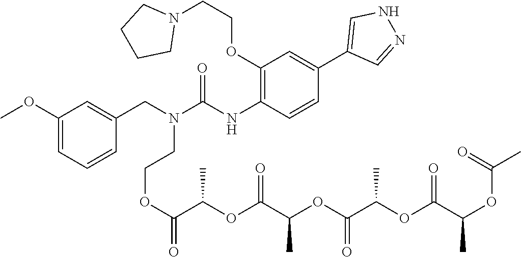 [SMILES: COc1cccc(CN(CCOC(=O)[C@H](C)OC(=O)[C@H](C)OC(=O)[C@H](C)OC(=O)[C@H](C)OC(C)=O)C(=O)Nc2ccc(-c3cn[nH]c3)cc2OCCN2CCCC2)c1] |
| 140 | 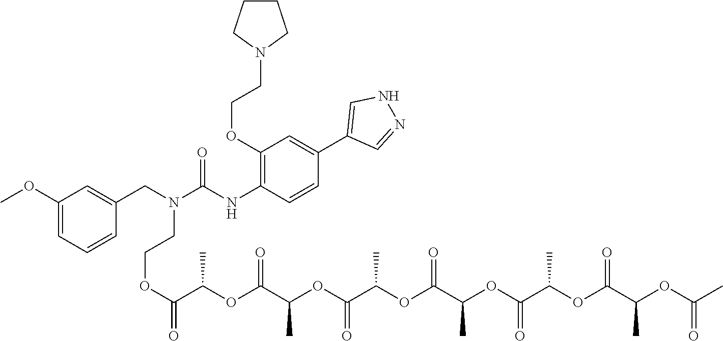 [SMILES: COc1cccc(CN(CCOC(=O)[C@H](C)OC(=O)[C@H](C)OC(=O)[C@H](C)OC(=O)[C@H](C)OC(=O)[C@H](C)OC(=O)[C@H](C)OC(C)=O)C(=O)Nc2ccc(-c3cn[nH]c3)cc2OCCN2CCCC2)c1] |
| 141 | 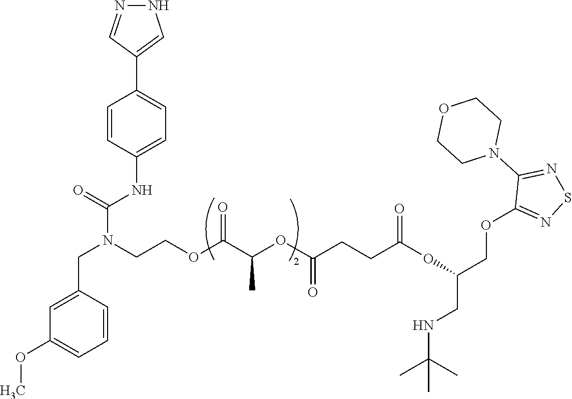 |
| 142 | 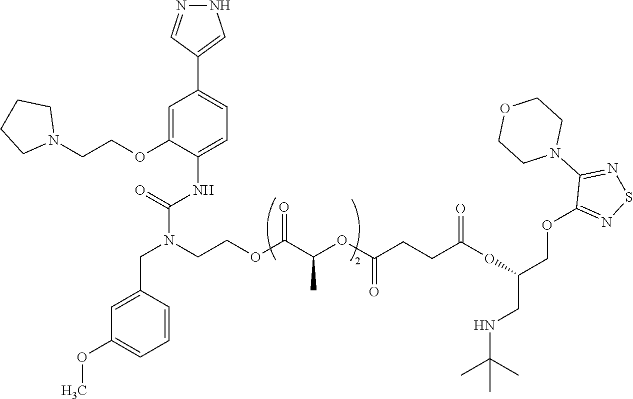 |
| 143 | 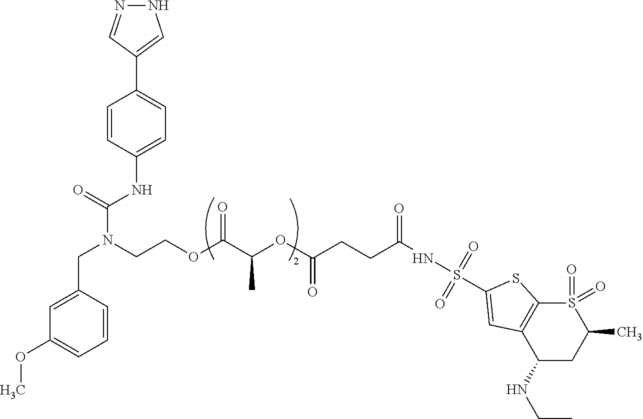 |
| 144 | 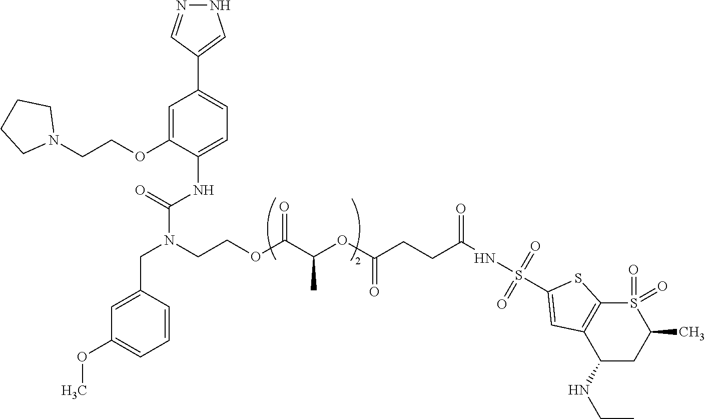 |
| 145 | 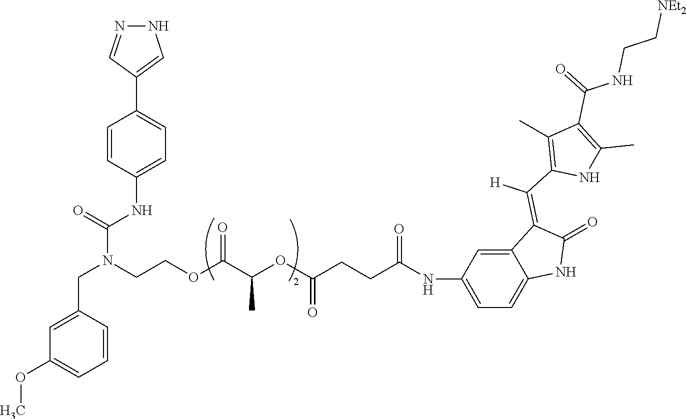 |
| 146 | 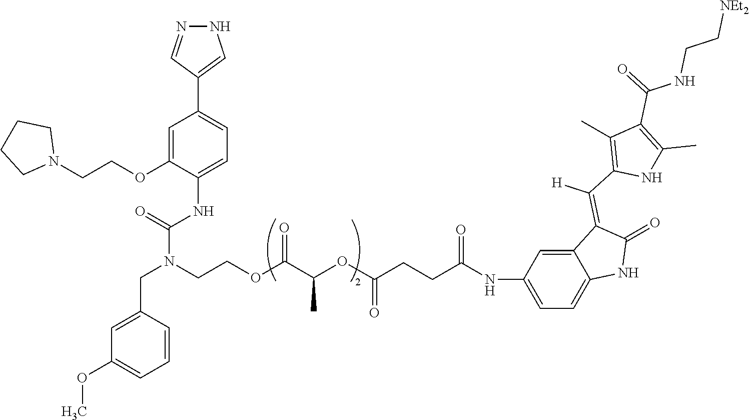 |
| 147 | 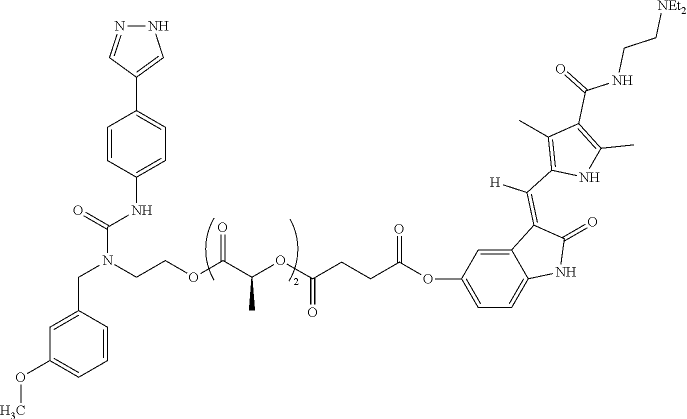 |
| 148 | 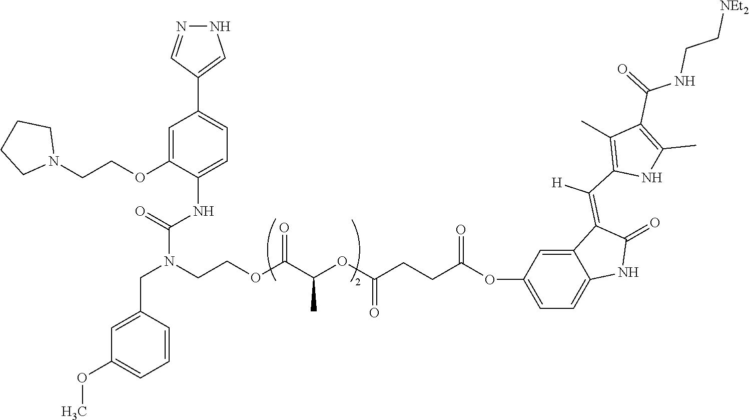 |
| 149 | 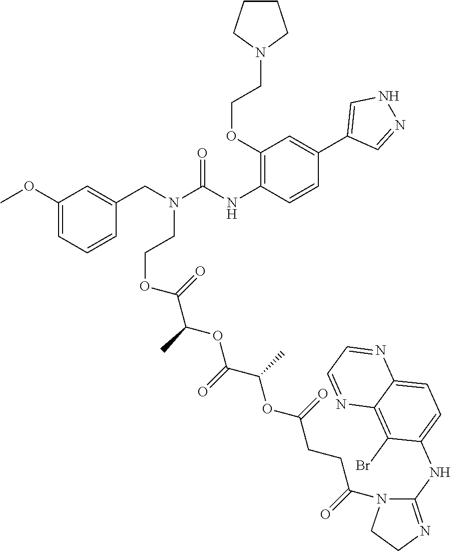 [SMILES: COc1cccc(CN(CCOC(=O)[C@H](C)OC(=O)[C@H](C)OC(=O)CCC(=O)N2CCN=C2Nc2ccc3nccnc3c2Br)C(=O)Nc2ccc(-c3cn[nH]c3)cc2OCCN2CCCC2)c1] |
| 150 | 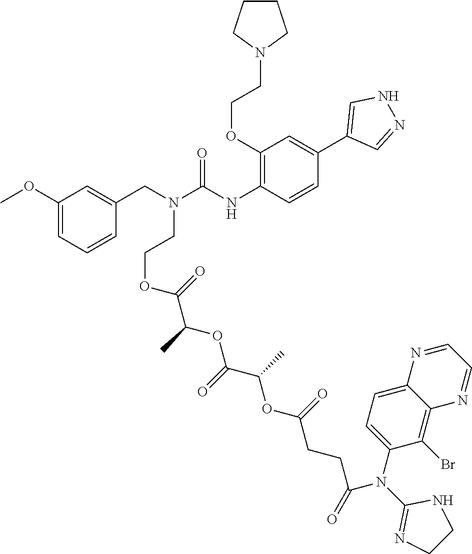 [SMILES: COc1cccc(CN(CCOC(=O)[C@H](C)OC(=O)[C@H](C)OC(=O)CCC(=O)N(C2=NCCN2)c2ccc3nccnc3c2Br)C(=O)Nc2ccc(-c3cn[nH]c3)cc2OCCN2CCCC2)c1] |
| 151 | 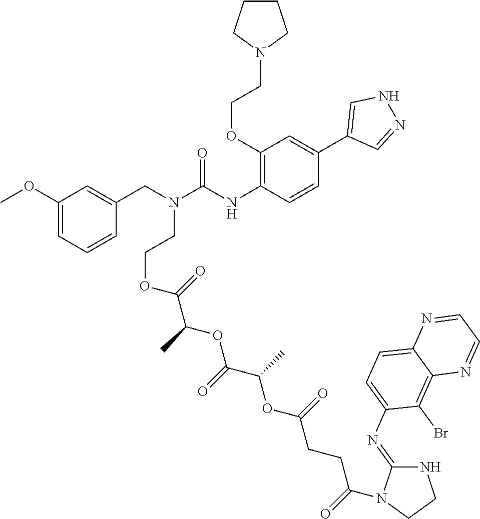 [SMILES: COc1cccc(CN(CCOC(=O)[C@H](C)OC(=O)[C@H](C)OC(=O)CCC(=O)N2CCN/C2=N\c2ccc3nccnc3c2Br)C(=O)Nc2ccc(-c3cn[nH]c3)cc2OCCN2CCCC2)c1] |
| 152 | 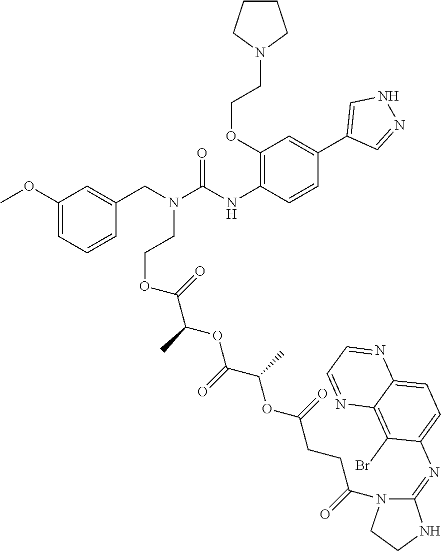 [SMILES: COc1cccc(CN(CCOC(=O)[C@H](C)OC(=O)[C@H](C)OC(=O)CCC(=O)N2CCN/C2=N/c2ccc3nccnc3c2Br)C(=O)Nc2ccc(-c3cn[nH]c3)cc2OCCN2CCCC2)c1] |
| 153 | 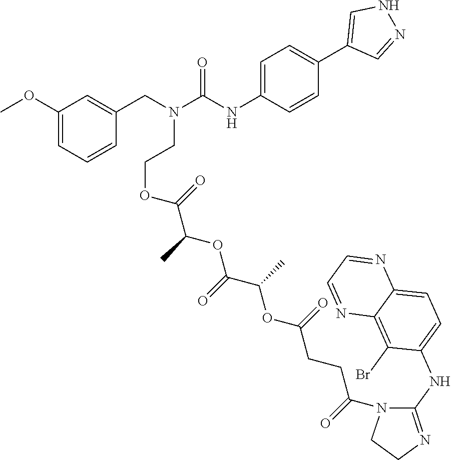 [SMILES: COc1cccc(CN(CCOC(=O)[C@H](C)OC(=O)[C@H](C)OC(=O)CCC(=O)N2CCN=C2Nc2ccc3nccnc3c2Br)C(=O)Nc2ccc(-c3cn[nH]c3)cc2)c1] |
| 154 | 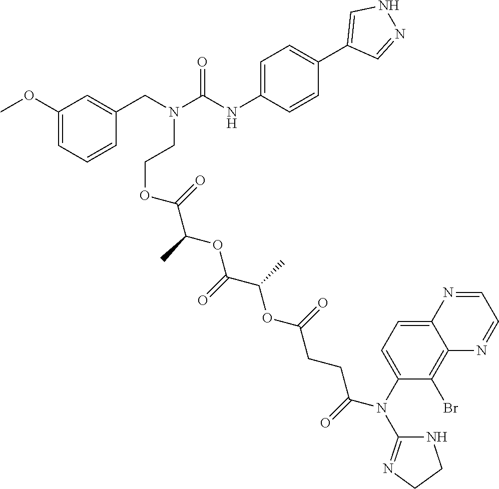 [SMILES: COc1cccc(CN(CCOC(=O)[C@H](C)OC(=O)[C@H](C)OC(=O)CCC(=O)N(C2=NCCN2)c2ccc3nccnc3c2Br)C(=O)Nc2ccc(-c3cn[nH]c3)cc2)c1] |
| 155 | 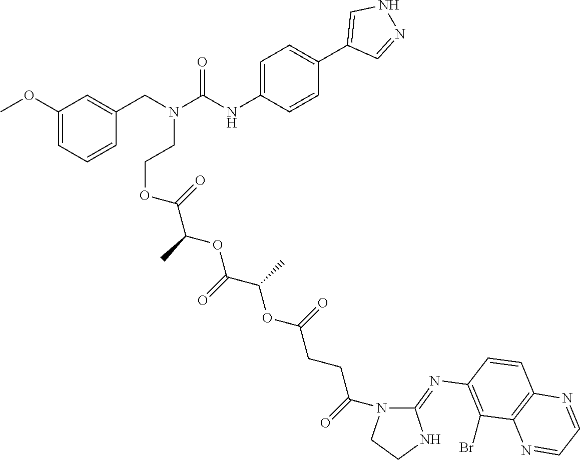 [SMILES: COc1cccc(CN(CCOC(=O)[C@H](C)OC(=O)[C@H](C)OC(=O)CCC(=O)N2CCN/C2=N\c2ccc3nccnc3c2Br)C(=O)Nc2ccc(-c3cn[nH]c3)cc2)c1] |
| 156 | 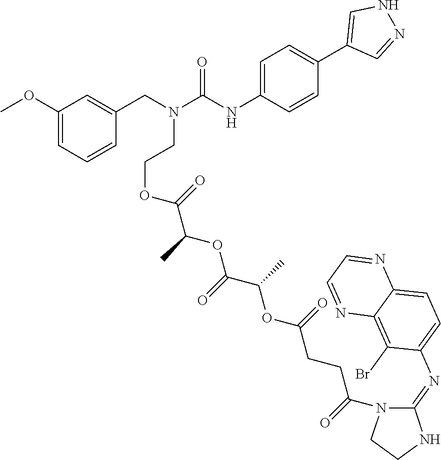 [SMILES: COc1cccc(CN(CCOC(=O)[C@H](C)OC(=O)[C@H](C)OC(=O)CCC(=O)N2CCN/C2=N/c2ccc3nccnc3c2Br)C(=O)Nc2ccc(-c3cn[nH]c3)cc2)c1] |
| 157 | 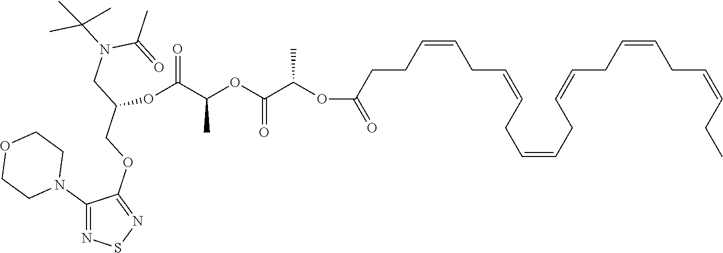 [SMILES: CC/C=C\C/C=C\C/C=C\C/C=C\C/C=C\C/C=C\CCC(=O)O[C@@H](C)C(=O)O[C@@H](C)C(=O)O[C@H](COc1nsnc1N1CCOCC1)CN(C(C)=O)C(C)(C)C] |
| 158 | 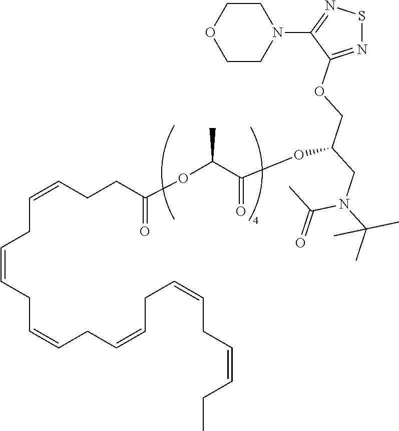 |
| 159 | 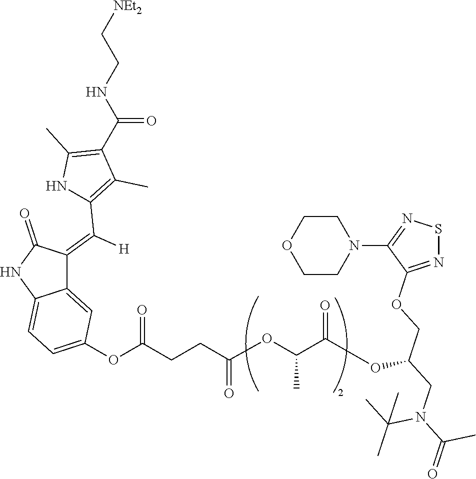 |
| 160 | 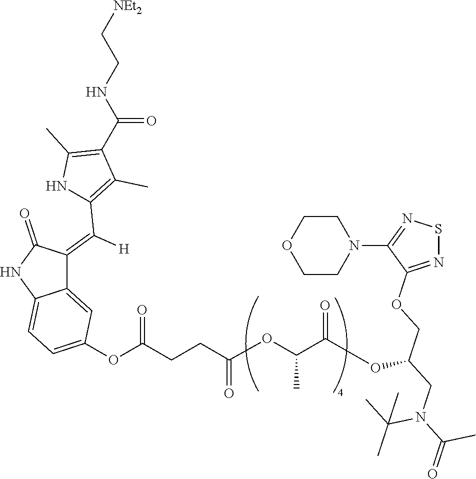 |
| 161 | 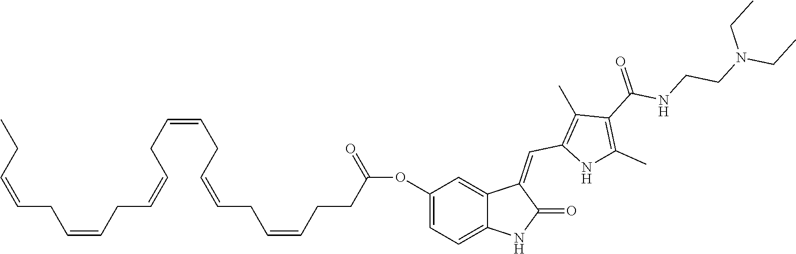 [SMILES: CC/C=C\C/C=C\C/C=C\C/C=C\C/C=C\C/C=C\CCC(=O)Oc1ccc2c(c1)/C(=C/c1[nH]c(C)c(C(=O)NCCN(CC)CC)c1C)C(=O)N2] |
| 162 | 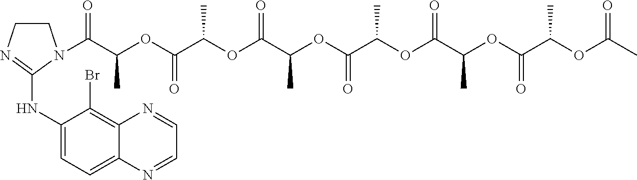 [SMILES: CC(=O)O[C@@H](C)C(=O)O[C@@H](C)C(=O)O[C@@H](C)C(=O)O[C@@H](C)C(=O)O[C@@H](C)C(=O)O[C@@H](C)C(=O)N1CCN=C1Nc1ccc2nccnc2c1Br] |
| 163 | 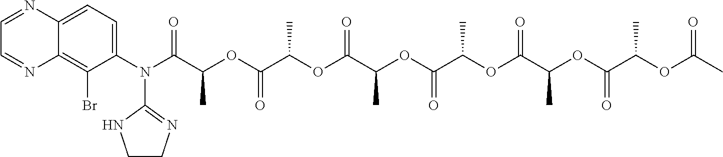 [SMILES: CC(=O)O[C@@H](C)C(=O)O[C@@H](C)C(=O)O[C@@H](C)C(=O)O[C@@H](C)C(=O)O[C@@H](C)C(=O)O[C@@H](C)C(=O)N(C1=NCCN1)c1ccc2nccnc2c1Br] |
| 164 | 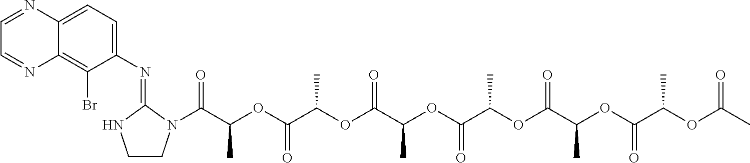 [SMILES: CC(=O)O[C@@H](C)C(=O)O[C@@H](C)C(=O)O[C@@H](C)C(=O)O[C@@H](C)C(=O)O[C@@H](C)C(=O)O[C@@H](C)C(=O)N1CCN/C1=N\c1ccc2nccnc2c1Br] |
| 165 | 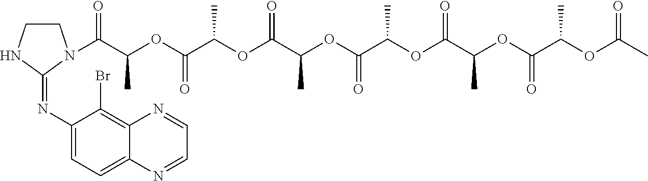 [SMILES: CC(=O)O[C@@H](C)C(=O)O[C@@H](C)C(=O)O[C@@H](C)C(=O)O[C@@H](C)C(=O)O[C@@H](C)C(=O)O[C@@H](C)C(=O)N1CCN/C1=N/c1ccc2nccnc2c1Br] |
| 166 | 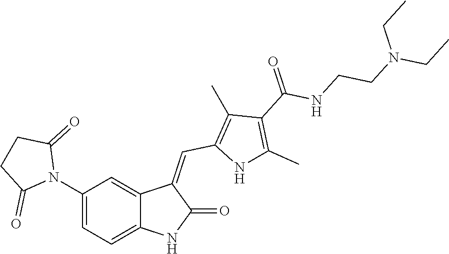 [SMILES: CCN(CC)CCNC(=O)c1c(C)[nH]c(/C=C2\C(=O)Nc3ccc(N4C(=O)CCC4=O)cc32)c1C] |
| 167 | 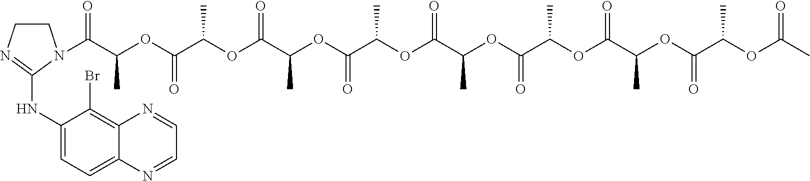 [SMILES: CC(=O)O[C@@H](C)C(=O)O[C@@H](C)C(=O)O[C@@H](C)C(=O)O[C@@H](C)C(=O)O[C@@H](C)C(=O)O[C@@H](C)C(=O)O[C@@H](C)C(=O)O[C@@H](C)C(=O)N1CCN=C1Nc1ccc2nccnc2c1Br] |
| 168 | 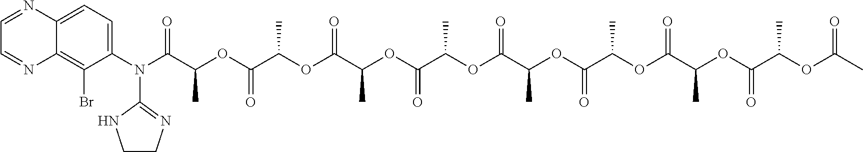 [SMILES: CC(=O)O[C@@H](C)C(=O)O[C@@H](C)C(=O)O[C@@H](C)C(=O)O[C@@H](C)C(=O)O[C@@H](C)C(=O)O[C@@H](C)C(=O)O[C@@H](C)C(=O)O[C@@H](C)C(=O)N(C1=NCCN1)c1ccc2nccnc2c1Br] |
| 169 | 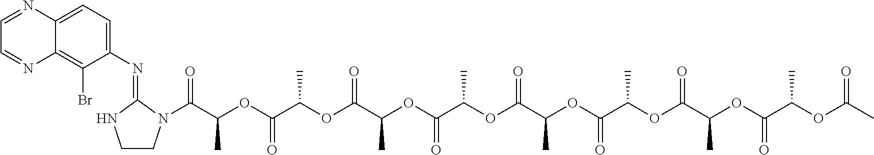 [SMILES: CC(=O)O[C@@H](C)C(=O)O[C@@H](C)C(=O)O[C@@H](C)C(=O)O[C@@H](C)C(=O)O[C@@H](C)C(=O)O[C@@H](C)C(=O)O[C@@H](C)C(=O)O[C@@H](C)C(=O)N1CCN/C1=N\c1ccc2nccnc2c1Br] |
| 170 | 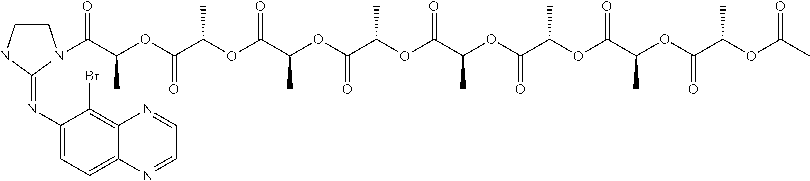 [SMILES: CC(=O)O[C@@H](C)C(=O)O[C@@H](C)C(=O)O[C@@H](C)C(=O)O[C@@H](C)C(=O)O[C@@H](C)C(=O)O[C@@H](C)C(=O)O[C@@H](C)C(=O)O[C@@H](C)C(=O)N1CCN/C1=N/c1ccc2nccnc2c1Br] |
| 171 | 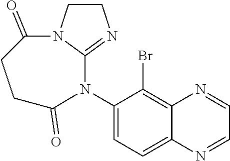 [SMILES: O=C1CCC(=O)N(c2ccc3nccnc3c2Br)C2=NCCN12] |
| 172 | 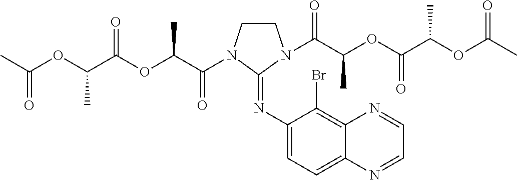 [SMILES: CC(=O)O[C@@H](C)C(=O)O[C@@H](C)C(=O)N1CCN(C(=O)[C@H](C)OC(=O)[C@H](C)OC(C)=O)C1=Nc1ccc2nccnc2c1Br] |
| 173 | 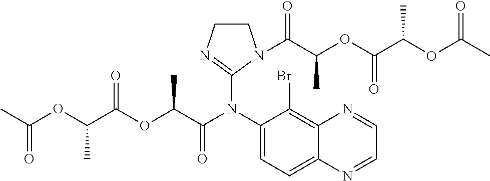 [SMILES: CC(=O)O[C@@H](C)C(=O)O[C@@H](C)C(=O)N1CCN=C1N(C(=O)[C@H](C)OC(=O)[C@H](C)OC(C)=O)c1ccc2nccnc2c1Br] |
| 174 | 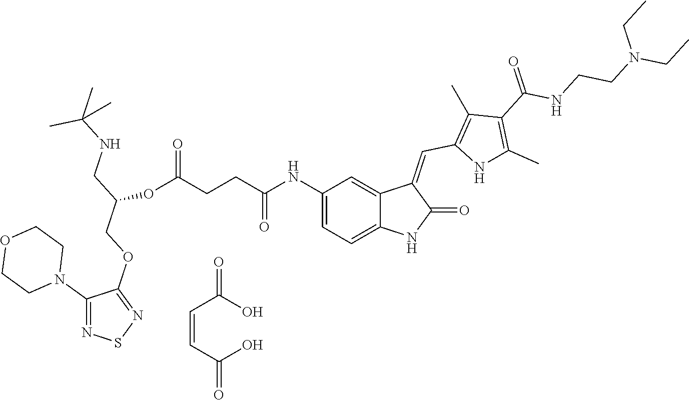 [SMILES: CCN(CC)CCNC(=O)c1c(C)[nH]c(/C=C2\C(=O)Nc3ccc(NC(=O)CCC(=O)O[C@@H](CNC(C)(C)C)COc4nsnc4N4CCOCC4)cc32)c1C ; O=C(O)/C=C\C(=O)O] |
| 175 | 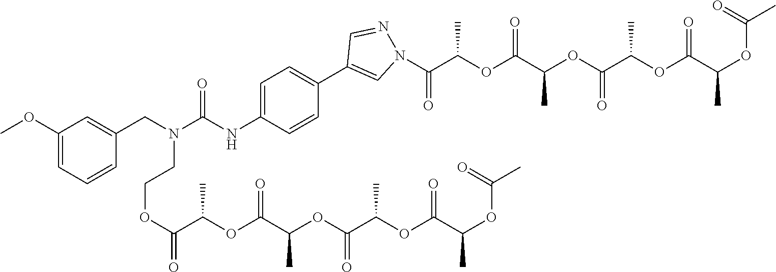 [SMILES: COc1cccc(CN(CCOC(=O)[C@H](C)OC(=O)[C@H](C)OC(=O)[C@H](C)OC(=O)[C@H](C)OC(C)=O)C(=O)Nc2ccc(-c3cnn(C(=O)[C@H](C)OC(=O)[C@H](C)OC(=O)[C@H](C)OC(=O)[C@H](C)OC(C)=O)c3)cc2)c1] |
| 176 | 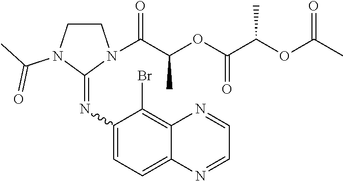 [SMILES: CC(=O)O[C@@H](C)C(=O)O[C@@H](C)C(=O)N1CCN(C(C)=O)C1=Nc1ccc2nccnc2c1Br] |
| 177 | 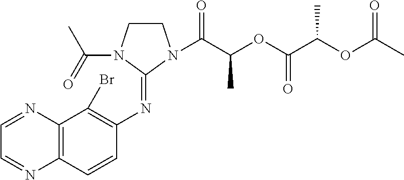 [SMILES: CC(=O)O[C@@H](C)C(=O)O[C@@H](C)C(=O)N1CCN(C(C)=O)/C1=N\c1ccc2nccnc2c1Br] |
| 178 | 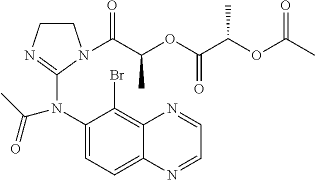 [SMILES: CC(=O)O[C@@H](C)C(=O)O[C@@H](C)C(=O)N1CCN=C1N(C(C)=O)c1ccc2nccnc2c1Br] |
| 179 | 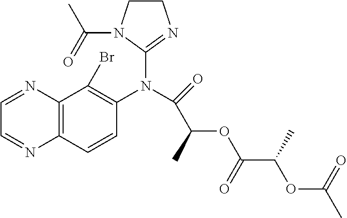 [SMILES: CC(=O)O[C@@H](C)C(=O)O[C@@H](C)C(=O)N(C1=NCCN1C(C)=O)c1ccc2nccnc2c1Br] |
| 180 | 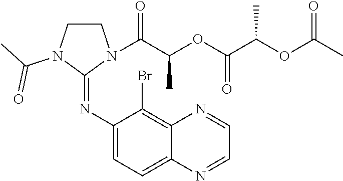 [SMILES: CC(=O)O[C@@H](C)C(=O)O[C@@H](C)C(=O)N1CCN(C(C)=O)/C1=N/c1ccc2nccnc2c1Br] |
| 181 | 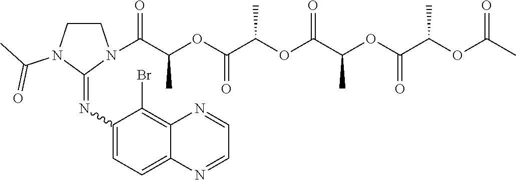 [SMILES: CC(=O)O[C@@H](C)C(=O)O[C@@H](C)C(=O)O[C@@H](C)C(=O)O[C@@H](C)C(=O)N1CCN(C(C)=O)C1=Nc1ccc2nccnc2c1Br] |
| 182 | 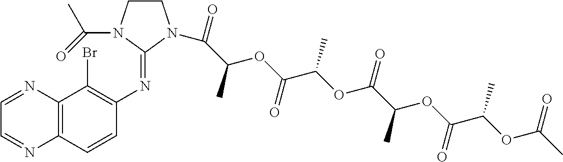 [SMILES: CC(=O)O[C@@H](C)C(=O)O[C@@H](C)C(=O)O[C@@H](C)C(=O)O[C@@H](C)C(=O)N1CCN(C(C)=O)/C1=N\c1ccc2nccnc2c1Br] |
| 183 | 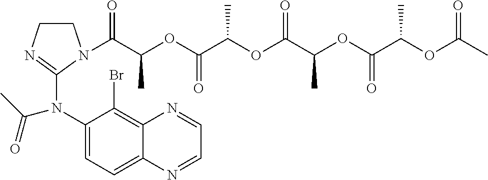 [SMILES: CC(=O)O[C@@H](C)C(=O)O[C@@H](C)C(=O)O[C@@H](C)C(=O)O[C@@H](C)C(=O)N1CCN=C1N(C(C)=O)c1ccc2nccnc2c1Br] |
| 184 | 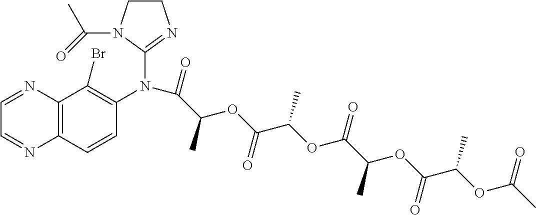 [SMILES: CC(=O)O[C@@H](C)C(=O)O[C@@H](C)C(=O)O[C@@H](C)C(=O)O[C@@H](C)C(=O)N(C1=NCCN1C(C)=O)c1ccc2nccnc2c1Br] |
| 185 | 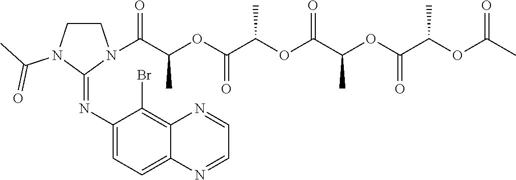 [SMILES: CC(=O)O[C@@H](C)C(=O)O[C@@H](C)C(=O)O[C@@H](C)C(=O)O[C@@H](C)C(=O)N1CCN(C(C)=O)/C1=N/c1ccc2nccnc2c1Br] |
| 186 | 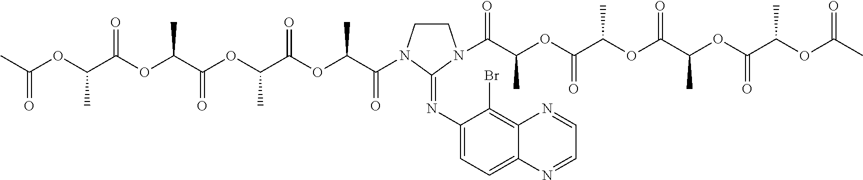 [SMILES: CC(=O)O[C@@H](C)C(=O)O[C@@H](C)C(=O)O[C@@H](C)C(=O)O[C@@H](C)C(=O)N1CCN(C(=O)[C@H](C)OC(=O)[C@H](C)OC(=O)[C@H](C)OC(=O)[C@H](C)OC(C)=O)C1=Nc1ccc2nccnc2c1Br] |
| 187 | 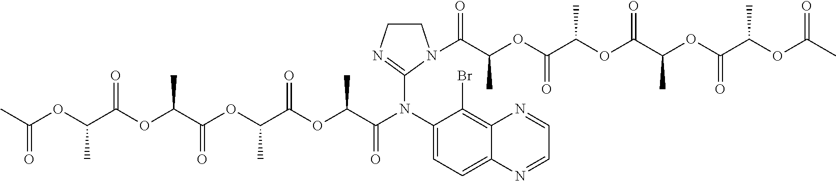 [SMILES: CC(=O)O[C@@H](C)C(=O)O[C@@H](C)C(=O)O[C@@H](C)C(=O)O[C@@H](C)C(=O)N1CCN=C1N(C(=O)[C@H](C)OC(=O)[C@H](C)OC(=O)[C@H](C)OC(=O)[C@H](C)OC(C)=O)c1ccc2nccnc2c1Br] |
| 188 | 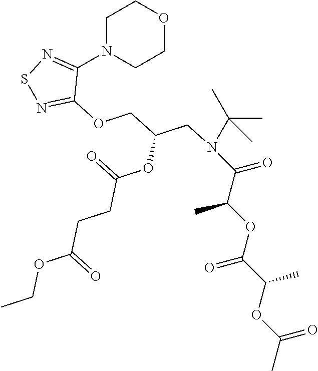 [SMILES: CCOC(=O)CCC(=O)O[C@H](COc1nsnc1N1CCOCC1)CN(C(=O)[C@H](C)OC(=O)[C@H](C)OC(C)=O)C(C)(C)C] |
| 189 | 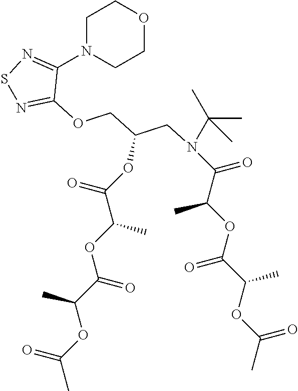 [SMILES: CC(=O)O[C@@H](C)C(=O)O[C@@H](C)C(=O)O[C@H](COc1nsnc1N1CCOCC1)CN(C(=O)[C@H](C)OC(=O)[C@H](C)OC(C)=O)C(C)(C)C] |
| 190 | 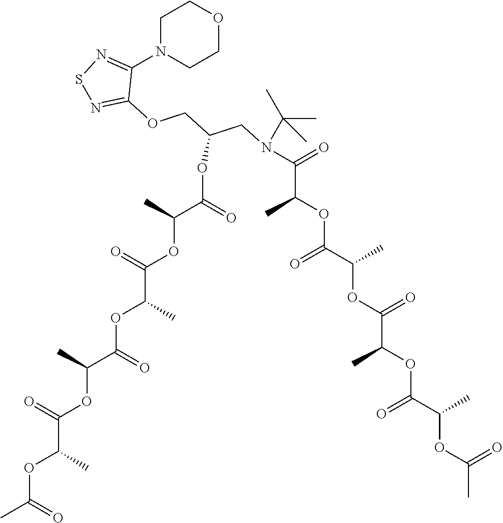 [SMILES: CC(=O)O[C@@H](C)C(=O)O[C@@H](C)C(=O)O[C@@H](C)C(=O)O[C@@H](C)C(=O)O[C@H](COc1nsnc1N1CCOCC1)CN(C(=O)[C@H](C)OC(=O)[C@H](C)OC(=O)[C@H](C)OC(=O)[C@H](C)OC(C)=O)C(C)(C)C] |
| 191 | 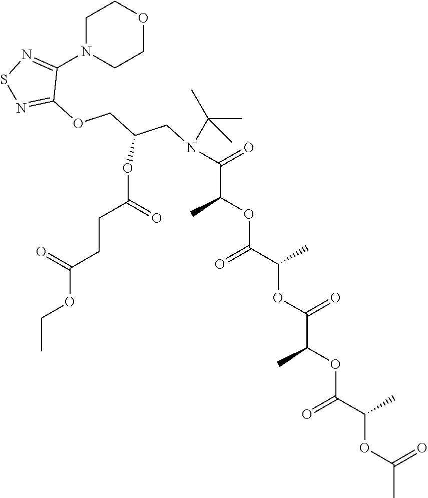 [SMILES: CCOC(=O)CCC(=O)O[C@H](COc1nsnc1N1CCOCC1)CN(C(=O)[C@H](C)OC(=O)[C@H](C)OC(=O)[C@H](C)OC(=O)[C@H](C)OC(C)=O)C(C)(C)C] |
| 192 | 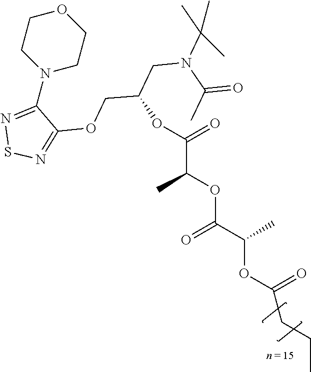 |
| 193 | 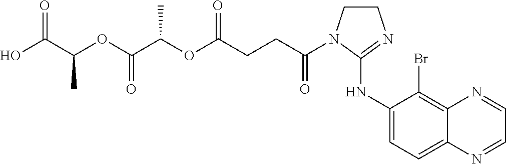 [SMILES: C[C@H](OC(=O)[C@H](C)OC(=O)CCC(=O)N1CCN=C1Nc1ccc2nccnc2c1Br)C(=O)O] |
| 194 | 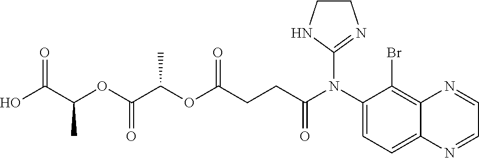 [SMILES: C[C@H](OC(=O)[C@H](C)OC(=O)CCC(=O)N(C1=NCCN1)c1ccc2nccnc2c1Br)C(=O)O] |
| 195 | 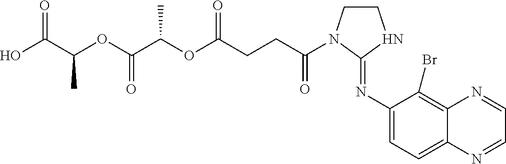 [SMILES: C[C@H](OC(=O)[C@H](C)OC(=O)CCC(=O)N1CCN/C1=N\c1ccc2nccnc2c1Br)C(=O)O] |
| 196 | 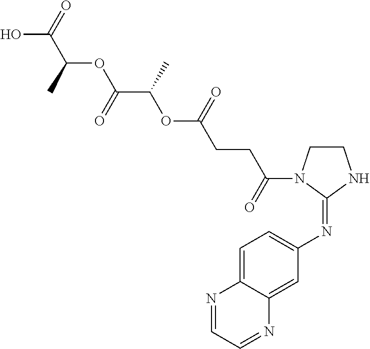 [SMILES: C[C@H](OC(=O)[C@H](C)OC(=O)CCC(=O)N1CCN/C1=N/c1ccc2nccnc2c1)C(=O)O] |
| 197 | 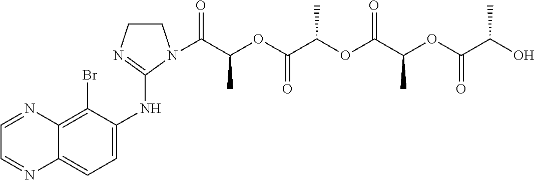 [SMILES: C[C@H](O)C(=O)O[C@@H](C)C(=O)O[C@@H](C)C(=O)O[C@@H](C)C(=O)N1CCN=C1Nc1ccc2nccnc2c1Br] |
| 198 | 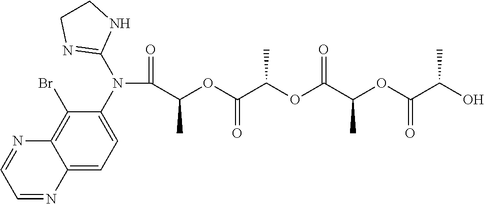 [SMILES: C[C@H](O)C(=O)O[C@@H](C)C(=O)O[C@@H](C)C(=O)O[C@@H](C)C(=O)N(C1=NCCN1)c1ccc2nccnc2c1Br] |
| 199 | 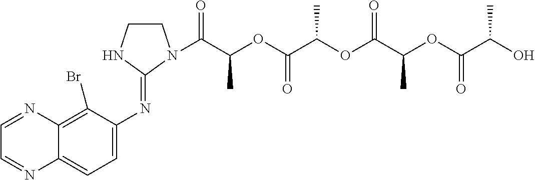 [SMILES: C[C@H](O)C(=O)O[C@@H](C)C(=O)O[C@@H](C)C(=O)O[C@@H](C)C(=O)N1CCN/C1=N\c1ccc2nccnc2c1Br] |
| 200 | 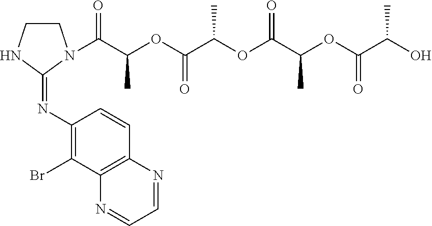 [SMILES: C[C@H](O)C(=O)O[C@@H](C)C(=O)O[C@@H](C)C(=O)O[C@@H](C)C(=O)N1CCN/C1=N/c1ccc2nccnc2c1Br] |
| 201 | 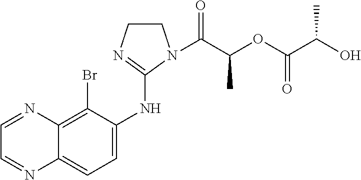 [SMILES: C[C@H](O)C(=O)O[C@@H](C)C(=O)N1CCN=C1Nc1ccc2nccnc2c1Br] |
| 202 | 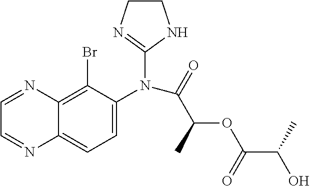 [SMILES: C[C@H](O)C(=O)O[C@@H](C)C(=O)N(C1=NCCN1)c1ccc2nccnc2c1Br] |
| 203 | 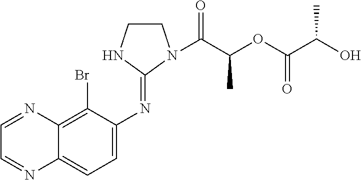 [SMILES: C[C@H](O)C(=O)O[C@@H](C)C(=O)N1CCN/C1=N\c1ccc2nccnc2c1Br] |
| 204 | 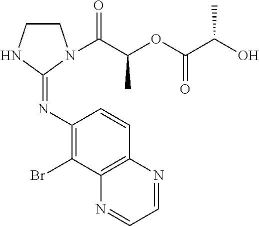 [SMILES: C[C@H](O)C(=O)O[C@@H](C)C(=O)N1CCN/C1=N/c1ccc2nccnc2c1Br] |

TABLE D
Non-limiting Examples of Prodrugs

| | |
|---|---|
| 205 | 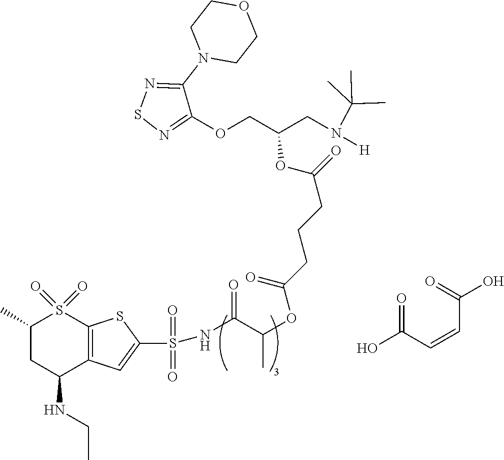 |
| 206 | 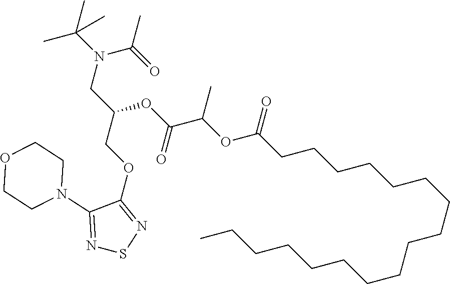 [SMILES: CCCCCCCCCCCCCCCCCC(=O)OC(C)C(=O)O[C@H](COc1nsnc1N1CCOCC1)CN(C(C)=O)C(C)(C)C] |
| 207 | 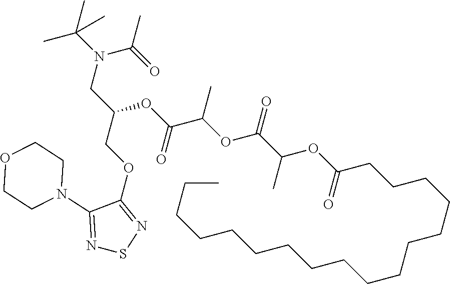 [SMILES: CCCCCCCCCCCCCCCCCC(=O)OC(C)C(=O)OC(C)C(=O)O[C@H](COc1nsnc1N1CCOCC1)CN(C(C)=O)C(C)(C)C] |
| 208 | 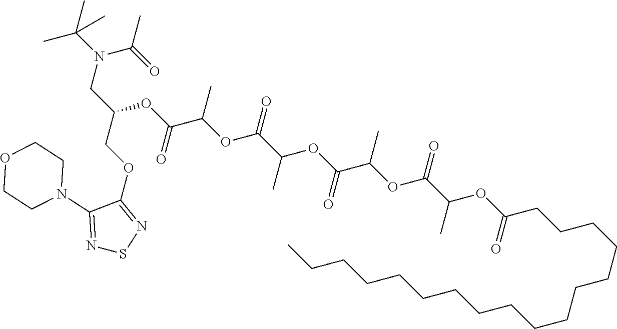 [SMILES: CCCCCCCCCCCCCCCCCC(=O)OC(C)C(=O)OC(C)C(=O)OC(C)C(=O)OC(C)C(=O)O[C@H](COc1nsnc1N1CCOCC1)CN(C(C)=O)C(C)(C)C] |
| 209 | 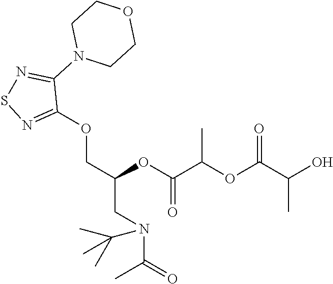 [SMILES: CC(=O)N(C[C@@H](COc1nsnc1N1CCOCC1)OC(=O)C(C)OC(=O)C(C)O)C(C)(C)C] |
| 210 | 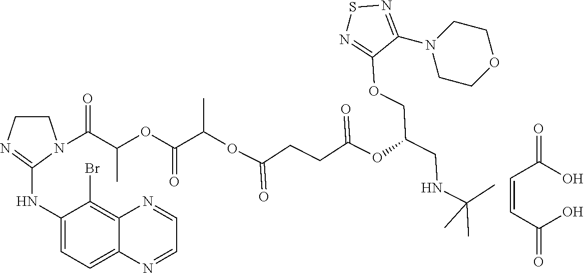 |
| 211 | 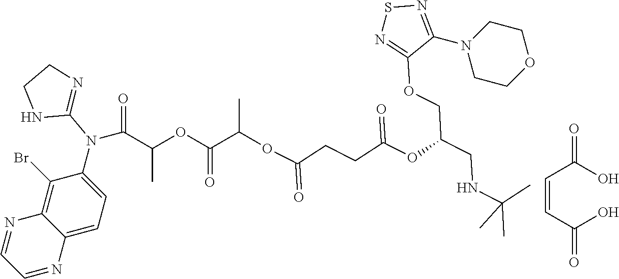 |
| 212 | 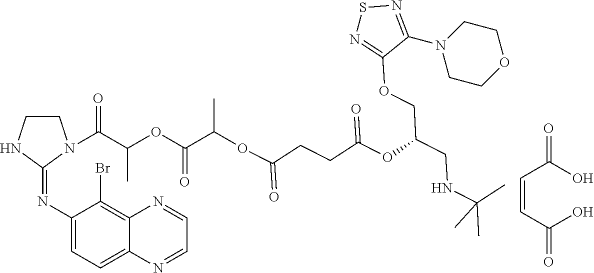 |
| 213 | 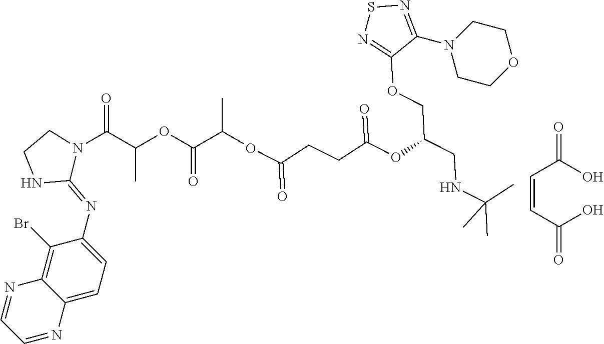 |
| 214 | 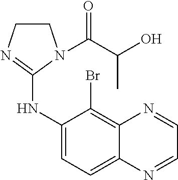 [SMILES: CC(O)C(=O)N1CCN=C1Nc1ccc2nccnc2c1Br] |
| 215 | 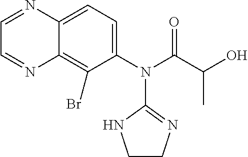 [SMILES: CC(O)C(=O)N(C1=NCCN1)c1ccc2nccnc2c1Br] |
| 216 | 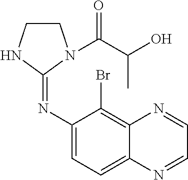 [SMILES: CC(O)C(=O)N1CCN/C1=N/c1ccc2nccnc2c1Br] |
| 217 | 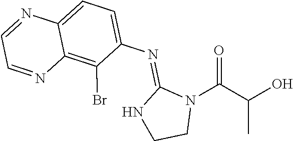 [SMILES: CC(O)C(=O)N1CCN/C1=N\c1ccc2nccnc2c1Br] |
| 218 | 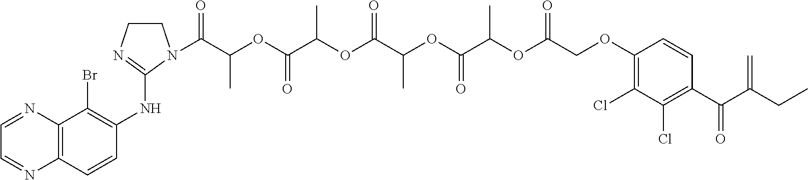 [SMILES: C=C(CC)C(=O)c1ccc(OCC(=O)OC(C)C(=O)OC(C)C(=O)OC(C)C(=O)OC(C)C(=O)N2CCN=C2Nc2ccc3nccnc3c2Br)c(Cl)c1Cl] |
| 219 | 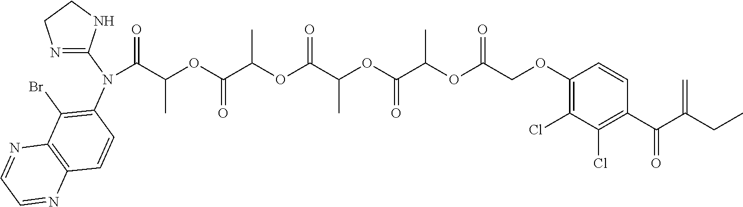 [SMILES: C=C(CC)C(=O)c1ccc(OCC(=O)OC(C)C(=O)OC(C)C(=O)OC(C)C(=O)OC(C)C(=O)N(C2=NCCN2)c2ccc3nccnc3c2Br)c(Cl)c1Cl] |
| 220 | 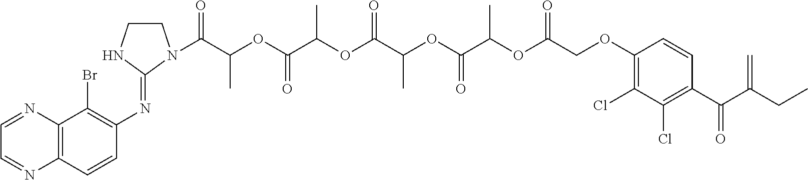 [SMILES: C=C(CC)C(=O)c1ccc(OCC(=O)OC(C)C(=O)OC(C)C(=O)OC(C)C(=O)OC(C)C(=O)N2CCN/C2=N\c2ccc3nccnc3c2Br)c(Cl)c1Cl] |
| 221 | 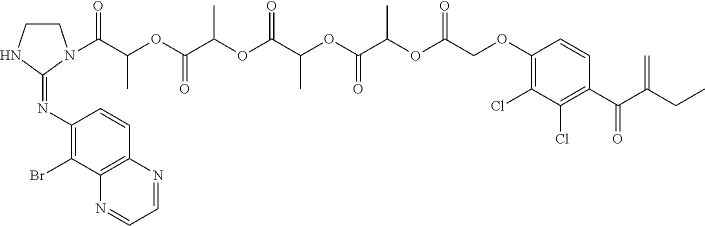 [SMILES: C=C(CC)C(=O)c1ccc(OCC(=O)OC(C)C(=O)OC(C)C(=O)OC(C)C(=O)OC(C)C(=O)N2CCN/C2=N/c2ccc3nccnc3c2Br)c(Cl)c1Cl] |
| 222 | 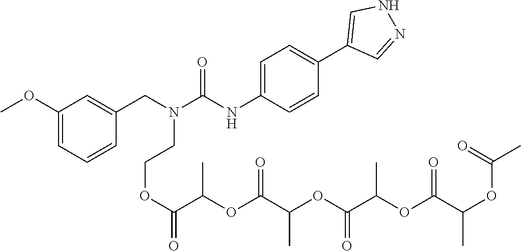 [SMILES: COc1cccc(CN(CCOC(=O)C(C)OC(=O)C(C)OC(=O)C(C)OC(=O)C(C)OC(C)=O)C(=O)Nc2ccc(-c3cn[nH]c3)cc2)c1] |
| 223 | 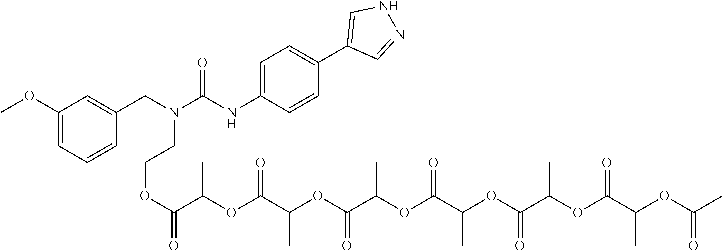 [SMILES: COc1cccc(CN(CCOC(=O)C(C)OC(=O)C(C)OC(=O)C(C)OC(=O)C(C)OC(=O)C(C)OC(=O)C(C)OC(C)=O)C(=O)Nc2ccc(-c3cn[nH]c3)cc2)c1] |
| 224 | 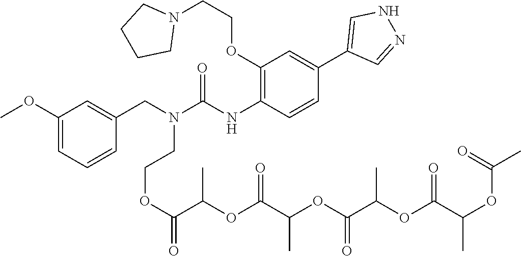 [SMILES: COc1cccc(CN(CCOC(=O)C(C)OC(=O)C(C)OC(=O)C(C)OC(=O)C(C)OC(C)=O)C(=O)Nc2ccc(-c3cn[nH]c3)cc2OCCN2CCCC2)c1] |
| 225 | 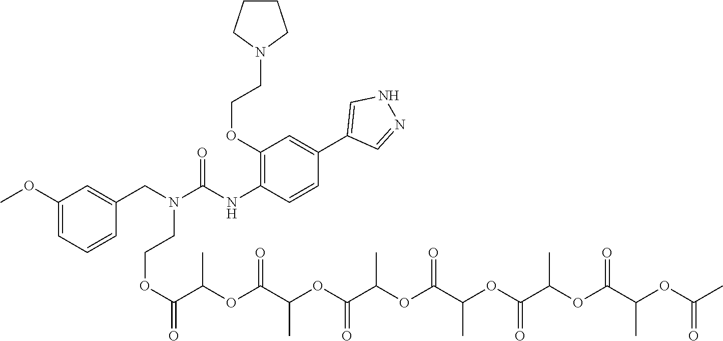 [SMILES: COc1cccc(CN(CCOC(=O)C(C)OC(=O)C(C)OC(=O)C(C)OC(=O)C(C)OC(=O)C(C)OC(=O)C(C)OC(C)=O)C(=O)Nc2ccc(-c3cn[nH]c3)cc2OCCN2CCCC2)c1] |
| 226 | 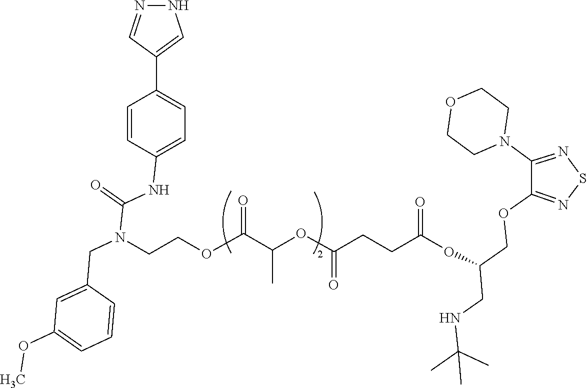 |
| 227 | 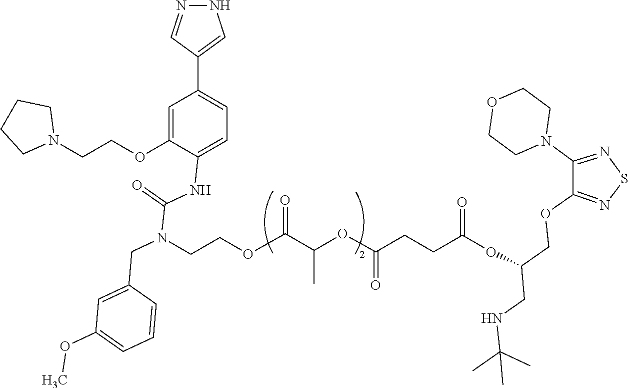 |
| 228 | 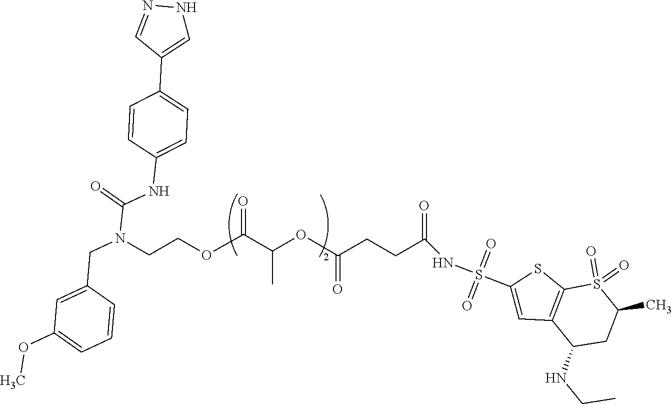 |
| 229 | 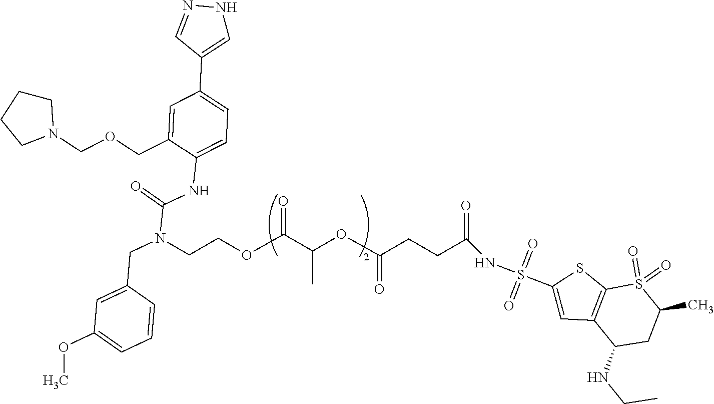 |
| 230 | 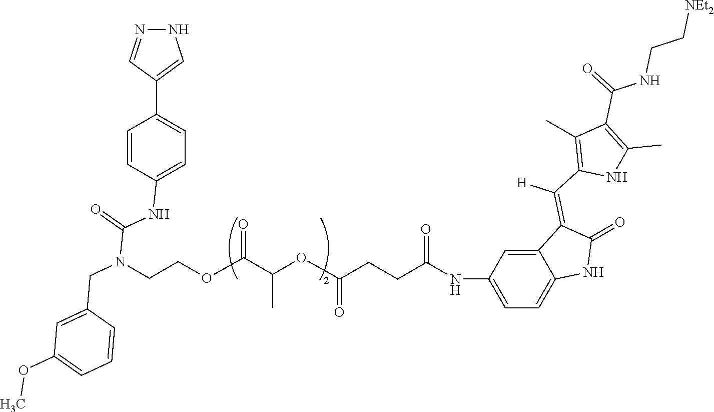 |
| 231 | 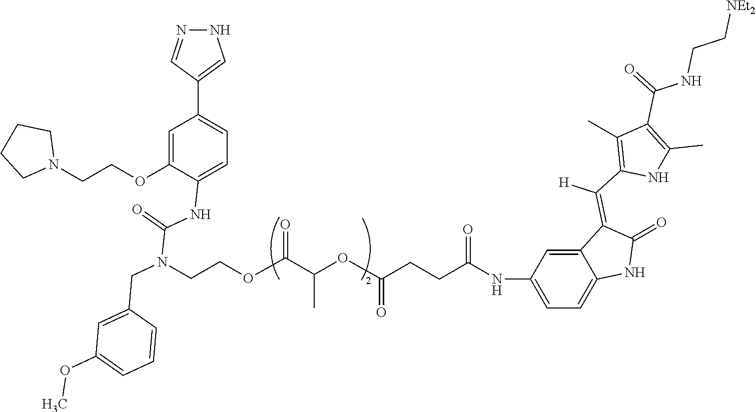 |
| 232 | 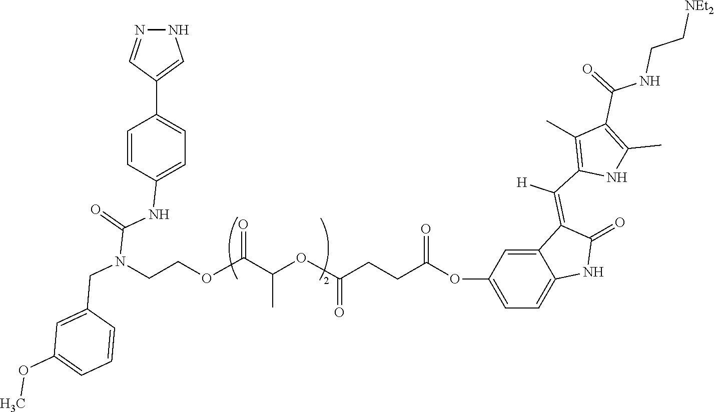 |
| 233 | 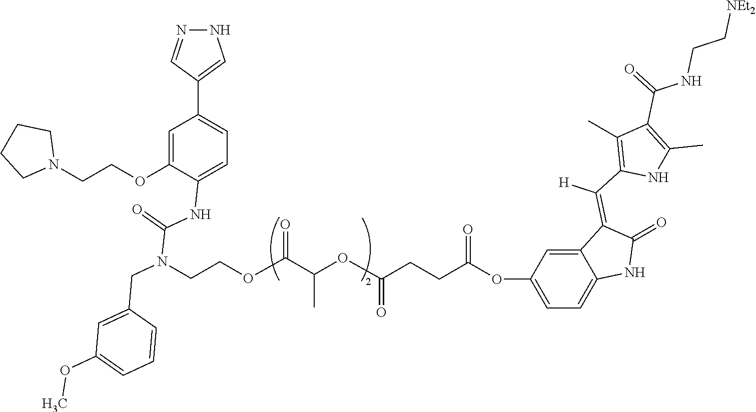 |
| 234 | 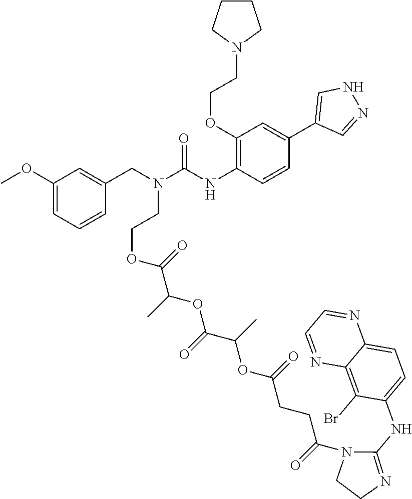 [SMILES: COc1cccc(CN(CCOC(=O)C(C)OC(=O)C(C)OC(=O)CCC(=O)N2CCN=C2Nc2ccc3nccnc3c2Br)C(=O)Nc2ccc(-c3cn[nH]c3)cc2OCCN2CCCC2)c1] |
| 235 | 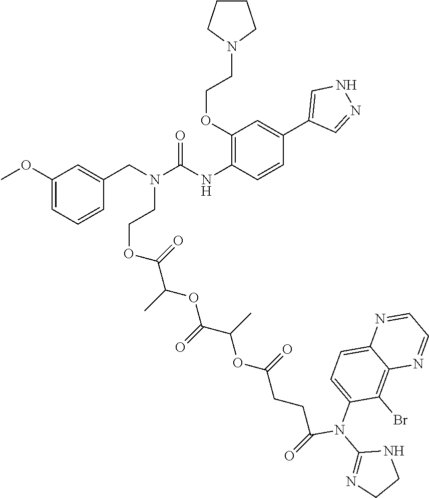 [SMILES: COc1cccc(CN(CCOC(=O)C(C)OC(=O)C(C)OC(=O)CCC(=O)N(C2=NCCN2)c2ccc3nccnc3c2Br)C(=O)Nc2ccc(-c3cn[nH]c3)cc2OCCN2CCCC2)c1] |
| 236 | 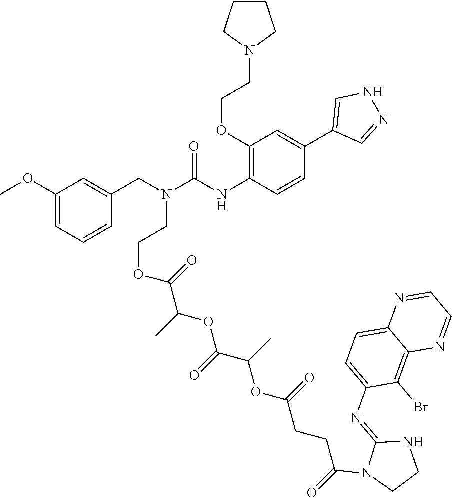 [SMILES: COc1cccc(CN(CCOC(=O)C(C)OC(=O)C(C)OC(=O)CCC(=O)N2CCN/C2=N\c2ccc3nccnc3c2Br)C(=O)Nc2ccc(-c3cn[nH]c3)cc2OCCN2CCCC2)c1] |
| 237 | 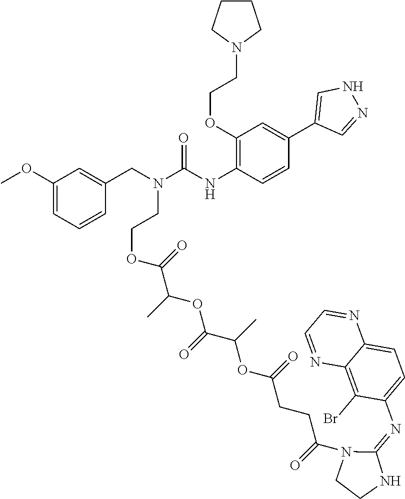 [SMILES: COc1cccc(CN(CCOC(=O)C(C)OC(=O)C(C)OC(=O)CCC(=O)N2CCN/C2=N/c2ccc3nccnc3c2Br)C(=O)Nc2ccc(-c3cn[nH]c3)cc2OCCN2CCCC2)c1] |
| 238 | 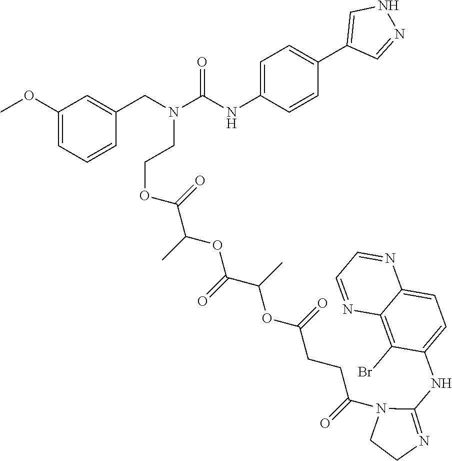 [SMILES: COc1cccc(CN(CCOC(=O)C(C)OC(=O)C(C)OC(=O)CCC(=O)N2CCN=C2Nc2ccc3nccnc3c2Br)C(=O)Nc2ccc(-c3cn[nH]c3)cc2)c1] |
| 239 | 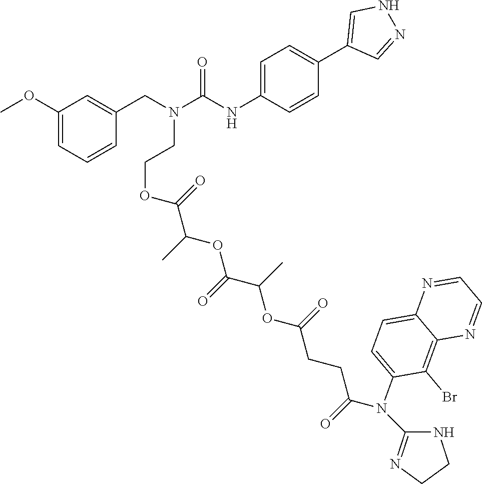 [SMILES: COc1cccc(CN(CCOC(=O)C(C)OC(=O)C(C)OC(=O)CCC(=O)N(C2=NCCN2)c2ccc3nccnc3c2Br)C(=O)Nc2ccc(-c3cn[nH]c3)cc2)c1] |
| 240 | 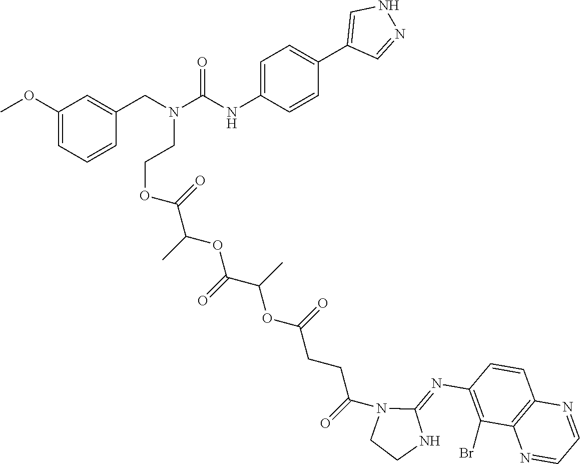 [SMILES: COc1cccc(CN(CCOC(=O)C(C)OC(=O)C(C)OC(=O)CCC(=O)N2CCN/C2=N\c2ccc3nccnc3c2Br)C(=O)Nc2ccc(-c3cn[nH]c3)cc2)c1] |
| 241 | 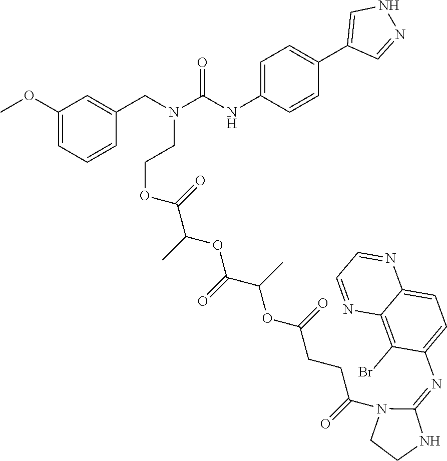 [SMILES: COc1cccc(CN(CCOC(=O)C(C)OC(=O)C(C)OC(=O)CCC(=O)N2CCN/C2=N/c2ccc3nccnc3c2Br)C(=O)Nc2ccc(-c3cn[nH]c3)cc2)c1] |
| 242 | 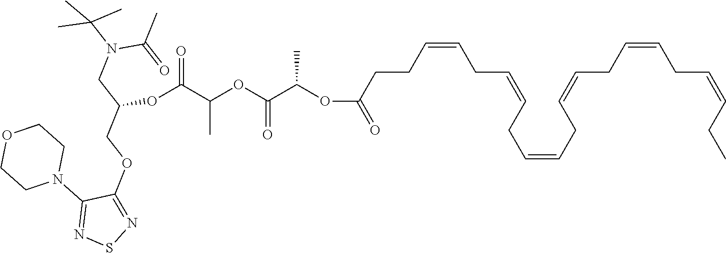 [SMILES: CC/C=C\C/C=C\C/C=C\C/C=C\C/C=C\C/C=C\CCC(=O)O[C@@H](C)C(=O)OC(C)C(=O)O[C@H](COc1nsnc1N1CCOCC1)CN(C(C)=O)C(C)(C)C] |
| 243 | 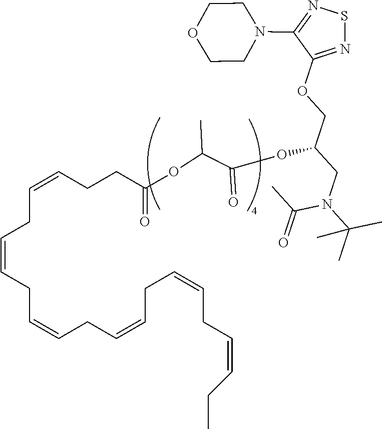 |
| 244 | 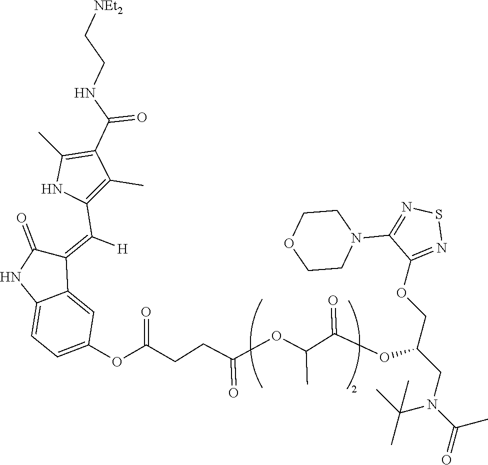 |
| 245 | 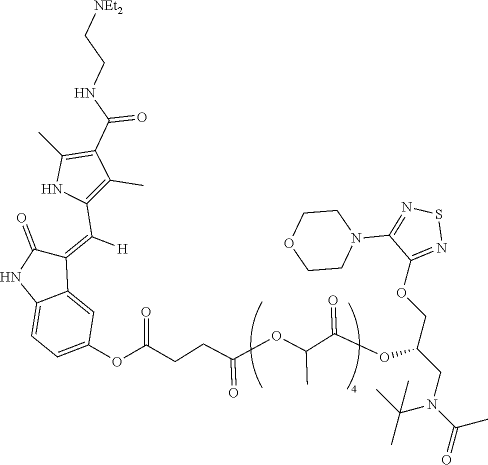 |
| 246 | 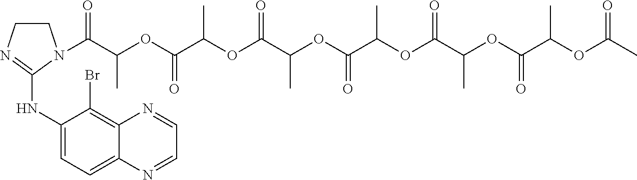 [SMILES: CC(=O)OC(C)C(=O)OC(C)C(=O)OC(C)C(=O)OC(C)C(=O)OC(C)C(=O)OC(C)C(=O)N1CCN=C1Nc1ccc2nccnc2c1Br] |
| 247 | 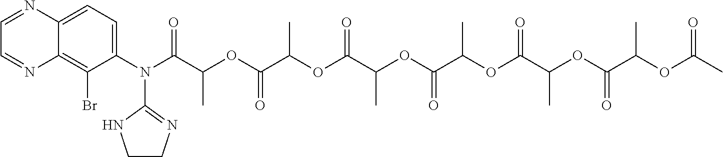 [SMILES: CC(=O)OC(C)C(=O)OC(C)C(=O)OC(C)C(=O)OC(C)C(=O)OC(C)C(=O)OC(C)C(=O)N(C1=NCCN1)c1ccc2nccnc2c1Br] |
| 248 | 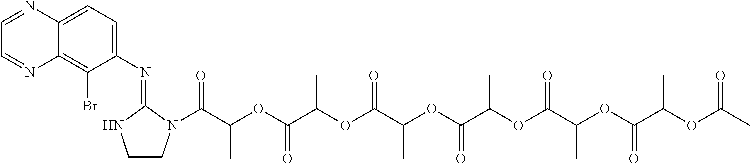 [SMILES: CC(=O)OC(C)C(=O)OC(C)C(=O)OC(C)C(=O)OC(C)C(=O)OC(C)C(=O)OC(C)C(=O)N1CCN/C1=N\c1ccc2nccnc2c1Br] |
| 249 | 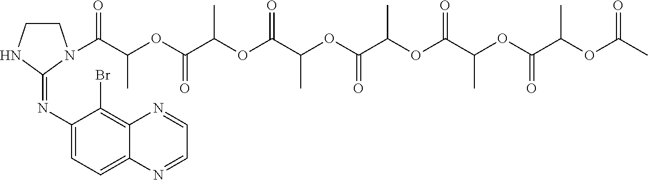 [SMILES: CC(=O)OC(C)C(=O)OC(C)C(=O)OC(C)C(=O)OC(C)C(=O)OC(C)C(=O)OC(C)C(=O)N1CCN/C1=N/c1ccc2nccnc2c1Br] |
| 250 | 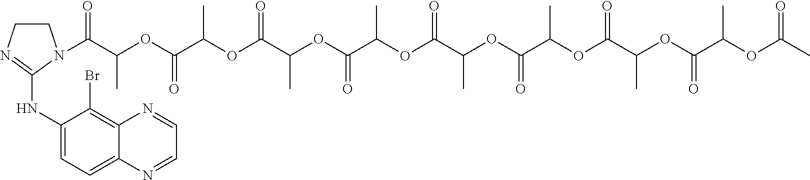 [SMILES: CC(=O)OC(C)C(=O)OC(C)C(=O)OC(C)C(=O)OC(C)C(=O)OC(C)C(=O)OC(C)C(=O)OC(C)C(=O)OC(C)C(=O)N1CCN=C1Nc1ccc2nccnc2c1Br] |
| 251 | 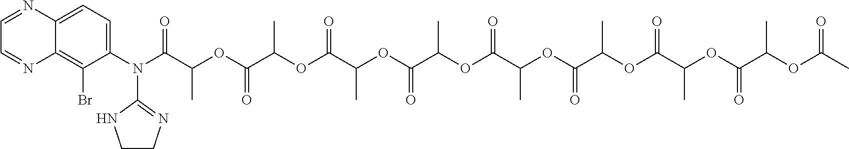 [SMILES: CC(=O)OC(C)C(=O)OC(C)C(=O)OC(C)C(=O)OC(C)C(=O)OC(C)C(=O)OC(C)C(=O)OC(C)C(=O)OC(C)C(=O)N(C1=NCCN1)c1ccc2nccnc2c1Br] |
| 252 | 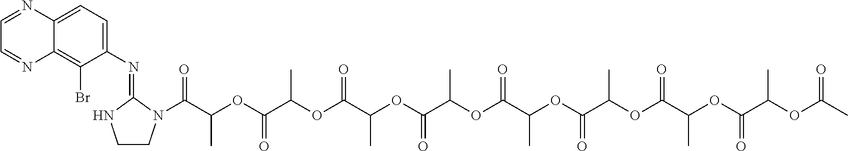 [SMILES: CC(=O)OC(C)C(=O)OC(C)C(=O)OC(C)C(=O)OC(C)C(=O)OC(C)C(=O)OC(C)C(=O)OC(C)C(=O)OC(C)C(=O)N1CCN/C1=N\c1ccc2nccnc2c1Br] |
| 253 | 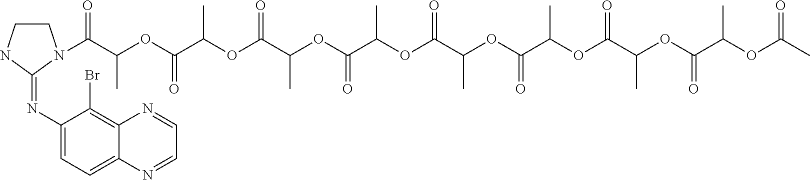 [SMILES: CC(=O)OC(C)C(=O)OC(C)C(=O)OC(C)C(=O)OC(C)C(=O)OC(C)C(=O)OC(C)C(=O)OC(C)C(=O)OC(C)C(=O)N1CCN/C1=N/c1ccc2nccnc2c1Br] |
| 254 | 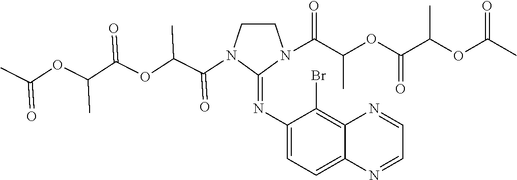 [SMILES: CC(=O)OC(C)C(=O)OC(C)C(=O)N1CCN(C(=O)C(C)OC(=O)C(C)OC(C)=O)C1=Nc1ccc2nccnc2c1Br] |
| 255 | 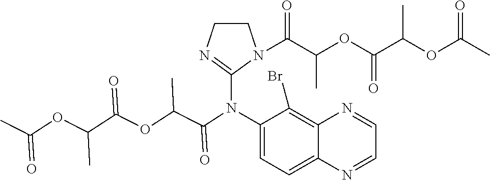 [SMILES: CC(=O)OC(C)C(=O)OC(C)C(=O)N1CCN=C1N(C(=O)C(C)OC(=O)C(C)OC(C)=O)c1ccc2nccnc2c1Br] |
| 256 | 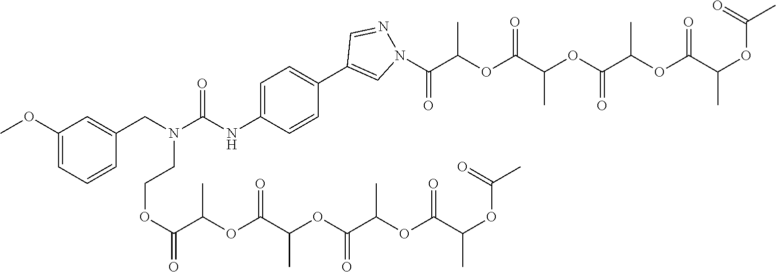 [SMILES: COc1cccc(CN(CCOC(=O)C(C)OC(=O)C(C)OC(=O)C(C)OC(=O)C(C)OC(C)=O)C(=O)Nc2ccc(-c3cnn(C(=O)C(C)OC(=O)C(C)OC(=O)C(C)OC(=O)C(C)OC(C)=O)c3)cc2)c1] |
| 257 | 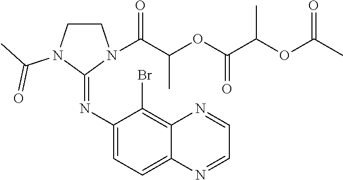 [SMILES: CC(=O)OC(C)C(=O)OC(C)C(=O)N1CCN(C(C)=O)/C1=N/c1ccc2nccnc2c1Br] |
| 258 | 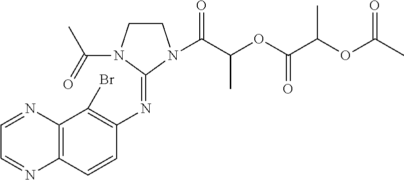 [SMILES: CC(=O)OC(C)C(=O)OC(C)C(=O)N1CCN(C(C)=O)/C1=N\c1ccc2nccnc2c1Br] |
| 259 | 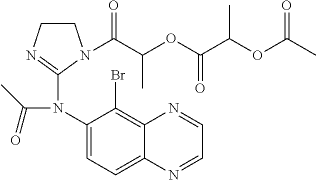 [SMILES: CC(=O)OC(C)C(=O)OC(C)C(=O)N1CCN=C1N(C(C)=O)c1ccc2nccnc2c1Br] |
| 260 | 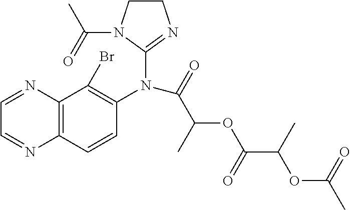 [SMILES: CC(=O)OC(C)C(=O)OC(C)C(=O)N(C1=NCCN1C(C)=O)c1ccc2nccnc2c1Br] |
| 261 | 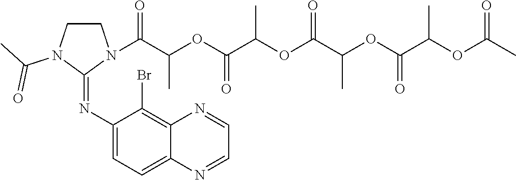 [SMILES: CC(=O)OC(C)C(=O)OC(C)C(=O)OC(C)C(=O)OC(C)C(=O)N1CCN(C(C)=O)/C1=N/c1ccc2nccnc2c1Br] |
| 262 | 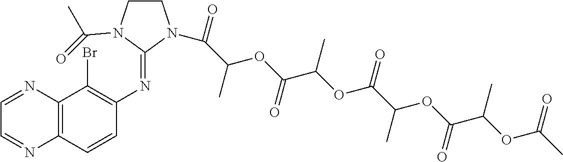 [SMILES: CC(=O)OC(C)C(=O)OC(C)C(=O)OC(C)C(=O)OC(C)C(=O)N1CCN(C(C)=O)/C1=N\c1ccc2nccnc2c1Br] |
| 263 | 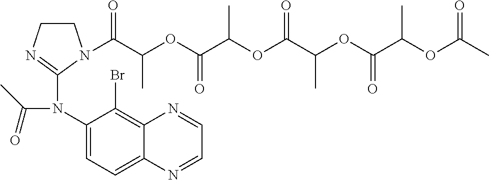 [SMILES: CC(=O)OC(C)C(=O)OC(C)C(=O)OC(C)C(=O)OC(C)C(=O)N1CCN=C1N(C(C)=O)c1ccc2nccnc2c1Br] |
| 264 | 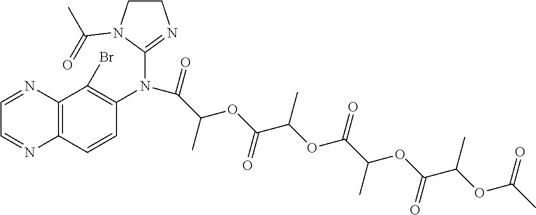 [SMILES: CC(=O)OC(C)C(=O)OC(C)C(=O)OC(C)C(=O)OC(C)C(=O)N(C1=NCCN1C(C)=O)c1ccc2nccnc2c1Br] |
| 265 | 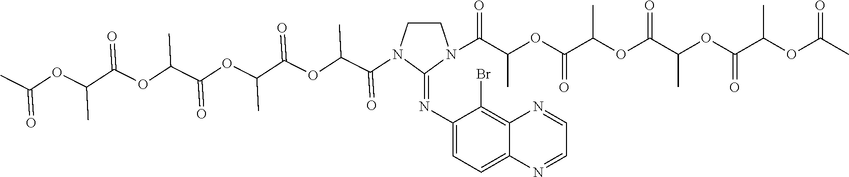 [SMILES: CC(=O)OC(C)C(=O)OC(C)C(=O)OC(C)C(=O)OC(C)C(=O)N1CCN(C(=O)C(C)OC(=O)C(C)OC(=O)C(C)OC(=O)C(C)OC(C)=O)C1=Nc1ccc2nccnc2c1Br] |
| 266 | 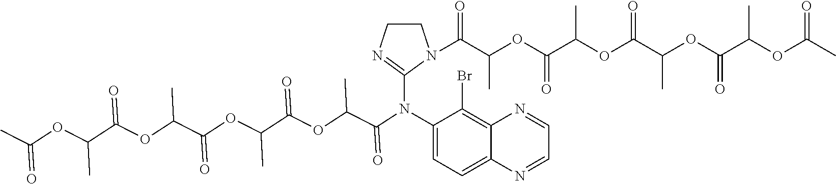 [SMILES: CC(=O)OC(C)C(=O)OC(C)C(=O)OC(C)C(=O)OC(C)C(=O)N1CCN=C1N(C(=O)C(C)OC(=O)C(C)OC(=O)C(C)OC(=O)C(C)OC(C)=O)c1ccc2nccnc2c1Br] |
| 267 | 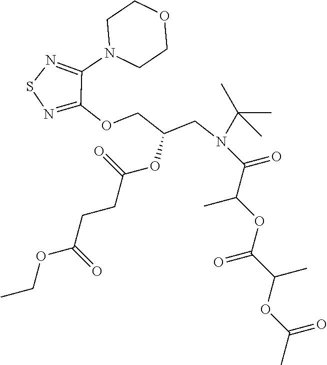 [SMILES: CCOC(=O)CCC(=O)O[C@H](COc1nsnc1N1CCOCC1)CN(C(=O)C(C)OC(=O)C(C)OC(C)=O)C(C)(C)C] |
| 268 | 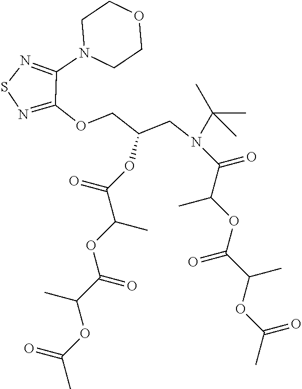 [SMILES: CC(=O)OC(C)C(=O)OC(C)C(=O)O[C@H](COc1nsnc1N1CCOCC1)CN(C(=O)C(C)OC(=O)C(C)OC(C)=O)C(C)(C)C] |
| 269 | 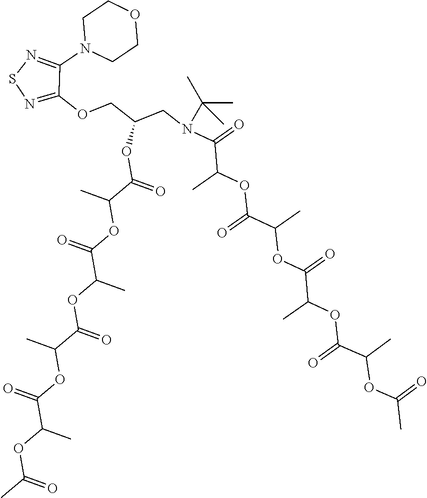 [SMILES: CC(=O)OC(C)C(=O)OC(C)C(=O)OC(C)C(=O)OC(C)C(=O)O[C@H](COc1nsnc1N1CCOCC1)CN(C(=O)C(C)OC(=O)C(C)OC(=O)C(C)OC(=O)C(C)OC(C)=O)C(C)(C)C] |
| 270 | 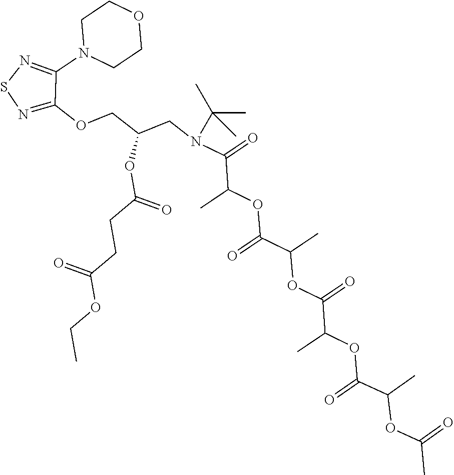 [SMILES: CCOC(=O)CCC(=O)O[C@H](COc1nsnc1N1CCOCC1)CN(C(=O)C(C)OC(=O)C(C)OC(=O)C(C)OC(=O)C(C)OC(C)=O)C(C)(C)C] |
| 271 | 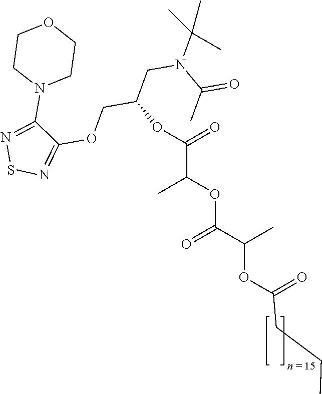 |
| 272 | 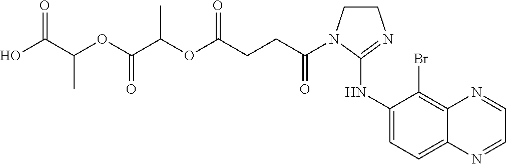 [SMILES: CC(OC(=O)C(C)OC(=O)CCC(=O)N1CCN=C1Nc1ccc2nccnc2c1Br)C(=O)O] |
| 273 | 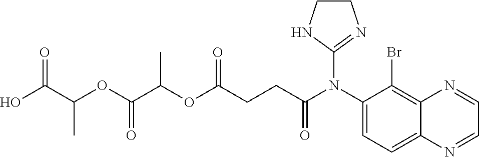 [SMILES: CC(OC(=O)C(C)OC(=O)CCC(=O)N(C1=NCCN1)c1ccc2nccnc2c1Br)C(=O)O] |
| 274 | 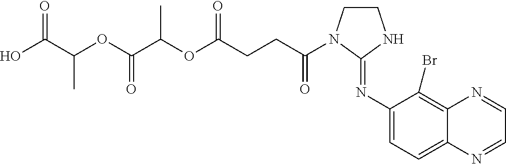 [SMILES: CC(OC(=O)C(C)OC(=O)CCC(=O)N1CCN/C1=N\c1ccc2nccnc2c1Br)C(=O)O] |
| 275 | 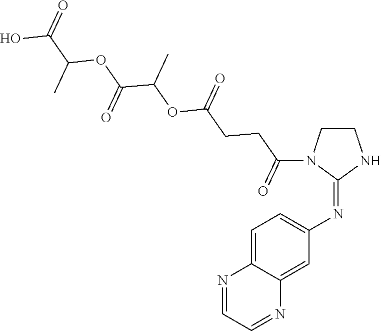 [SMILES: CC(OC(=O)C(C)OC(=O)CCC(=O)N1CCN/C1=N/c1ccc2nccnc2c1)C(=O)O] |
| 276 | 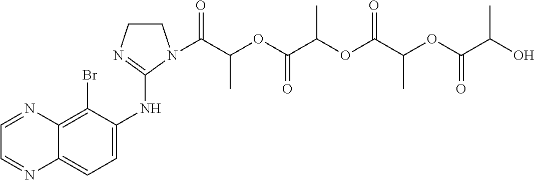 [SMILES: CC(O)C(=O)OC(C)C(=O)OC(C)C(=O)OC(C)C(=O)N1CCN=C1Nc1ccc2nccnc2c1Br] |
| 277 | 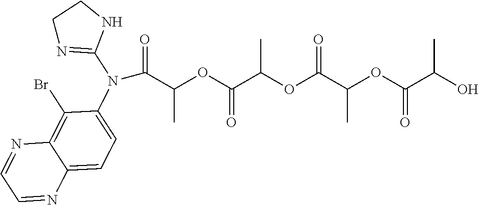 [SMILES: CC(O)C(=O)OC(C)C(=O)OC(C)C(=O)OC(C)C(=O)N(C1=NCCN1)c1ccc2nccnc2c1Br] |
| 278 | 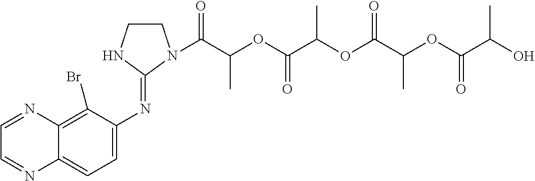 [SMILES: CC(O)C(=O)OC(C)C(=O)OC(C)C(=O)OC(C)C(=O)N1CCN/C1=N\c1ccc2nccnc2c1Br] |
| 279 | 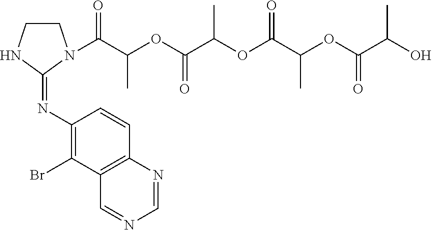 [SMILES: CC(O)C(=O)OC(C)C(=O)OC(C)C(=O)OC(C)C(=O)N1CCN/C1=N/c1ccc2ncncc2c1Br] |
| 280 | 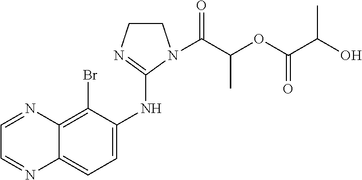 [SMILES: CC(O)C(=O)OC(C)C(=O)N1CCN=C1Nc1ccc2nccnc2c1Br] |
| 281 | 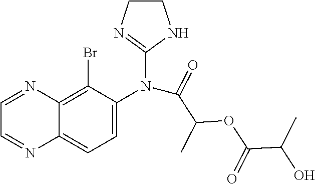 [SMILES: CC(O)C(=O)OC(C)C(=O)N(C1=NCCN1)c1ccc2nccnc2c1Br] |
| 282 | 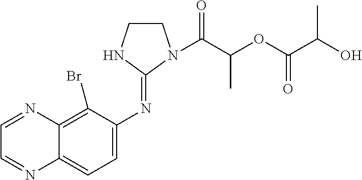 [SMILES: CC(O)C(=O)OC(C)C(=O)N1CCN/C1=N\c1ccc2nccnc2c1Br] |
| 283 | 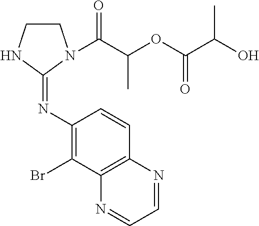 [SMILES: CC(O)C(=O)OC(C)C(=O)N1CCN/C1=N/c1ccc2nccnc2c1Br] |

TABLE E
Select Compounds of the Present Invention

| | |
|---|---|
| 284 | 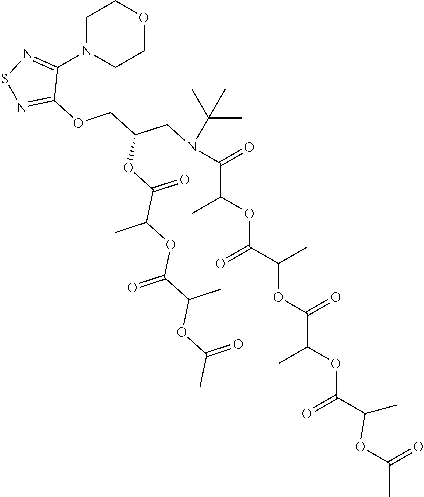 [SMILES: CC(=O)OC(C)C(=O)OC(C)C(=O)OC(C)C(=O)OC(C)C(=O)N(C[C@@H](COc1nsnc1N1CCOCC1)OC(=O)C(C)OC(=O)C(C)OC(C)=O)C(C)(C)C] |
| 285 | 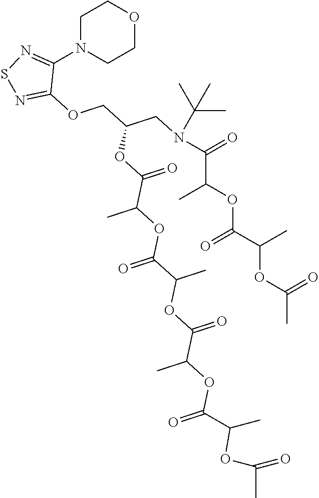 [SMILES: CC(=O)OC(C)C(=O)OC(C)C(=O)OC(C)C(=O)OC(C)C(=O)O[C@H](COc1nsnc1N1CCOCC1)CN(C(=O)C(C)OC(=O)C(C)OC(C)=O)C(C)(C)C] |
| 286 | 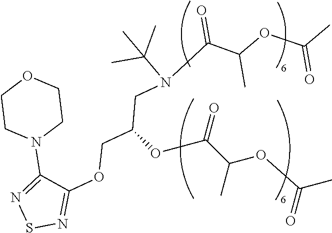 |
| 287 | 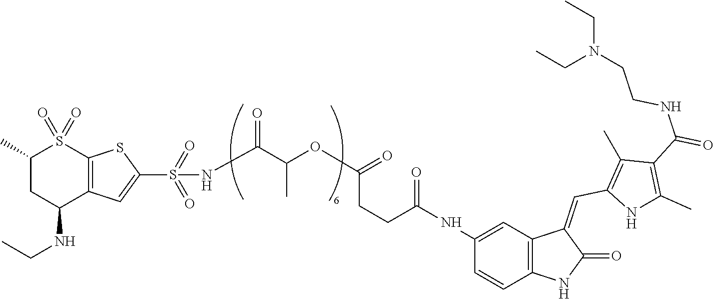 |
| 288 | 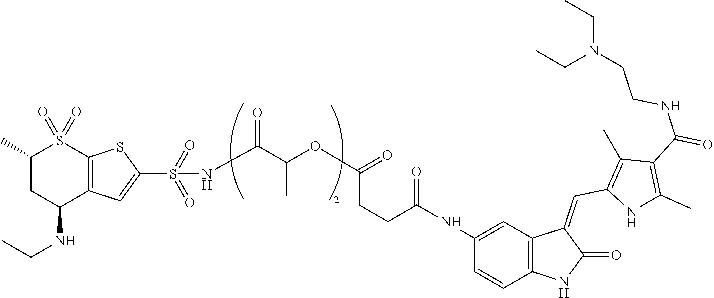 |
| 289 | 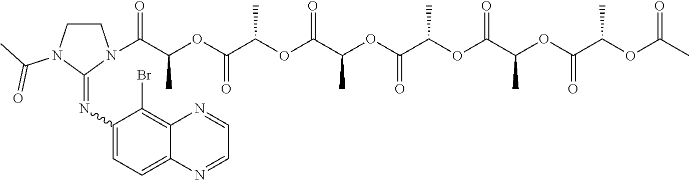 [SMILES: CC(=O)O[C@@H](C)C(=O)O[C@@H](C)C(=O)O[C@@H](C)C(=O)O[C@@H](C)C(=O)O[C@@H](C)C(=O)O[C@@H](C)C(=O)N1CCN(C(C)=O)C1=Nc1ccc2nccnc2c1Br] |
| 290 | 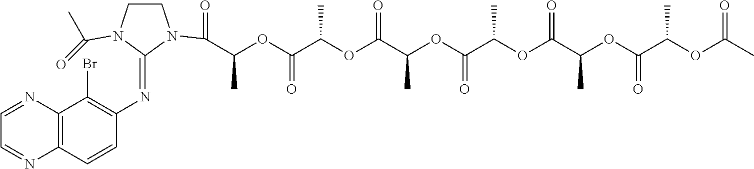 [SMILES: CC(=O)O[C@@H](C)C(=O)O[C@@H](C)C(=O)O[C@@H](C)C(=O)O[C@@H](C)C(=O)O[C@@H](C)C(=O)O[C@@H](C)C(=O)N1CCN(C(C)=O)/C1=N\c1ccc2nccnc2c1Br] |
| 291 | 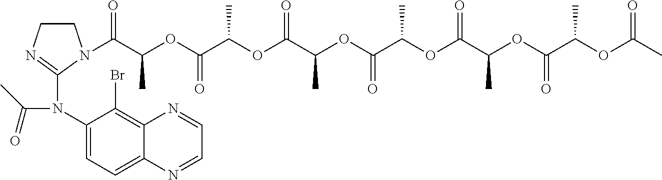 [SMILES: CC(=O)O[C@@H](C)C(=O)O[C@@H](C)C(=O)O[C@@H](C)C(=O)O[C@@H](C)C(=O)O[C@@H](C)C(=O)O[C@@H](C)C(=O)N1CCN=C1N(C(C)=O)c1ccc2nccnc2c1Br] |
| 292 | 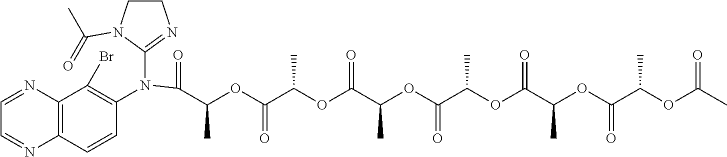 [SMILES: CC(=O)O[C@@H](C)C(=O)O[C@@H](C)C(=O)O[C@@H](C)C(=O)O[C@@H](C)C(=O)O[C@@H](C)C(=O)O[C@@H](C)C(=O)N(C1=NCCN1C(C)=O)c1ccc2nccnc2c1Br] |
| 293 | 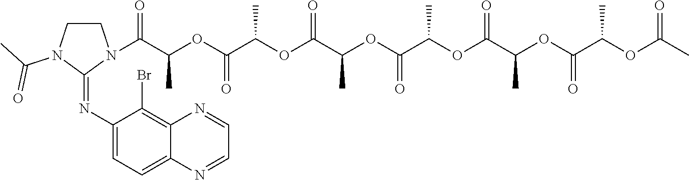 [SMILES: CC(=O)O[C@@H](C)C(=O)O[C@@H](C)C(=O)O[C@@H](C)C(=O)O[C@@H](C)C(=O)O[C@@H](C)C(=O)O[C@@H](C)C(=O)N1CCN(C(C)=O)/C1=N/c1ccc2nccnc2c1Br] |
| 294 | 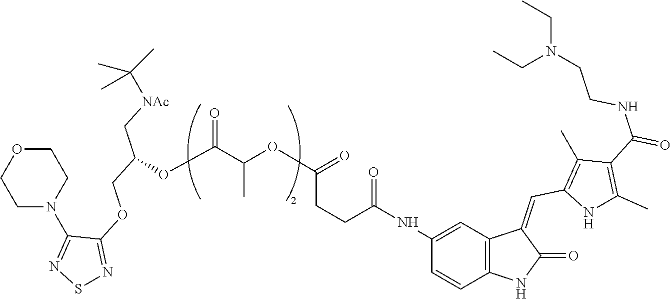 |
| 295 | 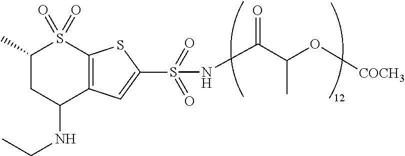 |
| 296 | 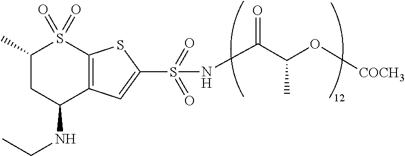 |
| 297 | 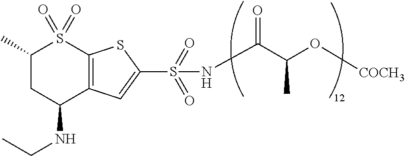 |
| 298 | 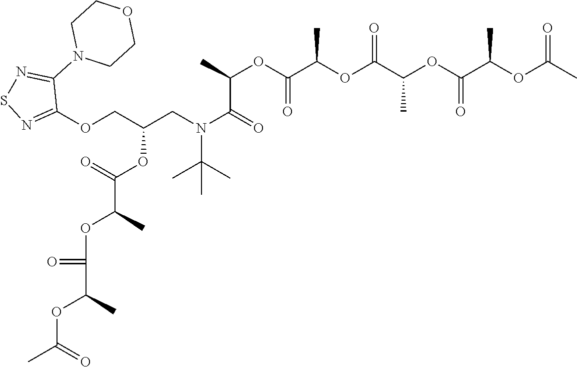 [SMILES: CC(=O)O[C@H](C)C(=O)O[C@H](C)C(=O)O[C@H](COc1nsnc1N1CCOCC1)CN(C(=O)[C@@H](C)OC(=O)[C@@H](C)OC(=O)[C@@H](C)OC(=O)[C@@H](C)OC(C)=O)C(C)(C)C] |
| 299 | 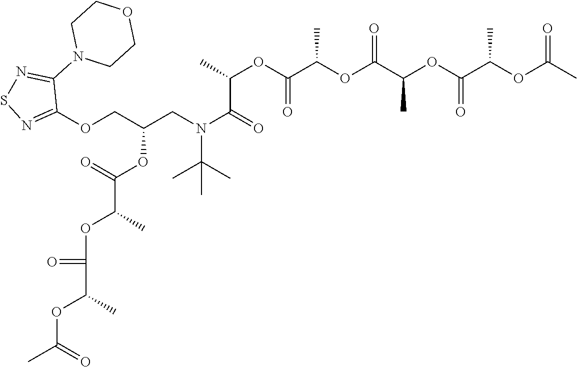 [SMILES: CC(=O)O[C@@H](C)C(=O)O[C@@H](C)C(=O)O[C@H](COc1nsnc1N1CCOCC1)CN(C(=O)[C@H](C)OC(=O)[C@H](C)OC(=O)[C@H](C)OC(=O)[C@H](C)OC(C)=O)C(C)(C)C] |
| 300 | 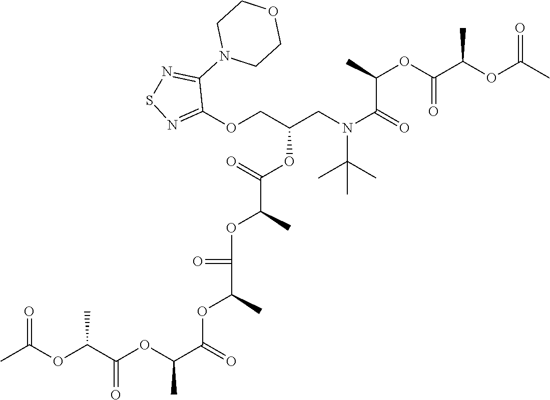 [SMILES: CC(=O)O[C@H](C)C(=O)O[C@H](C)C(=O)O[C@H](C)C(=O)O[C@H](C)C(=O)O[C@H](COc1nsnc1N1CCOCC1)CN(C(=O)[C@@H](C)OC(=O)[C@@H](C)OC(C)=O)C(C)(C)C] |
| 301 | 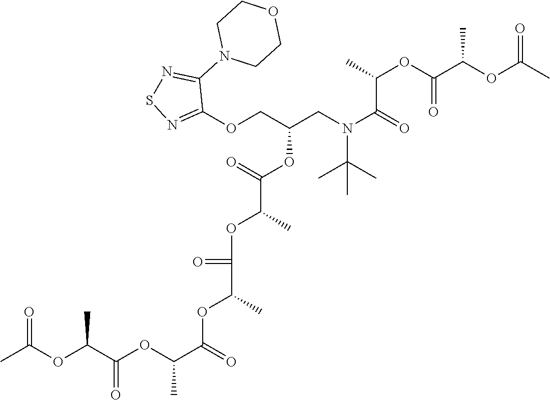 [SMILES: CC(=O)O[C@@H](C)C(=O)O[C@@H](C)C(=O)O[C@@H](C)C(=O)O[C@@H](C)C(=O)O[C@H](COc1nsnc1N1CCOCC1)CN(C(=O)[C@H](C)OC(=O)[C@H](C)OC(C)=O)C(C)(C)C] |
| 302 | 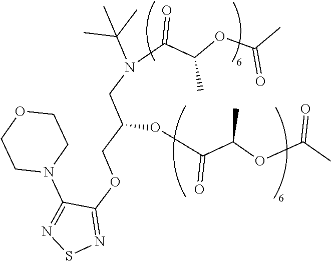 |
| 303 | 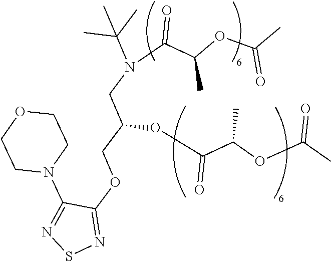 |
| 304 | 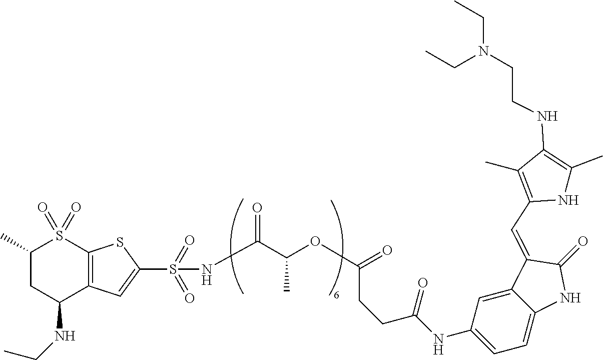 |
| 305 | 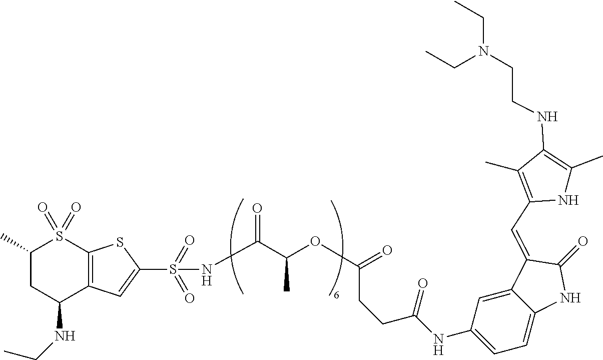 |
| 306 | 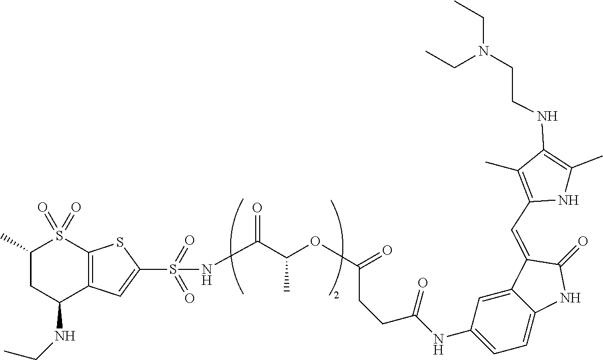 |
| 307 | 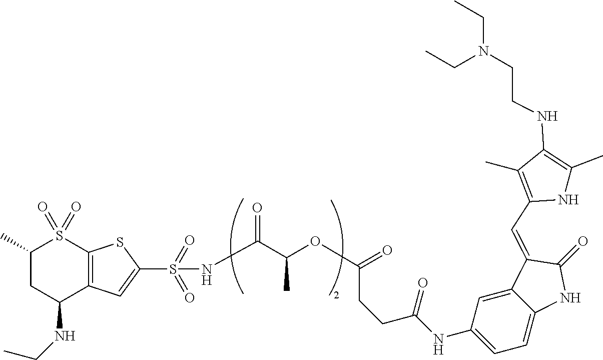 |
| 308 | 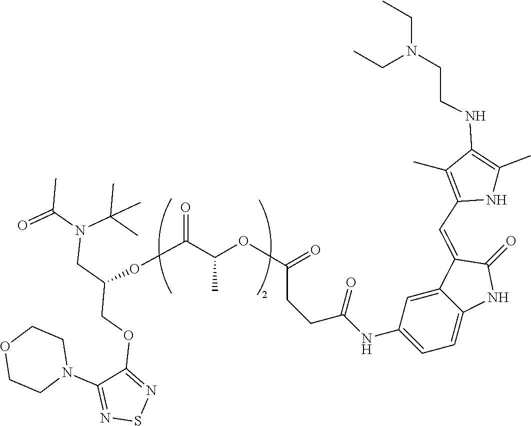 |
| 309 | 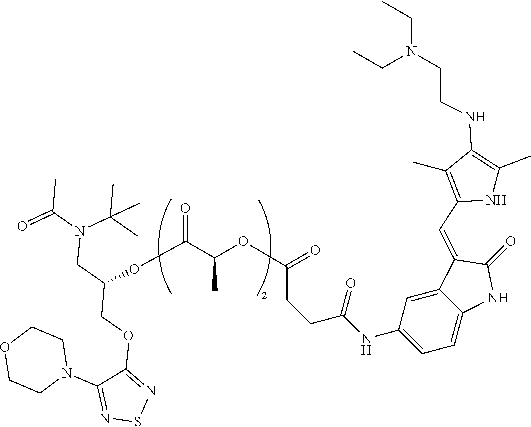 |
| 310 | 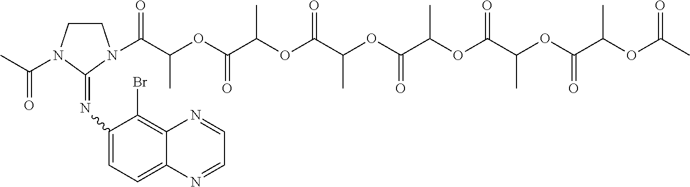 [SMILES: CC(=O)OC(C)C(=O)OC(C)C(=O)OC(C)C(=O)OC(C)C(=O)OC(C)C(=O)OC(C)C(=O)N1CCN(C(C)=O)C1=Nc1ccc2nccnc2c1Br] |
| 311 | 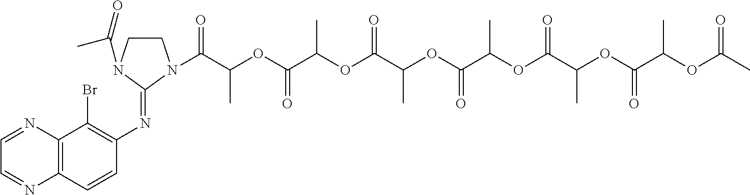 [SMILES: CC(=O)OC(C)C(=O)OC(C)C(=O)OC(C)C(=O)OC(C)C(=O)OC(C)C(=O)OC(C)C(=O)N1CCN(C(C)=O)/C1=N\c1ccc2nccnc2c1Br] |
| 312 | 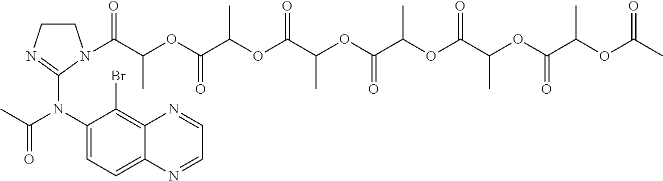 [SMILES: CC(=O)OC(C)C(=O)OC(C)C(=O)OC(C)C(=O)OC(C)C(=O)OC(C)C(=O)OC(C)C(=O)N1CCN=C1N(C(C)=O)c1ccc2nccnc2c1Br] |
| 313 | 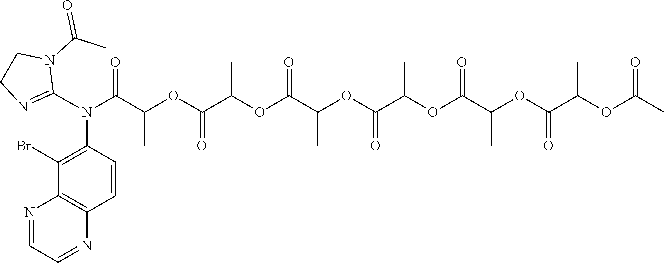 [SMILES: CC(=O)OC(C)C(=O)OC(C)C(=O)OC(C)C(=O)OC(C)C(=O)OC(C)C(=O)OC(C)C(=O)N(C1=NCCN1C(C)=O)c1ccc2nccnc2c1Br] |
| 314 | 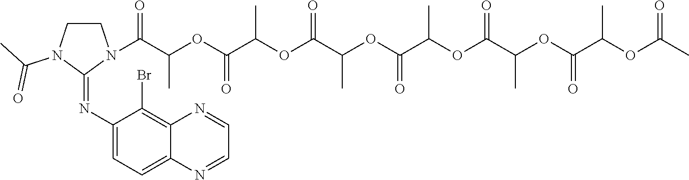 [SMILES: CC(=O)OC(C)C(=O)OC(C)C(=O)OC(C)C(=O)OC(C)C(=O)OC(C)C(=O)OC(C)C(=O)N1CCN(C(C)=O)/C1=N/c1ccc2nccnc2c1Br] |
| 315 | 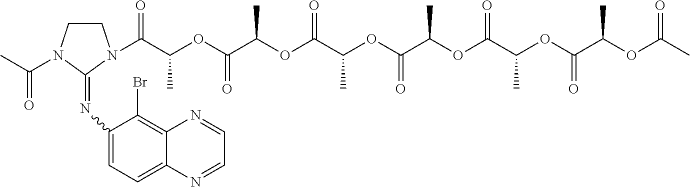 [SMILES: CC(=O)O[C@H](C)C(=O)O[C@H](C)C(=O)O[C@H](C)C(=O)O[C@H](C)C(=O)O[C@H](C)C(=O)O[C@H](C)C(=O)N1CCN(C(C)=O)C1=Nc1ccc2nccnc2c1Br] |
| 316 | 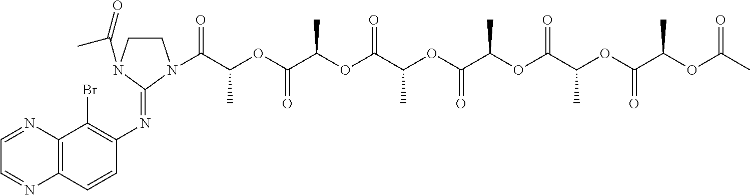 [SMILES: CC(=O)O[C@H](C)C(=O)O[C@H](C)C(=O)O[C@H](C)C(=O)O[C@H](C)C(=O)O[C@H](C)C(=O)O[C@H](C)C(=O)N1CCN(C(C)=O)/C1=N\c1ccc2nccnc2c1Br] |
| 317 | 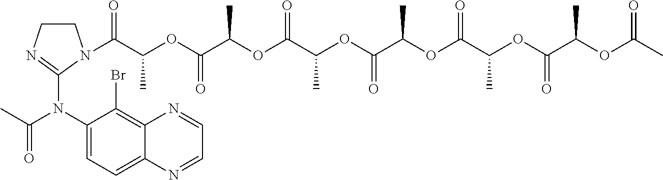 [SMILES: CC(=O)O[C@H](C)C(=O)O[C@H](C)C(=O)O[C@H](C)C(=O)O[C@H](C)C(=O)O[C@H](C)C(=O)O[C@H](C)C(=O)N1CCN=C1N(C(C)=O)c1ccc2nccnc2c1Br] |
| 318 | 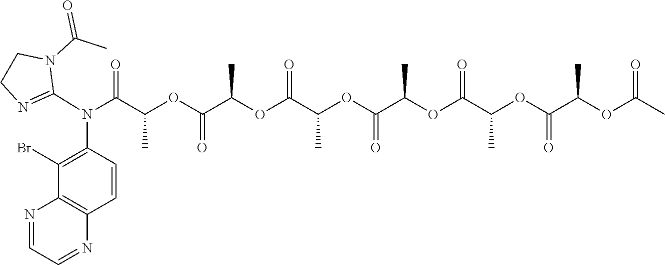 [SMILES: CC(=O)O[C@H](C)C(=O)O[C@H](C)C(=O)O[C@H](C)C(=O)O[C@H](C)C(=O)O[C@H](C)C(=O)O[C@H](C)C(=O)N(C1=NCCN1C(C)=O)c1ccc2nccnc2c1Br] |
| 319 | 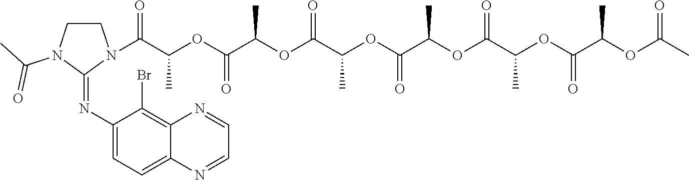 [SMILES: CC(=O)O[C@H](C)C(=O)O[C@H](C)C(=O)O[C@H](C)C(=O)O[C@H](C)C(=O)O[C@H](C)C(=O)O[C@H](C)C(=O)N1CCN(C(C)=O)/C1=N/c1ccc2nccnc2c1Br] |
| 320 | 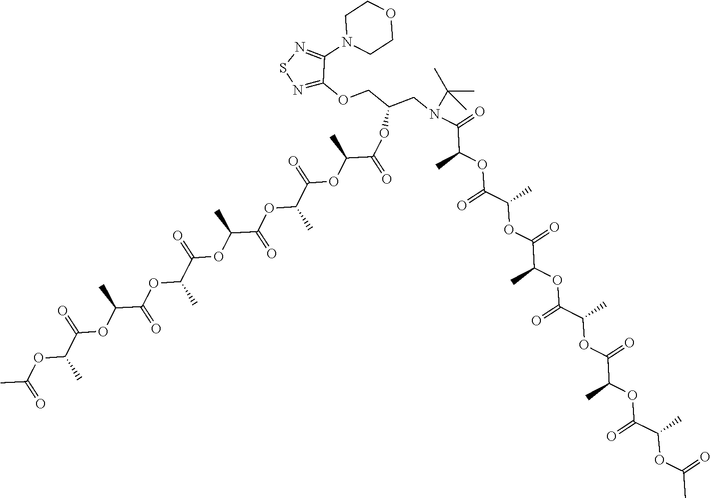 [SMILES: CC(=O)O[C@@H](C)C(=O)O[C@@H](C)C(=O)O[C@@H](C)C(=O)O[C@@H](C)C(=O)O[C@@H](C)C(=O)O[C@@H](C)C(=O)O[C@H](COc1nsnc1N1CCOCC1)CN(C(=O)[C@H](C)OC(=O)[C@H](C)OC(=O)[C@H](C)OC(=O)[C@H](C)OC(=O)[C@H](C)OC(=O)[C@H](C)OC(C)=O)C(C)(C)C] |

TABLE F
Compounds of the Present Invention

| Com- | |
|---|---|
| pd | |
| No. | Structure |
| 321 | 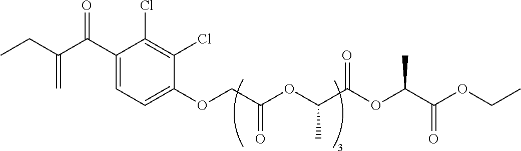 |
| 322 | 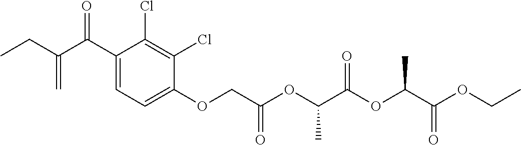 [SMILES: C=C(CC)C(=O)c1ccc(OCC(=O)O[C@@H](C)C(=O)O[C@@H](C)C(=O)OCC)c(Cl)c1Cl] |
| 323 | 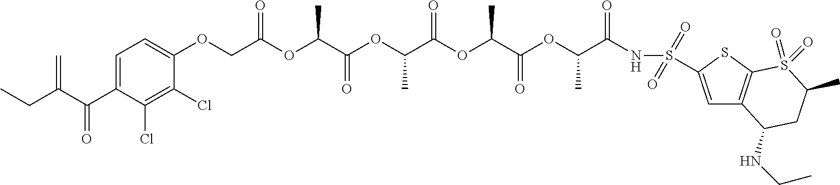 [SMILES: C=C(CC)C(=O)c1ccc(OCC(=O)O[C@@H](C)C(=O)O[C@@H](C)C(=O)O[C@@H](C)C(=O)O[C@@H](C)C(=O)NS(=O)(=O)c2cc3c(s2)S(=O)(=O)[C@@H](C)C[C@@H]3NCC)c(Cl)c1Cl] |
| 324 | 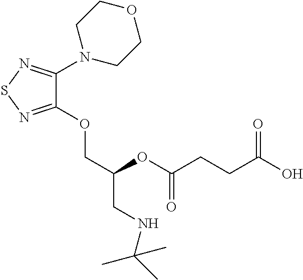 [SMILES: CC(C)(C)NC[C@@H](COc1nsnc1N1CCOCC1)OC(=O)CCC(=O)O] |
| 325 | 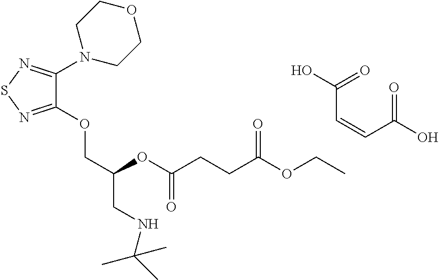 |
| 326 | 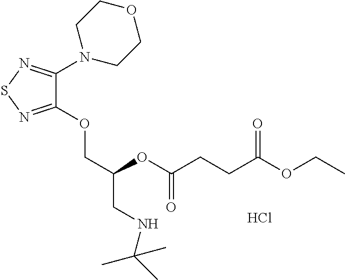 [SMILES: CCOC(=O)CCC(=O)O[C@@H](CNC(C)(C)C)COc1nsnc1N1CCOCC1 ; Cl] |
| 327 | 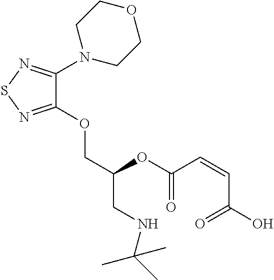 [SMILES: CC(C)(C)NC[C@@H](COc1nsnc1N1CCOCC1)OC(=O)/C=C\C(=O)O] |
| 328 | 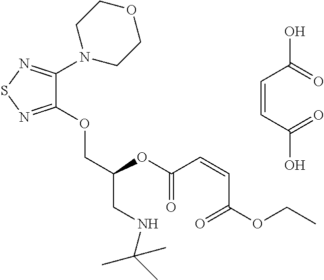 |
| 329 | 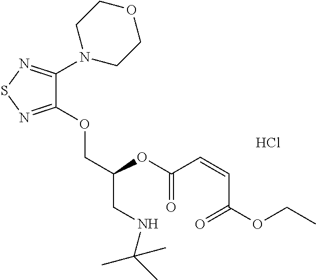 [SMILES: CCOC(=O)/C=C\C(=O)O[C@@H](CNC(C)(C)C)COc1nsnc1N1CCOCC1 ; Cl] |
| 330 | 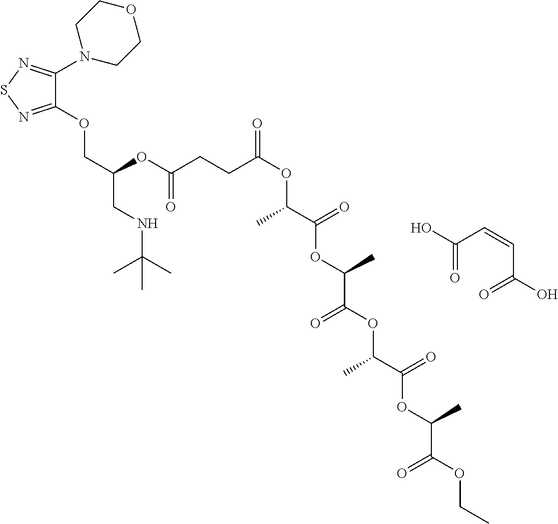 [SMILES: CCOC(=O)[C@H](C)OC(=O)[C@H](C)OC(=O)[C@H](C)OC(=O)[C@H](C)OC(=O)CCC(=O)O[C@@H](CNC(C)(C)C)COc1nsnc1N1CCOCC1 ; O=C(O)/C=C\C(=O)O] |
| 331 | 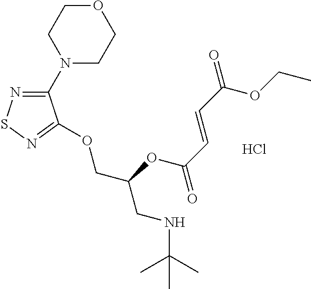 [SMILES: CCOC(=O)/C=C/C(=O)O[C@@H](CNC(C)(C)C)COc1nsnc1N1CCOCC1 ; Cl] |
| 332 | 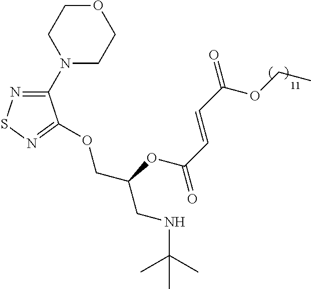 |
| 333 | 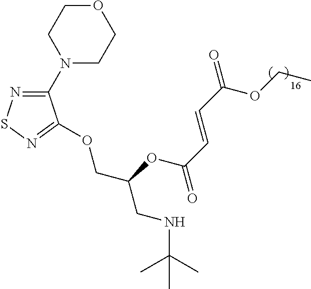 |
| 334 | 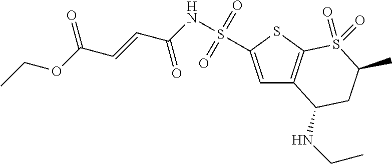 [SMILES: CCN[C@H]1C[C@H](C)S(=O)(=O)c2sc(S(=O)(=O)NC(=O)/C=C/C(=O)OCC)cc21] |
| 335 | 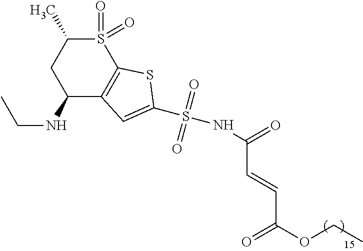 |
| 336 | 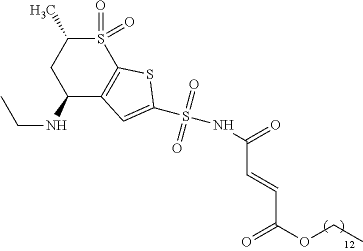 |
| 337 | 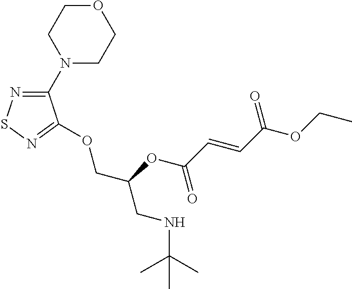 [SMILES: CCOC(=O)/C=C/C(=O)O[C@@H](CNC(C)(C)C)COc1nsnc1N1CCOCC1] |
| 338 | 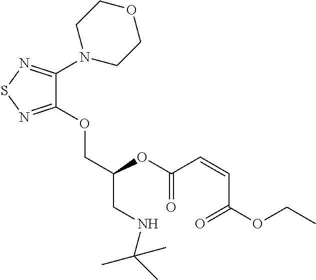 [SMILES: CCOC(=O)/C=C\C(=O)O[C@@H](CNC(C)(C)C)COc1nsnc1N1CCOCC1] |
| 339 | 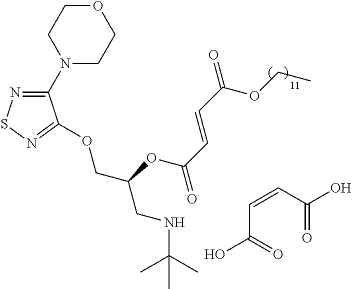 |
| 340 | 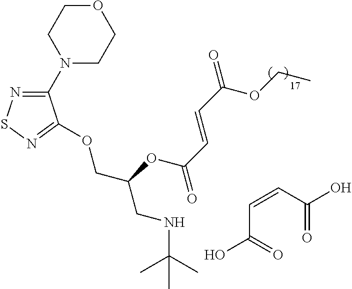 |
| 341 | 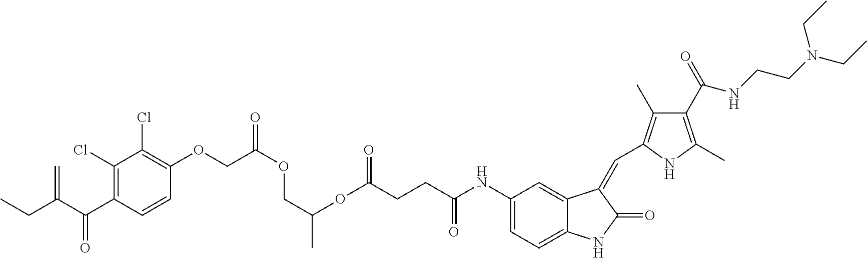 [SMILES: C=C(CC)C(=O)c1ccc(OCC(=O)OCC(C)OC(=O)CCC(=O)Nc2ccc3c(c2)/C(=C/c2[nH]c(C)c(C(=O)NCCN(CC)CC)c2C)C(=O)N3)c(Cl)c1Cl] |
| 342 | 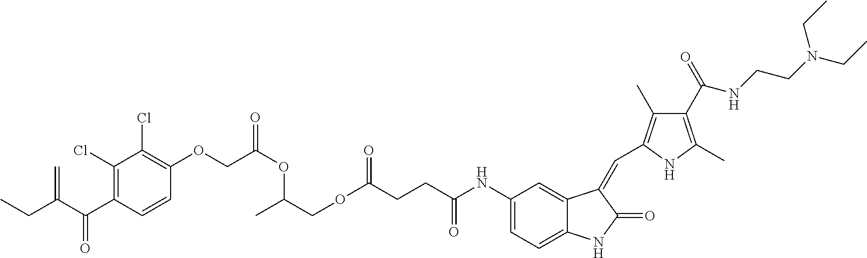 [SMILES: C=C(CC)C(=O)c1ccc(OCC(=O)OC(C)COC(=O)CCC(=O)Nc2ccc3c(c2)/C(=C/c2[nH]c(C)c(C(=O)NCCN(CC)CC)c2C)C(=O)N3)c(Cl)c1Cl] |
| 343 | 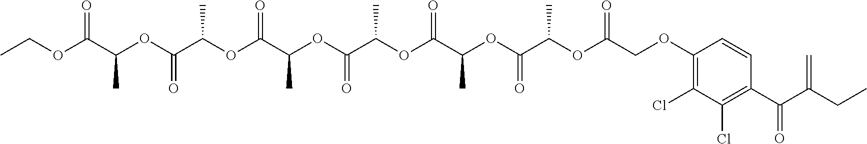 [SMILES: C=C(CC)C(=O)c1ccc(OCC(=O)O[C@@H](C)C(=O)O[C@@H](C)C(=O)O[C@@H](C)C(=O)O[C@@H](C)C(=O)O[C@@H](C)C(=O)O[C@@H](C)C(=O)OCC)c(Cl)c1Cl] |
| 344 | 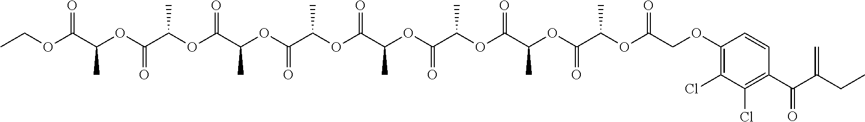 [SMILES: C=C(CC)C(=O)c1ccc(OCC(=O)O[C@@H](C)C(=O)O[C@@H](C)C(=O)O[C@@H](C)C(=O)O[C@@H](C)C(=O)O[C@@H](C)C(=O)O[C@@H](C)C(=O)O[C@@H](C)C(=O)O[C@@H](C)C(=O)OCC)c(Cl)c1Cl] |
| 345 | 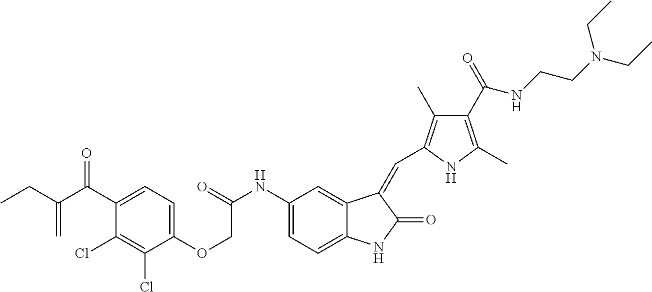 [SMILES: C=C(CC)C(=O)c1ccc(OCC(=O)Nc2ccc3c(c2)/C(=C/c2[nH]c(C)c(C(=O)NCCN(CC)CC)c2C)C(=O)N3)c(Cl)c1Cl] |
| 346 | 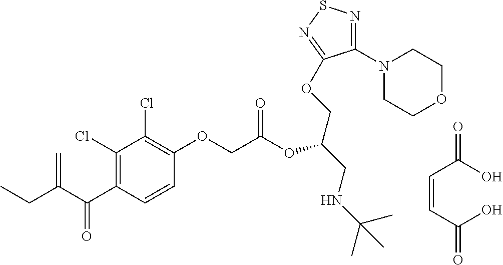 |
| 347 | 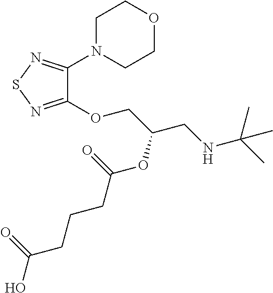 [SMILES: CC(C)(C)NC[C@@H](COc1nsnc1N1CCOCC1)OC(=O)CCCC(=O)O] |

TABLE G
Compounds of the Present Invention

| | |
|---|---|
| 348 | 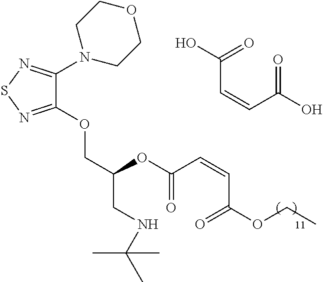 |
| 349 | 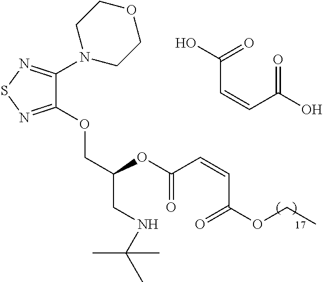 |
| 350 | 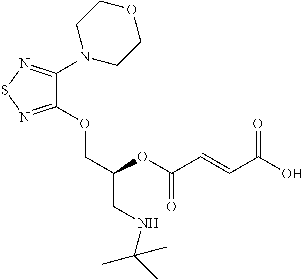 [SMILES: CC(C)(C)NC[C@@H](COc1nsnc1N1CCOCC1)OC(=O)/C=C/C(=O)O] |
| 351 | 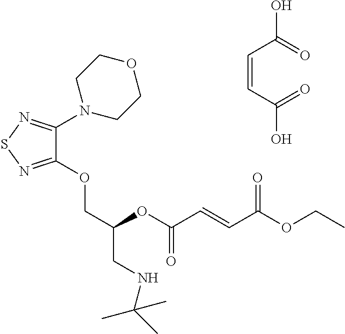 |
| 352 | 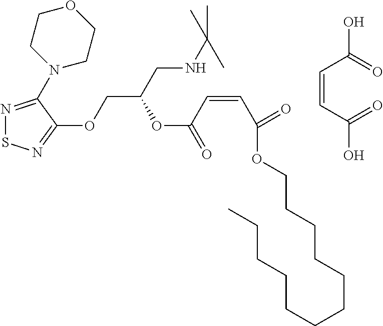 |
| 353 | 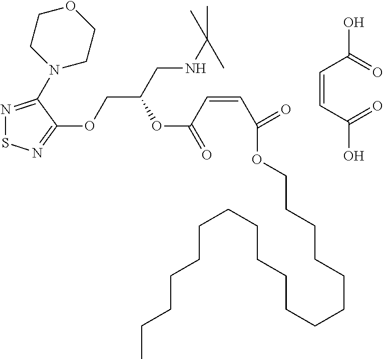 |
| 354 | 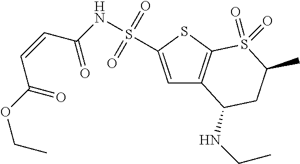 [SMILES: CCN[C@H]1C[C@H](C)S(=O)(=O)c2sc(S(=O)(=O)NC(=O)/C=C\C(=O)OCC)cc21] |
| 355 | 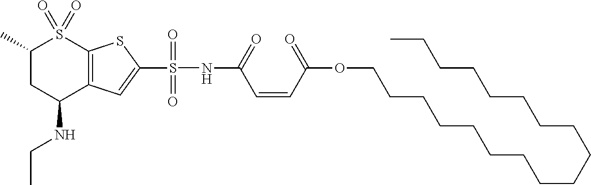 [SMILES: CCCCCCCCCCCCCCCCCCOC(=O)/C=C\C(=O)NS(=O)(=O)c1cc2c(s1)S(=O)(=O)[C@@H](C)C[C@@H]2NCC] |
| 356 | 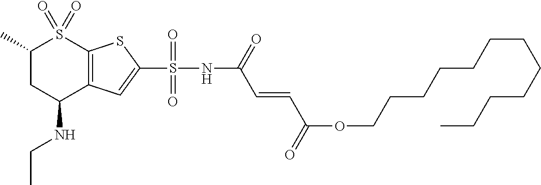 [SMILES: CCCCCCCCCCCCOC(=O)/C=C/C(=O)NS(=O)(=O)c1cc2c(s1)S(=O)(=O)[C@@H](C)C[C@@H]2NCC] |
| 357 | 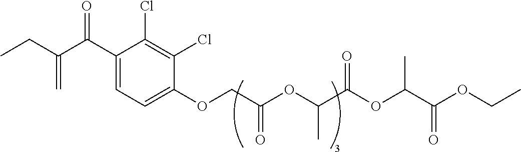 |
| 358 | 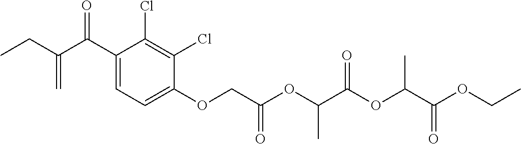 [SMILES: C=C(CC)C(=O)c1ccc(OCC(=O)OC(C)C(=O)OC(C)C(=O)OCC)c(Cl)c1Cl] |
| 359 | 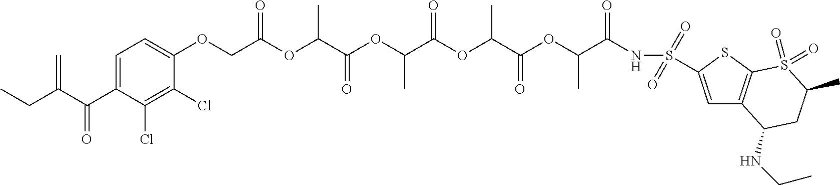 [SMILES: C=C(CC)C(=O)c1ccc(OCC(=O)OC(C)C(=O)OC(C)C(=O)OC(C)C(=O)OC(C)C(=O)NS(=O)(=O)c2cc3c(s2)S(=O)(=O)[C@@H](C)C[C@@H]3NCC)c(Cl)c1Cl] |
| 360 | 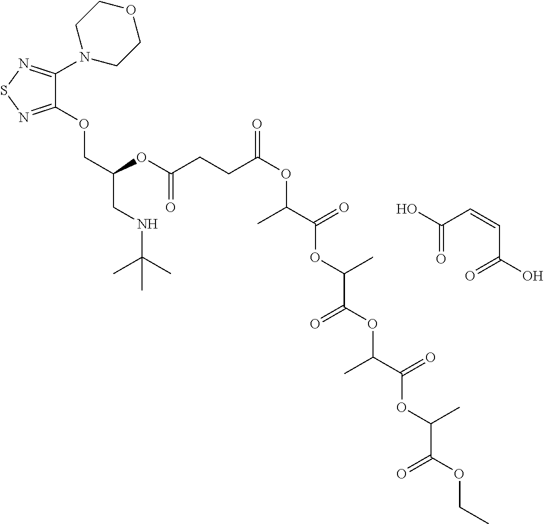 [SMILES: CCOC(=O)C(C)OC(=O)C(C)OC(=O)C(C)OC(=O)C(C)OC(=O)CCC(=O)O[C@@H](CNC(C)(C)C)COc1nsnc1N1CCOCC1 ; O=C(O)/C=C\C(=O)O] |
| 361 | 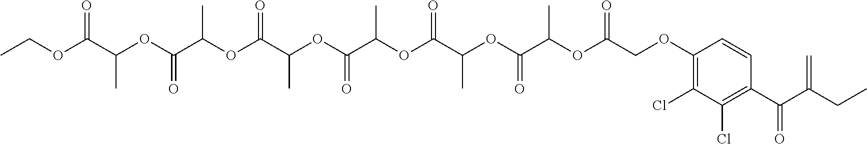 [SMILES: C=C(CC)C(=O)c1ccc(OCC(=O)OC(C)C(=O)OC(C)C(=O)OC(C)C(=O)OC(C)C(=O)OC(C)C(=O)OC(C)C(=O)OCC)c(Cl)c1Cl] |
| 362 | 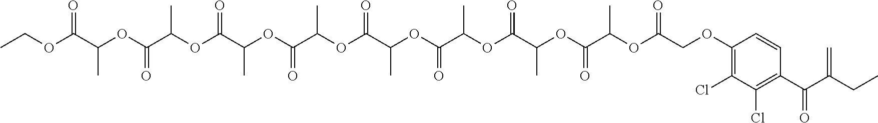 [SMILES: C=C(CC)C(=O)c1ccc(OCC(=O)OC(C)C(=O)OC(C)C(=O)OC(C)C(=O)OC(C)C(=O)OC(C)C(=O)OC(C)C(=O)OC(C)C(=O)OC(C)C(=O)OCC)c(Cl)c1Cl] |

TABLE H
Additional Compounds of the Present Invention

| | |
|---|---|
| 363 | 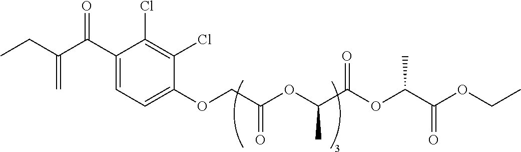 |
| 364 | 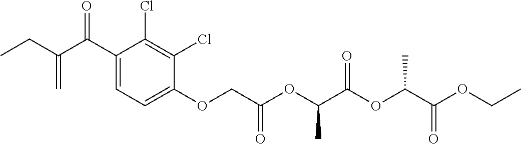 [SMILES: C=C(CC)C(=O)c1ccc(OCC(=O)O[C@H](C)C(=O)O[C@H](C)C(=O)OCC)c(Cl)c1Cl] |
| 365 | 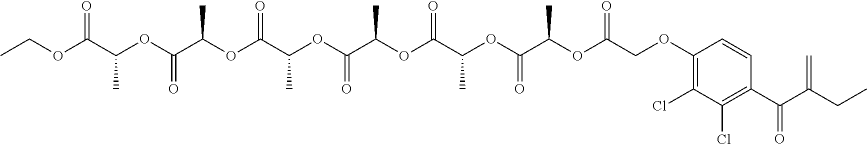 [SMILES: C=C(CC)C(=O)c1ccc(OCC(=O)O[C@H](C)C(=O)O[C@H](C)C(=O)O[C@H](C)C(=O)O[C@H](C)C(=O)O[C@H](C)C(=O)O[C@H](C)C(=O)OCC)c(Cl)c1Cl] |
| 366 | 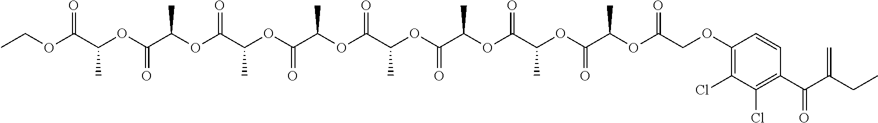 [SMILES: C=C(CC)C(=O)c1ccc(OCC(=O)O[C@H](C)C(=O)O[C@H](C)C(=O)O[C@H](C)C(=O)O[C@H](C)C(=O)O[C@H](C)C(=O)O[C@H](C)C(=O)O[C@H](C)C(=O)O[C@H](C)C(=O)OCC)c(Cl)c1Cl] |
| 367 | 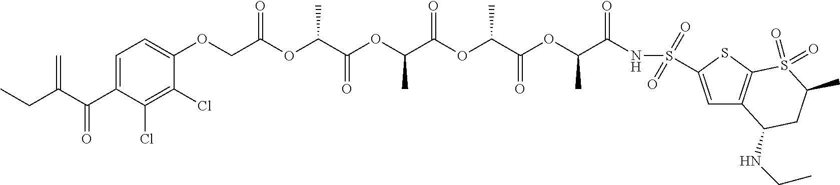 [SMILES: C=C(CC)C(=O)c1ccc(OCC(=O)O[C@H](C)C(=O)O[C@H](C)C(=O)O[C@H](C)C(=O)O[C@H](C)C(=O)NS(=O)(=O)c2cc3c(s2)S(=O)(=O)[C@@H](C)C[C@@H]3NCC)c(Cl)c1Cl] |

TABLE I
Additional Compounds of the Present Invention

| | |
|---|---|
| 368 | 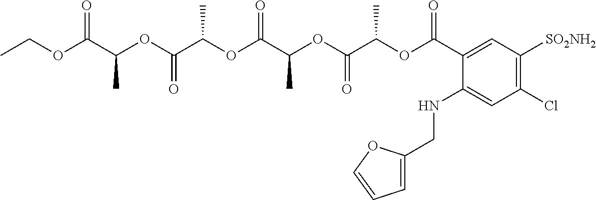 [SMILES: CCOC(=O)[C@H](C)OC(=O)[C@H](C)OC(=O)[C@H](C)OC(=O)[C@H](C)OC(=O)c1cc(S(N)(=O)=O)c(Cl)cc1NCc1ccco1] |
| 369 | 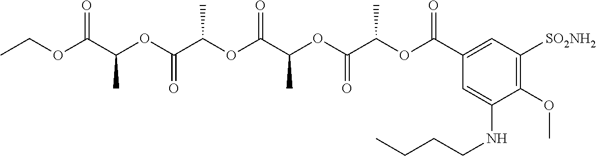 [SMILES: CCCCNc1cc(C(=O)O[C@@H](C)C(=O)O[C@@H](C)C(=O)O[C@@H](C)C(=O)O[C@@H](C)C(=O)OCC)cc(S(N)(=O)=O)c1OC] |
| 370 | 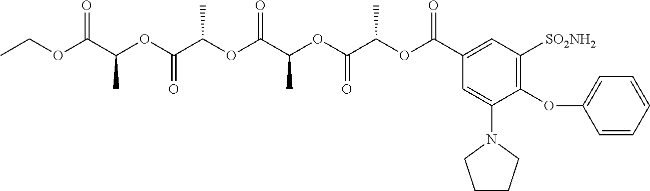 [SMILES: CCOC(=O)[C@H](C)OC(=O)[C@H](C)OC(=O)[C@H](C)OC(=O)[C@H](C)OC(=O)c1cc(N2CCCC2)c(Oc2ccccc2)c(S(N)(=O)=O)c1] |
| 371 | 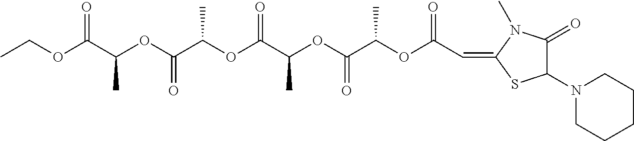 [SMILES: CCOC(=O)[C@H](C)OC(=O)[C@H](C)OC(=O)[C@H](C)OC(=O)[C@H](C)OC(=O)/C=C1/SC(N2CCCCC2)C(=O)N1C] |
| 372 | 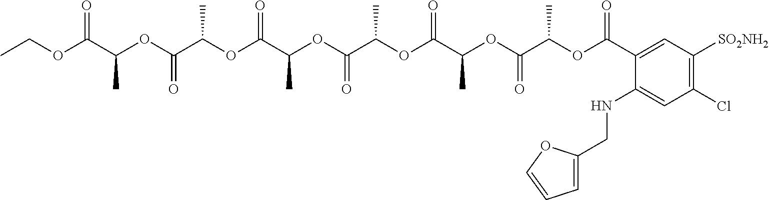 [SMILES: CCOC(=O)[C@H](C)OC(=O)[C@H](C)OC(=O)[C@H](C)OC(=O)[C@H](C)OC(=O)[C@H](C)OC(=O)[C@H](C)OC(=O)c1cc(S(N)(=O)=O)c(Cl)cc1NCc1ccco1] |
| 373 | 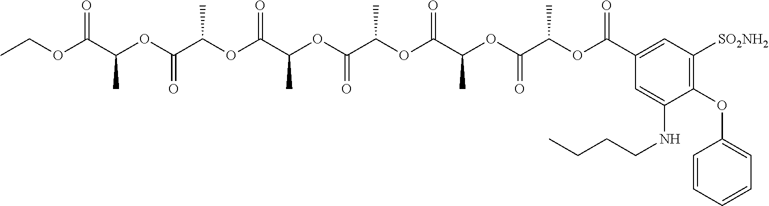 [SMILES: CCCCNc1cc(C(=O)O[C@@H](C)C(=O)O[C@@H](C)C(=O)O[C@@H](C)C(=O)O[C@@H](C)C(=O)O[C@@H](C)C(=O)O[C@@H](C)C(=O)OCC)cc(S(N)(=O)=O)c1Oc1ccccc1] |
| 374 | 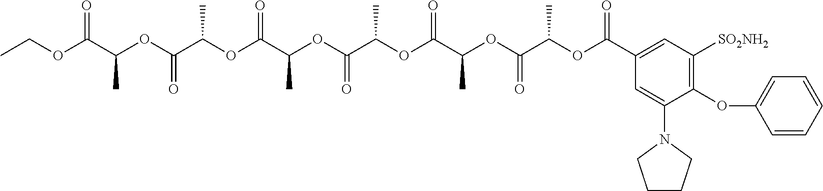 [SMILES: CCOC(=O)[C@H](C)OC(=O)[C@H](C)OC(=O)[C@H](C)OC(=O)[C@H](C)OC(=O)[C@H](C)OC(=O)[C@H](C)OC(=O)c1cc(N2CCCC2)c(Oc2ccccc2)c(S(N)(=O)=O)c1] |
| 375 | 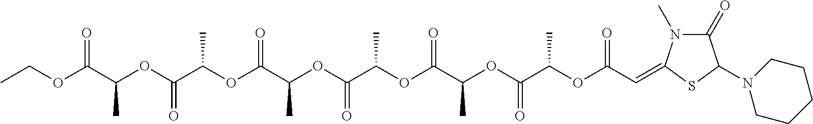 [SMILES: CCOC(=O)[C@H](C)OC(=O)[C@H](C)OC(=O)[C@H](C)OC(=O)[C@H](C)OC(=O)[C@H](C)OC(=O)[C@H](C)OC(=O)/C=C1/SC(N2CCCCC2)C(=O)N1C] |

TABLE J
Non-limiting Examples of Synthesized Compounds

| Compd | |
|---|---|
| No. | Structure |
| 376 | 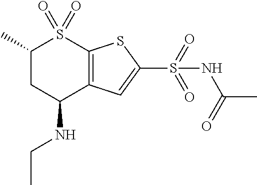 [SMILES: CCN[C@H]1C[C@H](C)S(=O)(=O)c2sc(S(=O)(=O)NC(C)=O)cc21] |
| 377 | 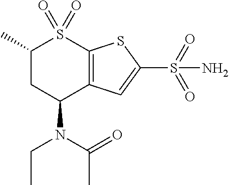 [SMILES: CCN(C(C)=O)[C@H]1C[C@H](C)S(=O)(=O)c2sc(S(N)(=O)=O)cc21] |
| 378 | 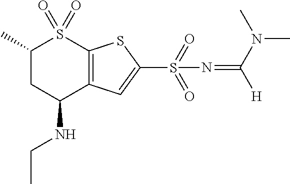 |
| 379 | 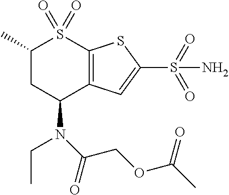 [SMILES: CCN(C(=O)COC(C)=O)[C@H]1C[C@H](C)S(=O)(=O)c2sc(S(N)(=O)=O)cc21] |
| 380 | 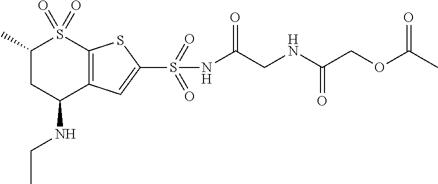 [SMILES: CCN[C@H]1C[C@H](C)S(=O)(=O)c2sc(S(=O)(=O)NC(=O)CNC(=O)COC(C)=O)cc21] |
| 381 | 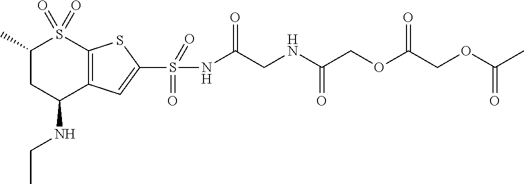 [SMILES: CCN[C@H]1C[C@H](C)S(=O)(=O)c2sc(S(=O)(=O)NC(=O)CNC(=O)COC(=O)COC(C)=O)cc21] |
| 382 | 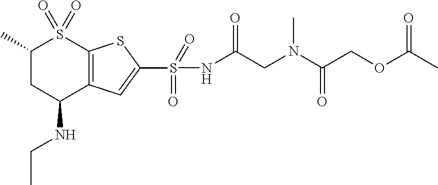 [SMILES: CCN[C@H]1C[C@H](C)S(=O)(=O)c2sc(S(=O)(=O)NC(=O)CN(C)C(=O)COC(C)=O)cc21] |
| 383 | 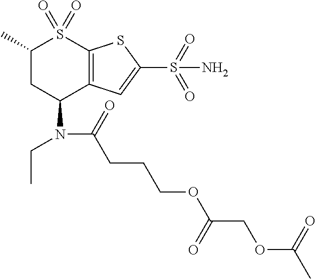 [SMILES: CCN(C(=O)CCCOC(=O)COC(C)=O)[C@H]1C[C@H](C)S(=O)(=O)c2sc(S(N)(=O)=O)cc21] |
| 384 | 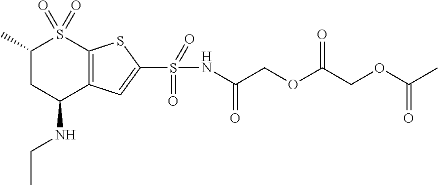 [SMILES: CCN[C@H]1C[C@H](C)S(=O)(=O)c2sc(S(=O)(=O)NC(=O)COC(=O)COC(C)=O)cc21] |
| 385 | 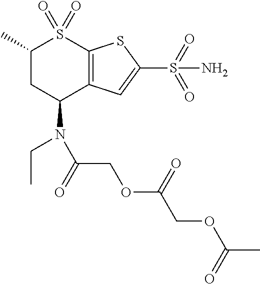 [SMILES: CCN(C(=O)COC(=O)COC(C)=O)[C@H]1C[C@H](C)S(=O)(=O)c2sc(S(N)(=O)=O)cc21] |
| 386 | 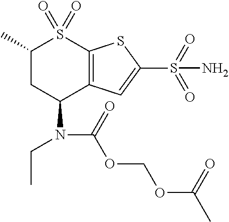 [SMILES: CCN(C(=O)OCOC(C)=O)[C@H]1C[C@H](C)S(=O)(=O)c2sc(S(N)(=O)=O)cc21] |
| 387 | 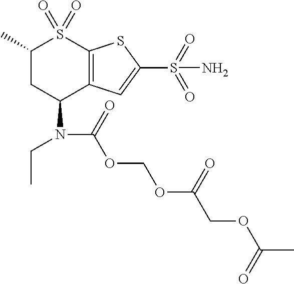 [SMILES: CCN(C(=O)OCOC(=O)COC(C)=O)[C@H]1C[C@H](C)S(=O)(=O)c2sc(S(N)(=O)=O)cc21] |
| 388 | 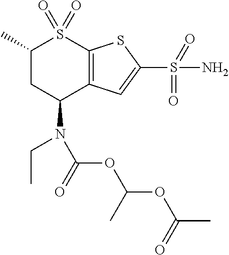 [SMILES: CCN(C(=O)OC(C)OC(C)=O)[C@H]1C[C@H](C)S(=O)(=O)c2sc(S(N)(=O)=O)cc21] |
| 389 | 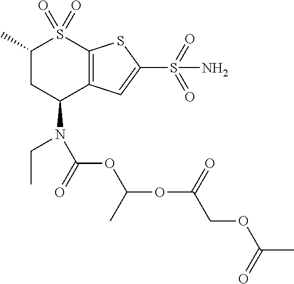 [SMILES: CCN(C(=O)OC(C)OC(=O)COC(C)=O)[C@H]1C[C@H](C)S(=O)(=O)c2sc(S(N)(=O)=O)cc21] |
| 390 | 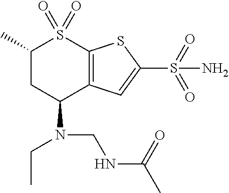 [SMILES: CCN(CNC(C)=O)[C@H]1C[C@H](C)S(=O)(=O)c2sc(S(N)(=O)=O)cc21] |
| 391 | 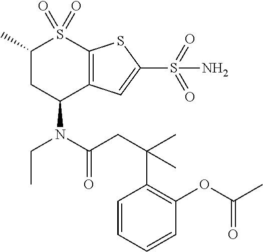 [SMILES: CCN(C(=O)CC(C)(C)c1ccccc1OC(C)=O)[C@H]1C[C@H](C)S(=O)(=O)c2sc(S(N)(=O)=O)cc21] |
| 392 | 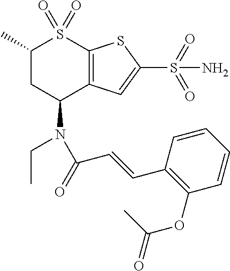 [SMILES: CCN(C(=O)/C=C/c1ccccc1OC(C)=O)[C@H]1C[C@H](C)S(=O)(=O)c2sc(S(N)(=O)=O)cc21] |
| 393 | 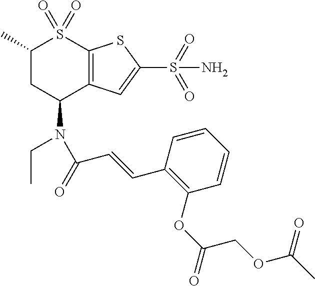 [SMILES: CCN(C(=O)/C=C/c1ccccc1OC(=O)COC(C)=O)[C@H]1C[C@H](C)S(=O)(=O)c2sc(S(N)(=O)=O)cc21] |
| 394 | 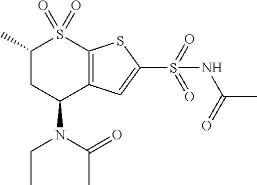 [SMILES: CCN(C(C)=O)[C@H]1C[C@H](C)S(=O)(=O)c2sc(S(=O)(=O)NC(C)=O)cc21] |
| 395 | 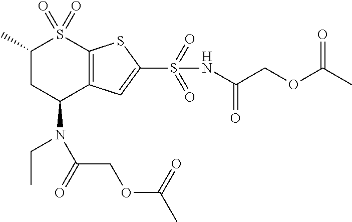 [SMILES: CCN(C(=O)COC(C)=O)[C@H]1C[C@H](C)S(=O)(=O)c2sc(S(=O)(=O)NC(=O)COC(C)=O)cc21] |
| 396 | 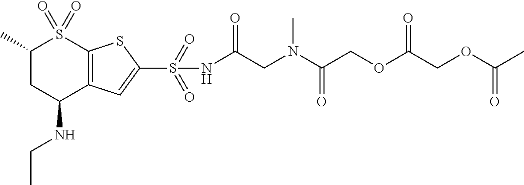 [SMILES: CCN[C@H]1C[C@H](C)S(=O)(=O)c2sc(S(=O)(=O)NC(=O)CN(C)C(=O)COC(=O)COC(C)=O)cc21] |
| 397 | 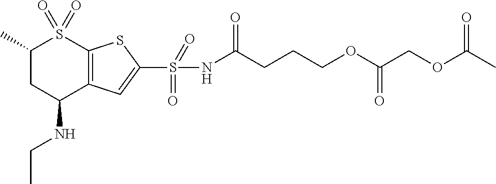 [SMILES: CCN[C@H]1C[C@H](C)S(=O)(=O)c2sc(S(=O)(=O)NC(=O)CCCOC(=O)COC(C)=O)cc21] |
| 398 | 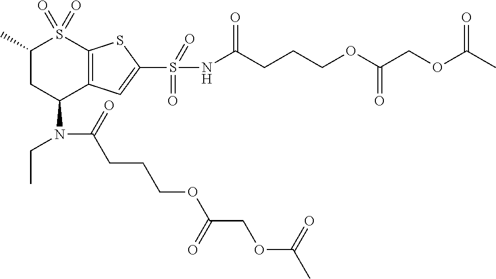 [SMILES: CCN(C(=O)CCCOC(=O)COC(C)=O)[C@H]1C[C@H](C)S(=O)(=O)c2sc(S(=O)(=O)NC(=O)CCCOC(=O)COC(C)=O)cc21] |
| 399 | 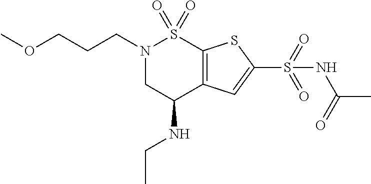 [SMILES: CCN[C@H]1CN(CCCOC)S(=O)(=O)c2sc(S(=O)(=O)NC(C)=O)cc21] |
| 400 | 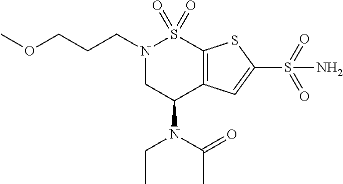 [SMILES: CCN(C(C)=O)[C@H]1CN(CCCOC)S(=O)(=O)c2sc(S(N)(=O)=O)cc21] |
| 401 | 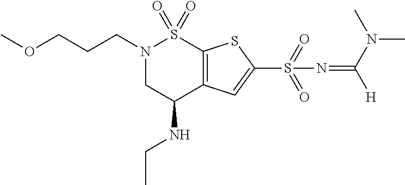 |
| 402 | 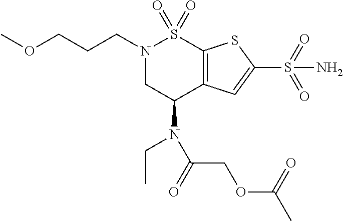 [SMILES: CCN(C(=O)COC(C)=O)[C@H]1CN(CCCOC)S(=O)(=O)c2sc(S(N)(=O)=O)cc21] |
| 403 | 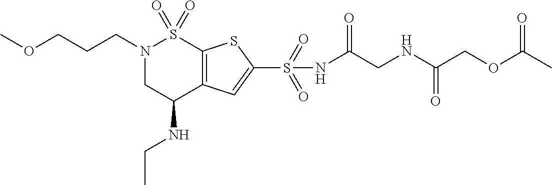 [SMILES: CCN[C@H]1CN(CCCOC)S(=O)(=O)c2sc(S(=O)(=O)NC(=O)CNC(=O)COC(C)=O)cc21] |
| 404 | 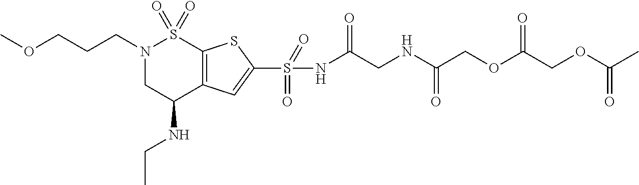 [SMILES: CCN[C@H]1CN(CCCOC)S(=O)(=O)c2sc(S(=O)(=O)NC(=O)CNC(=O)COC(=O)COC(C)=O)cc21] |
| 405 | 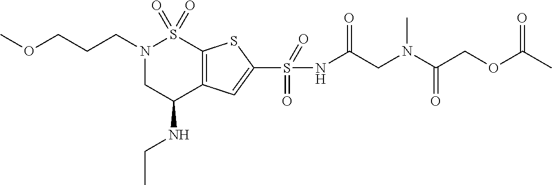 [SMILES: CCN[C@H]1CN(CCCOC)S(=O)(=O)c2sc(S(=O)(=O)NC(=O)CN(C)C(=O)COC(C)=O)cc21] |
| 406 | 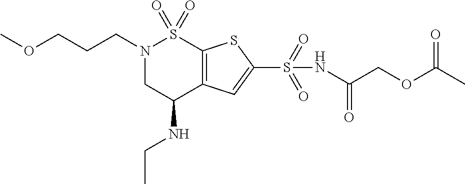 [SMILES: CCN[C@H]1CN(CCCOC)S(=O)(=O)c2sc(S(=O)(=O)NC(=O)COC(C)=O)cc21] |
| 407 | 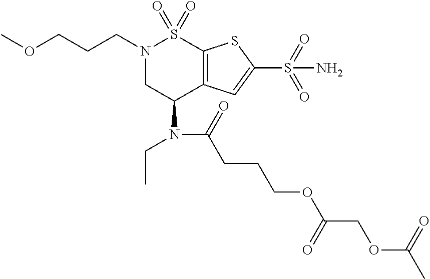 [SMILES: CCN(C(=O)CCCOC(=O)COC(C)=O)[C@H]1CN(CCCOC)S(=O)(=O)c2sc(S(N)(=O)=O)cc21] |
| 408 | 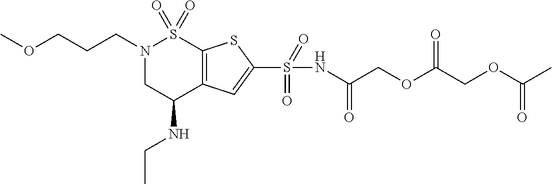 [SMILES: CCN[C@H]1CN(CCCOC)S(=O)(=O)c2sc(S(=O)(=O)NC(=O)COC(=O)COC(C)=O)cc21] |
| 409 | 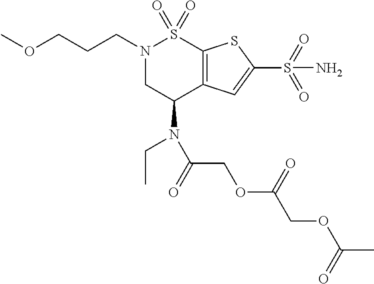 [SMILES: CCN(C(=O)COC(=O)COC(C)=O)[C@H]1CN(CCCOC)S(=O)(=O)c2sc(S(N)(=O)=O)cc21] |
| 410 | 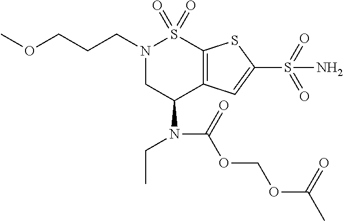 [SMILES: CCN(C(=O)OCOC(C)=O)[C@H]1CN(CCCOC)S(=O)(=O)c2sc(S(N)(=O)=O)cc21] |
| 411 | 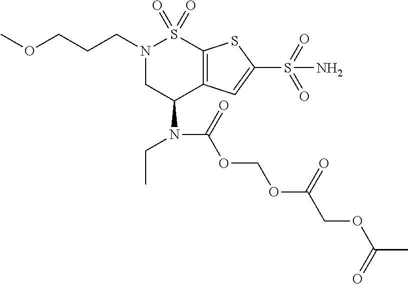 [SMILES: CCN(C(=O)OCOC(=O)COC(C)=O)[C@H]1CN(CCCOC)S(=O)(=O)c2sc(S(N)(=O)=O)cc21] |
| 412 | 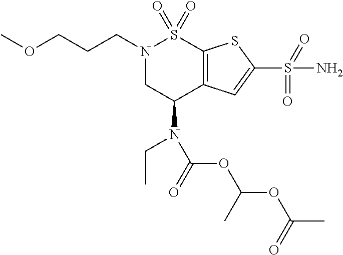 [SMILES: CCN(C(=O)OC(C)OC(C)=O)[C@H]1CN(CCCOC)S(=O)(=O)c2sc(S(N)(=O)=O)cc21] |
| 413 | 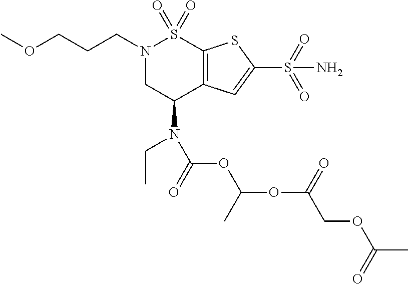 [SMILES: CCN(C(=O)OC(C)OC(=O)COC(C)=O)[C@H]1CN(CCCOC)S(=O)(=O)c2sc(S(N)(=O)=O)cc21] |
| 414 | 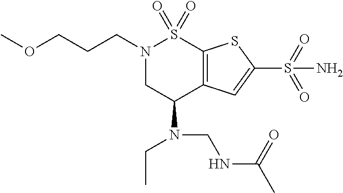 [SMILES: CCN(CNC(C)=O)[C@H]1CN(CCCOC)S(=O)(=O)c2sc(S(N)(=O)=O)cc21] |
| 415 | 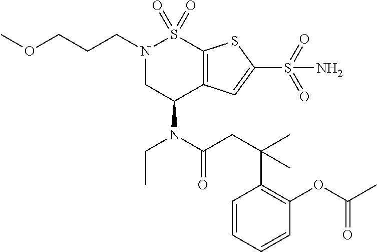 [SMILES: CCN(C(=O)CC(C)(C)c1ccccc1OC(C)=O)[C@H]1CN(CCCOC)S(=O)(=O)c2sc(S(N)(=O)=O)cc21] |
| 416 | 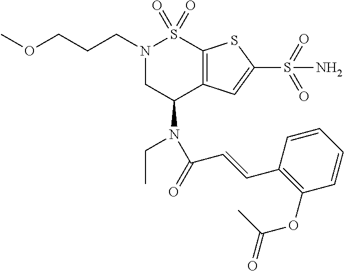 [SMILES: CCN(C(=O)/C=C/c1ccccc1OC(C)=O)[C@H]1CN(CCCOC)S(=O)(=O)c2sc(S(N)(=O)=O)cc21] |
| 417 | 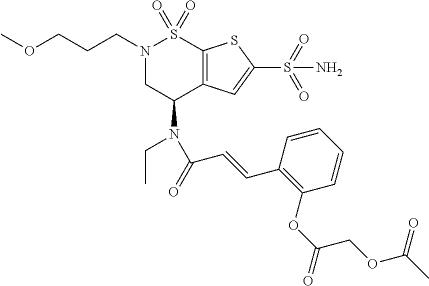 [SMILES: CCN(C(=O)/C=C/c1ccccc1OC(=O)COC(C)=O)[C@H]1CN(CCCOC)S(=O)(=O)c2sc(S(N)(=O)=O)cc21] |
| 418 | 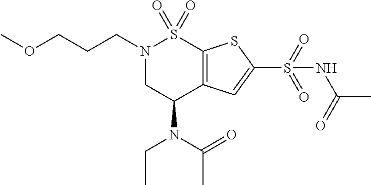 [SMILES: CCN(C(C)=O)[C@H]1CN(CCCOC)S(=O)(=O)c2sc(S(=O)(=O)NC(C)=O)cc21] |
| 419 | 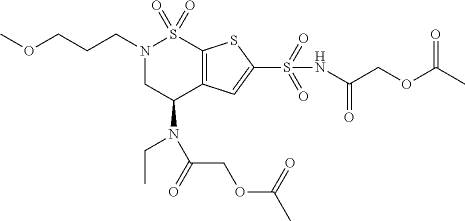 [SMILES: CCN(C(=O)COC(C)=O)[C@H]1CN(CCCOC)S(=O)(=O)c2sc(S(=O)(=O)NC(=O)COC(C)=O)cc21] |
| 420 | 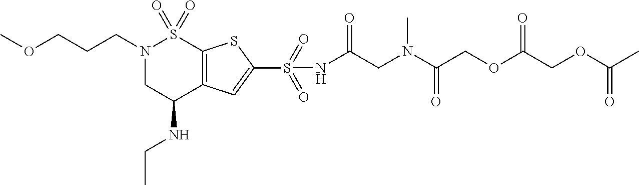 [SMILES: CCN[C@H]1CN(CCCOC)S(=O)(=O)c2sc(S(=O)(=O)NC(=O)CN(C)C(=O)COC(=O)COC(C)=O)cc21] |
| 421 | 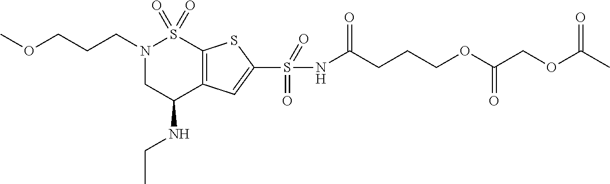 [SMILES: CCN[C@H]1CN(CCCOC)S(=O)(=O)c2sc(S(=O)(=O)NC(=O)CCCOC(=O)COC(C)=O)cc21] |
| 422 | 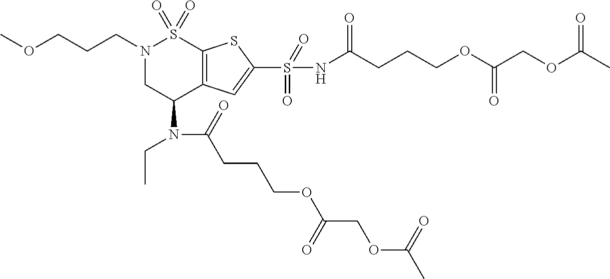 [SMILES: CCN(C(=O)CCCOC(=O)COC(C)=O)[C@H]1CN(CCCOC)S(=O)(=O)c2sc(S(=O)(=O)NC(=O)CCCOC(=O)COC(C)=O)cc21] |
| 423 | 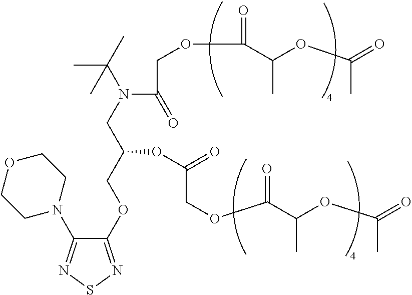 |

TABLE K
Additional Non-limiting Examples of Synthesized Compounds

| | |
|---|---|
| 424 | 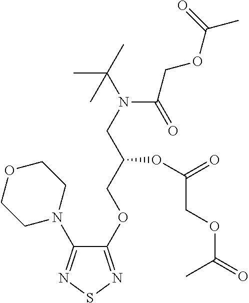 [SMILES: CC(=O)OCC(=O)O[C@H](COc1nsnc1N1CCOCC1)CN(C(=O)COC(C)=O)C(C)(C)C] |
| 425 | 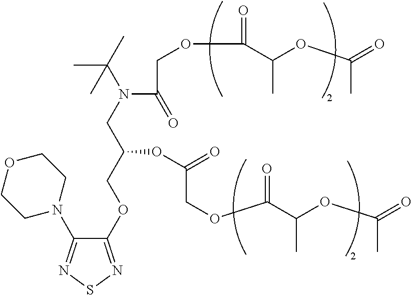 |
| 426 | 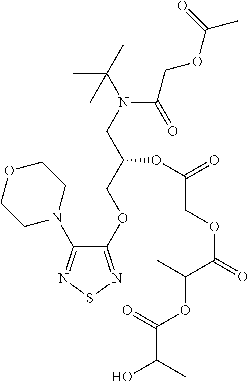 [SMILES: CC(=O)OCC(=O)N(C[C@@H](COc1nsnc1N1CCOCC1)OC(=O)COC(=O)C(C)OC(=O)C(C)O)C(C)(C)C] |
| 427 | 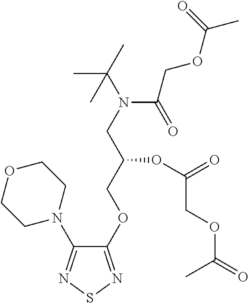 [SMILES: CC(=O)OCC(=O)O[C@H](COc1nsnc1N1CCOCC1)CN(C(=O)COC(C)=O)C(C)(C)C] |
| 428 | 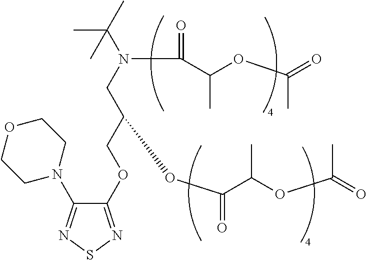 |
| 429 | 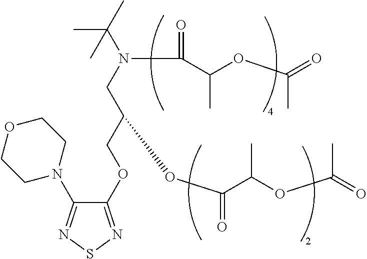 |
| 430 | 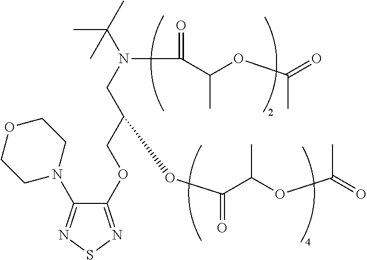 |

XV. Pharmaceutically Acceptable Carriers

Any suitable pharmaceutically acceptable carrier optionally comprising additive that improves particle aggregation, for example, ophthalmically acceptable viscous carrier, may be employed in accordance with the invention. The carrier is present in an amount effective in providing the desired viscosity to the drug delivery system. Advantageously, the viscous carrier is present in an amount in a range of from about 0.5 wt percent to about 95 wt percent of the drug delivery particles. The specific amount of the viscous carrier used depends upon a number of factors including, for example and without limitation, the specific viscous carrier used, the molecular weight of the viscous carrier used, the viscosity desired for the present drug delivery system being produced and/or used and like factors. Examples of useful viscous carriers include, but are not limited to, hyaluronic acid, sodium hyaluronate, carbomers, polyacrylic acid, cellulosic derivatives, polycarbophil, polyvinylpyrrolidone, gelatin, dextrin, polysaccharides, polyacrylamide, polyvinyl alcohol (which can be partially hydrolyzed polyvinyl acetate), polyvinyl acetate, derivatives thereof and mixtures thereof.

The carrier can also be an aqueous carrier. Example of aqueous carriers include, but are not limited to, an aqueous solution or suspension, such as saline, plasma, bone marrow aspirate, buffers, such as Hank's Buffered Salt Solution (HBSS), HEPES (4-(2-hydroxyethyl)-1-piperazineethanesulfonic acid), Ringers buffer, ProVisc®, diluted ProVisc®, ProVisc® diluted with PBS, Krebs buffer, Dulbecco's PBS, normal PBS; sodium hyaluronate solution (HA, 5 mg/mL in PBS), simulated body fluids, plasma platelet concentrate and tissue culture medium or an aqueous solution or suspension comprising an organic solvent.

In one embodiment, the carrier is PBS.

In one embodiment, the carrier is HA, 5 mg/mL in PBS.

In one embodiment, the carrier is ProVisc® diluted with water.

In one embodiment, the carrier is ProVisc® dilution in PBS.

In one embodiment, the carrier is ProVisc® 5-fold diluted with water.

In one embodiment, the carrier is ProVisc® 5-fold dilution in PBS.

In one embodiment, the carrier is ProVisc® 10-fold diluted with water.

In one embodiment, the carrier is ProVisc® 10-fold dilution in PBS.

In one embodiment, the carrier is ProVisc® 20-fold dilution with water.

In one embodiment, the carrier is ProVisc® 20-fold dilution in PBS.

In one embodiment, the carrier is HA, 1.25 mg/mL in an isotonic buffer solution with neutral pH.

In one embodiment, the carrier is HA, 0.625 mg/mL in an isotonic buffer solution with neutral pH.

In one embodiment, the carrier is HA, 0.1-5.0 mg/mL in PBS.

In one embodiment, the carrier is HA, 0.5-4.5 mg/mL in PBS.

In one embodiment, the carrier is HA, 1.0-4.0 mg/mL in PBS.

In one embodiment, the carrier is HA, 1.5-3.5 mg/mL in PBS.

In one embodiment, the carrier is HA, 2.0-3.0 mg/mL in PBS.

In one embodiment, the carrier is HA, 2.5-3.0 mg/mL in PBS.

The carrier may, optionally, contain one or more suspending agent. The suspending agent may be selected from carboxy methylcellulose (CMC), mannitol, polysorbate, poly propylene glycol, poly ethylene glycol, gelatin, albumin, alginate, hydroxyl propyl methyl cellulose (HPMC), hydroxyl ethyl methyl cellulose (HEMC), bentonite, tragacanth, dextrin, sesame oil, almond oil, sucrose, acacia gum and xanthan gum and combinations thereof.

In one embodiment, one or more additional excipients or delivery enhancing agents may also be included e.g., surfactants and/or hydrogels, in order to further influence release rate.

### XVI. Sustained Release of Pharmaceutically Active Compound

The rate of release of the pharmaceutically active compound can be related to the concentration of pharmaceutically active compound dissolved in the surface treated microparticle. In some embodiments, the polymeric composition of the surface treated microparticle includes non-therapeutic agents that are selected to provide a desired solubility of the pharmaceutically active compound. The selection of the polymeric composition can be made to provide the desired solubility of the pharmaceutically active compound in the surface treated microparticle, for example, a hydrogel may promote solubility of a hydrophilic material. In some embodiments, functional groups can be added to the polymer to increase the desired solubility of the pharmaceutically active compound in the surface treated microparticle. In some embodiments, additives may be used to control the release kinetics of the pharmaceutically active compound, for example, the additives may be used to control the concentration of the pharmaceutically active compound by increasing or decreasing the solubility of the pharmaceutically active compound in the polymer so as to control the release kinetics of the pharmaceutically active compound. The solubility may be controlled by including appropriate molecules and/or substances that increase and/or decrease the solubility of the dissolved form of the pharmaceutically active compound in the surface treated microparticle. The solubility of the pharmaceutically active compound may be related to the hydrophobic and/or hydrophilic properties of the surface treated microparticle and the pharmaceutically active compound. Oils and hydrophobic molecules can be added to the polymer(s) to increase the solubility of a pharmaceutically active compound in the surface treated microparticle.

Instead of, or in addition to, controlling the rate of migration based on the concentration of the pharmaceutically active compound dissolved in the surface treated microparticle, the surface area of the polymeric composition can be controlled to attain the desired rate of drug migration out of the surface treated microparticle comprising a pharmaceutically active compound. For example, a larger exposed surface area will increase the rate of migration of the pharmaceutically active compound to the surface, and a smaller exposed surface area will decrease the rate of migration of the pharmaceutically active compound to the surface. The exposed surface area can be increased in any number of ways, for example, by castellation of the exposed surface, a porous surface having exposed channels connected with the tear or tear film, indentation of the exposed surface, or protrusion of the exposed surface. The exposed surface can be made porous by the addition of salts that dissolve and leave a porous cavity once the salt dissolves. In the present invention, these trends can be used to decrease the release rate of the active material from the polymeric composition by avoiding these paths to quicker release. For example, the surface area can be minimized, or channels can be avoided.

Where more than one type of polymer is used, each surface treated microparticle may have a different solidifying or setting property. For example, the surface treated microparticles may be made from similar polymers but may have different gelling pHs or different melting temperatures or glass transition points.

In order for the surface treated microparticles to form a consolidated aggregate, the temperature around the particles, for example in the human or non-human animal where the composition is administered, is approximately equal to, or greater than, the glass transition temperature (T_{g}) of the polymer particles. At such temperatures the polymer particles will cross-link to one or more other polymer particles to form a consolidated aggregate. By cross-link it is meant that adjacent polymer particles become joined together. For example, the particles may cross-link due to entanglement of the polymer chains at the surface of one particle with polymer chains at the surface of another particle. There may be adhesion, cohesion or fusion between adjacent particles.

Typically, the injectable surface treated microparticles which are formed of a polymer or a polymer blend have a glass transition temperature (T_{g}) either close to or just above body temperature (such as from about 30° C. to 45° C., e.g., from about 35° C. to 40° C., for example, from about 37° C. to 40° C.). Accordingly, at room temperature the surface treated microparticles are below their T_{g} and behave as discrete particles, but in the body the surface treated microparticles soften and interact/stick to themselves. Typically, agglomeration begins within 20 seconds to about 15 minutes of the raise in temperature from room to body temperature.

The surface treated microparticles may be formed from a polymer which has a T_{g} from about 35° C. to 40° C., for example from about 37° C. to 40° C., wherein the polymer is a poly(α-hydroxyacid) (such as PLA, PGA, PLGA, or PDLLA or a combination thereof), or a blend thereof with PLGA-PEG. Typically, these particles will agglomerate at body temperature. The injectable surface treated microparticles may comprise only poly(α-hydroxyacid) particles or other particle types may be included. The microparticles can be formed from a blend of poly(D,L-lactide-co-glycolide)(PLGA), PLGA-PEG and PVA which has a T_{g} at or above body temperature. In one embodiment, at body temperature the surface treated microparticles will interact to form a consolidated aggregate. The injectable microparticle may comprise only PLGA/PLGA-PEG/PVA surface treated microparticles or other particle types may be included.

The composition may comprise a mixture of temperature sensitive surface treated microparticles and non-temperature sensitive surface treated microparticles. Non-temperature sensitive surface treated microparticles are particles with a glass transition temperature which is above the temperature at which the composition is intended to be used. Typically, in a composition comprising a mixture of temperature sensitive surface treated microparticles and non-temperature sensitive particles the ratio of temperature sensitive to non-temperature sensitive surface treated microparticles is about 3:1, or lower, for example, 4:3. The temperature sensitive surface treated microparticles are advantageously capable of crosslinking to each other when the temperature of the composition is raised to or above the glass transition of these microparticles. By controlling the ratio of temperature sensitive surface treated microparticles to non-temperature sensitive surface treated microparticles it may be possible to manipulate the porosity of the resulting consolidated aggregate. The surface treated microparticles may be solid, that is with a solid outer surface, or they may be porous. The particles may be irregular or substantially spherical in shape.

The surface treated microparticles can have a size in their longest dimension, or their diameter if they are substantially spherical, of less than about 100 μm and more than about 1 μm. The surface treated microparticles can have a size in their longest dimension, or their diameter, of less than about 100 μm. The surface treated microparticles can have a size in their longest dimension, or their diameter, of between about 1 μm and about 40 μm, more typically, between about 20 μm and about 40 μm. Polymer particles of the desired size will pass through a sieve or filter with a pore size of about 40 μm.

Formation of the consolidated aggregate from the composition, once administered to a human or non-human animal, typically takes from about 20 seconds to about 24 hours, for example, between about 1 minute and about 5 hours, between about 1 minute and about 1 hour, less than about 30 minutes, less than about 20 minutes. Typically, the solidification occurs in between about 1 minute and about 20 minutes from administration.

Typically, the composition comprises from about 20 percent to about 80 percent injectable surface treated microparticle material and from about 20 percent to about 80 percent carrier; from about 30 percent to about 70 percent injectable surface treated microparticle material and from about 30 percent to about 70 percent carrier; e.g., the composition may comprise from about 40 percent to about 60 percent injectable surface treated microparticle material and from about 40 percent to about 60 percent carrier; the composition may comprise about 50 percent injectable surface treated microparticle material and about 50 percent carrier. The aforementioned percentages all refer to percentage by weight.

The surface treated microparticles are loaded, for example, in the surface treated microparticle or as a coating on the surface treated microparticle, with a pharmaceutically active compound.

The system of the invention can allow for the pharmaceutically active compound release to be sustained for some time, for example, release can be sustained for at least about 2 hours, at least about 4 hours, at least about 6 hours, at least about 10 hours, at least about 12 hours, at least about 24 hours, at least 48 hours, at least a week, more than one week, at least a month, at least two months, at least three months, at least four months, at least five months, at least six months, at least seven months, at least eight months, at least nine months, at least ten months, at least eleven months, at least twelve months, or more.

In one embodiment, the solid aggregating microparticles that produce a pellet in vivo release the therapeutic agent without a burst of more than about 1 percent to about 5 percent of total payload over a 24 hour period.

In one embodiment, the solid aggregating microparticles that produce a pellet in vivo release the therapeutic agent without a burst of more than about 10 percent of total payload over a 24 hour period.

In one embodiment, the solid aggregating microparticles that produce a pellet in vivo release the therapeutic agent without a burst of more than about 15 percent of total payload over a 24 hour period.

In one embodiment, the solid aggregating microparticles that produce a pellet in vivo release the therapeutic agent without a burst of more than about 20 percent of total payload over a 24 hour period.

In one embodiment, the solid aggregating microparticles that produce a pellet in vivo release the therapeutic agent without a burst of more than about 1 percent to about 5 percent of total payload over a 12 hour period.

In one embodiment, the solid aggregating microparticles that produce a pellet in vivo release the therapeutic agent without a burst of more than about 5 percent to about 10 percent of total payload over a 12 hour period.

In one embodiment, the solid aggregating microparticles that produce a pellet in vivo release the therapeutic agent without a burst of more than about 10 percent of total payload over a 12 hour period.

In one embodiment, the solid aggregating microparticles that produce a pellet in vivo release the therapeutic agent without a burst of more than about 15 percent of total payload over a 12 hour period.

In one embodiment, the solid aggregating microparticles that produce a pellet in vivo release the therapeutic agent without a burst of more than about 20 percent of total payload over a 12 hour period.

In one embodiment, the pharmaceutically active compound is released in an amount effective to have a desired local or systemic physiological or pharmacologically effect.

In one embodiment, delivery of a pharmaceutically active compound means that the pharmaceutically active compound is released from the consolidated aggregate into the environment around the consolidated aggregate, for example, the vitreal fluid.

In one embodiment, a microparticle comprising a pharmaceutically active compound of the invention allows a substantially zero or first order release rate of the pharmaceutically active compound from the consolidated aggregate once the consolidated aggregate has formed. A zero order release rate is a constant release of the pharmaceutically active compound over a defined time; such release is difficult to achieve using known delivery methods.

### XVII. Non-Limiting Embodiments of Solid Aggregating Microparticles, Injections, and Suspensions Include

(I) A suspension of surface-modified aggregating microparticles in a diluent comprising additive that improves in vivo particle aggregation wherein the surface-modified solid aggregating microparticles comprise surface surfactant and a therapeutic agent disclosed herein encapsulated in at least one biodegradable polymer, wherein the microparticles:
(i) have a solid core with less than 10% porosity by ratio of void space to total volume;
(ii) have been surface-modified to contain less surfactant on the surface than a microparticle prior to surface modification and wherein the surface has been treated under mild conditions at a temperature less than about 18° C.;
(iii) have a mean diameter between 10 μm and 60 μm; and
(iv) are capable of aggregating in vivo to form at least one pellet of at least 500 μm in vivo capable of sustained drug delivery in vivo for at least three months.

(II) A suspension of surface-modified aggregating microparticles in a diluent comprising additive that improves in vivo particle aggregation wherein the surface-modified solid aggregating microparticles comprise surface surfactant and a therapeutic agent disclosed herein encapsulated in at least one biodegradable polymer selected from PLA and PLGA and at least one hydrophobic polymer covalently bound to a hydrophilic polymer, wherein the microparticles:
(i) have a solid core with less than 10% porosity by ratio of void space to total volume;
(ii) have been surface-modified to contain less surfactant on the surface than a microparticle prior to surface modification and wherein the surface has been treated under mild conditions at a temperature less than about 18° C.;
(iii) have a mean diameter between 10 μm and 60 μm; and
(iv) are capable of aggregating in vivo to form at least one pellet of at least 500 μm in vivo capable of sustained drug delivery in vivo for at least three months.

(III) A suspension of surface-modified aggregating microparticles in a diluent comprising additive that improves in vivo particle aggregation wherein the surface-modified solid aggregating microparticles comprise surface surfactant and a therapeutic agent disclosed herein encapsulated in at least one biodegradable polymer, wherein the microparticle:
(i) have a solid core with less than 10% porosity by ratio of void space to total volume;
(ii) have been surface-modified to contain less surfactant on the surface than a microparticle prior to surface modification and wherein the surface has been treated under mild conditions at a temperature less than about 18° C.;
(iii) have a mean diameter between 10 μm and 60 μm;
(iv) are capable of aggregating in vivo to form at least one pellet of at least 500 μm in vivo capable of sustained drug delivery in vivo for at least three months; and
(v) wherein the suspension has been treated with vacuum at a pressure of less than 40 Torr, less than 30 Torr, less than 25 Torr, less than 20 Torr, less than 10 Torr, or less than 5 Torr for between 1 and 90 minutes.

(IV) A suspension of surface-modified aggregating microparticles in a diluent comprising additive that improves in vivo particle aggregation wherein the surface-modified solid aggregating microparticles comprise surface surfactant and a therapeutic agent disclosed herein encapsulated in at least one biodegradable polymer, wherein the microparticles:
(i) have been surface-modified to contain less surfactant on the surface than a microparticle prior to surface modification and wherein the surface has been treated under mild conditions at a temperature less than about 18° C.;
(ii) have a mean diameter between 10 μm and 60 μm; and
(iii) are capable of aggregating in vivo to form at least one pellet of at least 500 μm in vivo capable of sustained drug delivery in vivo for at least three months.

(V) A suspension of surface-modified aggregating microparticles in a diluent comprising additive that improves in vivo particle aggregation wherein the surface-modified solid aggregating microparticles comprise surface surfactant and a therapeutic agent disclosed herein encapsulated in at least one biodegradable polymer selected from PLA and PLGA and at least one hydrophobic polymer covalently bound to a hydrophilic polymer, wherein the microparticles:
(i) have been surface-modified to contain less surfactant on the surface than a microparticle prior to surface modification and wherein the surface has been treated under mild conditions at a temperature less than about 18° C.;
(ii) have a mean diameter between 10 μm and 60 μm; and
(iii) are capable of aggregating in vivo to form at least one pellet of at least 500 μm in vivo capable of sustained drug delivery in vivo for at least three months.

(VI) A suspension of surface-modified aggregating microparticles in a diluent comprising additive that improves in vivo particle aggregation wherein the surface-modified solid aggregating microparticles comprise surface surfactant and a therapeutic agent disclosed herein encapsulated in at least one biodegradable polymer, wherein the microparticle:
(i) have been surface-modified to contain less surfactant on the surface than a microparticle prior to surface modification and wherein the surface has been treated under mild conditions at a temperature less than about 18° C.;
(ii) have a mean diameter between 10 μm and 60 μm;
(iii) are capable of aggregating in vivo to form at least one pellet of at least 500 μm in vivo capable of sustained drug delivery in vivo for at least three months; and
(iv) wherein the suspension has been treated with vacuum at a pressure of less than 40 Torr, less than 30 Torr, less than 25 Torr, less than 20 Torr, less than 10 Torr, or less than 5 Torr for between 1 and 90 minutes.

### Particular Embodiments Include:

a. The suspension of surface-modified solid aggregating microparticles of (I), (II), (III), (IV), (V), or (VI) suitable for a delivery route selected from the group consisting of intravitreal, intrastromal, intracameral, subtenon, sub-retinal, retrobulbar, peribulbar, suprachoroidal, subchoroidal, conjunctival, subconjunctival, episcleral, posterior juxtascleral, circumcorneal, and tear duct injections.
b. The suspension of surface-modified solid aggregating microparticles of (I), (II), (III), (IV), (V), or (VI) wherein the at least one pellet is capable of sustained delivery for at least four months, at least five months, at least six months, at least seven months, at least eight months, at least nine months, or at least ten months.
c. The suspension of surface-modified solid aggregating microparticles of (I), (II), (III), (IV), (V), or (VI) wherein the surface modification is carried out at a pH between about 14 and about 12.
d. The suspension of surface-modified solid aggregating microparticles of (I), (II), (III), (IV), (V), or (VI) wherein the surface modification is carried out at a pH between about 12 and about 10.
e. The suspension of surface-modified solid aggregating microparticles of (I), (II), (III), (IV), (V), or (VI) wherein the surface modification is carried out at a pH between about 10 and about 8.
f. The suspension of surface-modified solid aggregating microparticles of (I), (II), (III), (IV), (V), or (VI) wherein the surface modification is carried out at a pH between about 6.5 and about 7.5.
g. The suspension of surface-modified solid aggregating microparticles of (I), (II), (III), (IV), (V), or (VI) wherein the surface modification is carried out at a pH between about 1 and about 6.
h. The suspension of surface-modified solid aggregating microparticles of (I), (II), (III), (IV), (V), or (VI) wherein the surface modification is carried out at a pH not greater than 9.
i. The suspension of surface-modified solid aggregating microparticles of (I), (II), (III), (IV), (V), or (VI) wherein the surface modification is carried out at a temperature of less than about 16° C.
j. The suspension of surface-modified solid aggregating microparticles of (I), (II), (III), (IV), (V), or (VI) wherein the surface modification is carried out at a temperature of less than about 10° C.
k. The suspension of surface-modified solid aggregating microparticles of (I), (II), (III), (IV), (V), or (VI) wherein the surface modification is carried out at a temperature of less than about 8° C.
l. The suspension of surface-modified solid aggregating microparticles of (I), (II), (III), (IV), (V), or (VI) wherein the surface modification is carried out at a temperature of less than about 5° C.
m. The suspension of surface-modified solid aggregating microparticles of (I), (II), (III), (IV), (V), or (VI) wherein the surface modification is carried out at a temperature of less than about 2° C.
n. The suspension of surface-modified solid aggregating microparticles of (II), wherein the hydrophobic polymer covalently bound to a hydrophilic polymer is poly(D,L-lactide-co-glycolide) covalently bound to polyethylene glycol (PLGA-PEG).
o. The suspension of surface-modified solid aggregating microparticles of (I), (II), (III), (IV), (V), or (VI) wherein the microparticles comprise PLA and PLGA-PEG.
p. The suspension of surface-modified solid aggregating microparticles of (I), (II), (III), (IV), (V), or (VI) wherein the microparticles comprise PLA and PLGA-PEG and the ratio by weight of PLA to PLGA-PEG is between about 99/1.
q. The suspension of surface-modified solid aggregating microparticles of (I), (II), (III), (IV), (V), or (VI) wherein the microparticles comprise PLGA and PLGA-PEG.
r. The suspension of surface-modified solid aggregating microparticles of (I), (II), (III), (IV), (V), or (VI) wherein the microparticles comprise PLGA and PLGA-PEG and the ratio by weight of PLA to PLGA-PEG is between about 99/1.
s. The suspension of surface-modified solid aggregating microparticles of (I), (II), (III), (IV), (V), or (VI) wherein the microparticles comprise PLA/PLGA PLGA-PEG.
t. The suspension of surface-modified solid aggregating microparticles of (I), (II), (III), (IV), (V), or (VI) wherein the microparticles comprise PLA/PLGA PLGA-PEG and the ratio by weight of PLA/PLGA/PLGA-PEG is about 5/95/1, 10/90/1, 15/85/1, 20/80/1, 25/75/1, 30/70/1, 35/65/1, 40/60/1, 45/55/1, 50/50/1, 55/45/1, 60/40/1, 65/35/1, 70/30/1, 75/25/1, 80/20/1, 85/15/1, 90/10/1, 95/5/1, or 100/1/1.
u. The suspension of surface-modified solid aggregating microparticles of (I), (II), (III), (IV), (V), or (VI) wherein the microparticles comprise PLA/PLGA PLGA-PEG and the ratio by weight of PLA/PLGA/PLGA-PEG is about 95/5/1.
v. The suspension of surface-modified solid aggregating microparticles of (I), (II), (III), (IV), (V), or (VI) wherein the microparticles comprise PLA/PLGA PLGA-PEG and the ratio by weight of PLA/PLGA/PLGA-PEG is about 90/10/1.
w. The suspension of surface-modified solid aggregating microparticles of (I), (II), (III), (IV), (V), or (VI) wherein the microparticles comprise PLA/PLGA PLGA-PEG and the ratio by weight of PLA/PLGA/PLGA-PEG is about 70/30/1.
x. The suspension of surface-modified solid aggregating microparticles of (I), (II), (III), (IV), (V), or (VI) wherein the microparticles comprise (i) PLGA; (ii) PLGA wherein the PLGA in (ii) has a different ratio of lactide to glycolide than the PLGA in (i); and, PLGA-PEG.
y. The suspension of surface-modified solid aggregating microparticles of (I), (II), (III), (IV), (V), or (VI) wherein the microparticles comprise PLGA50:50, PLGA75:25, and PLGA-PEG.
z. The suspension of surface-modified solid aggregating microparticles of (I), (II), (III), (IV), (V), or (VI) wherein the microparticles comprise PLGA50:50, PLGA85:15, and PLGA-PEG.
aa. The suspension of surface-modified solid aggregating microparticles of (I), (II), (III), (IV), (V), or (VI) wherein the microparticles comprise PLGA85:15, PLGA75:25, and PLGA-PEG.
bb. The suspension of surface-modified solid aggregating microparticles of (I), (II), (III), (IV), (V), or (VI) wherein the PLA is ester end-capped.
cc. The suspension of surface-modified solid aggregating microparticles of (I), (II), (III), (IV), (V), or (VI) wherein the PLA is acid end-capped.
dd. The suspension of surface-modified solid aggregating microparticles of (I), (II), (III), (IV), (V), or (VI) wherein the microparticles have a mean diameter between about 20 and 30 μm.
ee. The suspension of surface-modified solid aggregating microparticles of (I), (II), (III), (IV), (V), or (VI) wherein the microparticles have a mean diameter between about 20 and 50 μm.
ff. The suspension of surface-modified solid aggregating microparticles of (I), (II), (III), (IV), (V), or (VI) wherein the microparticles have a mean diameter between about 25 and 35 μm.
gg. The suspension of surface-modified solid aggregating microparticles of (I), (II), (III), (IV), (V), or (VI) wherein the microparticles have a mean diameter between about 20 and 40 μm.
hh. The suspension of surface-modified solid aggregating microparticles of (I), (II), (III), (IV), (V), or (VI) wherein the microparticles have a mean diameter between about 25 and 40 μm.
ii. The suspension of surface-modified solid aggregating microparticles of (I), (II), (III), (IV), (V), or (VI) wherein the therapeutic agent is a pharmaceutical drug.
jj. The suspension of surface-modified solid aggregating microparticles of (I), (II), (III), (IV), (V), or (VI) wherein the therapeutic agent is a prodrug as described herein.
kk. The suspension of surface-modified solid aggregating microparticles of (I), (II), (III), (IV), (V), or (VI) wherein the therapeutic agent is sunitinib or a pharmaceutically acceptable salt thereof.
ll. The suspension of surface-modified solid aggregating microparticles of (I), (II), (III), (IV), (V), or (VI) wherein the therapeutic agent is sunitinib malate.
mm. The suspension of surface-modified solid aggregating microparticles of ((I), (II), (III), (IV), (V), or (VI) wherein the therapeutic agent is atropine, pilocarpine, or alpha lipoic acid.
nn. The suspension of surface-modified solid aggregating microparticles of (I), (II), (III), (IV), (V), or (VI) wherein the therapeutic agent is selected from Tivosinib, Imatinib, Gefitinib, and Erlotinib.
oo. The suspension of surface-modified solid aggregating microparticles of (I), (II), (III), (IV), (V), or (VI) wherein the therapeutic agent is selected from Lapatinib, Canertinib, Semaxinib, and Vatalaninib,
pp. The suspension of surface-modified solid aggregating microparticles of (I), (II), (III), (IV), (V), or (VI) wherein the therapeutic agent is selected from Sorafenib, Axitinib, Pazopanib, and Dasatinib.
qq. The suspension of surface-modified solid aggregating microparticles of (I), (II), (III), (IV), (V), or (VI) wherein the therapeutic agent is selected from Nilotinib, Crizotinib, Ruxolitinib, Vandetanib, and Vemurafenib.
rr. The suspension of surface-modified solid aggregating microparticles of (I), (II), (III), (IV), (V), or (VI) wherein the therapeutic agent is selected from Bosutinib, Cabozantinib, Regorafenib, Vismodegib, and Ponatinib.
ss. The suspension of surface-modified solid aggregating microparticles of (I), (II), (III), (IV), (V), or (VI) wherein the therapeutic agent is selected furosemide, bumetanide, piretanide, ethacrynic acid, etozolin, and ozolinone.
tt. The suspension of surface-modified solid aggregating microparticles of (I), (II), (III), (IV), (V), or (VI) wherein the surface surfactant is polyvinyl alcohol.
uu. The suspension of surface-modified solid aggregating microparticles of (I), (II), (III), (IV), (V), or (VI) wherein the therapeutic agent is selected from Tables A-I or a pharmaceutically acceptable salt thereof.
vv. The suspension of surface-modified solid aggregating microparticles of ((I), (II), (III), (IV), (V), or (VI) wherein the therapeutic agent is

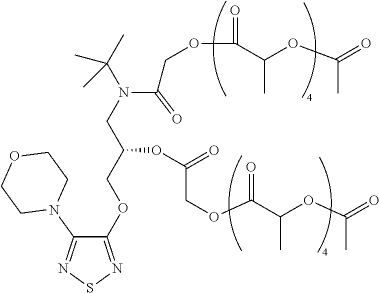

or a pharmaceutically acceptable salt thereof.
ww. The suspension of surface-modified solid aggregating microparticles of (I), (II), (III), (IV), (V), or (VI), wherein the therapeutic agent is

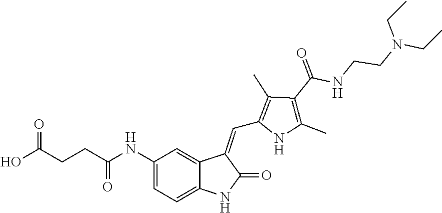 [SMILES: CCN(CC)CCNC(=O)c1c(C)[nH]c(/C=C2\C(=O)Nc3ccc(NC(=O)CCC(=O)O)cc32)c1C]

or a pharmaceutically acceptable salt thereof
xx. The suspension of surface-modified solid aggregating microparticles of (I), (II), (III), (IV), (V), or (VI) wherein the therapeutic agent is

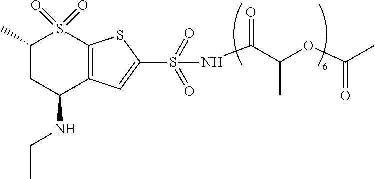

or a pharmaceutically acceptable salt thereof.
yy. The suspension of surface-modified solid aggregating microparticles of (I), (II), (III), (IV), (V), or (VI), wherein the additive is benzyl alcohol.
zz. The suspension of surface-modified solid aggregating microparticles of (I), (II), (III), (IV), (V), or (VI), wherein the additive is triethyl citrate.
aaa. The suspension of surface-modified solid aggregating microparticles of (I), (II), (III), (IV), (V), or (VI), wherein the additive is selected from polyethylene glycol, N-methyl-2-pyrrolidone (NMP), 2-pyrrolidone, and DMSO.
bbb. The suspension of surface-modified solid aggregating microparticles of (I), (II), (III), (IV), (V), or (VI), wherein the additive is selected from triacetin, benzyl acetate, benzyl benzoate, and acetyltributyl citrate.
ccc. The suspension of surface-modified solid aggregating microparticles of (I), (II), (III), (IV), (V), or (VI), wherein the additive is selected from dibutyl sebacate, dimethylphthalate, tributyl O-acetylcitrate, ethanol, and methanol.
ddd. The suspension of surface-modified solid aggregating microparticles of (I), (II), (III), (IV), (V), or (VI), wherein the additive is selected from polysorbate 80, ethyl acetate, propylene carbonate, and isopropyl acetate.
eee. The suspension of surface-modified solid aggregating microparticles of (I), (II), (III), (IV), (V), or (VI), wherein the additive is selected from methyl acetate, methyl ethyl ketone, butyl lactate, and isovaleric acid.
fff. The suspension of surface-modified solid aggregating microparticles of (I), (II), (III), (IV), (V), or (VI), wherein the diluent further comprises a viscosity enhancer.
ggg. The suspension of surface-modified solid aggregating microparticles of (I), (II), (III), (IV), (V), or (VI), wherein the diluent is hyaluronic acid.
hhh. The suspension of surface-modified solid aggregating microparticles of (I), (II), (III), (IV), (V), or (VI), wherein the diluent is sodium hyaluronate.
iii. The suspension of surface-modified solid aggregating microparticles of (I), (II), (III), (IV), (V), or (VI), wherein the microparticles contain from about 0.001 percent to about 1 percent surfactant
jjj. The suspension of surface-modified solid aggregating microparticles of (I), (II), (III), (IV), (V), or (VI), in a diluent comprising additive wherein the surface-modified solid aggregating microparticle comprises surface surfactant and sunitinib or a pharmaceutically acceptable salt thereof encapsulated in PLGA and PLGA-PEG.
kkk. The suspension of surface-modified solid aggregating microparticles of (I), (II), (III), (IV), (V), or (VI), in a diluent comprising benzyl alcohol wherein the surface-modified solid aggregating microparticle comprises surface surfactant and sunitinib or a pharmaceutically acceptable salt thereof encapsulated in PLGA and PLGA-PEG.
lll. The suspension of surface-modified solid aggregating microparticles of (I), (II), (III), (IV), (V), or (VI), in a diluent comprising triethyl citrate wherein the surface-modified solid aggregating microparticle comprises surface surfactant and sunitinib or a pharmaceutically acceptable salt thereof encapsulated in PLGA and PLGA-PEG.
mmm. The suspension of surface-modified solid aggregating microparticles of (I), (II), (III), (IV), (V), or (VI), in a diluent comprising additive wherein the surface-modified solid aggregating microparticle comprises surface surfactant and sunitinib or a pharmaceutically acceptable salt thereof encapsulated in PLA and PLGA-PEG.
nnn. The suspension of surface-modified solid aggregating microparticles of (I), (II), (III), (IV), (V), or (VI), in a diluent comprising benzyl alcohol wherein the surface-modified solid aggregating microparticle comprises surface surfactant and sunitinib or a pharmaceutically acceptable salt thereof encapsulated in PLA and PLGA-PEG.
ooo. The suspension of surface-modified solid aggregating microparticles of (I), (II), (III), (IV), (V), or (VI), in a diluent comprising triethyl citrate wherein the surface-modified solid aggregating microparticle comprises surface surfactant and sunitinib or a pharmaceutically acceptable salt thereof encapsulated in PLA and PLGA-PEG.
ppp. The suspension of surface-modified solid aggregating microparticles of (I), (II), (III), (IV), (V), or (VI), in a diluent comprising additive wherein the surface-modified solid aggregating microparticle comprises surface surfactant and sunitinib or a pharmaceutically acceptable salt thereof encapsulated in PLGA, PLA and PLGA-PEG.
qqq. The suspension of surface-modified solid aggregating microparticles of (I), (II), (III), (IV), (V), or (VI), in a diluent comprising benzyl alcohol wherein the surface-modified solid aggregating microparticle comprises surface surfactant and sunitinib or a pharmaceutically acceptable salt thereof encapsulated in PLGA, PLA and PLGA-PEG.
rrr. The suspension of surface-modified solid aggregating microparticles of (I), (II), (III), (IV), (V), or (VI), in a diluent comprising triethyl citrate wherein the surface-modified solid aggregating microparticle comprises surface surfactant and sunitinib or a pharmaceutically acceptable salt thereof encapsulated in PLGA, PLA and PLGA-PEG.
sss. The suspension of surface-modified solid aggregating microparticles of (I), (II), or (III), wherein the surface-modified solid aggregating microparticle have a solid core with less than 10% porosity by ratio of void space to total volume.
ttt. The suspension of surface-modified solid aggregating microparticles of (I), (II), or (III), wherein the surface-modified solid aggregating microparticle have a solid core with less than 8% porosity by ratio of void space to total volume.
uuu. The suspension of surface-modified solid aggregating microparticles of (I), (II), or (III), wherein the surface-modified solid aggregating microparticle have a solid core with less than 5% porosity by ratio of void space to total volume.
vvv. The suspension of surface-modified solid aggregating microparticles of (I), (II), or (III), wherein the surface-modified solid aggregating microparticle have a solid core with less than 3% porosity by ratio of void space to total volume.
www. The suspension of surface-modified solid aggregating microparticles of (I), (II), or (III), wherein the surface-modified solid aggregating microparticle have a solid core with less than 2% porosity by ratio of void space to total volume.
xxx. A pharmaceutical composition of any one of the above embodiments suitable for injection.
yyy. A pharmaceutical composition of any one of the above embodiments suitable for a delivery route selected from the group consisting of intravitreal, intrastromal, intracameral, subtenon, sub-retinal, retrobulbar, peribulbar, suprachoroidal, subchoroidal, conjunctival, subconjunctival, episcleral, posterior juxtascleral, circumcorneal, and tear duct injections.
zzz. The suspension of surface-modified solid aggregating microparticles of (I), (II), (III), (IV), (V), or (VI), for use in treating or preventing disorders related to an ocular disorder such as glaucoma, a disorder mediated by carbonic anhydrase, a disorder or abnormality related to an increase in intraocular pressure (IOP), a disorder mediated by nitric oxide synthase (NOS), a disorder requiring neuroprotection such as to regenerate/repair optic nerves, allergic conjunctivitis, anterior uveitis, cataracts, dry or wet age-related macular degeneration (AMD), neovascular age-related macular degeneration (NVAMD), geographic atrophy or diabetic retinopathy;
aaaa. A method to treat or prevent disorders related to an ocular disorder such as glaucoma, a disorder mediated by carbonic anhydrase, a disorder or abnormality related to an increase in intraocular pressure (IOP), a disorder mediated by nitric oxide synthase (NOS), a disorder requiring neuroprotection such as to regenerate/repair optic nerves, allergic conjunctivitis, anterior uveitis, cataracts, dry or wet age-related macular degeneration (AMD), neovascular age-related macular degeneration (NVAMD), geographic atrophy or diabetic retinopathy using the suspension of surface-modified solid aggregating microparticles of (I), (II), (III), (IV), (V), or (VI);
bbbb. Use of a suspension of surface-modified solid aggregating microparticles of (I), (II), (III), (IV), (V), or (VI) in the manufacture of a medicament for use in treating or preventing disorders related to an ocular disorder such as glaucoma, a disorder mediated by carbonic anhydrase, a disorder or abnormality related to an increase in intraocular pressure (IOP), a disorder mediated by nitric oxide synthase (NOS), a disorder requiring neuroprotection such as to regenerate/repair optic nerves, allergic conjunctivitis, anterior uveitis, cataracts, dry or wet age-related macular degeneration (AMD), neovascular age-related macular degeneration (NVAMD), geographic atrophy or diabetic retinopathy.

### XVIII. Manufacture of Microparticles

### Microparticle Formation

Microparticles can be formed using any suitable method for the formation of polymer microparticles known in the art. The method employed for particle formation will depend on a variety of factors, including the characteristics of the polymers present in the drug or polymer matrix, as well as the desired particle size and size distribution. The type of drug(s) being incorporated in the microparticles may also be a factor as some drugs are unstable in the presence of certain solvents, in certain temperature ranges, and/or in certain pH ranges.

Particles having an average particle size of between 1 micron and 100 microns are useful in the compositions described herein. In typical embodiments, the particles have an average particle size of between 1 micron and 40 microns, more typically between about 10 micron and about 40 microns, more typically between about 20 micron and about 40 microns. The particles can have any shape but are generally spherical in shape.

In circumstances where a monodisperse population of particles is desired, the particles may be formed using a method which produces a monodisperse population of microparticles. Alternatively, methods producing polydispersed microparticle distributions can be used, and the particles can be separated using methods known in the art, such as sieving, following particle formation to provide a population of particles having the desired average particle size and particle size distribution.

Common techniques for preparing microparticles include, but are not limited to, solvent evaporation, hot melt particle formation, solvent removal, spray drying, phase inversion, coacervation, and low temperature casting. Suitable methods of particle formulation are briefly described below. Pharmaceutically acceptable excipients, including pH modifying agents, disintegrants, preservatives, and antioxidants, can optionally be incorporated into the particles during particle formation.

In one embodiment, surface treated microparticles are prepared using continuous chemistry manufacturing processes. In one embodiment, surface treated microparticles are prepared using step-wise manufacturing processes.

In one embodiment, microparticles containing a therapeutic agent can be prepared as described in PCT/US2015/065894. In one embodiment, the microparticles are prepared by:
(i) dissolving or dispersing the therapeutic agent or its salt in an organic solvent optionally with an alkaline agent;
(ii) mixing the solution/dispersion of step (i) with a polymer solution that has a viscosity of at least about 300 cPs (or perhaps at least about 350, 400, 500, 600, 700 or 800 or more cPs);
(iii) mixing the therapeutic agent polymer solution/dispersion of step (ii) with an aqueous non-acidic or alkaline solution (for example at least approximately a pH of 7, 8, or 9 and typically not higher than about 10) optionally with a surfactant or emulsifier, to form a solvent-laden therapeutic agent encapsulated microparticle,
(iv) isolating the microparticles.
   In one embodiment, the therapeutic agent is sunitinib.

It has been found that it may be useful to include the alkaline agent in the organic solvent. However, as described in PCT/US2015/065894, it has been found that adding an acid to the organic solvent can improve drug loading of the microparticle. Examples demonstrate that polyesters such as PLGA, PEG-PLGA(PLA) and PEG-PLGA/PLGA blend microparticles display sustained release of the therapeutic agent or its pharmaceutically acceptable salt. Polymer microparticles composed of PLGA and PEG covalently conjugated to PLGA (Mw 45 kDa) (PLGA45k-PEG5k) loaded with the therapeutic agent were prepared using a single emulsion solvent evaporation method. The therapeutic agent loading was further increased by increasing the pH of the aqueous solution. Still further significant increases in therapeutic agent loading in the microparticles was achieved by increasing polymer concentration or viscosity. In one embodiment, the therapeutic agent is sunitinib.

### Solvent Evaporation

In this method, the drug (or polymer matrix and drug) is dissolved in a volatile organic solvent, such as methylene chloride, acetone, acetonitrile, 2-butanol, 2-butanone, t-butyl alcohol, benzene, chloroform, cyclohexane, 1,2-dichloroethane, diethyl ether, ethanol, ethyl acetate, heptane, hexane, methanol, methyl tert-butyl ether, pentane, petroleum ether, iso-propanol, n-propanol, tetrahydrofuran, or mixtures thereof. The organic solution containing the drug is then suspended in an aqueous solution that contains a surface active agent such as poly(vinyl alcohol). The resulting emulsion is stirred until most of the organic solvent is evaporated, leaving solid microparticles. The resulting microparticles are washed with water and dried overnight in a lyophilizer. Microparticles with different sizes and morphologies can be obtained by this method.

Microparticles which contain labile polymers, such as certain polyanhydrides, may degrade during the fabrication process due to the presence of water. For these polymers, the following two methods, which are performed in completely anhydrous organic solvents, can be used.

### Oil-In-Oil Emulsion Technique

Solvent removal can also be used to prepare particles from drugs that are hydrolytically unstable. In this method, the drug (or polymer matrix and drug) is dispersed or dissolved in a volatile organic solvent such as methylene chloride, acetone, acetonitrile, benzene, 2-butanol, 2-butanone, t-butyl alcohol, chloroform, cyclohexane, 1,2-dichloroethane, diethyl ether, ethanol, ethyl acetate, heptane, hexane, methanol, methyl tert-butyl ether, pentane, petroleum ether, iso-propanol, n-propanol, tetrahydrofuran, or mixtures thereof. This mixture is then suspended by stirring in an organic oil (such as silicon oil, castor oil, paraffin oil, or mineral oil) to form an emulsion. Solid particles form from the emulsion, which can subsequently be isolated from the supernatant. The external morphology of spheres produced with this technique is highly dependent on the identity of the drug.

### Oil-In-Water Emulsion Technique

In this method, the drug (or polymer matrix and drug) is dispersed or dissolved in a volatile organic solvent such as methylene chloride, acetone, acetonitrile, benzene, 2-butanol, 2-butanone, t-butyl alcohol, chloroform, cyclohexane, 1,2-dichloroethane, diethyl ether, ethanol, ethyl acetate, heptane, hexane, methanol, methyl tert-butyl ether, pentane, petroleum ether, iso-propanol, n-propanol, tetrahydrofuran, or mixtures thereof. This mixture is then suspended by stirring in an aqueous solution of surface active agent, such as poly(vinyl alcohol), to form an emulsion. Solid particles form from the emulsion, which can subsequently be isolated from the supernatant. The external morphology of spheres produced with this technique is highly dependent on the identity of the drug.

As described in PCT/US2015/065894, microparticles with a therapeutic agent can be prepared using the oil-in-water emulsion method. In one example, sunitinib microparticles were prepared by dissolving 100 mg PEG-PLGA (5K, 45) in 1 mL methylene chloride, and dissolving 20 mg sunitinib malate in 0.5 mL DMSO and triethylamine. The solutions were then mixed together, homogenized at 5000 rpm, 1 minute into an aqueous solution containing 1% polyvinyl alcohol (PVA) and stirred for 2 hours. The particles were collected, washed with double distilled water, and freeze dried. In another example, sunitinib microparticles were also prepared according to PCT/US2015/065894 by dissolving 200 mg PLGA (2 A, Alkermers) in 3 mL methylene chloride, and 40 mg sunitinib malate in 0.5 mL DMSO and triethylamine. The solutions were then mixed together and homogenized at 5000 rpm, 1 minute in 1% PVA and stirred for 2 hours. The particles were collected, washed with double distilled water, and freeze dried.

### Spray Drying

In this method, the drug (or polymer matrix and drug) is dissolved in an organic solvent such as methylene chloride, acetone, acetonitrile, 2-butanol, 2-butanone, t-butyl alcohol, benzene, chloroform, cyclohexane, 1,2-dichloroethane, diethyl ether, ethanol, ethyl acetate, heptane, hexane, methanol, methyl tert-butyl ether, pentane, petroleum ether, iso-propanol, n-propanol, tetrahydrofuran, or mixtures thereof. The solution is pumped through a micronizing nozzle driven by a flow of compressed gas, and the resulting aerosol is suspended in a heated cyclone of air, allowing the solvent to evaporate from the microdroplets, forming particles. Particles ranging between 0.1-10 microns can be obtained using this method.

### Phase Inversion

Particles can be formed from drugs using a phase inversion method. In this method, the drug (or polymer matrix and drug) is dissolved in a solvent, and the solution is poured into a strong non solvent for the drug to spontaneously produce, under favorable conditions, microparticles or nanoparticles. The method can be used to produce nanoparticles in a wide range of sizes, including, for example, about 100 nanometers to about 10 microns, typically possessing a narrow particle size distribution.

### Coacervation

Techniques for particle formation using coacervation are known in the art, for example, in GB-B-929 406; GB-B-929 40 1; and U.S. Pat. Nos. 3,266,987, 4,794,000, and 4,460,563. Coacervation involves the separation of a drug (or polymer matrix and drug) solution into two immiscible liquid phases. One phase is a dense coacervate phase, which contains a high concentration of the drug, while the second phase contains a low concentration of the drug. Within the dense coacervate phase, the drug forms nanoscale or microscale droplets, which harden into particles. Coacervation may be induced by a temperature change, addition of a non-solvent or addition of a micro-salt (simple coacervation), or by the addition of another polymer thereby forming an interpolymer complex (complex coacervation).

### Low Temperature Casting

Methods for very low temperature casting of controlled release microspheres are described in U.S. Pat. No. 5,019,400 to Gombotz et al. In this method, the drug (or polymer matrix and sunitinib) is dissolved in a solvent. The mixture is then atomized into a vessel containing a liquid non-solvent at a temperature below the freezing point of the drug solution which freezes the drug droplets. As the droplets and non-solvent for the drug are warmed, the solvent in the droplets thaws and is extracted into the non-solvent, hardening the microspheres.

### Scale Up

The processes for producing microparticles described in the Examples are amenable to scale up by methods known in the art. Examples of such methods include U.S. Pat. Nos. 4,822,534; 5,271,961; 5,945,126; 6,270,802; 6,361,798; U.S. Pat. No. 8,708,159; and U.S. publication 2010/0143479. U.S. Pat. No. 4,822,534 describes a method of manufacture to provide solid microspheres that involves the use of dispersions. These dispersions could be produced industrially and allowed for scale up. U.S. Pat. No. 5,271,961 disclosed the production of protein microspheres which involved the use of low temperatures, usually less than 45° C. U.S. Pat. No. 5,945,126 describes the method of manufacture to produce microparticles on full production scale while maintaining size uniformity observed in laboratory scale. U.S. Pat. Nos. 6,270,802 and 6,361,798 describe the large scale method of manufacture of polymeric microparticles whilst maintaining a sterile field. U.S. Pat. No. 8,708,159 describes the processing of microparticles on scale using a hydrocyclone apparatus. U.S. publication 2010/0143479 describes the method of manufacture of microparticles on large scale specifically for slow release microparticles.

XSpray has disclosed a device and the use of supercritical fluids to produce particles of a size below 10 μM (U.S. Pat. No. 8,167,279). Additional patents to XSpray include U.S. Pat. Nos. 8,585,942 and 8,585,943. Sun Pharmaceuticals has disclosed a process for the manufacture of microspheres or microcapsules, WO 2006/123359, herein incorporated by reference. As an example, Process A involves five steps that include 1) the preparation of a first dispersed phase comprising a therapeutically active ingredient, a biodegradable polymer and an organic solvent 2) mixing the first dispersed phase with an aqueous phase to form an emulsion 3) spraying the emulsion into a vessel equipped to remove an organic solvent and 4) passing the resulting microspheres or microcapsules through a first and second screen thereby collecting a fractionated size of the microspheres or microcapsules and 5) drying the microspheres or microcapsules.

Xu, Q. et al. have disclosed the preparation of monodispersed biodegradable polymer microparticles using a microfluidic flow-focusing device (Xu, Q., et al “Preparation of Monodispersed Biodegradable Polymer Microparticles Using a Microfluidic Flow-Focusing Device for Controlled Drug Delivery”, Small, Vol 5(13): 1575-1581, 2009).

Duncanson, W. J. et al. have disclosed the use of microfluidic devices to generate microspheres (Duncanson, W. J. et al. “Microfluidic Synthesis of Monodisperse Porous Microspheres with Size-tunable Pores”, Soft Matter, Vol 8, 10636-10640, 2012).

U.S. Pat. No. 8,916,196 to Evonik describes an apparatus and method for the production of emulsion based microparticles that can be used in connection with the present invention.

### XIX. Examples

### Abbreviations

DCM, CH₂Cl₂ Dichloromethane
DL Drug loading
DMSO Dimethyl sulfoxide
EtOH Ethanol
HA Sodium hyaluronate
hr, h Hour
min Minute
NaOH Sodium hydroxide
NSTMP Non-surface treated microparticles
PBS Dulbecco's phosphate-buffered saline
PCL Polycaprolactone
PEG Polyethylene glycol
PLA Poly(lactic acid)
PLGA Poly(lactic-co-glycolic acid)
PVA Polyvinyl alcohol
Rpm Revolutions per minute
RT, r.t. Room temperature
SD Standard deviation
STMP Surface treated microparticles
UV Ultraviolet

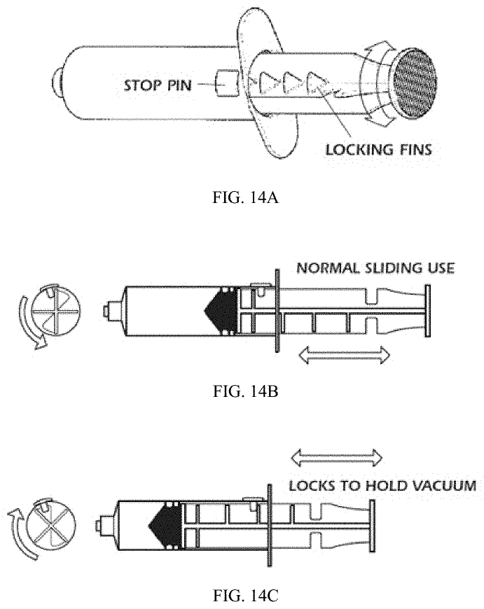

Examples 1-4 were first presented in U.S. Ser. No. 15/349,985 and PCT/US16/61706 and are provided again herein for background information for the improved invention described herein. FIGS. 14A-14C and FIG. 15 were first presented in U.S. Ser. No. 15/976,847, and PCT/US18/32167 and are provided again herein for background information for the improved invention described herein.

### General Methods

All non-aqueous reactions were performed under an atmosphere of dry argon or nitrogen gas using anhydrous solvents. The structure of starting materials, intermediates, and final products was confirmed by standard analytical techniques, including NMR spectroscopy and mass spectrometry.

### Materials

Sodium hydroxide (NaOH, catalog #: 5318-1, Fisher Chemical), ethanol (EtOH, catalog #: A405-20, Fisher Chemical), Dulbecco's phosphate-buffered saline (PBS, catalog #: SH3085003, GE Healthcare HyClone™), sodium hyaluronate (HA, catalog #: AC251770010, Acros Organics) and Tween 20 (catalog #: BP337-100, Fisher BioReagents) were purchased from Fisher Scientific. Polyvinyl alcohol (PVA) (88 percent hydrolyzed, MW approximately 25 kD) (catalog #: 02975) was purchased from Polysciences, Inc. Sunitinib malate was purchased from LC Laboratories (catalog #: S-8803). ProVisc® (10 mg/mL, 0.85 mL, catalog #: 21989, Alcon) was purchased from Besse Medical. Poly(lactic-co-glycolic acid) (PLGA) polymer, poly(lactic-acid) (PLA) polymer, and diblock co-polymers of PLGA and polyethylene glycol (PLGA-PEG) were purchased from the Evonik Corporation (RESOMER Select 5050 DLG mPEG 5000 (10 wt percent PEG)). A FreeZone 4.5 liter benchtop freeze dry system was used for lyophilization.

ProVisc® OVD (Ophthalmic Viscosurgical Device) is a sterile, non-pyrogenic, high molecular weight, non-inflammatory highly purified fraction of sodium hyaluronate dissolved in physiological sodium chloride phosphate buffer. It is FDA approved and indicated for use as an ophthalmic surgical aid. Sodium hyaluronate is a derivative of hyaluronan for clinical use. Hyaluronan, also known as hyaluronic acid, is a naturally occurring glycosaminoglycan found throughout the body including in the aqueous and vitreous humors of the eye.

### Example 1. Preparation of Biodegradable Non-Surface Treated Microparticles (NSTMP) Containing PLGA

Polymer microparticles comprising PLGA and diblock copolymer of PLGA and PEG with or without sunitinib malate were prepared using a single emulsion solvent evaporation method. As an example, PLGA (560 mg) and PLGA-PEG (5.6 mg) were co-dissolved in dichloromethane (DCM) (4 mL). Sunitinib malate (90 mg) was dissolved in dimethyl sulfoxide (DMSO) (2 mL). The polymer solution and the drug solution were mixed to form a homogeneous solution (organic phase). For empty NSTMP, DMSO (2 mL) without drug was used. For drug-loaded NSTMP, the organic phase was added to an aqueous 1% PVA solution in PBS (200 mL) and homogenized at 5,000 rpm for 1 minute using an L5M-A laboratory mixer (Silverson Machines Inc., East Longmeadow, Mass.) to obtain an emulsion. For empty NSTMP, 1 percent PVA solution in water (200 mL) was used.

The emulsion (solvent-laden microparticles) was then hardened by stirring at room temperature for more than 2 hours to allow the DCM to evaporate. The microparticles were collected by sedimentation and centrifugation, washed three times in water, and filtered through a 40-μm sterile Falcon® cell strainer (Corning Inc., Corning, N.Y.). The non-surface treated microparticles (NSTMP) were either used directly in the surface treatment process or dried by lyophilization and stored as a dry powder at −20° C. until used.

### Example 2. Surface Treatment of Non-Surface Treated Microparticles (NSTMP) Using NaOH(aq)/EtOH

A pre-chilled solution containing 0.25 M NaOH (aq) and ethanol at a predetermined ratio was added to microparticles in a glass vial under stirring in an ice bath at approximately 4° C. to form a suspension at 100 mg/mL. The suspension was then stirred for a predetermined time (e.g., 3, 6 or 10 minutes) on ice and poured into a pre-chilled filtration apparatus to remove the NaOH (aq)/EtOH solution. The microparticles were further rinsed with pre-chilled water and transferred to a 50-mL centrifuge tube. The particles were then suspended in pre-chilled water and kept in a refrigerator for 30 minutes to allow the particles to settle. Following removal of the supernatant, the particles were resuspended and filtered through a 40-μm cell strainer to remove large aggregates. Subsequently, the particles were washed twice with water at room temperature and freeze-dried overnight.

### Example 3. Preparation of Sunitinib Microparticles (not Surface Treated)

PLGA (555 mg) and PLGA-PEG5K (5.6 mg) were dissolved in DCM (4 mL). Sunitinib malate (90 mg) was dissolved in DMSO (2 mL). The polymer and drug solutions were then mixed. The resulting reaction mixture was filtered through a 0.22 μm PTFE syringe filter. The resulting reaction mixture was diluted with 1% PVA in PBS (200 mL) in a 250 mL beaker and then homogenized at 5,000 rpm for 1 minute. (The polymer/drug solution was poured into the aqueous phase using homogenization conditions and homogenized at 5,000 rpm for 1 minute) The reaction was next stirred at 800 rpm at room temperature for 3 hours in a biosafety cabinet. The particles were allowed to settle in the beaker for 30 minutes and approximately 150 mL of the supernatant was decanted off. The microparticle suspension underwent centrifugation at 56×g for 4.5 minutes, the solvent was removed, and the microparticles were then washed three times with water. The microparticle size and size distribution was determined using a Coulter Multisizer IV prior to lyophilization. The microparticles were lyophilized using a FreeZone 4.5-liter benchtop lyophilizer. Light exposure was avoided throughout the entire process.

### Example 4. General Procedure for the Preparation of Surface Treated Sunitinib Microparticles

Microparticle dry powder was weighed and placed in a small beaker and a stirring bar was added. The beaker was placed in an ice bath and cooled to about 4° C. A NaOH/EtOH solution was prepared by mixing NaOH in water (0.25M) with EtOH at 3:7 (v/v) and cooling to about 4° C. The cold NaOH/EtOH solution was added with stirring to the beaker containing the microparticles to afford a particle suspension of 100 mg/mL. The suspension was stirred for 3 minutes at about 4° C. and poured into a filtration apparatus to quickly remove the NaOH/EtOH solution. (The filtration apparatus needed to be pre-chilled in a −20° C. freezer prior to use.) Following filtration, the microparticles were rinsed in the filtration apparatus with ice cold deionized water and transferred to 50 mL centrifuge tubes. Each 50 mL centrifuge tube with filled with cold water to afford a 40 mL particle suspension at a concentration of 5-10 mg/mL. The centrifuge tubes were placed in a regenerator and the particles were allowed to settle for 30 minutes. The supernatant was then decanted. The particles were resuspended in cold water and filtered through a 40 μm cell strainer to remove any large aggregates. The particles were collected by centrifugation (56×g for 4.5 minutes) and washed twice with water. The product was lyophilized using a FreeZone 4.5 liter benchtop lyophilizer. The surface treatment process was conducted at approximately 4° C. and light exposure was avoided throughout the entire process.

### Example 5. Production of Surface-Treated Microparticles (STMP) on a Larger Scale (100 g and Higher)

NSTMP were produced using a continuous flow, oil-in-water emulsification method. The scale of the batches was 200 g. Detailed formulation parameters including surface treatment conditions are listed in Table 1.

A dispersed phase (DP) and a continuous phase (CP) were first prepared. For placebo microparticles, the DP was prepared by co-dissolving PLGA and PLGA-PEG polymers in DCM and the CP was a 0.25% PVA solution in water. For drug-loaded microparticles, the DP was prepared by dissolving sunitinib malate in DMSO and mixing with the polymer solution in DCM. The CP was a 0.25% PVA solution in PBS (pH approximately 7).

An emulsion was produced by mixing the DP and the CP using a high shear homogenizer with in-line assembling. The solvents in the DP were diluted by the CP, causing the emulsion droplets to solidify and become polymer microparticles. The microparticles from Lots A-H were subjected to centrifugation (the microparticles of Lot AA were subjected to sedimentation to separate the small particles). Microparticles were separated and collected in a continuous centrifugation system, while small particles in supernatant were removed through continuous centrifugation. Microparticles were then discharged from the centrifugation system and washed with fresh water to remove solvent-containing water, non-encapsulated free drug, and any remaining small particles using the centrifugation system. The washed microparticles were subsequently suspended in a solution containing NaOH and ethanol for surface modification of the NSTMP. This step was performed in a jacketed vessel and the temperature of the suspension was maintained around 8-11° C. In an alternative embodiment, the surface-treatment was conducted between 5-12° C.

Following additional washing in water, microparticle suspensions were sieved through a 50 μm filter. After measuring the microparticle mass concentration or drug concentration of in-process samples, the STMP suspension was adjusted to target concentration prior to filling in glass vials. Mannitol was also added to the final suspension as an excipient. The vials were then lyophilized and sealed. The manufacturing process can be completed aseptically and the final product in vials may also be terminally sterilized by E-Beam or gamma irradiation.

TABLE 1
Formulation and process parameters of STMP produced on 200 g scale

| | NSTMP | | | | | | | | | |
|---|---|---|---|---|---|---|---|---|---|---|
| | DP | | | | | | | | | |
| | PLGA | PLGA- | | Sunitinib | | Mixing | Surface Treatment | | | |
| | 7525 4A | PEG5k | DCM | Malate | DMSO | speed | Time | | NaOH | |
| Lot | (g) | (g) | (g) | (g) | (g) | (rpm) | (min) | EtOH | (mM) | Excipient |
| A | 172 | 1.72 | 1273 | 33.5 | 526 | 3600 | 30 | 60% | 0.75 | Mannitol |
| B | 172 | 1.72 | 1273 | 33.5 | 526 | 3600 | 30 | 70% | 7.5 | Mannitol |
| C | 172 | 1.72 | 1273 | 33.5 | 526 | 3600 | 30 | 70% | 7.5 | Mannitol |
| D | 172 | 1.72 | 1273 | 33.5 | 526 | 3600 | 30 | 70% | 4.0 | Mannitol |
| E | 172 | 1.72 | 1273 | 33.5 | 526 | 3600 | 30 | 70% | 2.0 | Mannitol |
| F | 172 | 1.72 | 1273 | 33.5 | 526 | 3600 | 30 | 70% | 1.0 | Mannitol |
| G | 172 | 1.72 | 1273 | 33.5 | 526 | 3600 | 30 | 70% | 3.0 | Mannitol |
| H | 172 | 1.72 | 1273 | 33.5 | 526 | 3600 | 30 | 70% | 2.5 | Mannitol |
| AA | 172 | 1.72 | 1273 | 33.5 | 526 | 3600 | 30 | 60% | 0.75 | Mannitol |

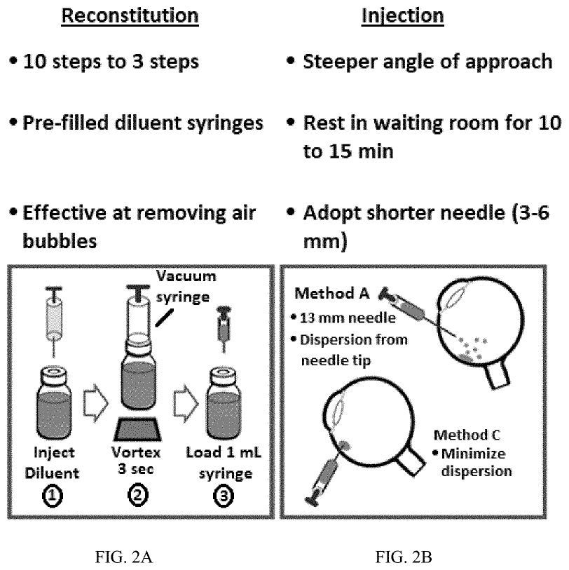

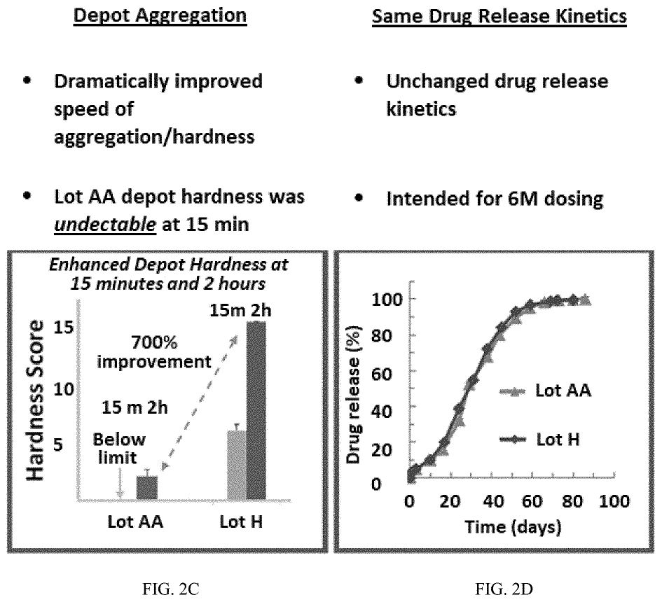

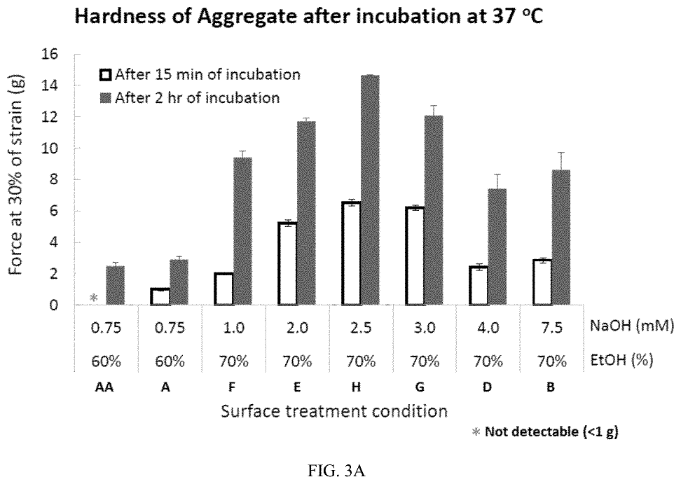

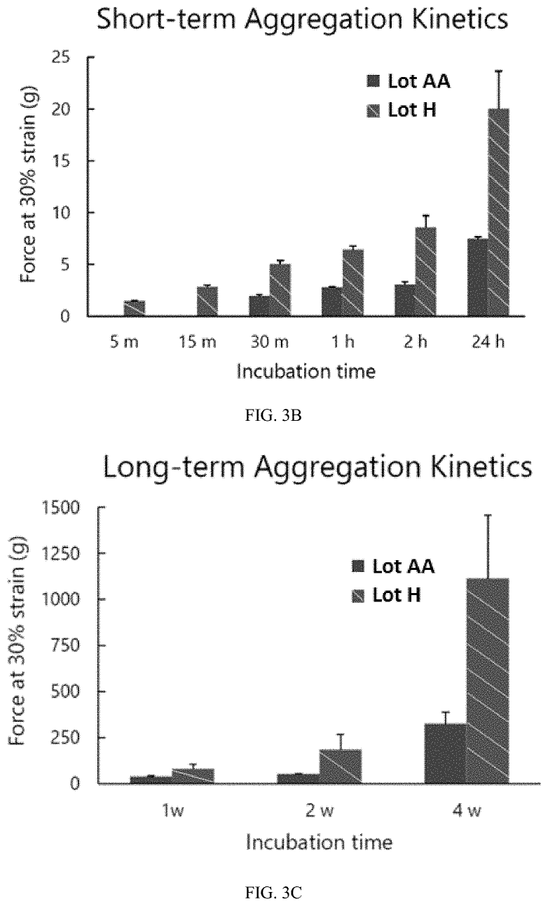

The procedure for measuring the strength of aggregates is described below. The strength of aggregates formed by the STMP is illustrated in FIG. 3A. Microparticles were incubated for 15 minutes or 2 hours. As shown in FIG. 3A, the hardness of the aggregate was affected by the concentration of NaOH and the percentage of EtOH. FIG. 2C is a graph of the hardness of Lot H and Lot AA. As shown in FIG. 2C, a 700% o improvement in hardness was observed between the microparticles of Lot A that were subjected to sedimentation and surface-treatment conditions of 0.75 mM NaOH and 60% o EtOH and Lot H that were subjected to centrifugation and surface-treatment conditions of 2.5 mM NaOH and 70% o EtOH.

Lot H and Lot AA were then tested in short-term (FIG. 3B) and long-term (FIG. 3C) aggregation experiments. As shown in FIG. 3B, the microparticles were incubated for up to 24 hours in the short-term experiments and up to 4 weeks in long-term experiments (FIG. 3C). In every time point in both the short-term and long-term experiments, Lot H was stronger than Lot AA.

### Example 6. Mechanical Testing of Particle Aggregates

Particles were suspended to a concentration of 200 mg/ml or 400 mg/ml in sodium hyaluronate (HA) solution. 400 ul of the particle suspension was injected into 1.8 mL of PBS pre-warmed at 37° C. in a 2 mL flat-bottomed clear glass vial. The vial was then incubated in a water bath at 37° C. At predetermined time points, the strength of the aggregate was measured with a TA.XT plus C Texture Analyser (Stable Micro Systems, UK) with a 5 mm ball probe and 5 kg load cell. The test was performed at a speed of 0.4 mm/s. The force required to compress the aggregate at 30% strain was recorded.

### Example 7. Continuous Centrifugation as a Separation Process to Remove Small Particles

Continuous centrifugation was incorporated in the production of surface treated particles (STP) as a separation method in order to remove to small particles as well as to wash and concentrate the particles. This process separates out small particles continuously from the larger particles by centrifugation and discharges the retained larger particles at the end of the cycle. The continuous centrifugation was performed with the UniFuge Pilot separation system from Pneumatic Scale Angelus.

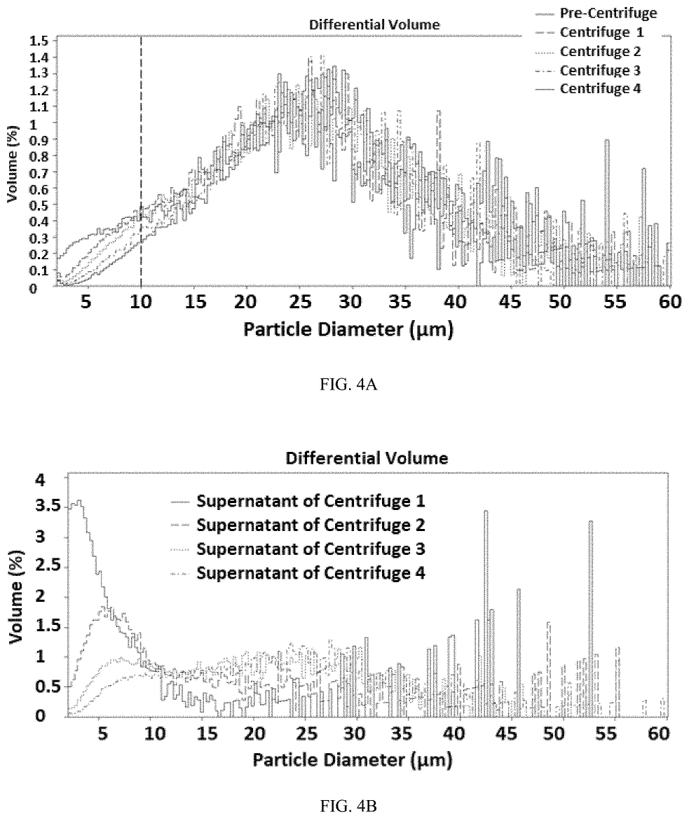

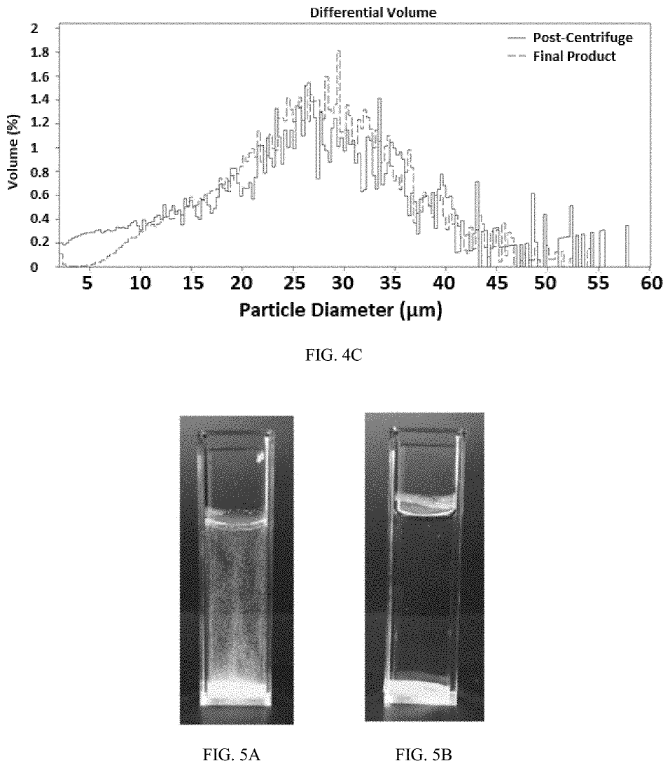

Continuous centrifugation effectively removed small particles. For example, before any centrifugation, particles less than 10 μm comprised 6.8% of the total particle size distribution (FIG. 4A). The percent of particles less than 10 μm was decreased by 21% after only one round of centrifugation. The fraction of small particles was further reduced with subsequent centrifugation and after three rounds particles less than 10 μm comprised only 2.7% of the total particles. This corresponded to a 60% reduction in the percent of particles less than 10 μm compared with no centrifugation.

The particle size of the supernatant removed by each round of centrifugation (FIG. 4B) showed the effectiveness of small particle removal in each centrifugation round.

During production, particles were washed again with the continuous centrifugation system following surface treatment, which can further reduce the fraction of small particles. As can be seen in FIG. 4C, the amount of small particles less than 10 μm in the final product was 69% lower than that immediately following homogenization and prior to any centrifugation. This is also reflected in the shift in the d10 size from 11.6 μm before centrifugation to 15.30 μm in the final product.

### Example 8. Light Transmittance and Depot Hardness of Microparticle Samples

The percentage of light transmittance for two samples of microparticle suspensions was calculated and the results are shown in Table 2. The assay for light transmittance was conducted by suspending microparticles to a concentration of 200 mg/ml or 400 mg/ml in sodium hyaluronate (HA) solution. 50 ul of the particle suspension was injected into 3 mL of PBS pre-warmed at 37 C in a 4.5 mL plastic cuvette. PBS was used as the blank. The light transmittance was measured with a Genesys 10S UV-Vis (Thermo Scientific) at 650 nm using the kinetics test and percent transmittance measurement mode. The measurement was performed at 1 second intervals over a total time of 1 minute. The average percent transmittance over the 1 minute time period was recorded. The measurement was performed in triplicates for each lot.

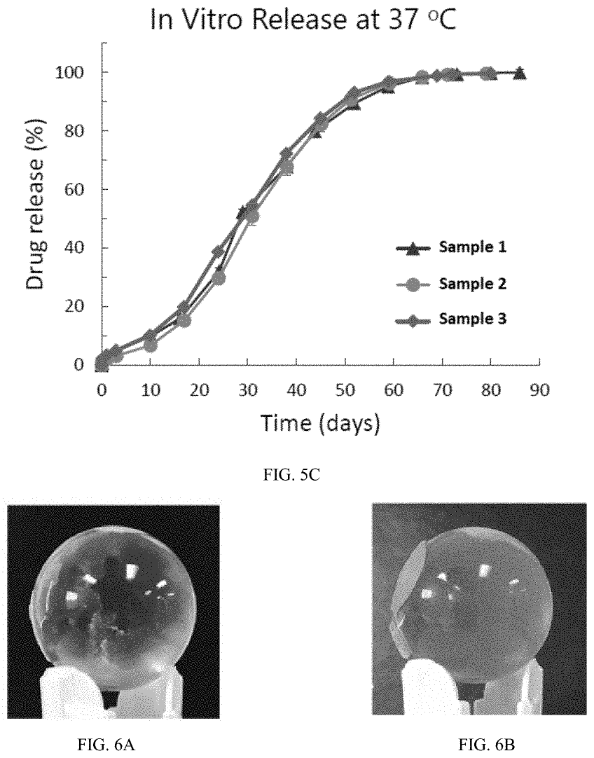

Sample 1 are microparticles that were prepared via a similar method as those of Lot AA (Example 5). Sample 2 are the microparticles of the present invention that have improved hardness and/or durability. FIG. 5A is an image of the 2 mg dose microparticles from Sample 1 and FIG. 5B is an image of the 2 mg dose of microparticles from Sample 2. As shown in Table 2, microparticles from Sample 2 consistently exhibited higher light transmittance at each dose.

TABLE 2
Light Transmittance of Microparticle Suspensions

Light Transmission

| Dose | Sample 1 | Sample 2 |
|---|---|---|
| 0.5 mg | 96.6% | 99.4% |
| 1 mg | 92.6% | 99.3% |
| 2 mg | 85.9% | 99.6% |

The depot hardness, light transmittance, drug loading, and size of three different samples of microparticles are shown in Table 3. Sample 1 are microparticles that were prepared via a similar method as those of Lot AA (Example 5). Sample 2 and Sample 3 are the microparticles of the present invention that have improved hardness and/or durability. As shown in Table 3, the depot hardness of microparticles of the present invention after 2 hours of incubation at 37° C. were 16 gram-force and 19 gram-force, while the microparticles that were prepared via the method of those of Lot AA only had a hardness of 2.5 gram-force after 2 hours of incubation at 37° C. The microparticles of the present invention were significantly harder than the microparticles prepared by previous methods (the procedure for determining hardness is discussed in Example 6).

The light transmittance of Samples 2 and 3 was also higher than the light transmittance of Sample 1. Light transmittance was conducted as described above in Example 8. The light transmittance of a solution of Sample 1 microparticles was only 92.6%, but the light transmittance of Sample 2 and Sample 3 microparticles was 99.7% and 99.8%, respectively.

The drug loading and size for all three samples are comparable, and therefore modifying the surface-treatment and/or subjecting the microparticles to continuous centrifugation does not affect the drug load or the size. Microparticles from all three samples had a drug loading of approximately 10% and a size in the range of 24-29 μm. FIG. 5C is the drug release in vitro at 37° C. of microparticles from Sample 1, Sample 2, and Sample 3. As shown in FIG. 5C, all three samples exhibited similar drug release rates. To measure the drug release, 10 mg of dried particles were added to a glass scintillation vial containing 4 mL of the release medium (1% Tween 20 in 1×PBS) and the particles were fully suspended by vortex. The vials were incubated at 37° C. under rotation at 150 rpm. At pre-determined time points, 3 mL of the release medium were carefully collected and replaced with 3 ml of fresh release medium. The UV absorbance of the collected release medium was measured and the concentration of drug at each time point was determined by comparison to a standard curve for the drug in release medium. After the last collection of release medium, the remaining content in the glass scintillation vial was lyophilized to quantify any remaining drug in the vials. The lyophilized solid was dissolved in 1 mL of DMSO with 5 min sonication followed by centrifugation at 1000 rpm for 5 min to remove any undissolved salts. The supernatant of the suspension was collected for UV absorbance measurement. The concentration of drug in DMSO was determined by comparison to a standard curve for the drug in DMSO.

TABLE 3
Summary of Depot Hardness and Light Transmittance
of Select Microparticle Samples

| | Sample 1 | Sample 2 | Sample 3 |
|---|---|---|---|
| Depot Hardness after 2 | 2.5 ± 0.2 | 16.8 ± 0.1 | 19.6 ± 0.3 |
| hours of incubation | | | |
| (gram-force) | | | |
| Light Transmittance (%) | 92.6 ± 7.4 | 99.7 ± 0.02 | 99.8 ± 0.2 |
| Drug Loading (%) | 9.04 | 10.2 | 10.3 |
| Size (μm) | 26.8 ± 9.0 | 28.2 ± 9.9 | 24.5 ± 8.7 |

### Example 9. Comparison of Lot AA and Lot E in a Glass Eye Model

Human vitreous humor accounts for the largest domain in the posterior segment of the eye. The human eye contains approximately 4 mL of vitreous humor that serves as a primary reservoir for the treatment of ocular diseases. The vitreous is composed primarily of water (greater than 98%), collagen and proteoglycans that provide the structural support to the vitreous compartment. In young eyes, the liquid phase is composed of approximately 80% water content entrapped within collagen rich hyaluronan gel phase with 20% existing as free water. However, with aging, the hyaluronan degenerates and segregates as the polymer chains depolymerize resulting in a higher free water content. Age-related macular degeneration occurs in elderly patients (>60 years old) with significant deterioration in the vitreous gel microstructure. This results in high free water content (>50%) that has significant implications in the deposition and movement of injected materials within the posterior ocular space. However, preclinical models of AMD typically rely on young animal models with intact vitreous gel microstructure. In this study, reproducible liquified vitreous models (˜60% water content) were generated to provide qualitative analysis and assessment of microparticle aggregation and disposition in an aged-eye.

Cow vitreous was harvested and placed into a 50 mL conical tube. The vitreous gel was homogenized using a Polytron PT 1600 E benchtop homogenizer at 25000 rpms for 1 min with up and down motion to break apart the gel phase and release the water content. The gel content was measured by weighing the total homogenized vitreous fluid and extracting the gel content to determine the free water content.

Glass orbs with a diameter of 2.5 cm were filled with liquefied cow vitreous through a 4 mm opening that was sealed with a transparent rubber septum. Internal pressure within the orb was measured using a traceable manometer and pressurized to be between 15-18 mm Hg. The eyes were incubated at 37° C. for 1 hour.

Lot AA and Lot E microparticle formulations were prepared and loaded into 1 mL syringes (200 mg/mL particle concentration). The glass eye was tilted at a vertical pitch of 60-70° upward beyond the horizontal starting plane. The injection site was 3-4 mm from the bottom of the cornealscleral limbus aimed downward at 15-20° angle toward the inferior region of the glass eye. A total volume of 50 uL of particle suspension was injected through a 13 mm 27 G thin-wall needle into the glass eye through the rubber septum. The eye was immediately reoriented facing horizontally by tilting it back down 60-70°. The eye was incubated at 37° C. for 15 minutes. Post incubation, ocular movement was introduced by orienting the eye upward 90°, downward 90°, left 90° and right 90°. This was immediately proceeded by walking 30 steps. Images were taken post injection, pre-movement, and post-movement.

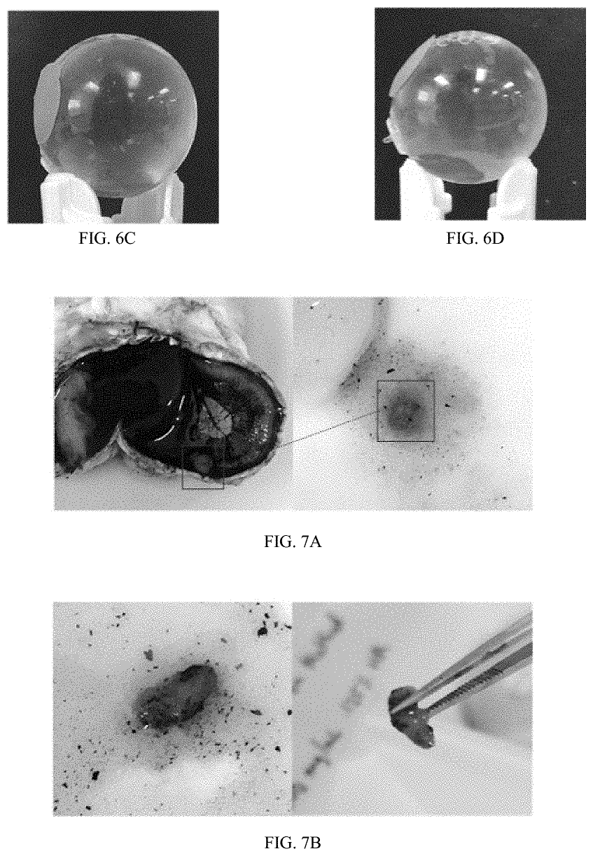

Immediately post injection, clear differences in the aggregation of the Lot AA (FIG. 6A) and Lot E (FIG. 6C) microparticles were observed. In the Lot AA-treated glass eyes, free floating particles were prominent near the primary depot. Trailing effects from the needle tracking were also very prominent in the Lot AA-glass eyes (FIG. 6A). In contrast, there was no observable free floating microparticles in the immediate post injection of the Lot E microparticles (FIG. 6C). The aggregate formed quickly near the back of the eye and as the eye was rotated back down to horizontal orientation, the entire intact depot shifted to the bottom of the eye where it remained.

Post movement, Lot AA microparticles (FIG. 6B) were unable to remain as a single aggregate due to the forces exerted on the depot with the movement protocol. This resulted in a dispersion of particles within the glass eye. In contrast, the Lot E microparticles (FIG. 6D) remained as a single intact depot even after the introduction of movement forces and no free-floating particles observed.

### Example 10. Comparison of Lot AA and Lot E Microparticles in an In-Situ Porcine Vitreous Liquefaction Model

Adult porcine eyes were injected with 20 uL of hyaluronidase (0.5 IU/mL) for enzymatic liquefaction of vitreous. The eyes were placed in a beaker containing PBS with 1% penicillin/streptomycin and incubated at 37° C. for 24 hours. Post incubation, the vitreous contained approximately 57-74% free water content as determined by weighing the gel and water content.

The pig eye was tilted upward 30-45° and the microparticles (200 mg/mL particle concentration) were injected 3-4 mm from the bottom of the cornealscleral limbus aimed upward at a 45° angle toward the inferior space through a 6 mm 27 G thin wall needle. Post-injection, the eye was immediately placed in the beaker containing PBS with 1% penicillin/streptomycin and incubated at 37° C. for 2 hours. Post incubation, the eyes were dissected and the depot was visualized and compared.

The depot was located within the inferior vitreous cavity near the site of injection. The Lot AA microparticles formed an intact depot upon initial observation (FIG. 7A, left). However, when the depot was excised from the vitreous gel, the primary depot readily broke apart with significant free-floating particles and smaller satellite aggregates embedded within the vitreous gel surrounding the primary depot site (FIG. 7A, right). The primary depot could not be picked up with forceps without disintegrating on contact. In contrast, the Lot E microparticles formed a solid depot that could easily be manipulated with forceps (FIG. 7B, left) and only a small amount of free floating microparticles could be observed near the primary aggregate (FIG. 7B, right).

Lot E microparticles form significantly stronger aggregates that are resistant to dispersion with the introduction of forces due to ocular movement in the glass eye model. In addition, in the pig eye model, Lot E microparticles form strong, solid, intact depots can be manipulated with forceps without falling apart. These significant improvements in microparticle formulation have resulted in a superior aggregation that may result in improved clinical outcomes.

### Example 11. Methods for the Injection of Aggregating Microparticles

The effect of several variables including the site of injection, angle of insertion, depth of insertion, gauge of needle, length of needle, and speed of injection on ocular damage, efflux, particle distribution, deposition, and performance were studied. Three methods, Method A, Method B, and Method C were compared to assess their effect on particle deposition and aggregation using the glass eye and in-situ porcine liquefied vitreous model.

FIG. 8A, FIG. 8B, FIG. 8C, and FIG. 8D depict the general procedure for Method A. As shown in FIG. 8A, in Method A, the patient tilts her head back approximately 450 and looks upward approximately 20-25°. The 13 mm needle is then injected. This method is also shown in FIG. 8B. Again the patient is instructed to tilt her head back 450 and look upward approximately 20-25° at the time of the injection. The microparticles are injected toward the back of the eye (point A in FIG. 8B). Once the patient sits up and reorients her eye back to the starting position along the vertical axis, the microparticles slide from the point A to the bottom the vitreous chamber (point B in FIG. 8B). During the movement of the microparticles from point A to point B, the microparticles disperse or spread, resulting in a particle “track.” FIG. 8C and FIG. 8D are additional representations of Method A. FIG. 8C is the position of the eye at the time of injection. The patient's head is titled back approximately 450 and the eye is titled upward approximately 20°. Point A in FIG. 8C represents the point of injection and the arrows represent two potential angles at which the needle could be inserted. Point B represents the bottom of the vitreous chamber and Point C represents the back of the eye. During Method A, the microparticles are directed toward the back of the eye and must move to reach the bottom of the vitreous chamber. FIG. 8D is a representation of the eye post-injection where the microparticles are still at the back of the eye and must slide down to the bottom of the vitreous to be effective.

FIG. 9A-FIG. 9F depict the general procedure for Method B. The patient sits in an upright position and gazes upward at an angle of about 20-30° to expose the inferior bulbar surface. A 13 mm needle is injected downward at approximately a 10° angle through the pars plana (3 to 4 mm from the limbal margin) at approximately 6 o'clock with respect to the pupil looking straight. The patient then reorients her eye back to the starting position along the vertical axis and the microparticles move from point A to point B. FIG. 9B and FIG. 9C are additional representations of Method B. FIG. 9A is the position of the eye at the time of injection. The patient's eye is titled upward approximately 25°. Point A in FIG. 9A represents the point of injection and the arrows represent two potential angles at which the needle could be inserted. Point B represents the bottom of the vitreous chamber. During Method B, the microparticles are directed toward the bottom of the vitreous chamber and minimal movement is required to reach the bottom of the vitreous chamber. FIG. 9B is a representation of the eye post-injection. The microparticles are at the bottom of the vitreous chamber, and minimal sliding was required. This results in less trailing and dispersion when the microparticles reach the final depot location, the bottom of the vitreous chamber.

Method B is also depicted in FIGS. 9D and 9E. The site of injection is the 6 o'clock position with respect to the pupil looking straight (as shown in FIG. 9G) and the needle is pointed downward at an angle of about 10°. The patient is looking upward with no head tilt at an angle of about 20-30°. This deposits the microparticles close to the bottom of the vitreous chamber. FIG. 9F is a patient undergoing the injection procedure shown in FIGS. 9D and 9E.

FIG. 10A, FIG. 10B, FIG. 10C, and FIG. 10D depict the general procedure for Method C. As shown in FIG. 10A, in Method C, the patient does not tilt her head back, but looks upward approximately 20-30°. The 6 mm needle is then injected. This method is also shown in FIG. 10B. Again the patient is instructed to look upward approximately 20-30° at the time of the injection. The microparticles are injected at or near the bottom of the vitreous chamber (point A in FIG. 10B). Once the patient sits up and reorients her eye back to the starting position along the vertical axis, the microparticles only need to minimally move from point A to point B, resulting in minimal sliding and dispersing. FIG. 10C and FIG. 10D are additional representations of Method C. FIG. 10C is the position of the eye at the time of injection. The patient's eye is titled upward approximately 20°. Point A in FIG. 10C represents the point of injection and the arrows represent two potential angles at which the needle could be inserted. Point B represents the bottom of the vitreous chamber. During Method C, the microparticles are directed toward the bottom of the vitreous chamber and minimal movement is required to reach the bottom of the vitreous chamber. FIG. 10D is a representation of the eye post-injection. The microparticles are at the bottom of the vitreous chamber, and minimal sliding was required from Point A to Point B. This results in less trailing and dispersion when the microparticles reach the final depot location, the bottom of the vitreous chamber.

Glass eyes were filled with liquefied bovine vitreous and incubated at 37° C. for 1 hour.

Microparticles (200 mg/mL) from Lot G were loaded into 1 mL syringes and injected into the glass eyes as described in Table 4. Immediately following injection, the eyes were reoriented to the horizontal starting position and incubated for 15 minutes at 37° C. Post incubation, ocular movement was introduced by orienting the eye upward 90°, downward 90°, left 90° and right 900 followed by walking 30 steps. Images were taken post injection, pre-movement and post-movement.

TABLE 4
A Comparison of Method A, Method
B, and Method C Injection Methods

| | Method A | Method B | Method C |
|---|---|---|---|
| Upward eye-roll angle | 25° | 25° | 25° |
| Head tilt angle | 45° | none | none |
| Injection site | ~3-4 mm | ~3-4 mm | ~3-4 mm |
| | from limbus | from limbus | from limbus |
| Needle penetration | ~10 mm | ~10 mm | ~3 mm |
| depth | | | |
| Injection angle | ~15-20° | 0-10° | 15-20° |
| | downward | downward | upward |
| Needle gauge | 27 G | 27 G | 27 G |
| | thin-wall | thin-wall | thin-wall |
| Needle length | 13 mm | 13 mm | 6 mm |

A marked difference in microparticle aggregation and deposition within the vitreous chamber can be observed between Method A compared to Method B and Method C. Method A has a longer penetration depth of 10 mm with the 13 mm needle. The length of the needle and the depth of penetration combined with the angle of the eye results in a depot that is deposited directly at the back of the eye. Post injection, as the eye is reoriented back to the starting position along the vertical axis, the depot slides from the back of the eye to the bottom of the eye. This results in a particle ‘track’ along the vertical axis as particle residue is deposited along the slide path. In addition, due to the longer depth of penetration of the needle and the long distance between the particle deposition and the initial site of injection, as the needle is withdrawn, particle tailing occurs resulting in a strand of particle extending from the primary depot to the site of injection. FIG. 12A is a picture of the bottom view of a glass eye wherein the microparticles were injected via Method A. FIG. 12B is a picture of the bottom view of a glass eye wherein the microparticles were injected via Method A. FIG. 12C and FIG. 12D are bottom and side views, respectively, of a glass eye injected with microparticles via Method B. FIG. 12E and FIG. 12F are bottom and side views, respectively, of a glass eye injected with microparticles via Method C.

An example of the steps of the Method B injection is described below:

1. Anesthetic is applied to the eye of the patient and a sterile, solid eyelid speculum is used to stabilize the eyelids and to help expose the inferior ocular surface.

2. The patient gazes upward about 20-30° to optimally expose the inferior bulbar surface.

3. A 0.05 mL volume for injection in a syringe is injected 3 to 4 mm from the limbal margin at approximately the 6 o'clock position with the needle pointed downward at an angel of approximately 10°. The needle is injected toward the back of the vitreous cavity and injected slowly over the course of approximately 5 seconds.

4. After approximately 5 seconds, the needle is removed from the eye.

5. The patient returns his gaze to the vertical position but should remain seated upright quietly for 15 minutes, avoiding any major head movement or significant eye movement in order for the depot to fully cure and solidify.

### Example 12. An Evaluation of Different Intravitreal Injection Methods in an Ex Vivo Porcine Vitreous Liquefaction Model

The vitreous humor in adult porcine eyes was liquefied as described in Example 10. Surface treated microparticles (200 mg/mL) were injected intravitreally using Method A, Method B, or Method C as described in Table 4.

Post injection, the porcine eyes were incubated for 2 hours at 37° C. in a beaker containing PBS with 1% penicillin/streptomycin. The eyes were subsequently dissected and the microparticle depot location and aggregation integrity were analyzed.

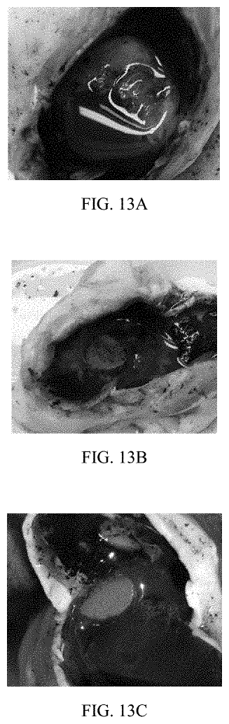

In the ex vivo porcine vitreous liquefaction model, the results obtained demonstrate particle aggregation and deposition between Method B (FIG. 13B) and Method C (FIG. 13C). A single solid depot was found near the bottom of the vitreous chamber. In comparison, the Method A depot was localized nearer to the posterior region of eye owing to the angle of injection and the longer needle length (FIG. 13A). In addition, the primary depot was more likely to be segmented or irregular in shape in comparison to depots generated through the Method B or Method C injections.

### Example 13: Particle Vacuum Treatment Procedure

Particles were filled into 2 mL glass vial with rubber septum. A vial adapter with a luer-lock opening was attached to the vial and diluent (e.g., hyaluronic solution (HA)) was injected into the vial through the vial adapter. A 60 mL VacLok syringe (Merit Medical, South Jordan, Utah) was attached to the vial adapter and its plunger was pulled to a predetermined volume and locked by turning the plunger per the manufacturer's instruction (FIG. 14A, FIG. 14C, and FIG. 15).

This created a negative pressure in the vials as low as approximately 30 Torr depending on the plunger locking position. Particles were mixed with the diluent in the vial by manual tapping or vortexing under the vacuum created by the VacLok syringe to yield a homogeneous suspension. Due to the vacuum, less air bubbles were generated in the suspension upon mixing. The vial was then rested in an upright position for a predetermined period (i.e., 10-60 minutes). This further allowed formed air bubbles to be pulled out of the suspension, thus reducing particle floatation upon injection later. After the vacuuming step, the plunger of the 60 mL syringe was released and the syringe was detached from the vial adapter. The suspension was remixed by gentle tapping and loaded into a dosing syringe for injection.

### Example 14. Impact of Benzyl Alcohol on Particle Aggregation

A representative lot of surface treated microparticles (STMP) suspended at a particle concentration of 200 mg/ml or 400 mg/ml were prepared in sodium hyaluronate (HA) solutions containing 0%, 0.25%, 0.5%, 0.75% or 1% benzyl alcohol (BA) by weight. The particle suspensions were vacuumed as described in Example 13. The suspensions were injected into PBS pre-warmed at 37° C. and then incubated in a water bath at 37° C. for 2 minutes, 5 minutes, 15 minutes, 2 hours or 24 hours. The strength of the resulting aggregates was measured as described in Example 6.

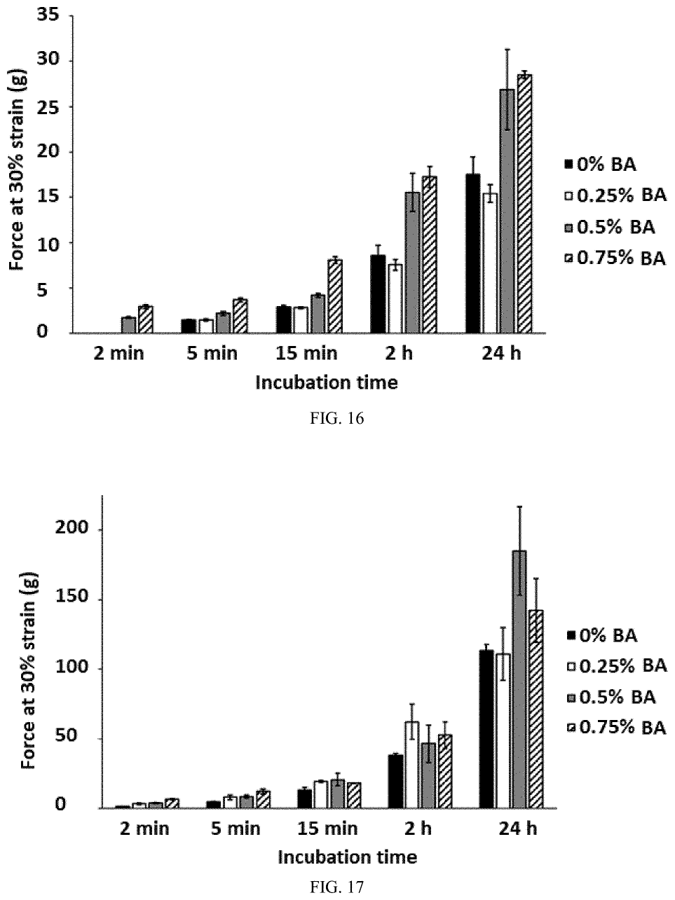

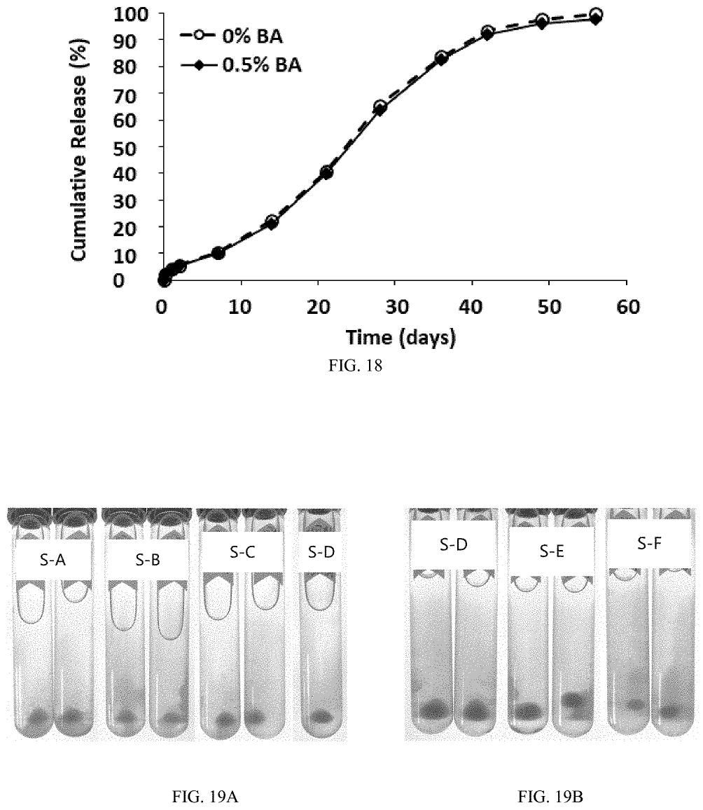

The addition of BA in HA diluent was shown to improve the aggregation strength in a concentration-dependent manner. For example, for the 200 mg/ml particle suspension, addition of BA at concentrations of 0.5% and 0.75% in the diluent resulted in a stronger aggregate as early as 2 minutes (FIG. 16). After only 2 minutes of incubation, the particle suspension in HA containing 0.75% BA formed an aggregate as strong as the particle suspension in HA alone at 15 minutes of incubation. The aggregate with 0.5% or 0.75% BA was shown to be stronger than that with no or only 0.25% BA for at least 24 hours of incubation. For particles suspended at a concentration of 400 mg/ml, the addition of BA in the diluent at all concentrations tested increased the aggregation kinetics resulting in a stronger aggregate as early as 2 minutes. The improvement in aggregate strength was sustained for at least 2 hours of incubation (FIG. 17). Addition of 1% BA in the diluent for both particle concentrations resulted in poor injectability due to particle clumping and clogging in the syringe, suggesting that this concentration of BA is too high. Further, the addition of BA in the diluent had minimal impact on the drug release profile of the particles (FIG. 18).

Suspensions of non-surface treated microparticles (NSTMP) at 200 mg/ml or 400 mg/ml were prepared in HA solutions containing 0%, 0.5%, 1%, or 2% BA by weight. Addition of BA in the diluent did not improve the aggregation strength of NSTMP at either particle concentration. The strength of the NSTP particle depot at both 200 and 400 mg/ml was still below the detection limit after 2 hours of incubation. In addition, as shown in FIG. 19A and FIG. 19B, when injected into a test tube of PBS pre-warmed at 37° C. and incubated for 15 minutes, the NSTMP with BA concentrations of 0% (S-A), 0.5% (S-B), 1% (S-C), and 2% (S-D) suspended at a particle concentration of 200 mg/ml (FIG. 7A) and BA concentrations of 0% (S-D), 0.5% (S-E), and 1% (S-F) suspended at a particle concentration of 400 mg/ml (FIG. 19B) settled at the bottom of the vial but did not form a strong aggregate, and became dispersed with gentle agitation.

The effect of BA on particle aggregation was tested on five different particle lots produced under various surface treatment conditions as detailed in Table 5. Addition of BA in the diluent increased aggregation strength for all particle lots with surface treatment while there was no improvement in aggregation strength with NSTMP.

TABLE 5
Particle lots tested with benzyl alcohol (BA) in diluent

| | Surface treatment | | Particle | BA |
|---|---|---|---|---|
| Particle | NaOH | % | Concentration | Concentration |
| ID | (mM) | EtOH | (mg/ml) | (% w/w) |
| 1 | — | — | 200 | 0, 0.5, 1, 2 |
| | | | 400 | 0, 0.5, 1, 2 |
| 2 | 0.75 | 70 | 200 | 0, 0.5 |
| | | | 400 | 0, 0.5 |
| 3 | 7.5 | 70 | 200 | 0, 0.25, 0.5, 0.75, 1 |
| | | | 400 | 0, 0.25, 0.5, 0.75, 1 |
| 4 | 2 | 70 | 200 | 0, 0.5 |
| | | | 400 | 0, 0.5, 1 |
| 5 | 0.75 | 60 | 200 | 0, 0.25, 0.5, 0.75 |

### Example 15. Impact of Triethyl Citrate on Particle Aggregation

A representative lot of surface treated microparticles (STMP) suspended at a particle concentration of 200 mg/ml was prepared in HA containing 0% or 0.57% triethyl citrate (TEC) by weight. The particle suspensions were vacuumed as described in Example 13. The suspensions were injected into PBS pre-warmed at 37° C. and then incubated in a water bath at 37° C. for 2, 5 or 15 minutes. The strength of the resulting aggregates was measured as described in Example 6.

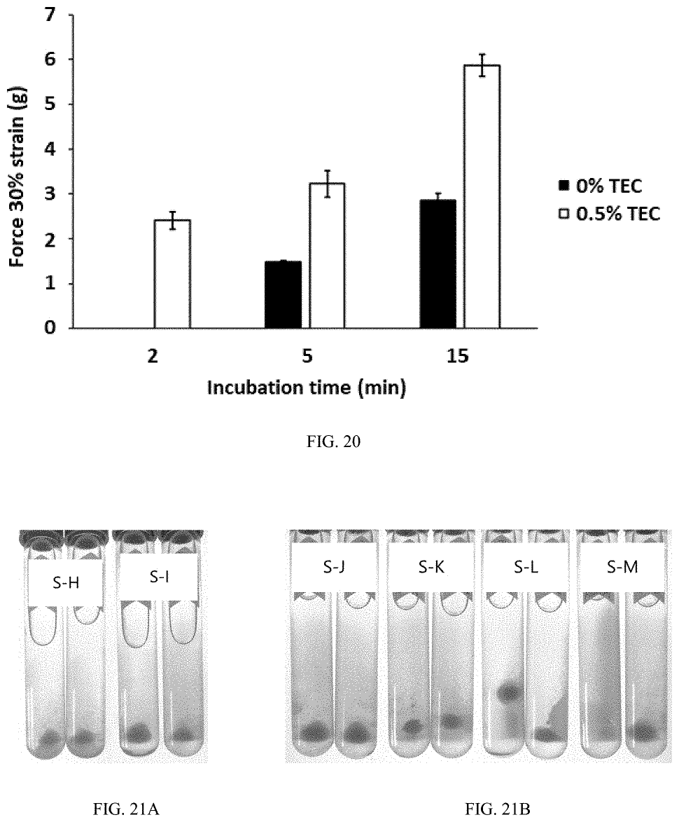

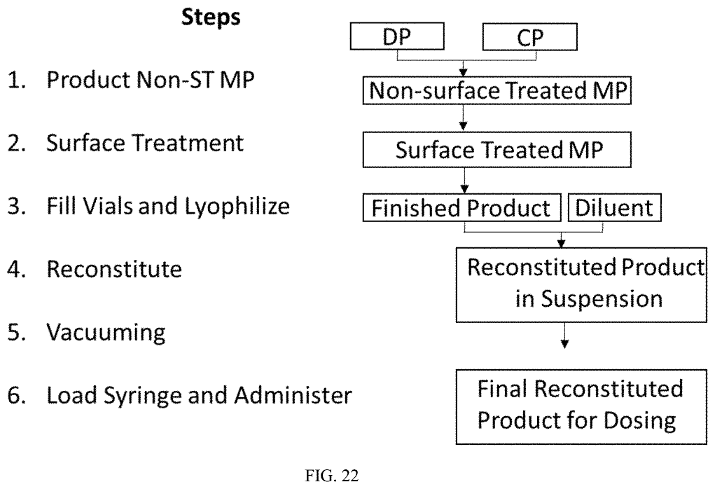

Addition of TEC in the diluent resulted in a stronger aggregate that formed more quickly than particles suspended in HA only. The particle suspension with TEC formed an aggregate after only 2 minutes of incubation, whereas the aggregation strength of particles suspended in HA alone was below detection limit. After only 5 minutes of incubation, the particle suspension with TEC formed an aggregate as strong as that with HA alone after 15 minutes of incubation (FIG. 20).

Addition of TEC in the diluent did not improve the aggregation of non-surface treated microparticles (NSTMP) at particle concentrations of both 200 and 400 mg/ml. The strength of the NSTP particle depot after 2 hours of incubation was below the detection limit for both concentrations. In addition, as show in FIG. 21A and FIG. 21B, when injected into a test tube of PBS pre-warmed at 37° C. and incubated for 15 minutes, the NSTMP with TEC concentrations of 0% (S-H) and 0.5% (S-I) suspended at a particle concentration of 200 mg/ml (FIG. 21A) and TEC concentrations of 0% (S-J), 0.5% (S-K), 1% (S-L), and 2% (S-M) suspended at a particle concentration of 400 mg/ml (FIG. 21B) did not remain aggregated and became dispersed upon gentle agitation.

The effect of TEC on particle aggregation was tested on three different particle lots produced with various surface treatment conditions as detailed in Table 6. Addition of TEC in the diluent increased aggregation strength for all particle lots with surface treatment, while there was no improvement in aggregation strength with NSTMP.

TABLE 6
Particle lots tested with triethyl citrate (TEC) in diluent

| | Surface treatment | | Particle | TEC |
|---|---|---|---|---|
| Particle | NaOH | % | Concentration | Concentration |
| ID | (mM) | EtOH | (mg/ml) | (% w/w) |
| 1 | — | — | 200 | 0, 0.5, 1, 2 |
| | | | 400 | 0, 0.5, 1, 2 |
| 3 | 7.5 | 70 | 200 | 0, 0.5 |
| 5 | 0.75 | 60 | 200 | 0, 0.5 |

This specification has been described with reference to embodiments of the invention. However, one of ordinary skill in the art appreciates that various modifications and changes can be made without departing from the scope of the invention as set forth herein. Accordingly, the specification is to be regarded in an illustrative rather than a restrictive sense, and all such modifications are intended to be included within the scope of invention.

We claim:

1. A biodegradable aggregated polymeric microparticle of at least 500 microns comprising a therapeutic agent that exhibits a hardness rating of at least 5 gram-force needed to compress the particle at 30% of strain in a fluid selected from vitreous, water, phosphate buffered saline, and an aqueous physiologically acceptable solution with a viscosity not more than about 4 times that of water.

2. The biodegradable aggregated polymeric microparticle of claim 1 that exhibits a hardness rating of at least 10 gram-force needed to compress the particle at 30% of strain.

3. The biodegradable aggregated polymeric microparticle of claim 1 that exhibits a hardness rating of at least 20 gram-force needed to compress the particle at 30% of strain.

4. The biodegradable aggregated polymeric microparticle of claim 1 wherein the hardness of the microparticles increases at least two-fold in four hours or less after injection.

5. The biodegradable aggregated polymeric microparticle of claim 4, wherein the hardness increases at least four-fold.

6. The biodegradable aggregated polymeric microparticle of claim 4, wherein the hardness increases at least six-fold.

7. The biodegradable aggregated polymeric microparticle of claim 4, wherein the hardness increases in two hours or less.

8. The biodegradable aggregated polymeric microparticle of claim 1, wherein the microparticles comprise poly(lactide-co-glycolide).

9. The biodegradable aggregated polymeric microparticle of claim 1, wherein the microparticles comprise poly(lactide-co-glycolide) and poly(lactide-co-glycolide) covalently linked to polyethylene glycol.

10. The biodegradable aggregated polymeric microparticle of claim 1, wherein the microparticles comprise poly(lactic acid).

11. The biodegradable aggregated polymeric microparticle of claim 1, wherein the microparticles comprise poly(lactide-co-glycolide), poly(lactic acid), and poly(lactide-co-glycolide) covalently linked to polyethylene glycol.

12. The biodegradable aggregated polymeric microparticle of claim 1, wherein the therapeutic agent comprises sunitinib or a pharmaceutically acceptable salt.

13. The biodegradable aggregated polymeric microparticle of claim 12, wherein the therapeutic agent comprises sunitinib malate.

14. The biodegradable aggregated polymeric microparticle of claim 1, wherein the therapeutic agent comprises timolol or a pharmaceutically acceptable salt.

15. The biodegradable aggregated polymeric microparticle of claim 1, wherein the therapeutic agent comprises a prodrug of timolol.

16. The biodegradable aggregated polymeric microparticle of claim 15, wherein the prodrug of timolol is selected from 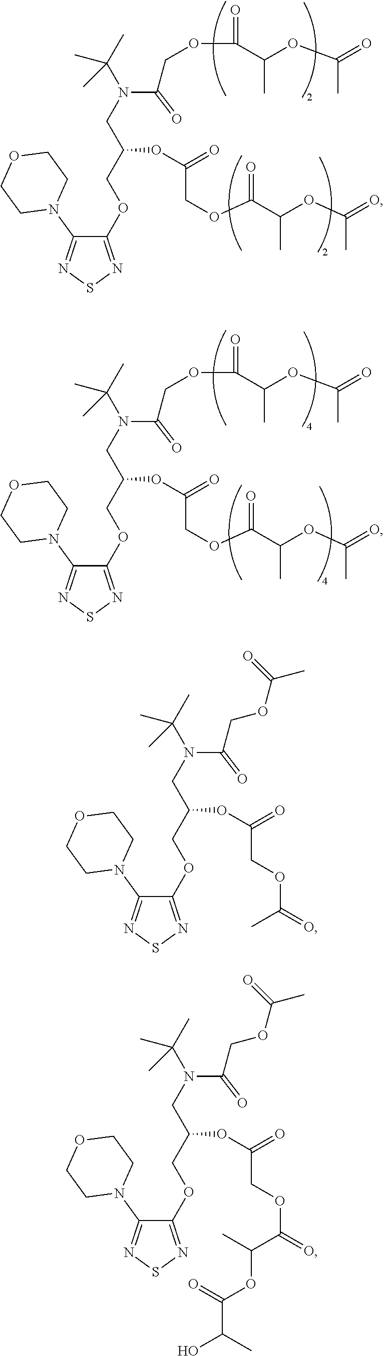 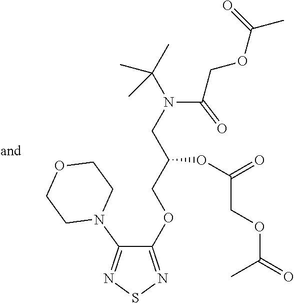 [SMILES: CC(=O)OCC(=O)O[C@H](COc1nsnc1N1CCOCC1)CN(C(=O)COC(C)=O)C(C)(C)C]
or a pharmaceutically acceptable salt thereof.

17. The biodegradable aggregated polymeric microparticle of claim 16, wherein the prodrug of timolol is selected from 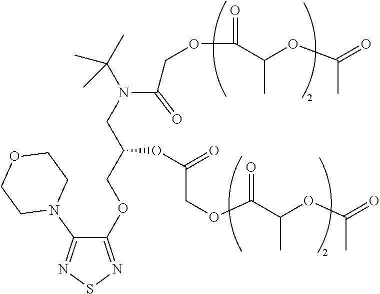
or a pharmaceutically acceptable salt thereof.

18. The biodegradable aggregated polymeric microparticle of claim 16, wherein the prodrug of timolol is selected from

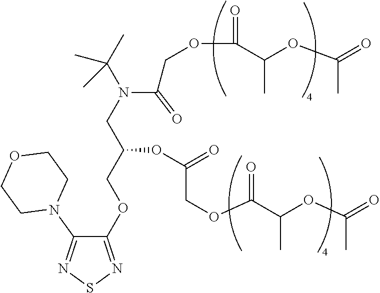

19. A suspension of surface-modified solid aggregating microparticles in a diluent comprising a plasticizer that improves in vivo particle aggregation wherein the surface-modified solid aggregating microparticles comprise surface surfactant and a therapeutic agent encapsulated in at least one biodegradable polymer selected from PLA and PLGA and at least one hydrophobic polymer covalently bound to a hydrophilic polymer selected from PLGA-PEG and PLA-PEG, wherein the microparticles:
(i) have been surface-modified at a temperature less than about 18° C.;
(ii) have a mean diameter between 10 μm and 60 μm;
(iii) aggregate in vivo to form at least one pellet of at least 500 μm in vivo which provides sustained drug delivery in vivo for at least one month; and
(iv) wherein the plasticizer softens the surface of the microparticles prior to administration to prepare the microparticles for aggregation.

20. The suspension of surface modified solid aggregating microparticles of claim 19 in a dosage form for an ocular delivery route selected from the group consisting of intravitreal, intrastromal, intracameral, subtenon, sub-retinal, retrobulbar, peribulbar, suprachoroidal, subchoroidal, conjunctival, subconjunctival, episcleral, posterior juxtascleral, circumcorneal, and tear duct injections.

21. The suspension of surface-modified solid aggregating microparticles of claim 19, wherein the diluent is sodium hyaluronate.

22. The suspension of surface-modified solid aggregating of claim 19, wherein the diluent is hyaluronic acid.

23. The suspension of surface modified solid aggregating microparticles of claim 19 wherein the plasticizer is selected from triethyl citrate, benzyl alcohol, polyethylene glycol, N-methyl-2-pyrrolidone (NMP), 2-pyrrolidone, triacetin, benzyl acetate, benzyl benzoate, acetyltributyl citrate, dibutyl sebacate, dimethylphthalate, tributyl O-acetylcitrate, polysorbate 80, propylene carbonate, butyl lactate, and isovaleric acid.

24. The suspension of surface-modified solid aggregating microparticles of claim 23, wherein the additive is benzyl alcohol.

25. The suspension of surface-modified solid aggregating microparticles of claim 23, wherein the additive is triethyl citrate.

26. The suspension of surface-modified solid aggregating microparticles of claim 19, wherein the microparticles comprise poly(lactide-co-glycolide).

27. The suspension of surface-modified solid aggregating microparticles of claim 19, wherein the microparticles comprise poly(lactide-co-glycolide) and poly(lactide-co-glycolide) covalently linked to polyethylene glycol.

28. The suspension of surface-modified solid aggregating microparticles of claim 19, wherein the microparticles comprise poly(lactic acid).

29. The suspension of surface-modified solid aggregating microparticles of claim 19, wherein the microparticles comprise poly(lactide-co-glycolide), poly(lactic acid), and poly(lactide-co-glycolide) covalently linked to polyethylene glycol.

30. The suspension of surface-modified solid aggregating microparticles of claim 19, wherein the therapeutic agent comprises sunitinib or a pharmaceutically acceptable salt thereof.

31. The suspension of surface-modified solid aggregating microparticles of claim 30, wherein the therapeutic agent comprises sunitinib malate.

32. The suspension of surface-modified solid aggregating microparticles of claim 19, wherein the therapeutic agent comprises timolol or a pharmaceutically acceptable salt thereof.

33. The suspension of surface-modified solid aggregating microparticles of claim 19, wherein the therapeutic agent comprises a prodrug of sunitinib, timolol, brimonidine, brinzolamide, dorzolamide, or pharmaceutically acceptable salt thereof.

34. The suspension of surface-modified solid aggregating microparticles of claim 19, wherein the therapeutic agent comprises a prodrug of timolol or pharmaceutically acceptable salt thereof.

35. The suspension of surface-modified solid aggregating microparticles of claim 34, wherein the prodrug of timolol is selected from 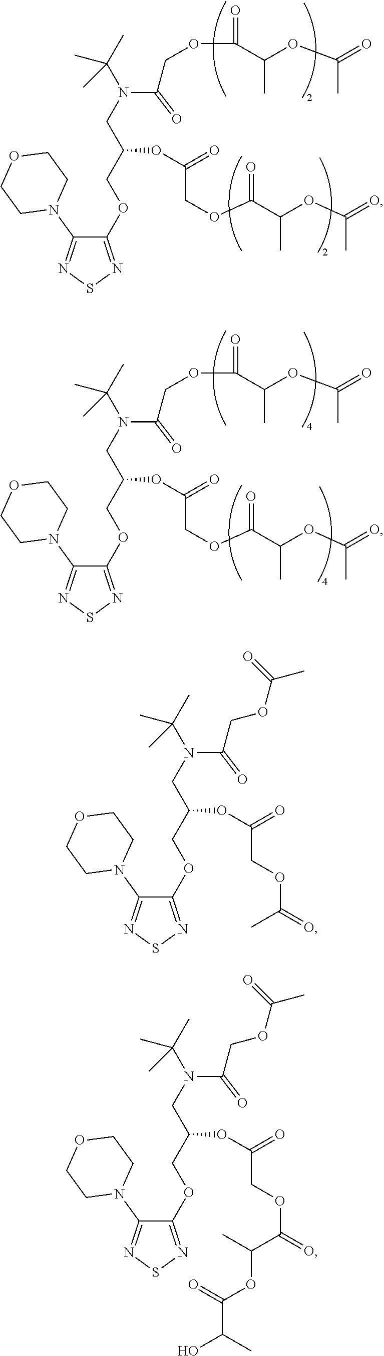 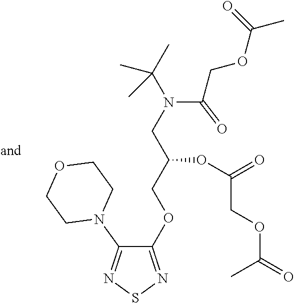 [SMILES: CC(=O)OCC(=O)O[C@H](COc1nsnc1N1CCOCC1)CN(C(=O)COC(C)=O)C(C)(C)C]
or a pharmaceutically acceptable salt thereof.

36. The suspension of surface-modified solid aggregating microparticles of claim 35, wherein the prodrug of timolol is selected from 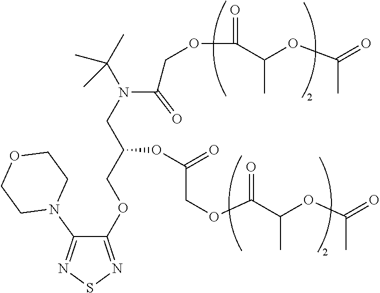
or a pharmaceutically acceptable salt thereof.

37. The suspension of surface-modified solid aggregating microparticles of claim 35, wherein the prodrug of timolol is selected from 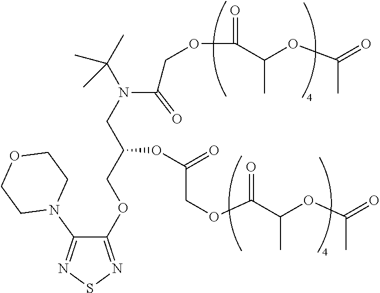
a pharmaceutically acceptable salt thereof.

38. The suspension of surface-modified solid aggregating microparticles of claim 34, wherein the prodrug of timolol is selected from 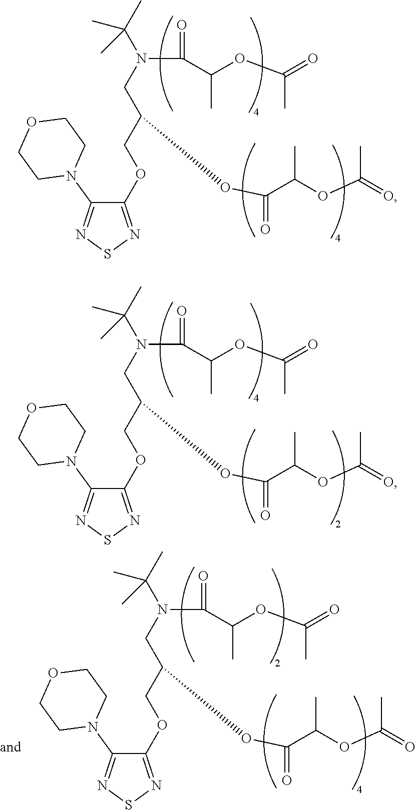
or a pharmaceutically acceptable salt thereof.

39. The suspension of surface modified solid aggregating microparticles of claim 19, wherein at least one pellet provides sustained drug delivery for at least two months.

40. The suspension of surface modified solid aggregating microparticles of claim 19, wherein at least one pellet provides sustained drug delivery for at least four months.

41. The suspension of surface modified solid aggregating microparticles of claim 19, wherein at least one pellet provides sustained drug delivery for at least six months.

42. The suspension of surface modified solid aggregating microparticles of claim 19, wherein the microparticles have a mean diameter between 20 and 30 μm.

43. The suspension of surface modified solid aggregating microparticles of claim 19, wherein the microparticles have a mean diameter between 20 and 50 μm.

44. The suspension of surface modified solid aggregating microparticles of claim 19, wherein the microparticles include a therapeutic agent of 5-25 percent by weight.

45. The suspension of surface modified solid aggregating microparticles of claim 19, wherein the microparticles include a therapeutic agent of 1-40 percent by weight.

46. The suspension of surface modified solid aggregating microparticles of claim 19, wherein the pellet of at least 500 μm exhibits a hardness rating of at least 5 gram-force needed to compress the particle at 30% of strain in a fluid selected from vitreous, water, phosphate buffered saline, and an aqueous physiologically acceptable solution with a viscosity not more than about 4 times that of water.

47. The suspension of surface modified solid aggregating microparticles of claim 46, wherein the pellet of at least 500 μm exhibits a hardness rating of at least 15 gram-force needed to compress the particle at 30% of strain.

48. The suspension of surface modified solid aggregating microparticles of claim 46, wherein the pellet of at least 500 μm exhibits a hardness rating of at least 20 gram-force needed to compress the particle at 30% of strain.

49. A pharmaceutical composition comprising the suspension of surface modified solid aggregating microparticles of claim 19 in a pharmaceutically acceptable carrier.

50. A method to treat an ocular disorder selected from glaucoma, a disorder related to an increase in intraocular pressure (IOP), dry or wet age-related macular degeneration (AMD), neovascular age-related macular degeneration (NVAMD), geographic atrophy and diabetic retinopathy comprising administering the suspension of surface-modified solid aggregating microparticles of claim 19 to a host in need thereof.

51. The method of claim 50 wherein the disorder is glaucoma.

52. The method of claim 50 wherein the host is a human.
